(19) United States
(12) Patent Application Publication
(10) Pub. No.: US 2019/0359756 A1
(43) Pub. Date: Nov. 28, 2019

(54) OPTICAL MEMBER FORMING COMPOSITION

(71) Applicant: Mitsubishi Gas Chemical Company, Inc., Tokyo (JP)

(72) Inventor: Masatoshi ECHIGO, Chiyoda-ku, Tokyo (JP)

(21) Appl. No.: 16/332,694

(22) PCT Filed: Sep. 13, 2017
(86) PCT No.: PCT/JP2017/033013

§ 371 Date: Mar. 12, 2019

(30) Foreign Application Priority Data
Sep. 13, 2016 (JP) 2016-178447

(51) Int. Cl. C08G 16/02; G02B 1/04; C07C 39/15; C07D 311/78

(52) U.S. Cl. CPC C08G 16/0256; C07D 311/78; C07C 39/15; G02B 1/04

(57) ABSTRACT

The present invention provides an optical member forming composition comprising a compound represented by the following formula (0):

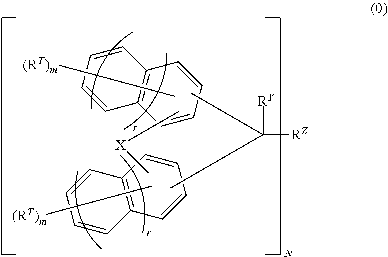

### TECHNICAL FIELD

The present invention relates to an optical member forming composition.

### BACKGROUND ART

In recent years, various optical member forming compositions have been proposed, and, for example, acrylic resins, epoxy based resins or anthracene derivatives have been used (see Patent Literatures 1 to 4).

### CITATION LIST

### Patent Literature

Patent Literature 1: Japanese Patent Application Laid-Open No. 2016-12061

Patent Literature 2: Japanese Patent Application Laid-Open No. 2015-174877

Patent Literature 3: Japanese Patent Application Laid-Open No. 2014-73986

Patent Literature 4: Japanese Patent Application Laid-Open No. 2010-138393

### SUMMARY OF INVENTION

### Technical Problem

Although a large number of compositions intended for optical members have heretofore been proposed as mentioned above, none of these compositions achieve all of heat resistance, transparency and refractive index at high dimensions. Thus, the development of novel materials is demanded.

An object of the present invention is to provide an optical member forming composition which achieves all of heat resistance, transparency and refractive index at high dimensions.

### Solution to Problem

The present inventors have conducted diligent studies to attain the object and consequently found that the object can be attained by use of a compound or a resin having a specific structure, completing the present invention.

More specifically, the present invention is as follows.

[1]

An optical member forming composition comprising a compound represented by the following formula (0):

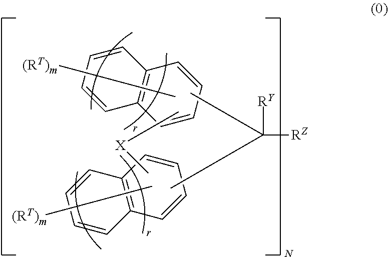

wherein

R^{Y} is a hydrogen atom;

R^{Z} is an N-valent group of 1 to 60 carbon atoms or a single bond;

each R^{T} is independently an alkyl group of 1 to 30 carbon atoms optionally having a substituent, an aryl group of 6 to 40 carbon atoms optionally having a substituent, an alkenyl group of 2 to 30 carbon atoms optionally having a substituent, an alkoxy group of 1 to 30 carbon atoms optionally having a substituent, a halogen atom, a nitro group, an amino group, a thiol group, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond;

X is an oxygen atom, a sulfur atom, a single bond or non-crosslinked state;

each m is independently an integer of 0 to 9, wherein at least one of m is an integer of 1 to 9;

N is an integer of 1 to 4, wherein when N is an integer of 2 or larger, structural formulae indicated within N parentheses are the same or different; and

each r is independently an integer of 0 to 2.

[2]

The optical member forming composition according to the above [1], wherein the compound represented by the above formula (0) is a compound represented by the following formula (0-1):

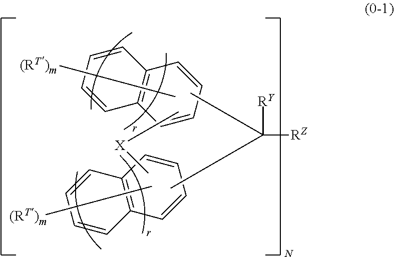

wherein

R^{Y} is a hydrogen atom;

R^{Z} is an N-valent group of 1 to 60 carbon atoms or a single bond;

each R^{T′} is independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a thiol group, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond, and at least one of

R^{T′} is a hydroxy group or a group in which a hydrogen atom of a hydroxy group is substituted with an acid crosslinking group or an acid dissociation group;

X is an oxygen atom, a sulfur atom, a single bond or non-crosslinked state;

each m is independently an integer of 0 to 9, wherein at least one of m is an integer of 1 to 9;

N is an integer of 1 to 4, wherein when N is an integer of 2 or larger, structural formulae indicated within N parentheses are the same or different; and

each r is independently an integer of 0 to 2.

[3]

The optical member forming composition according to the above [2], wherein the compound represented by the above formula (0-1) is a compound represented by the following formula (1):

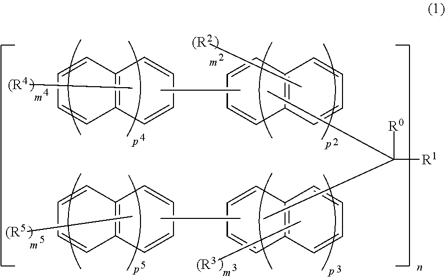

wherein

R⁰ is as defined in the R^{Y};

R¹ is an n-valent group of 1 to 60 carbon atoms or a single bond;

R² to R⁵ are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a thiol group, a hydroxy group or a group in which a hydrogen atom of a hydroxy group is substituted with an acid dissociation group, wherein the alkyl group, the alkenyl group and the aryl group each optionally have an ether bond, a ketone bond or an ester bond, and at least one of R² to R⁵ is a hydroxy group or a group in which a hydrogen atom of a hydroxy group is substituted with an acid dissociation group;

m² and m³ are each independently an integer of 0 to 8;

m⁴ and m⁵ are each independently an integer of 0 to 9,

provided that m², m³, m⁴ and m⁵ are not 0 at the same time;

n is as defined in the N, wherein when n is an integer of 2 or larger, structural formulae indicated within n parentheses are the same or different; and

p² to p⁵ are each as defined in the r.

[4]

The optical member forming composition according to the above [2], wherein the compound represented by the above formula (0-1) is a compound represented by the following formula (2):

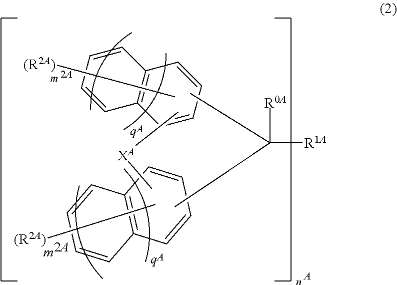

wherein

R^{0A} is as defined in the R^{Y};

R^{1A} is an n^{A}-valent group of 1 to 60 carbon atoms or a single bond;

each R^{2A} is independently an alkyl group of 1 to 30 carbon atoms optionally having a substituent, an aryl group of 6 to 30 carbon atoms optionally having a substituent, an alkenyl group of 2 to 10 carbon atoms optionally having a substituent, a halogen atom, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond, and at least one of R^{2A} is a hydroxy group or a group in which a hydrogen atom of a hydroxy group is substituted with an acid crosslinking group or an acid dissociation group;

n^{A} is as defined in the N, wherein when n^{A} is an integer of 2 or larger, structural formulae indicated within n^{A} parentheses are the same or different;

X^{A} is an oxygen atom, a sulfur atom, a single bond or non-crosslinked state;

each m^{2A} is independently an integer of 0 to 7, provided that at least one of m^{2A} is an integer of 1 to 7; and

each q^{A} is independently 0 or 1.

[5]

The optical member forming composition according to the above [3], wherein the compound represented by the above formula (1) is a compound represented by the following formula (1-1):

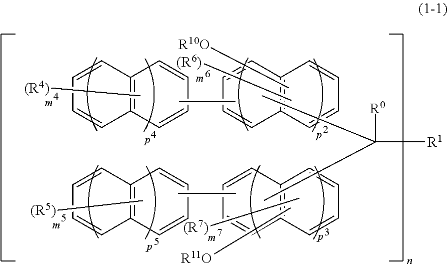

wherein

R⁰, R¹, R⁴, R⁵, n, p² to p⁵, m⁴ and m⁵ are as defined above;

R⁶ and R⁷ are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, or a thiol group, wherein the alkyl group, the alkenyl group and the aryl group each optionally have an ether bond, a ketone bond or an ester bond;

R¹⁰ and R¹¹ are each independently a hydrogen atom, an acid crosslinking group or an acid dissociation group; and

m⁶ and m⁷ are each independently an integer of 0 to 7,

provided that m⁴, m⁵, m⁶ and m⁷ are not 0 at the same time.

[6]

The optical member forming composition according to the above [5], wherein the compound represented by the above formula (1-1) is a compound represented by the following formula (1-2):

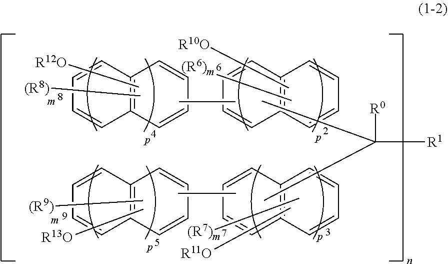

wherein

R⁰, R¹, R⁶, R⁷, R¹⁰, R¹¹, n, p² to p⁵, m⁶ and m⁷ are as defined above;

R⁸ and R⁹ are as defined in the R⁶ and the R⁷;

R¹² and R¹³ are as defined in the R¹⁰ and the R¹¹; and

m⁸ and m⁹ are each independently an integer of 0 to 8,

provided that m⁶, m⁷, m⁸ and m⁹ are not 0 at the same time.

[7]

The optical member forming composition according to the above [4], wherein the compound represented by the above formula (2) is a compound represented by the following formula (2-1):

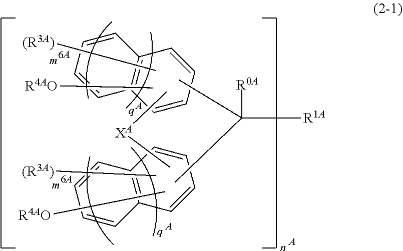

wherein

R^{0A}, R^{1A}, n^{A}, q^{A} and X^{A} are as defined above;

each R^{3A} is independently a halogen atom, an alkyl group of 1 to 30 carbon atoms optionally having a substituent, an aryl group of 6 to 30 carbon atoms optionally having a substituent, or an alkenyl group of 2 to 30 carbon atoms optionally having a substituent, wherein the alkyl group, the alkenyl group and the aryl group each optionally have an ether bond, a ketone bond or an ester bond;

each R^{4A} is independently a hydrogen atom, an acid crosslinking group or an acid dissociation group; and

each m^{6A} is independently an integer of 0 to 5.

[8]

An optical member forming composition comprising a resin obtained with a compound represented by the following formula (0) as a monomer:

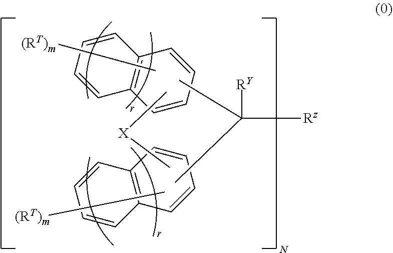

wherein

R^{Y} is a hydrogen atom;

R^{Z} is an N-valent group of 1 to 60 carbon atoms or a single bond;

each R^{T} is independently an alkyl group of 1 to 30 carbon atoms optionally having a substituent, an aryl group of 6 to 40 carbon atoms optionally having a substituent, an alkenyl group of 2 to 30 carbon atoms optionally having a substituent, an alkoxy group of 1 to 30 carbon atoms optionally having a substituent, a halogen atom, a nitro group, an amino group, a thiol group, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond;

X is an oxygen atom, a sulfur atom, a single bond or non-crosslinked state;

each m is independently an integer of 0 to 9, wherein at least one of m is an integer of 1 to 9;

N is an integer of 1 to 4, wherein when N is an integer of 2 or larger, structural formulae indicated within N parentheses are the same or different; and

each r is independently an integer of 0 to 2.

[9]

An optical member forming composition comprising a resin obtained with a compound represented by the following formula (1) as a monomer:

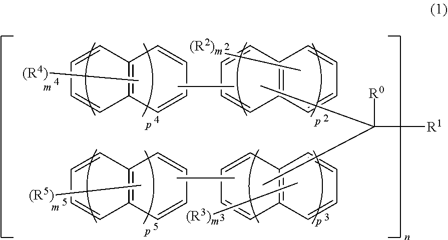

wherein

R⁰ is a hydrogen atom;

R¹ is an n-valent group of 1 to 60 carbon atoms or a single bond;

R² to R⁵ are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a thiol group, a hydroxy group or a group in which a hydrogen atom of a hydroxy group is substituted with an acid dissociation group, wherein the alkyl group, the alkenyl group and the aryl group each optionally have an ether bond, a ketone bond or an ester bond, and at least one of R² to R⁵ is a hydroxy group or a group in which a hydrogen atom of a hydroxy group is substituted with an acid dissociation group;

m² and m³ are each independently an integer of 0 to 8;

m⁴ and m⁵ are each independently an integer of 0 to 9,

provided that m², m³, m⁴ and m⁵ are not 0 at the same time;

n is an integer of 1 to 4, wherein when n is an integer of 2 or larger, structural formulae indicated within n parentheses are the same or different; and

p² to p⁵ are each independently an integer of 0 to 2.

[10]

The optical member forming composition according to the above [9], wherein the resin obtained with the compound represented by the above formula (1) as a monomer is a resin having a structure represented by the following formula (3):

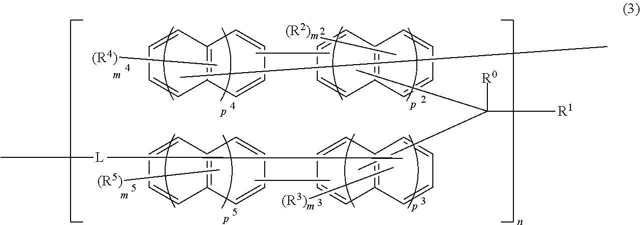

wherein

L is a linear or branched alkylene group of 1 to 30 carbon atoms optionally having a substituent or a single bond;

R⁰ is a hydrogen atom;

R¹ is an n-valent group of 1 to 60 carbon atoms or a single bond;

R² to R⁵ are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a thiol group, a hydroxy group or a group in which a hydrogen atom of a hydroxy group is substituted with an acid dissociation group, wherein the alkyl group, the alkenyl group and the aryl group each optionally have an ether bond, a ketone bond or an ester bond, and at least one of R² to R⁵ is a hydroxy group or a group in which a hydrogen atom of a hydroxy group is substituted with an acid dissociation group;

m² and m³ are each independently an integer of 0 to 8;

m⁴ and m⁵ are each independently an integer of 0 to 9, provided that m², m³, m⁴ and m⁵ are not 0 at the same time;

n is an integer of 1 to 4, wherein when n is an integer of 2 or larger, structural formulae indicated within n parentheses are the same or different; and

p² to p⁵ are each independently an integer of 0 to 2.

[11]

An optical member forming composition comprising a resin obtained with a compound represented by the following formula (2) as a monomer:

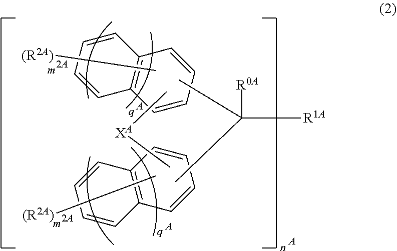

wherein

R^{0A} is a hydrogen atom;

R^{1A} is an n^{A}-valent group of 1 to 60 carbon atoms or a single bond;

each R^{2A} is independently an alkyl group of 1 to 30 carbon atoms optionally having a substituent, an aryl group of 6 to 30 carbon atoms optionally having a substituent, an alkenyl group of 2 to 10 carbon atoms optionally having a substituent, a halogen atom, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond, and at least one of R^{2A} is a hydroxy group or a group in which a hydrogen atom of a hydroxy group is substituted with an acid crosslinking group or an acid dissociation group;

n^{A} is an integer of 1 to 4, wherein when n^{A} is an integer of 2 or larger, structural formulae indicated within n^{A} parentheses are the same or different;

X^{A} is an oxygen atom, a sulfur atom, a single bond or non-crosslinked state;

each m^{2A} is independently an integer of 0 to 7, provided that at least one of m^{2A} is an integer of 1 to 7; and

each q^{A} is independently 0 or 1.

[12]

The optical member forming composition according to the above [11], wherein the resin obtained with the compound represented by the above formula (2) as a monomer is a resin having a structure represented by the following formula (4):

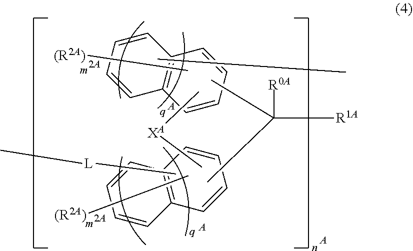

wherein

L is a linear or branched alkylene group of 1 to 30 carbon atoms optionally having a substituent or a single bond;

R^{0A} is a hydrogen atom;

R^{1A} is an n^{A}-valent group of 1 to 30 carbon atoms or a single bond;

each R^{2A} is independently an alkyl group of 1 to 30 carbon atoms optionally having a substituent, an aryl group of 6 to 30 carbon atoms optionally having a substituent, an alkenyl group of 2 to 10 carbon atoms optionally having a substituent, a halogen atom, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond, and at least one of R^{2A} is a hydroxy group or a group in which a hydrogen atom of a hydroxy group is substituted with an acid crosslinking group or an acid dissociation group;

n^{A} is an integer of 1 to 4, wherein when n^{A} is an integer of 2 or larger, structural formulae indicated within n^{A} parentheses are the same or different;

X^{A} is an oxygen atom, a sulfur atom, a single bond or non-crosslinked state;

each m^{2A} is independently an integer of 0 to 6, provided that at least one of m^{2A} is an integer of 1 to 6; and

each q^{A} is independently 0 or 1.

[13]

The optical member forming composition according to any of the above [1] to [12], further comprising a solvent.

[14]

The optical member forming composition according to any of [1] to [13], further comprising an acid generating agent.

[15]

The optical member forming composition according to the above [13] or [14], further comprising a crosslinking agent.

[16]

The optical member forming composition according to the above [15], wherein the crosslinking agent is at least one selected from the group consisting of a phenol compound, an epoxy compound, a cyanate compound, an amino compound, a benzoxazine compound, a melamine compound, a guanamine compound, a glycoluril compound, a urea compound, an isocyanate compound and an azide compound.

[17]

The optical member forming composition according to the above [15] or [16], wherein the crosslinking agent has at least one allyl group.

[18]

The optical member forming composition according to any of the above [15] to [17], wherein the content of the crosslinking agent is 0.1 to 50% by mass of the total mass of the solid components.

[19]

The optical member forming composition according to any of the above [15] to [18], further comprising a crosslinking promoting agent.

[20]

The optical member forming composition according to the above [19], wherein the crosslinking promoting agent is at least one selected from the group consisting of an amine, an imidazole, an organic phosphine, and a Lewis acid.

[21]

The optical member forming composition according to the above [19] or [20], wherein the content of the crosslinking promoting agent is 0.1 to 10% by mass of the total mass of the solid components.

[22]

The optical member forming composition according to any of the above [13] to [21], further comprising a radical polymerization initiator.

[23]

The optical member forming composition according to the above [22], wherein the radical polymerization initiator is at least one selected from the group consisting of a ketone based photopolymerization initiator, an organic peroxide based polymerization initiator and an azo based polymerization initiator.

[24]

The optical member forming composition according to the above [22] or [23], wherein the content of the radical polymerization initiator is 0.1 to 10% by mass of the total mass of the solid components.

### Advantageous Effects of Invention

The present invention can provide an optical member forming composition which achieves all of heat resistance, transparency and refractive index at high dimensions.

### DESCRIPTION OF EMBODIMENTS

Hereinafter, an embodiment of the present invention (hereinafter, also referred to as “present embodiment”) will be described. The present embodiment described below is given in order to illustrate the present invention. The present invention is not limited to only the present embodiment.

### [Optical Member Forming Composition]

The optical member forming composition according to the present embodiment comprises at least one selected from the group consisting of a compound described below and/or a resin obtained by polymerizing the compound.

### [Compound Represented by Formula (0)]

The optical member forming composition according to the present embodiment comprises a compound represented by the following formula (0):

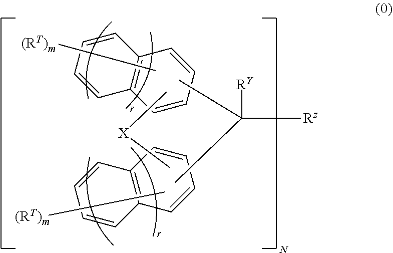

(In the formula (0), R^{Y} is a hydrogen atom;

R^{Z} is an N-valent group of 1 to 60 carbon atoms or a single bond;

each R^{T} is independently an alkyl group of 1 to 30 carbon atoms optionally having a substituent, an aryl group of 6 to 40 carbon atoms optionally having a substituent, an alkenyl group of 2 to 30 carbon atoms optionally having a substituent, an alkoxy group of 1 to 30 carbon atoms optionally having a substituent, a halogen atom, a nitro group, an amino group, a thiol group, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond;

X is an oxygen atom, a sulfur atom, a single bond or non-crosslinked state;

each m is independently an integer of 0 to 9, wherein at least one of m is an integer of 1 to 9;

N is an integer of 1 to 4, wherein when N is an integer of 2 or larger, structural formulae indicated within N parentheses are the same or different; and

each r is independently an integer of 0 to 2).

### [Compound Represented by Formula (0-1)]

The compound represented by the formula (0) according to the present embodiment is preferably a compound represented by the following formula (0-1):

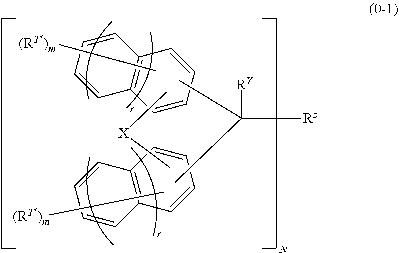

(In the formula (0-1), R^{Y} is a hydrogen atom;

R^{Z} is an N-valent group of 1 to 60 carbon atoms or a single bond;

each R^{T′} is independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a thiol group, a hydroxy group or a group in which a hydrogen atom of a hydroxy group is substituted with an acid dissociation group, wherein the alkyl group, the alkenyl group and the aryl group each optionally have an ether bond, a ketone bond or an ester bond, and at least one of R^{T′} is a hydroxy group or a group in which a hydrogen atom of a hydroxy group is substituted with an acid crosslinking group or an acid dissociation group;

X is an oxygen atom, a sulfur atom, a single bond or non-crosslinked state;

each m is independently an integer of 0 to 9, wherein at least one of m is an integer of 1 to 9;

N is an integer of 1 to 4, wherein when N is an integer of 2 or larger, structural formulae indicated within N parentheses are the same or different; and

each r is independently an integer of 0 to 2).

The compound represented by the formula (0-1) according to the present embodiment is preferably a compound represented by the following formula (1) from the viewpoint of heat resistance and solvent solubility:

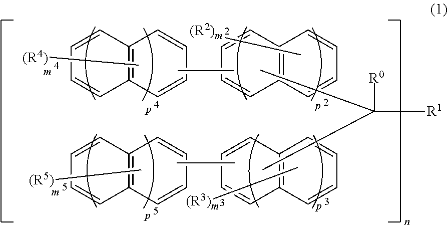

In the above formula (1), R⁰ is a hydrogen atom.

R¹ is an n-valent group of 1 to 60 carbon atoms or a single bond, and the aromatic rings are bonded to each other via this R¹.

R² to R⁵ are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a thiol group, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond. However, in the formula (1), at least one of R² to R⁵ is a hydroxy group or a group in which a hydrogen atom of a hydroxy group is substituted with an acid dissociation group.

m² and m³ are each independently an integer of 0 to 8.

m⁴ and m⁵ are each independently an integer of 0 to 9.

However, m², m³, m⁴ and m⁵ are not 0 at the same time.

n is as defined in the N, wherein when n is an integer of 2 or larger, structural formulae indicated within n parentheses are the same or different.

p² to p⁵ are each independently an integer of 0 to 2.

The n-valent group refers to an alkyl group of 1 to 60 carbon atoms (n=1), an alkylene group of 1 to 30 carbon atoms (n=2), an alkanepropayl group of 2 to 60 carbon atoms (n=3), and an alkanetetrayl group of 3 to 60 carbon atoms (n=4). Examples of the n-valent group include groups having a linear hydrocarbon group, a branched hydrocarbon group or an alicyclic hydrocarbon group. Herein, the alicyclic hydrocarbon group also includes bridged alicyclic hydrocarbon groups. Also, the n-valent group may have an aromatic group of 6 to 60 carbon atoms.

The n-valent hydrocarbon group may have an alicyclic hydrocarbon group, a double bond, a heteroatom or an aromatic group of 6 to 60 carbon atoms. Herein, the alicyclic hydrocarbon group also includes bridged alicyclic hydrocarbon groups.

The compound represented by the above formula (1) has high refractive index. Also, the compound represented by the above formula (1) has high heat resistance attributed to its rigidity of the structure despite a relatively low molecular weight and can therefore be used even under high temperature baking conditions. Furthermore, the compound represented by the above formula (1) has high solubility in a safe solvent, exhibits suppressed crystallinity, and has good heat resistance and etching resistance. Hence, the optical member forming composition comprising the compound represented by the above formula (1) can impart a good shape to an optical member. The optical member forming composition is relatively prevented from being stained by heat treatment in a wide range from a low temperature to a high temperature and is therefore useful as various optical member forming compositions. The optical member forming composition is useful for an optical component in a film form or a sheet form as well as a plastic lens (prism lens, lenticular lens, microlens, Fresnel lens, viewing angle control lens, contrast improving lens, etc.), a phase difference film, a film for electromagnetic wave shielding, a prism, an optical fiber, a solder resist for flexible printed wiring, a plating resist, an interlayer insulating film for multilayer printed circuit boards, and a photosensitive optical waveguide.

The compound represented by the above formula (1) is preferably a compound represented by the following formula (1-1) from the viewpoint of easy crosslinking and solubility in an organic solvent:

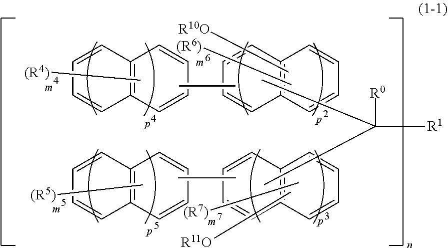

In the formula (1-1),

R⁰, R¹, R⁴, R⁵, n, p² to p⁵, m⁴ and m⁵ are as defined above;

R⁶ and R⁷ are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, or a thiol group, wherein the alkyl group, the alkenyl group and the aryl group each optionally have an ether bond, a ketone bond or an ester bond;

R¹⁰ and R¹¹ are each independently a hydrogen atom, an acid crosslinking group or an acid dissociation group; and

m⁶ and m⁷ are each independently an integer of 0 to 7,

provided that m⁴, m⁵, m⁶ and m⁷ are not 0 at the same time.

The compound represented by the above formula (1-1) is preferably a compound represented by the following formula (1-2) from the viewpoint of easier crosslinking and further solubility in an organic solvent:

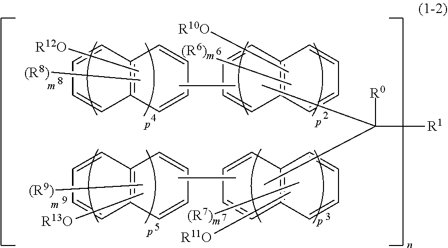

In the formula (1-2),

R⁰, R¹, R⁶, R⁷, R¹⁰, R¹¹, n, p² to p⁵, m⁶ and m⁷ are as defined above;

R⁸ and R⁹ are as defined in the R⁶ and the R⁷;

R¹² and R¹³ are as defined in the R¹⁰ and the R¹¹; and

m⁸ and m⁹ are each independently an integer of 0 to 8,

provided that m⁶, m⁷, m⁸ and m⁹ are not 0 at the same time.

The compound represented by the above formula (1-1) is preferably a compound represented by the following formula (1a) from the viewpoint of the supply of raw materials:

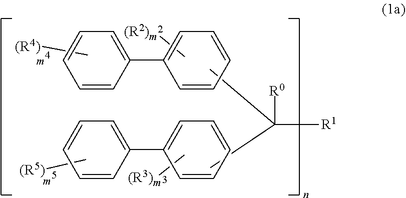

In the above formula (1a), R⁰ to R⁵, m² to m⁵ and n are as defined in those described in the above formula (1).

The compound represented by the above formula (1a) is more preferably a compound represented by the following formula (1b) from the viewpoint of solubility in an organic solvent:

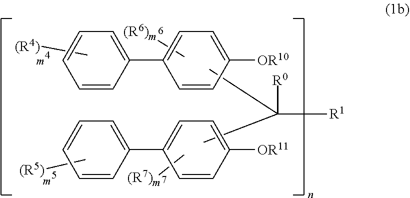

In the above formula (1b), R⁰, R¹, R⁴, R⁵, m⁴, m⁵, and n are as defined in those described in the above formula (1), and R⁶, R⁷, R¹⁰, R¹, m⁶, and m⁷ are as defined in those described in the above formula (1-1).

The compound represented by the above formula (1b) is still more preferably a compound represented by the following formula (1c) from the viewpoint of solubility in an organic solvent:

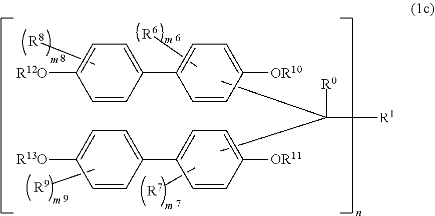

In the above formula (c), R⁰, R¹, R⁶ to R¹³, m⁶ to m⁹, and n are as defined in those described in the above formula (1-2).

Specific examples of the compound represented by the above formula (0) will be listed below. However, the compound represented by the formula (0) is not limited to the specific examples listed below.

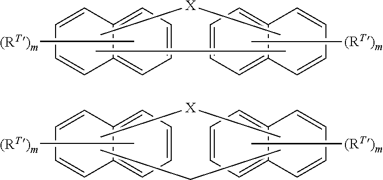

In the above formulae, X is as defined in that described in the above formula (0); R^{T′} is as defined in R^{T′} described in the above formula (0-1); and each m is independently an integer of 0 to 9, wherein at least one of m is an integer of 1 to 9. At least one of R^{T′} is a hydroxy group or a group in which a hydrogen atom of a hydroxy group is substituted with an acid crosslinking group or an acid dissociation group.

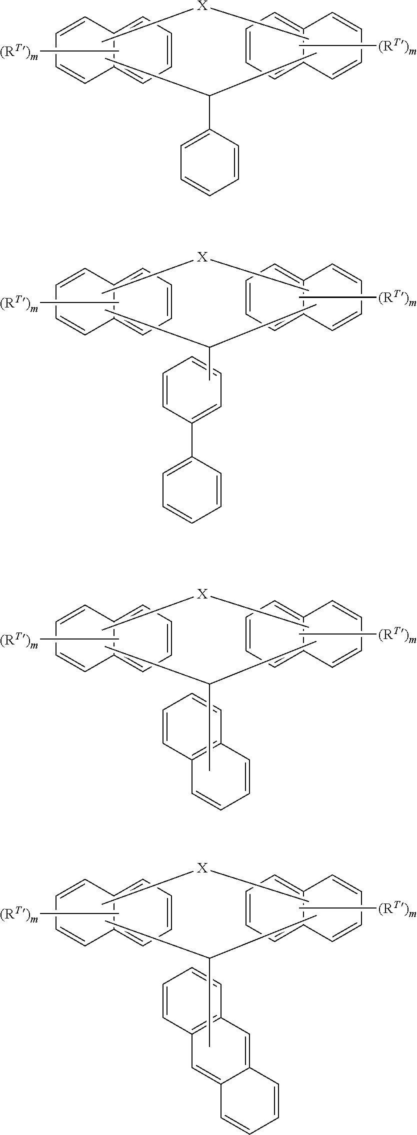

In the above formulae, X is as defined in that described in the above formula (0); R^{T′} is as defined in R^{T′} described in the above formula (0-1); and each m is independently an integer of 0 to 9, wherein at least one of m is an integer of 1 to 9. At least one of R^{T′} is a hydroxy group or a group in which a hydrogen atom of a hydroxy group is substituted with an acid crosslinking group or an acid dissociation group.

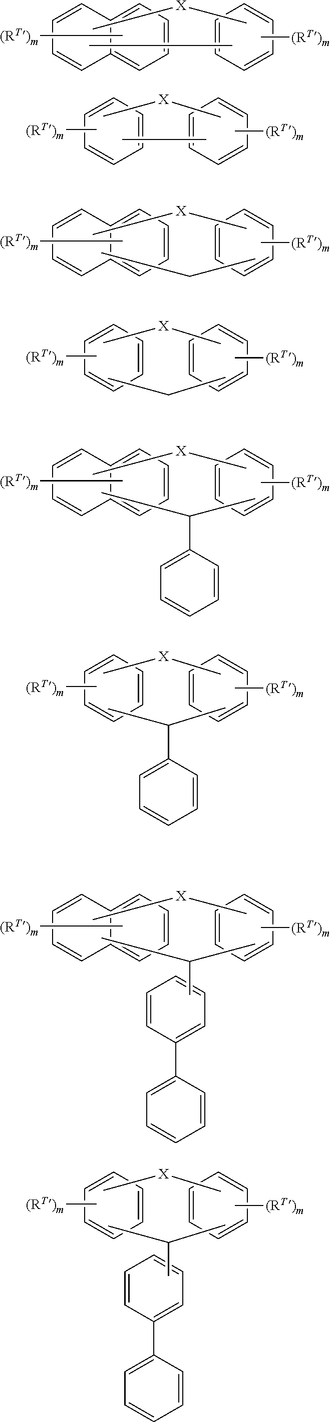

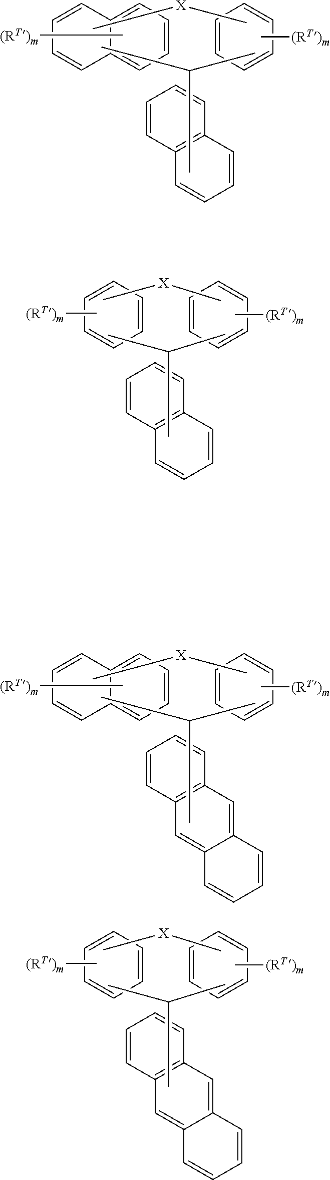

In the above formulae, X is as defined in that described in the above formula (0); R^{T′} is as defined in R^{T′} described in the above formula (0-1); and each m is independently an integer of 0 to 9, wherein at least one of m is an integer of 1 to 9. At least one of R^{T′} is a hydroxy group or a group in which a hydrogen atom of a hydroxy group is substituted with an acid crosslinking group or an acid dissociation group.

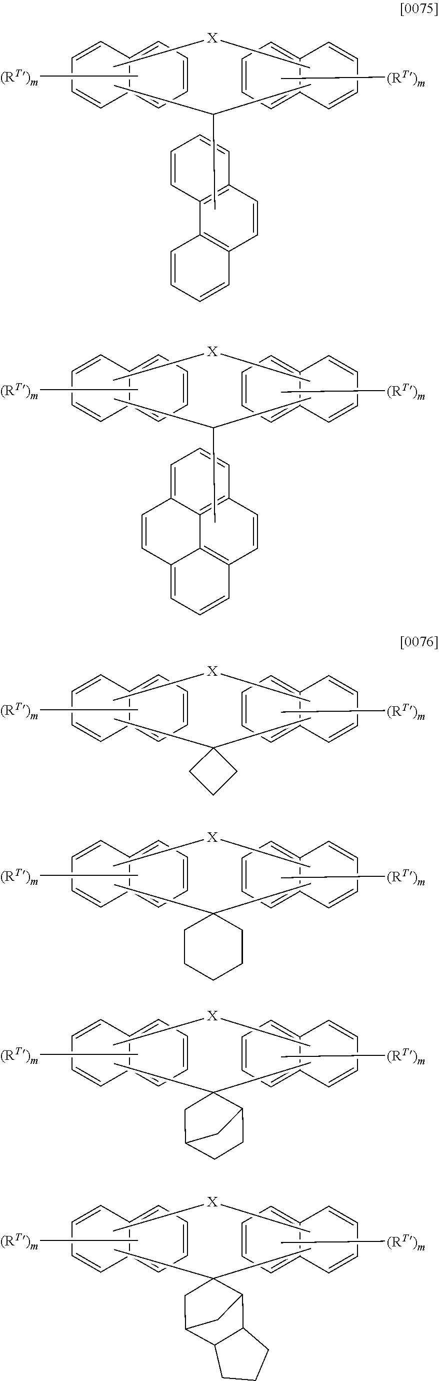

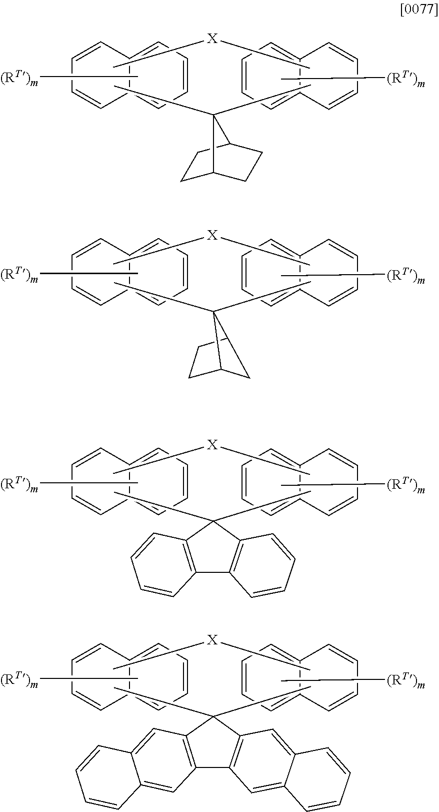

In the above formulae, X is as defined in that described in the above formula (0); R^{T′} is as defined in R^{T′} described in the above formula (0-1); and each m is independently an integer of 0 to 9, wherein at least one of m is an integer of 1 to 9. At least one of R^{T′} is a hydroxy group or a group in which a hydrogen atom of a hydroxy group is substituted with an acid crosslinking group or an acid dissociation group.

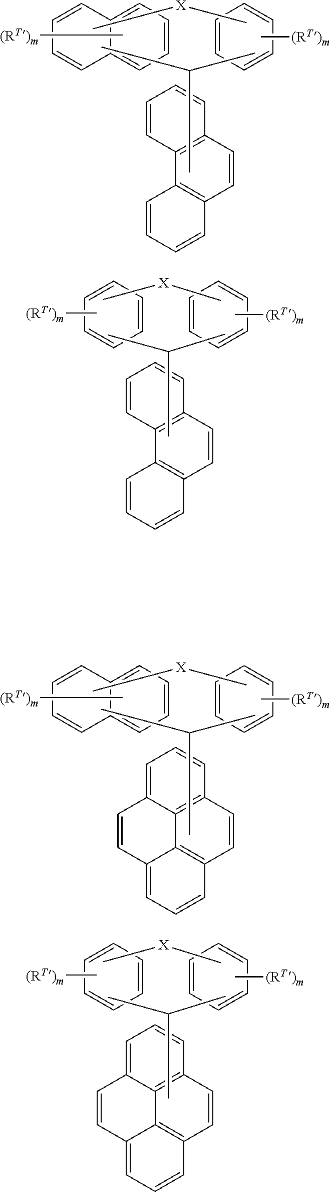

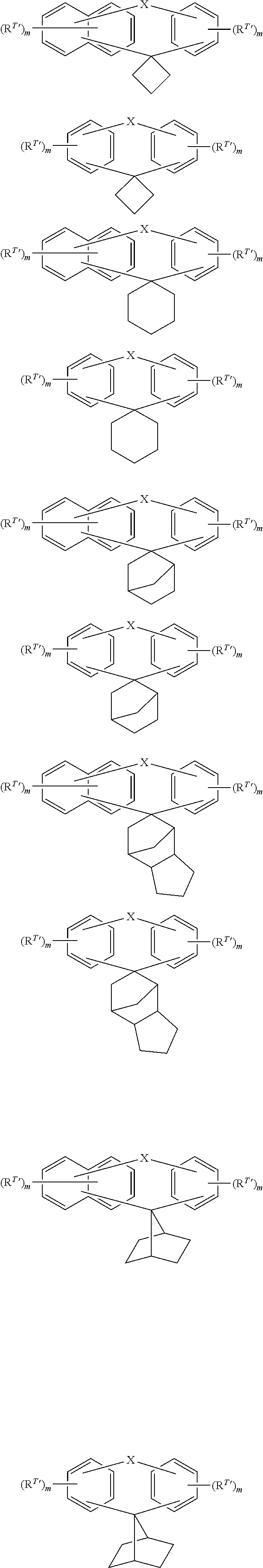

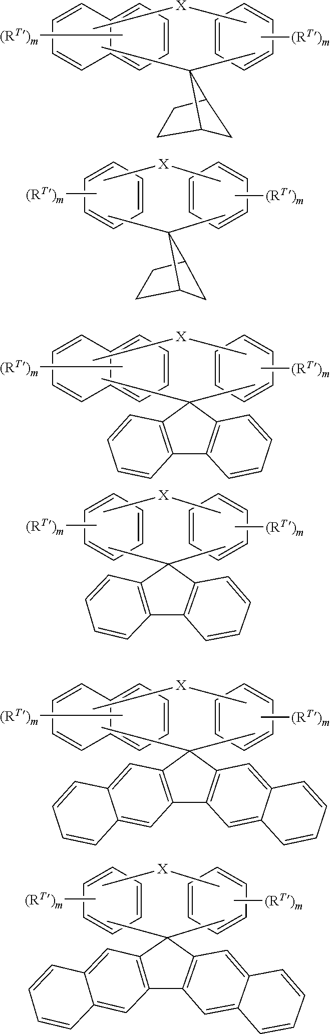

In the above formulae, X is as defined in that described in the above formula (0); R^{T′} is as defined in R^{T′} described in the above formula (0-1); and each m is independently an integer of 0 to 9, wherein at least one of m is an integer of 1 to 9. At least one of R^{T′} is a hydroxy group or a group in which a hydrogen atom of a hydroxy group is substituted with an acid crosslinking group or an acid dissociation group.

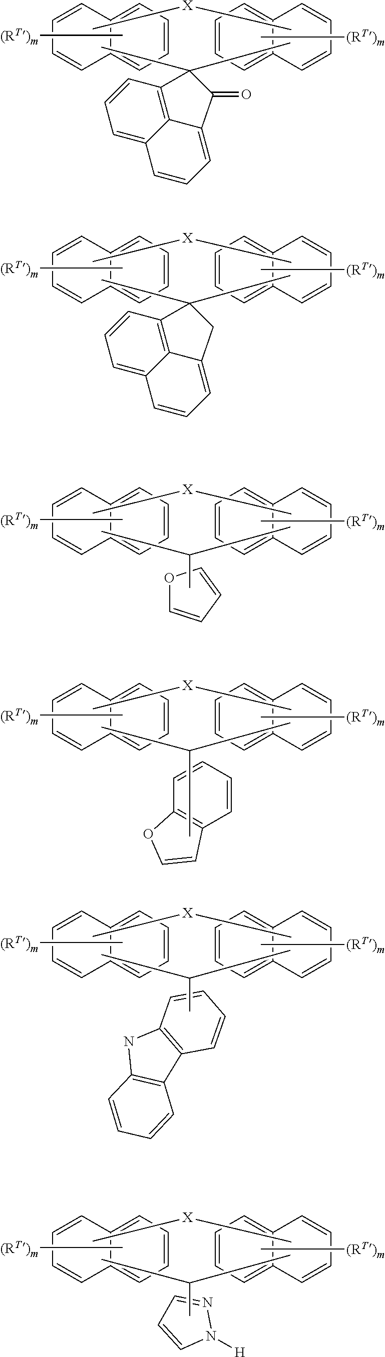

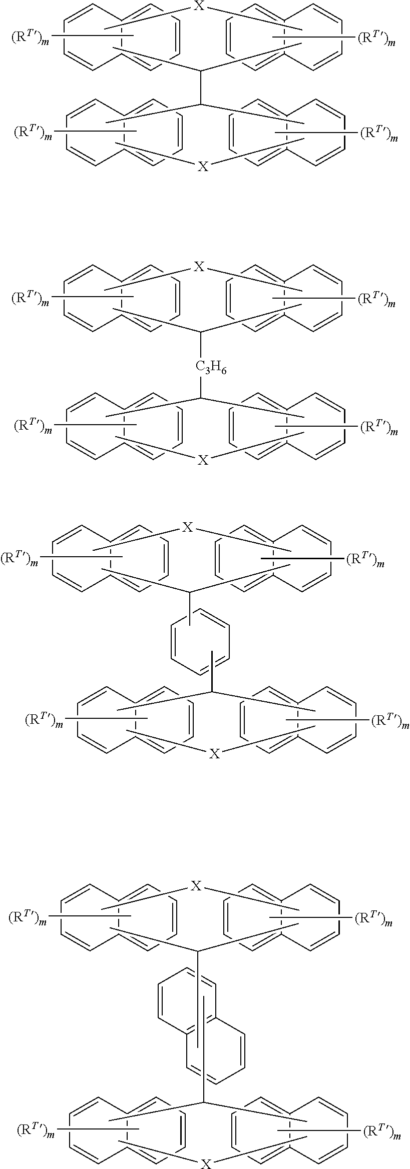

In the above formulae, X is as defined in that described in the above formula (0); R^{T′} is as defined in R^{T′} described in the above formula (0-1); and each m is independently an integer of 0 to 9, wherein at least one of m is an integer of 1 to 9. At least one of R^{T′} is a hydroxy group or a group in which a hydrogen atom of a hydroxy group is substituted with an acid crosslinking group or an acid dissociation group.

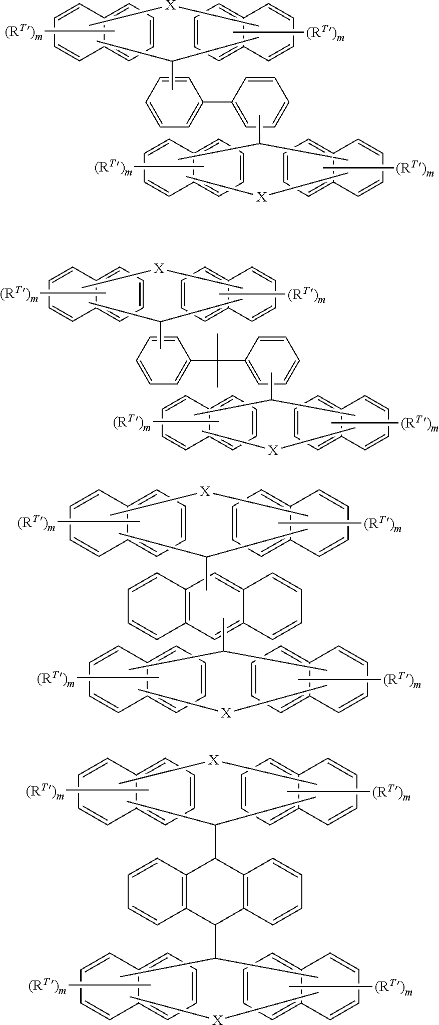

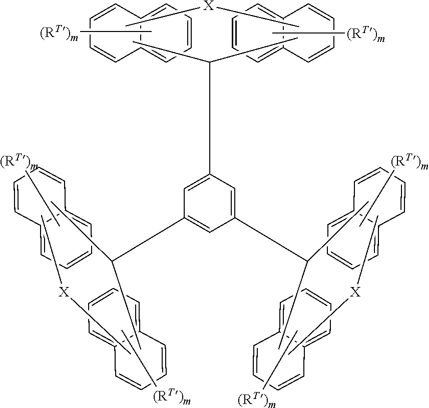

In the above formulae, X is as defined in that described in the above formula (0); R^{T′} is as defined in R^{T′} described in the above formula (0-1); and each m is independently an integer of 0 to 9, wherein at least one of m is an integer of 1 to 9. At least one of R^{T′} is a hydroxy group or a group in which a hydrogen atom of a hydroxy group is substituted with an acid crosslinking group or an acid dissociation group.

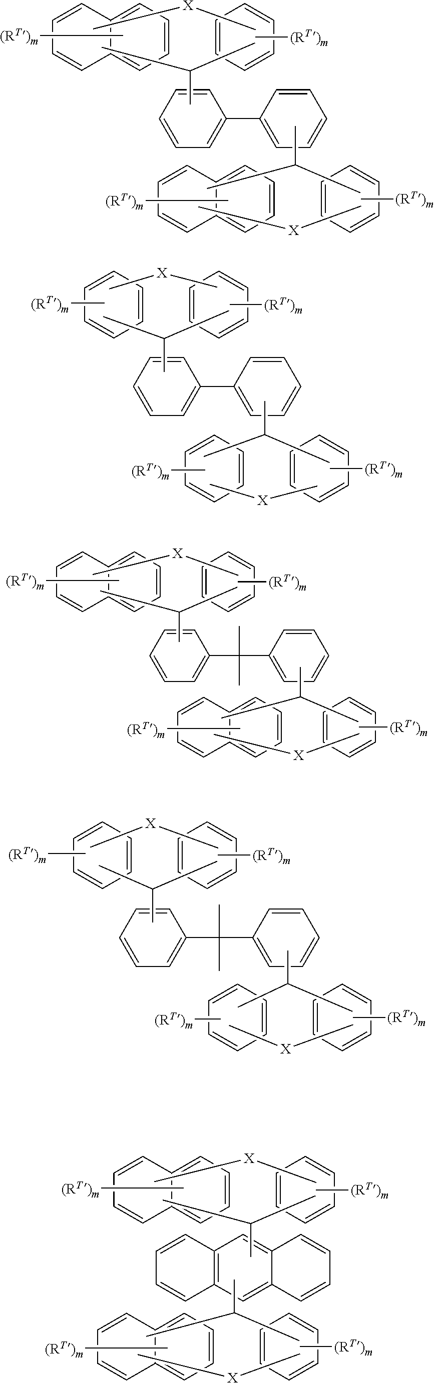

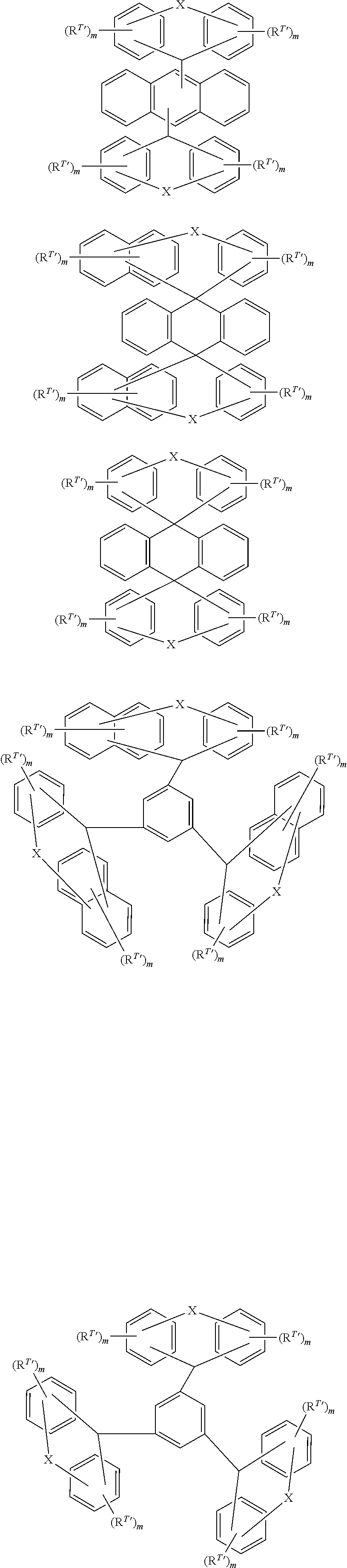

In the above formulae, X is as defined in that described in the above formula (0); R^{T′} is as defined in R^{T′} described in the above formula (0-1); and each m is independently an integer of 0 to 9, wherein at least one of m is an integer of 1 to 9. At least one of R^{T′} is a hydroxy group or a group in which a hydrogen atom of a hydroxy group is substituted with an acid crosslinking group or an acid dissociation group.

Specific examples of the compound represented by the above formula (0) further include, but not limited to:

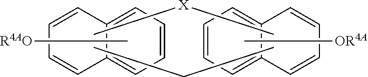

In the above formula, X is as defined in that described in the above formula (0); R^{Y′} and R^{Z′} are as defined in R^{Y} and R^{Z} described in the above formula (0); and each R^{4A} is independently a hydrogen atom, an acid crosslinking group or an acid dissociation group.

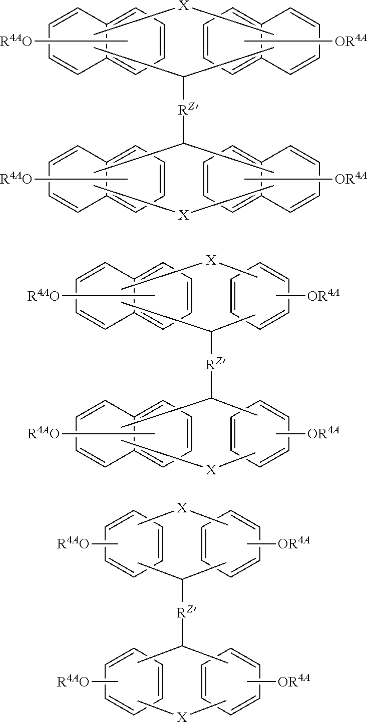

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

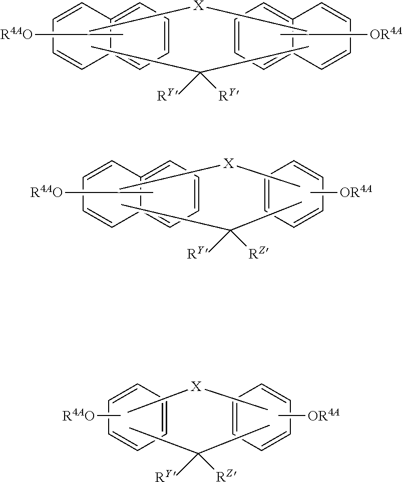

In the above formulae, X is as defined in that described in the above formula (0); R^{Y′} and R^{Z′} are as defined in R^{Y} and R^{Z} described in the above formula (0); and each R^{4A} is independently a hydrogen atom, an acid crosslinking group or an acid dissociation group.

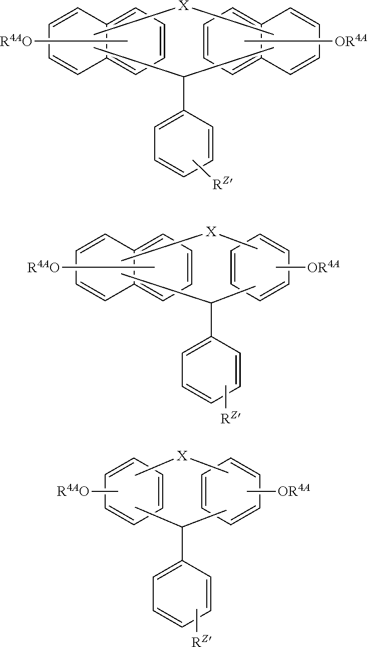

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

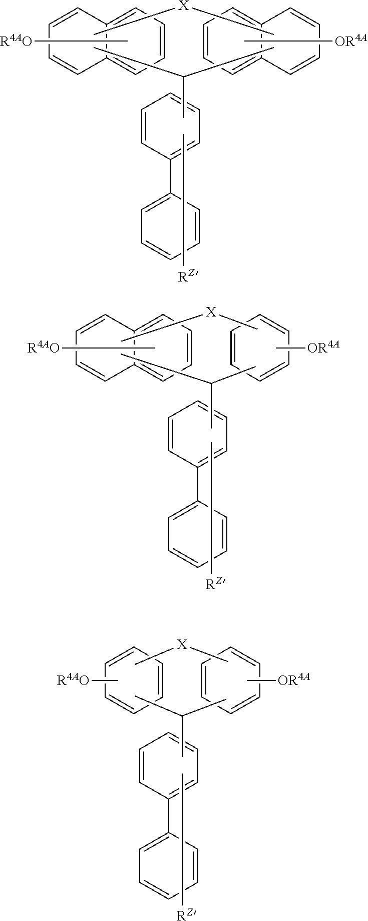

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

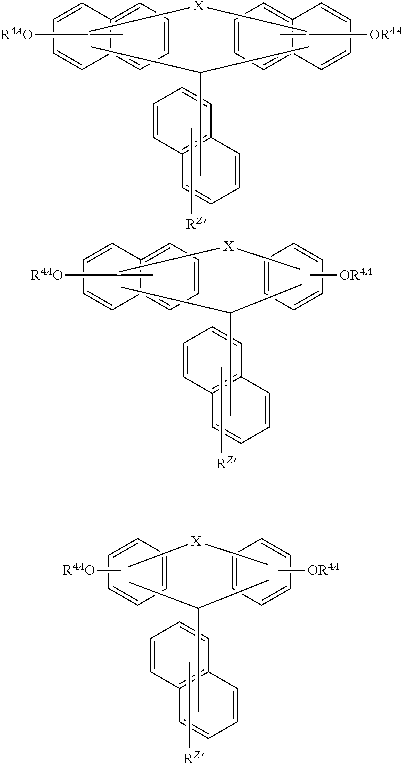

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

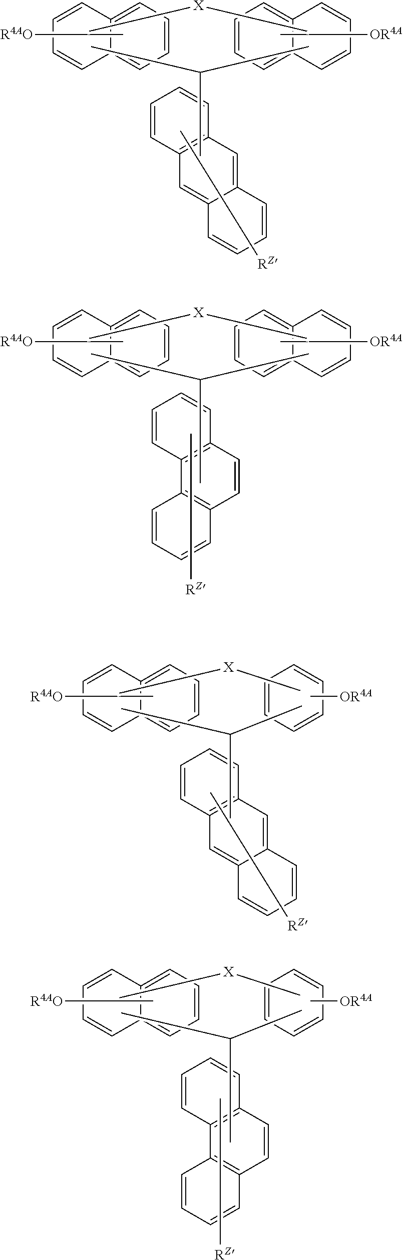

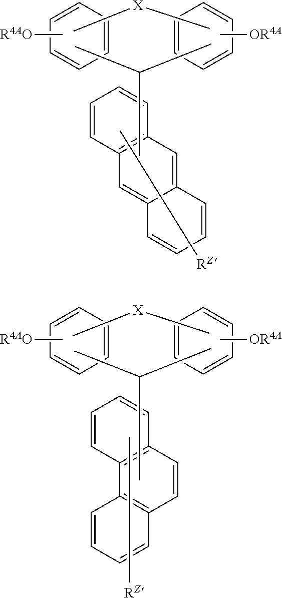

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

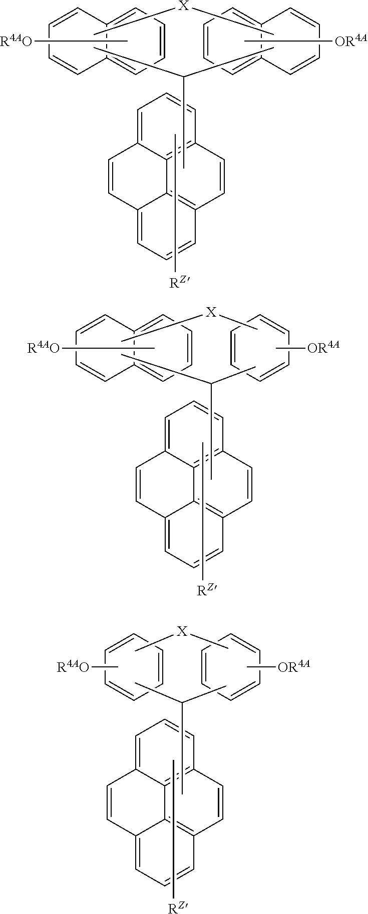

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

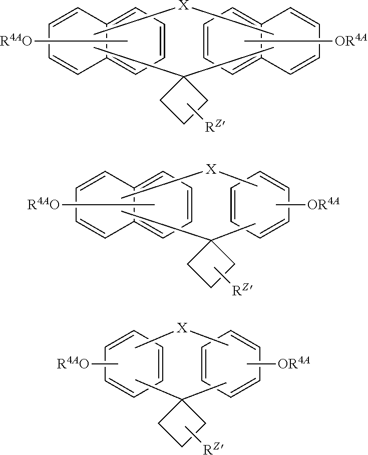

In the above formulae, X is as defined in that described in

the above formula (0); R^{Z′} is as defined in R^{Z} described in the above formula (0); and each R^{4A} is independently a hydrogen atom, an acid crosslinking group or an acid dissociation group.

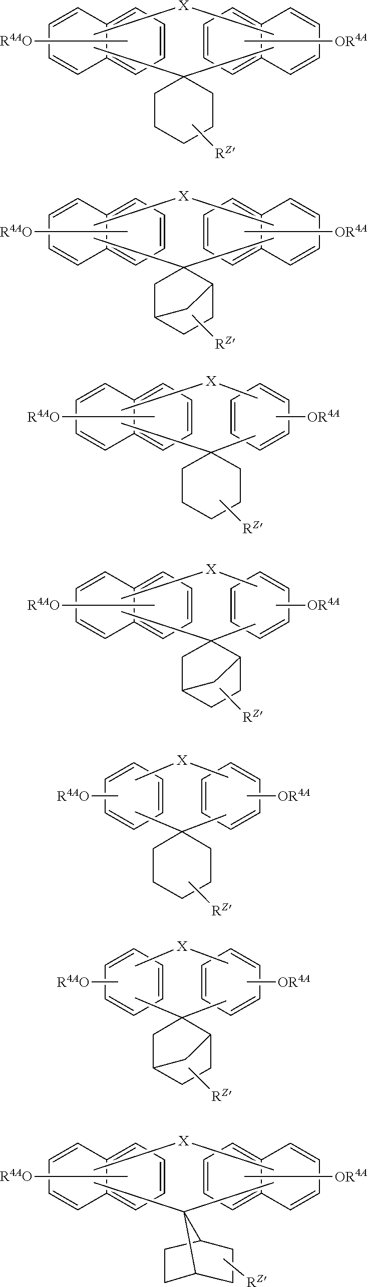

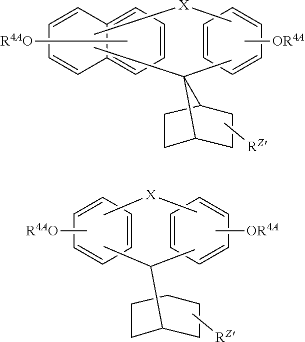

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

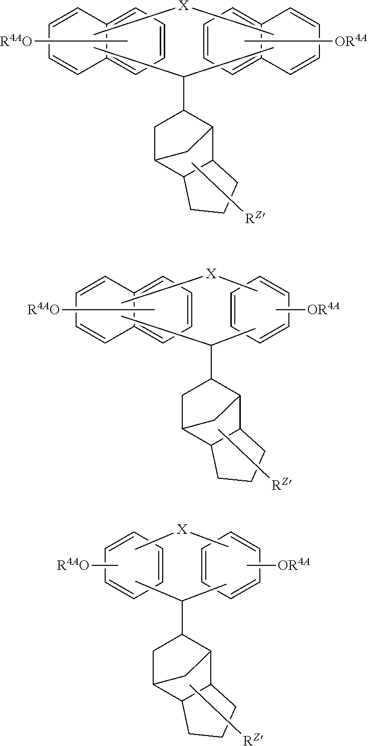

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

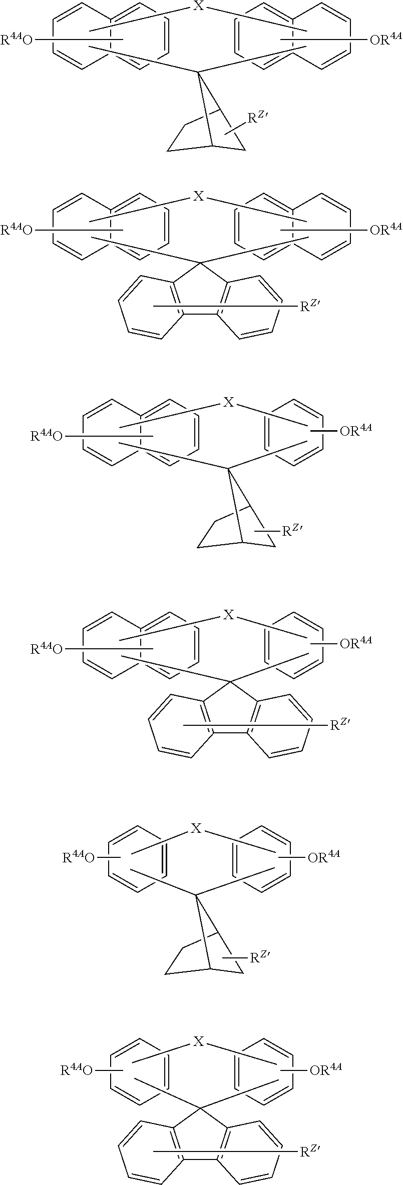

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

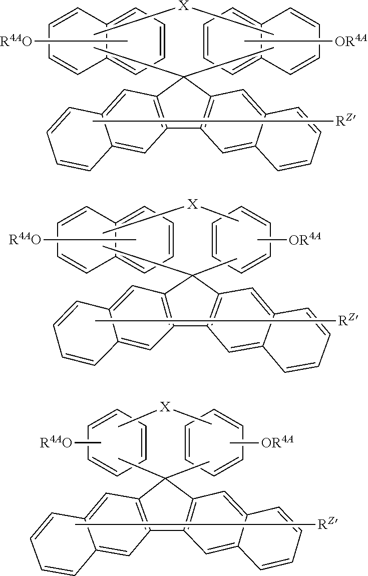

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

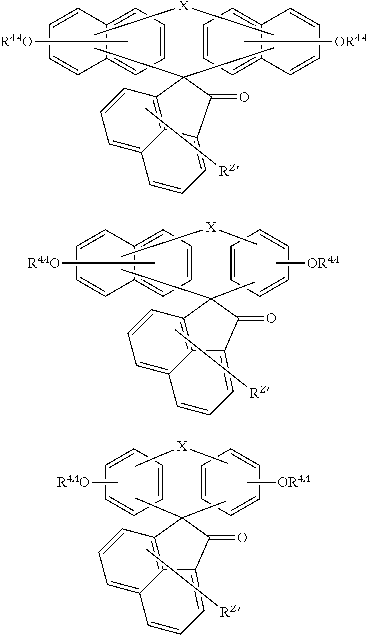

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

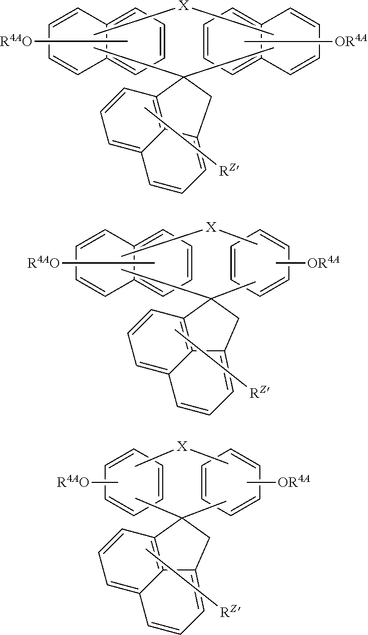

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

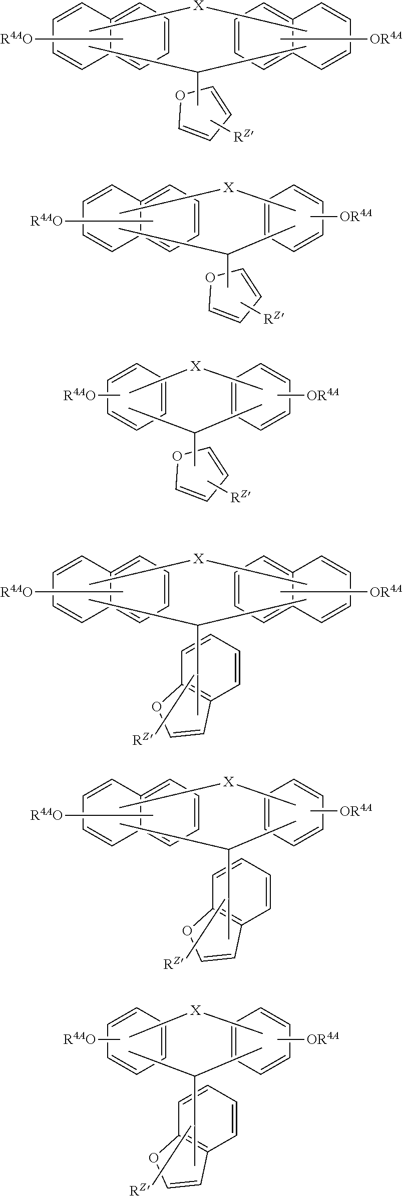

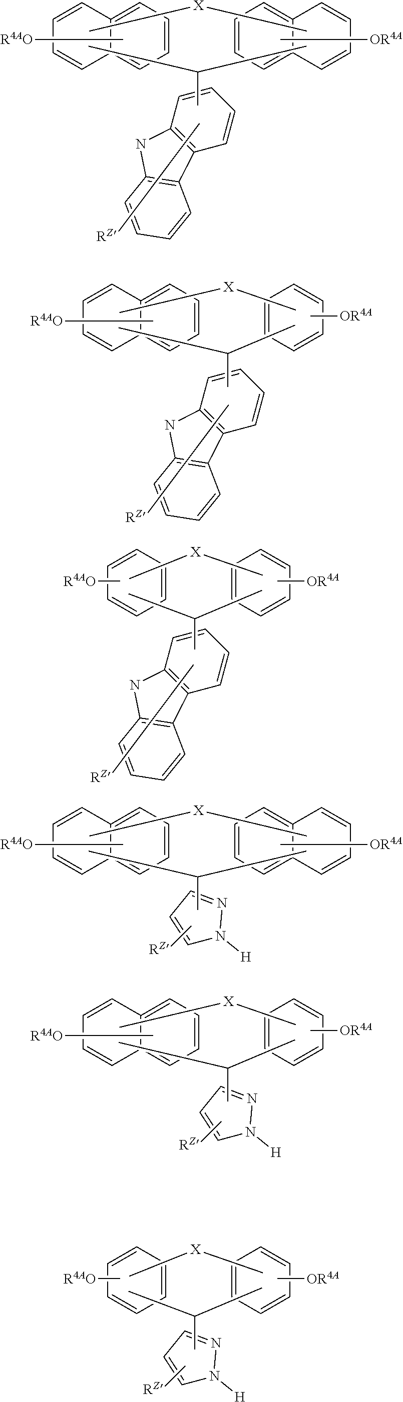

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

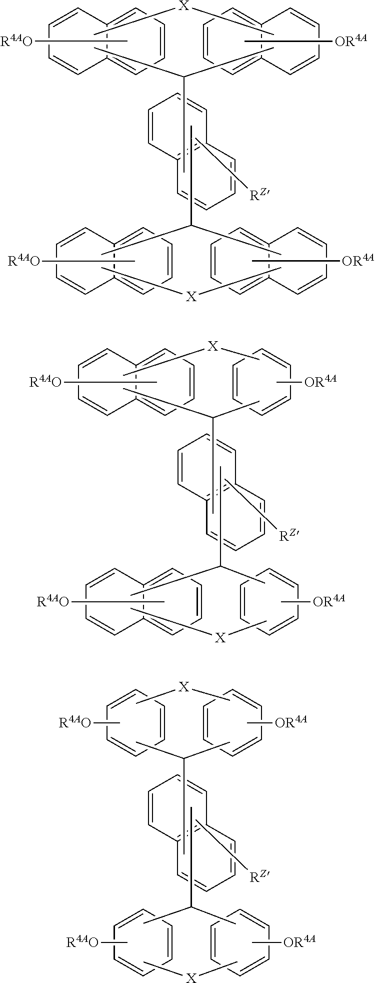

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

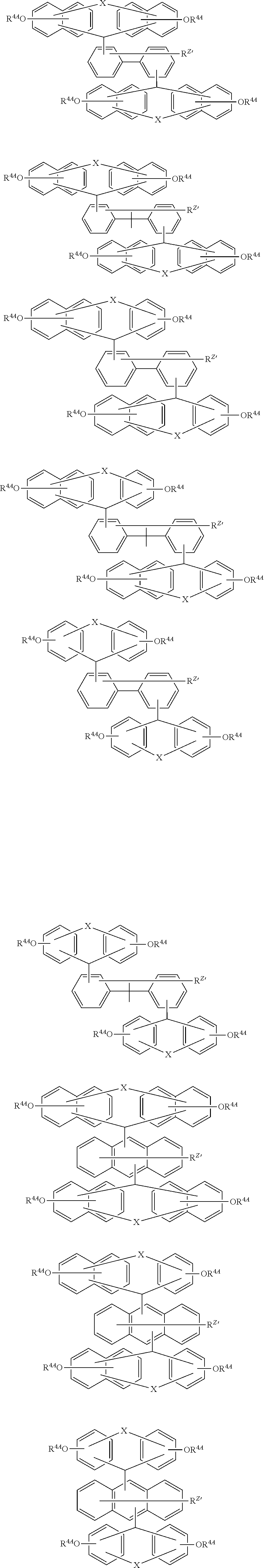

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

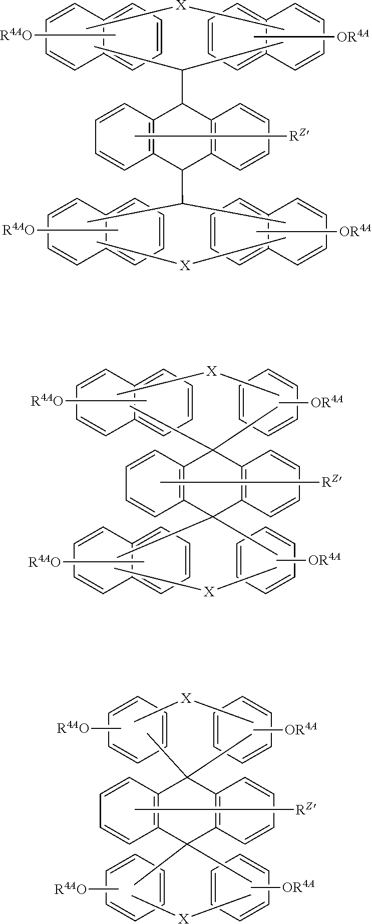

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

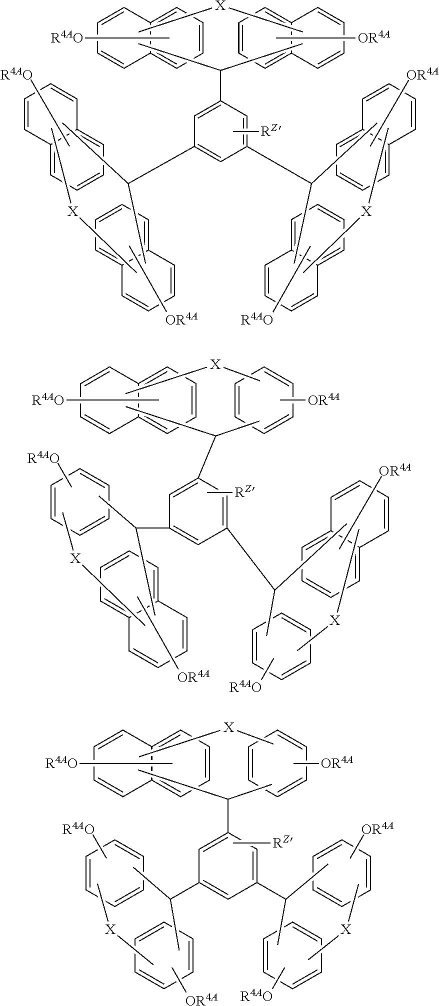

In the above formulae, X is as defined in that described in the above formula (0); R^{Z′} is as defined in R^{Z} described in the above formula (0); and each R^{4A} is independently a hydrogen atom, an acid crosslinking group or an acid dissociation group.

Specific examples of the compound represented by the above formula (1) include, but not limited to:

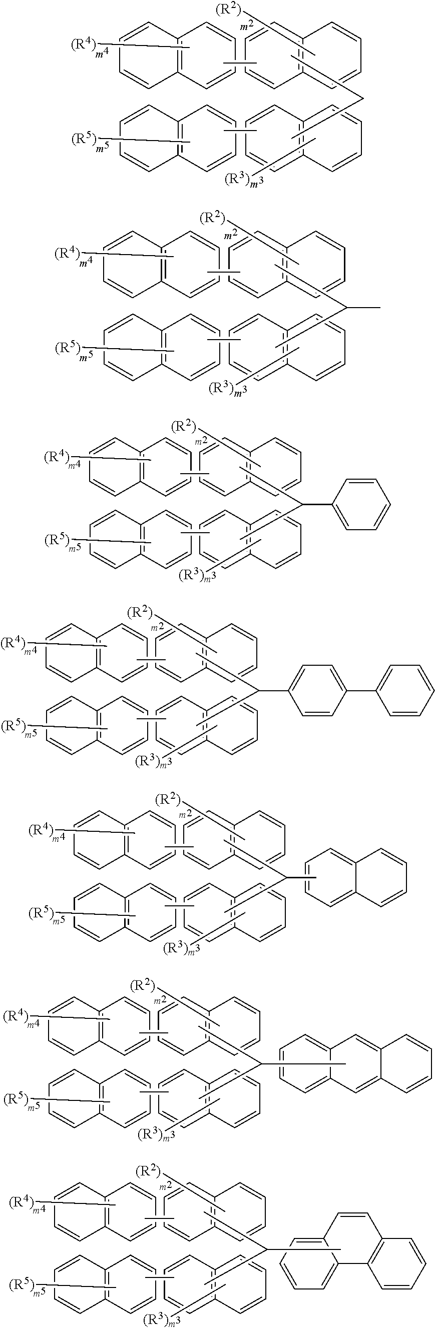

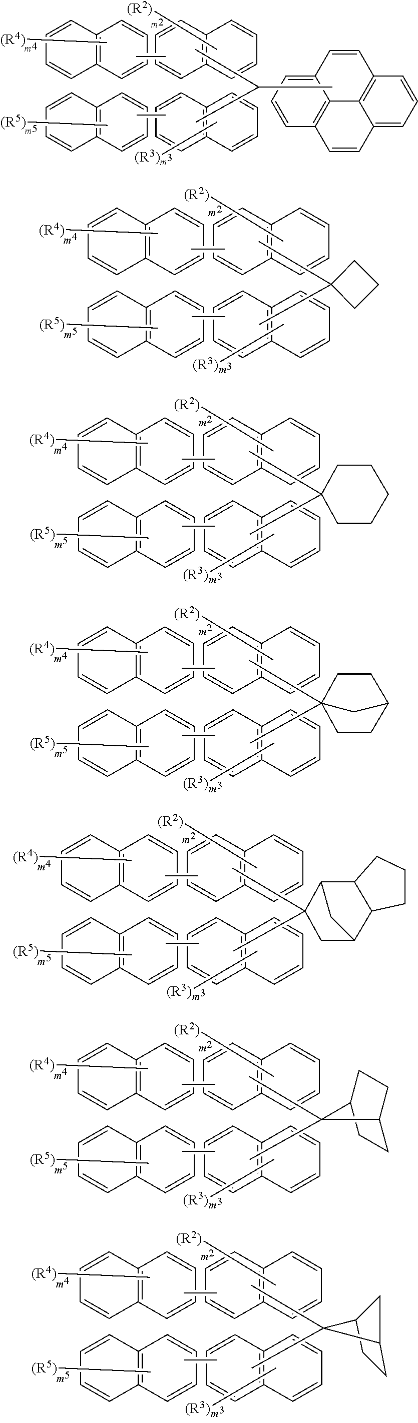

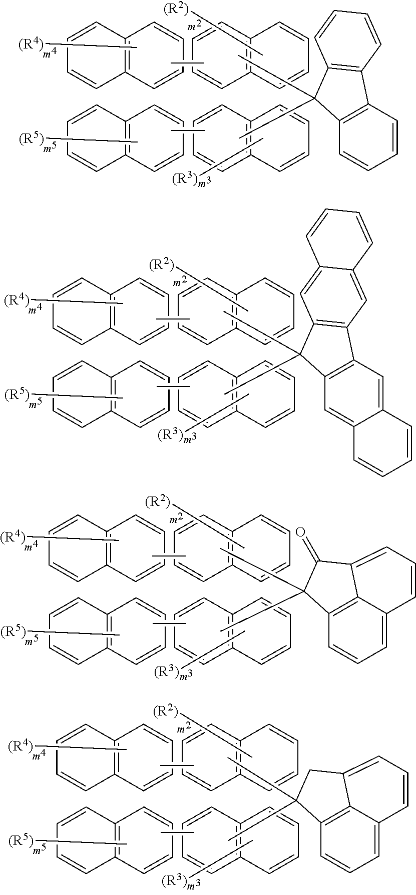

In the above compounds, R², R³, R⁴, and R⁵ are as defined in those described in the above formula (1), and at least one of R², R³, R⁴, and R⁵ is a hydroxy group or a group in which a hydrogen atom of a hydroxy group is substituted with an acid dissociation group. m² and m³ are each independently an integer of 0 to 8, and m⁴ and m⁵ are each independently an integer of 0 to 9. However, m², m³, m⁴, and m⁵ are not 0 at the same time.

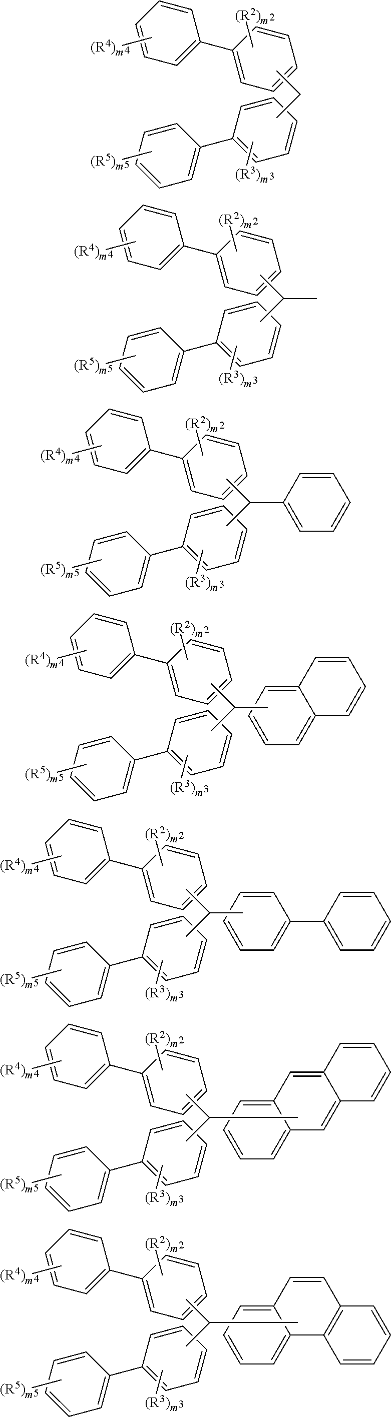

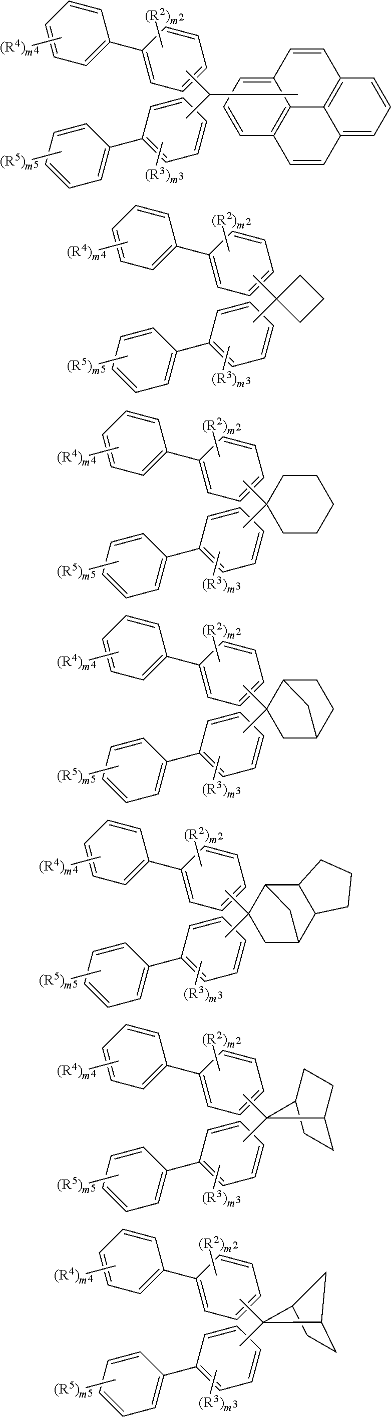

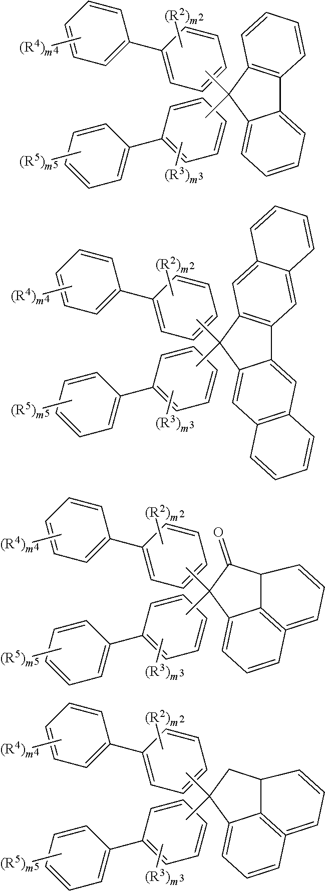

In the above compounds, R², R³, R⁴, and R⁵ are as defined in those described in the above formula (1), and at least one of R², R³, R⁴, and R⁵ is a hydroxy group or a group in which a hydrogen atom of a hydroxy group is substituted with an acid dissociation group. m² and m³ are each independently an integer of 0 to 8, and m⁴ and m⁵ are each independently an integer of 0 to 9. However, m², m³, m⁴, and m⁵ are not 0 at the same time.

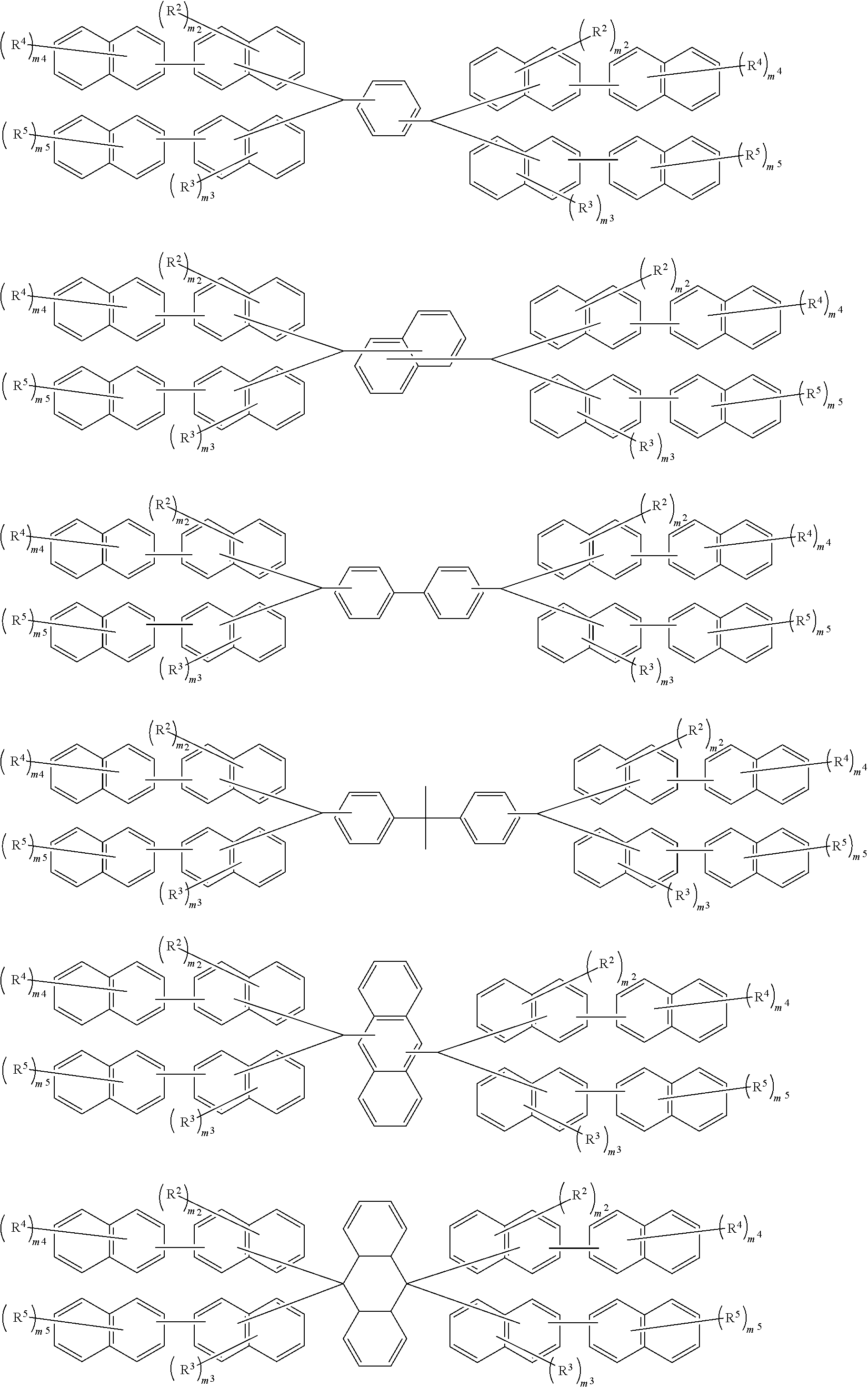

In the above compounds, R², R³, R⁴, and R⁵ are as defined in those described in the above formula (1), and at least one of R², R³, R⁴, and R⁵ is a hydroxy group or a group in which a hydrogen atom of a hydroxy group is substituted with an acid dissociation group. m² and m³ are each independently an integer of 0 to 8, and m⁴ and m⁵ are each independently an integer of 0 to 9. However, m², m³, m⁴, and m⁵ are not 0 at the same time.

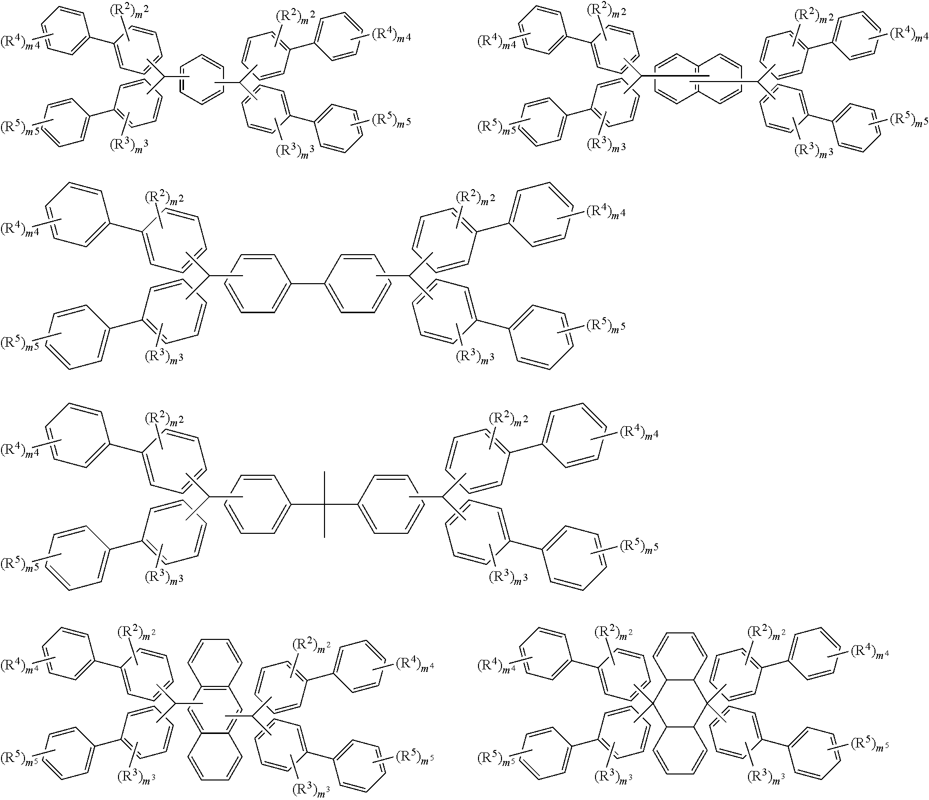

In the above compounds, R², R³, R⁴, and R⁵ are as defined in those described in the above formula (1), and at least one of R², R³, R⁴, and R⁵ is a hydroxy group or a group in which a hydrogen atom of a hydroxy group is substituted with an acid dissociation group. m² and m³ are each independently an integer of 0 to 8, and m⁴ and m⁵ are each independently an integer of 0 to 9. However, m², m³, m⁴, and m⁵ are not 0 at the same time.

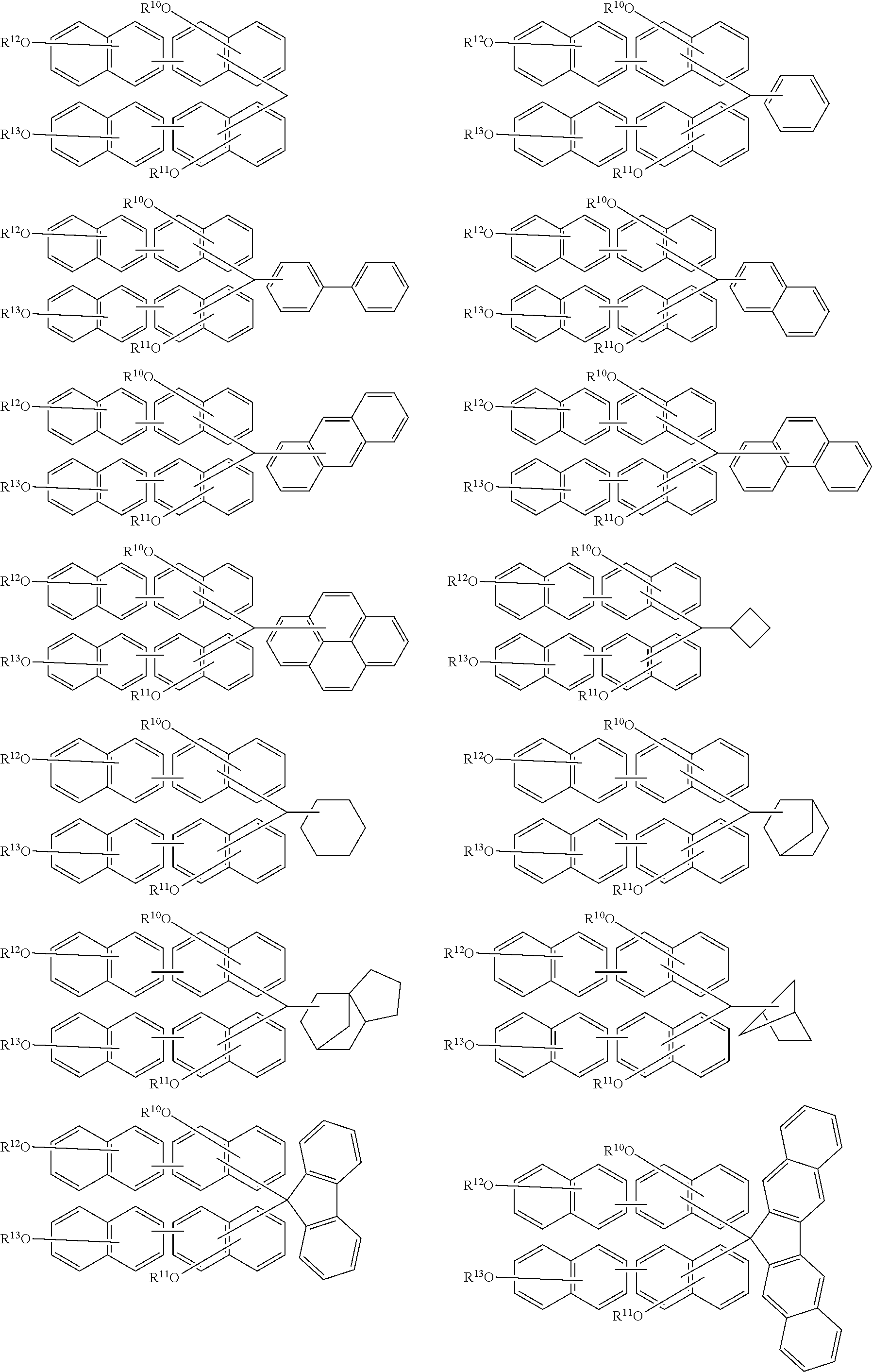

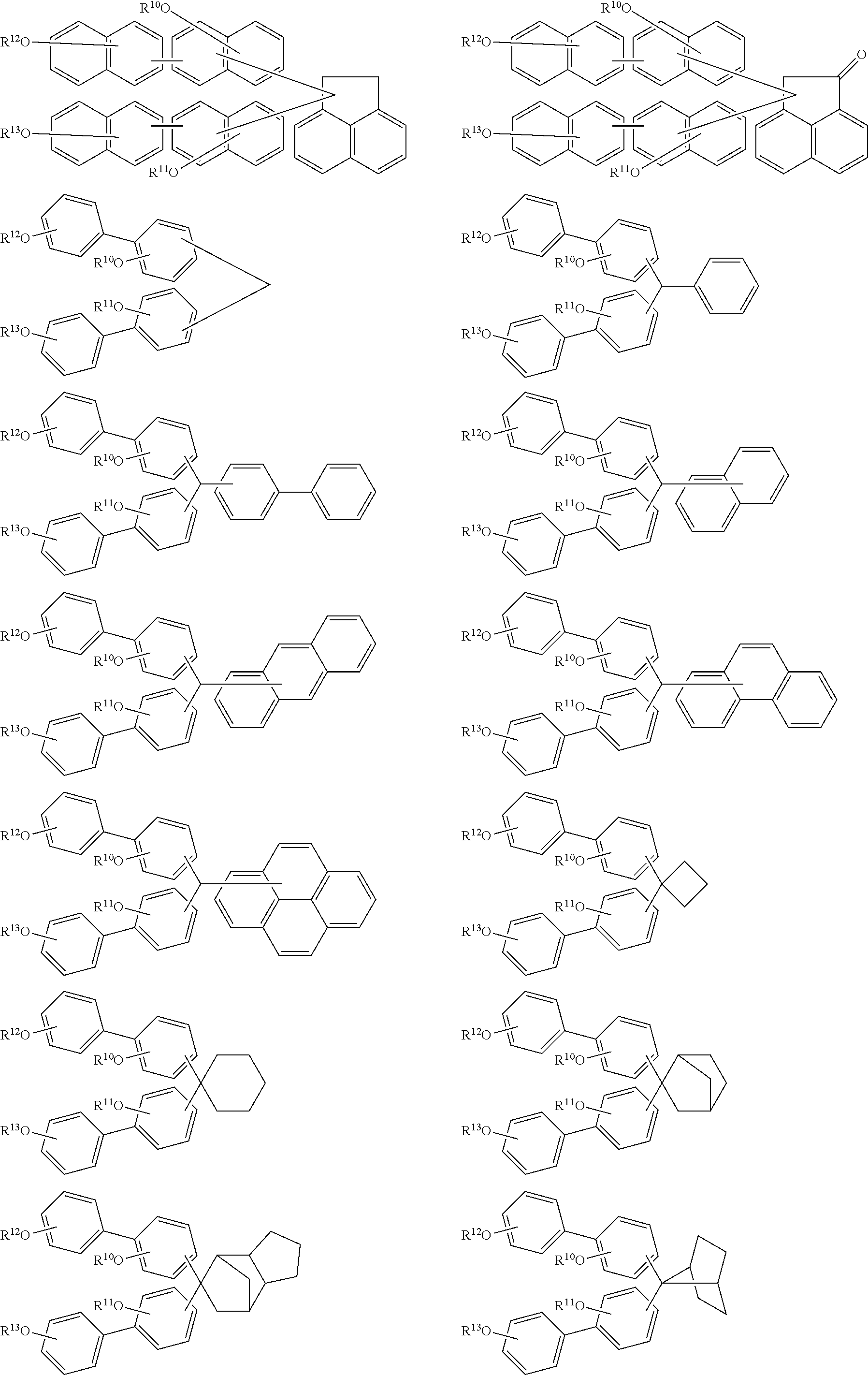

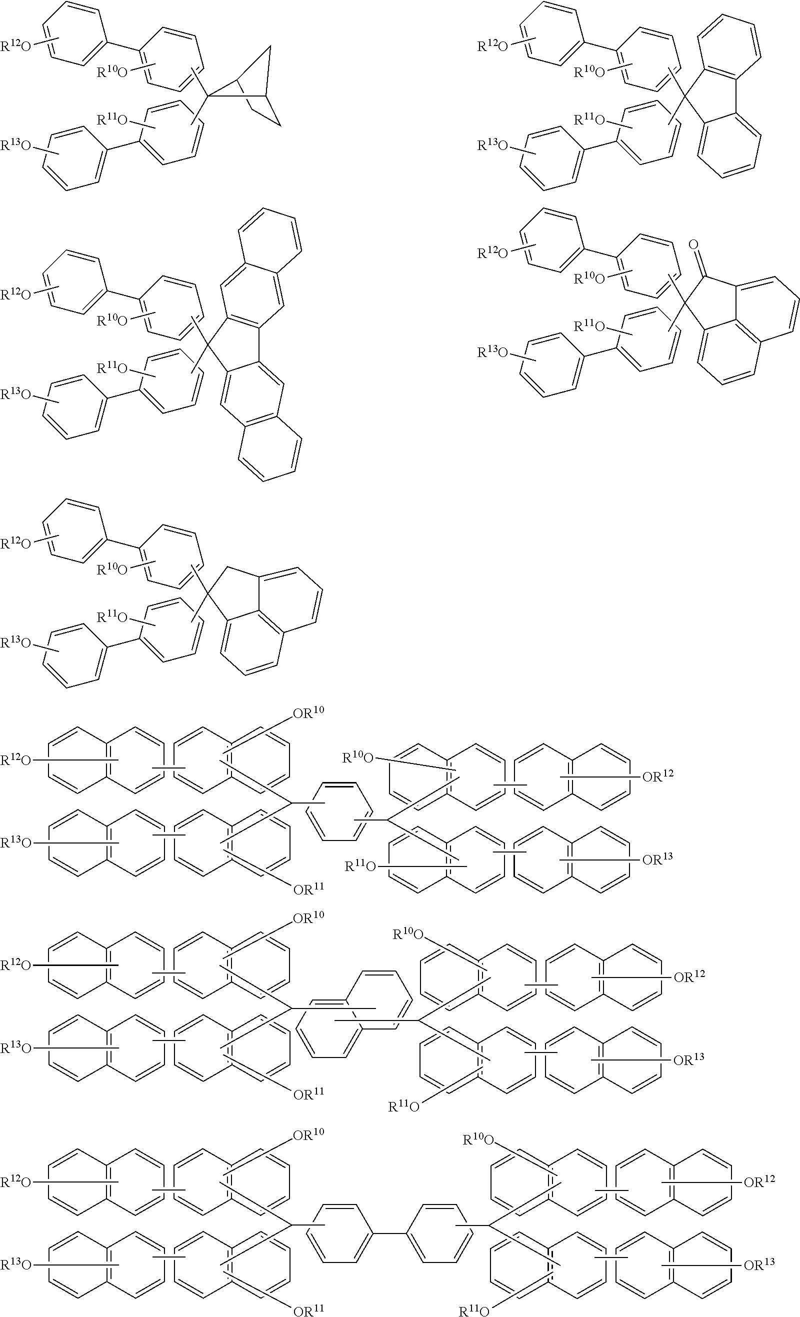

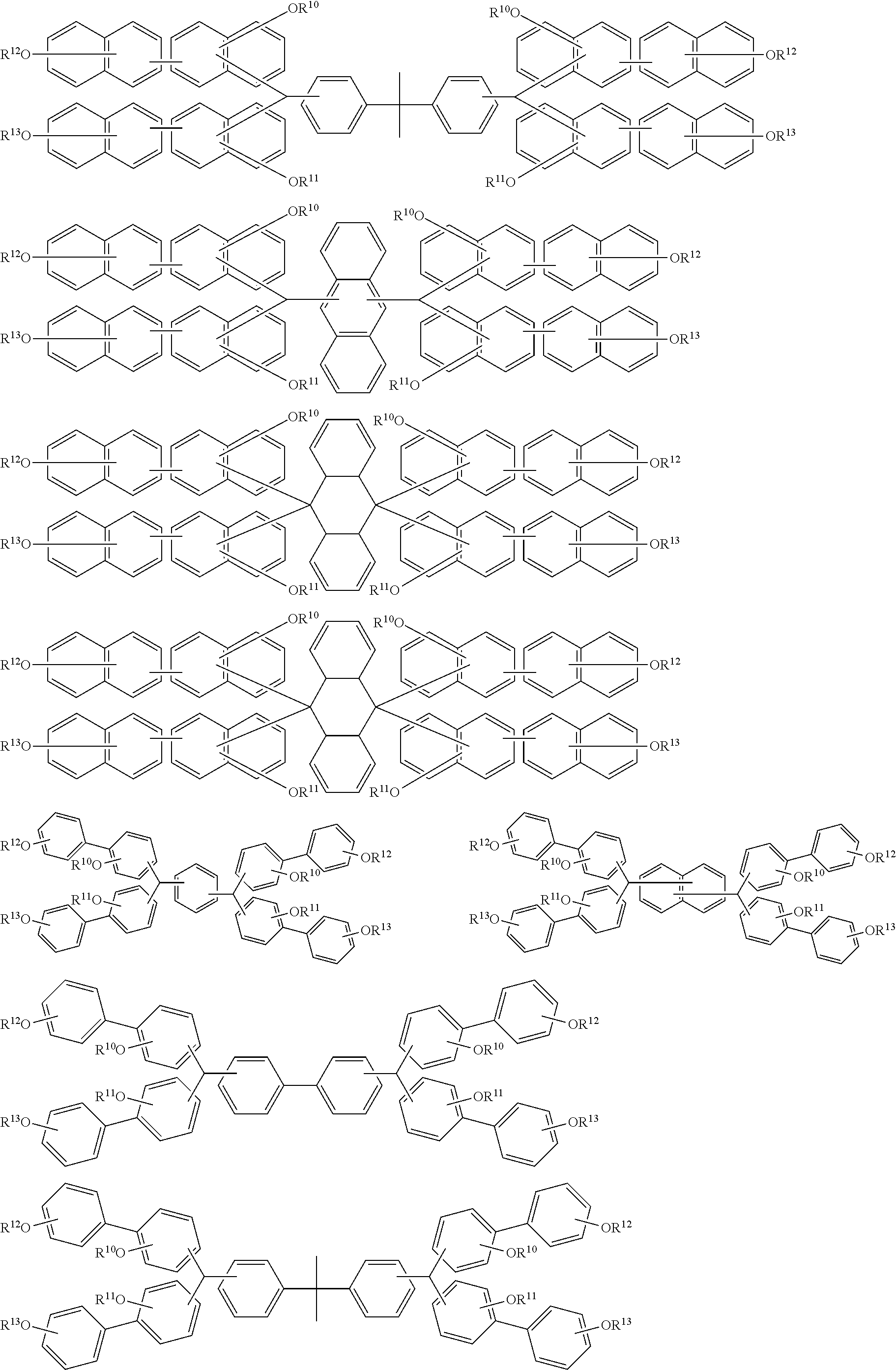

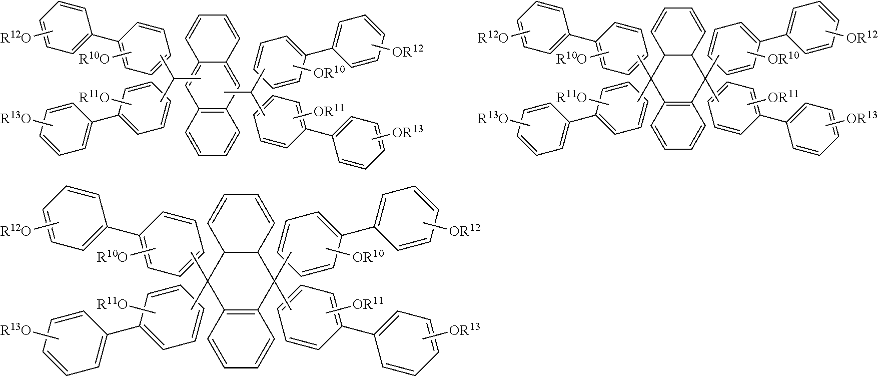

In the above compounds, R¹⁰, R¹¹, R¹², and R¹³ are as defined in those described in the above formula (1-2).

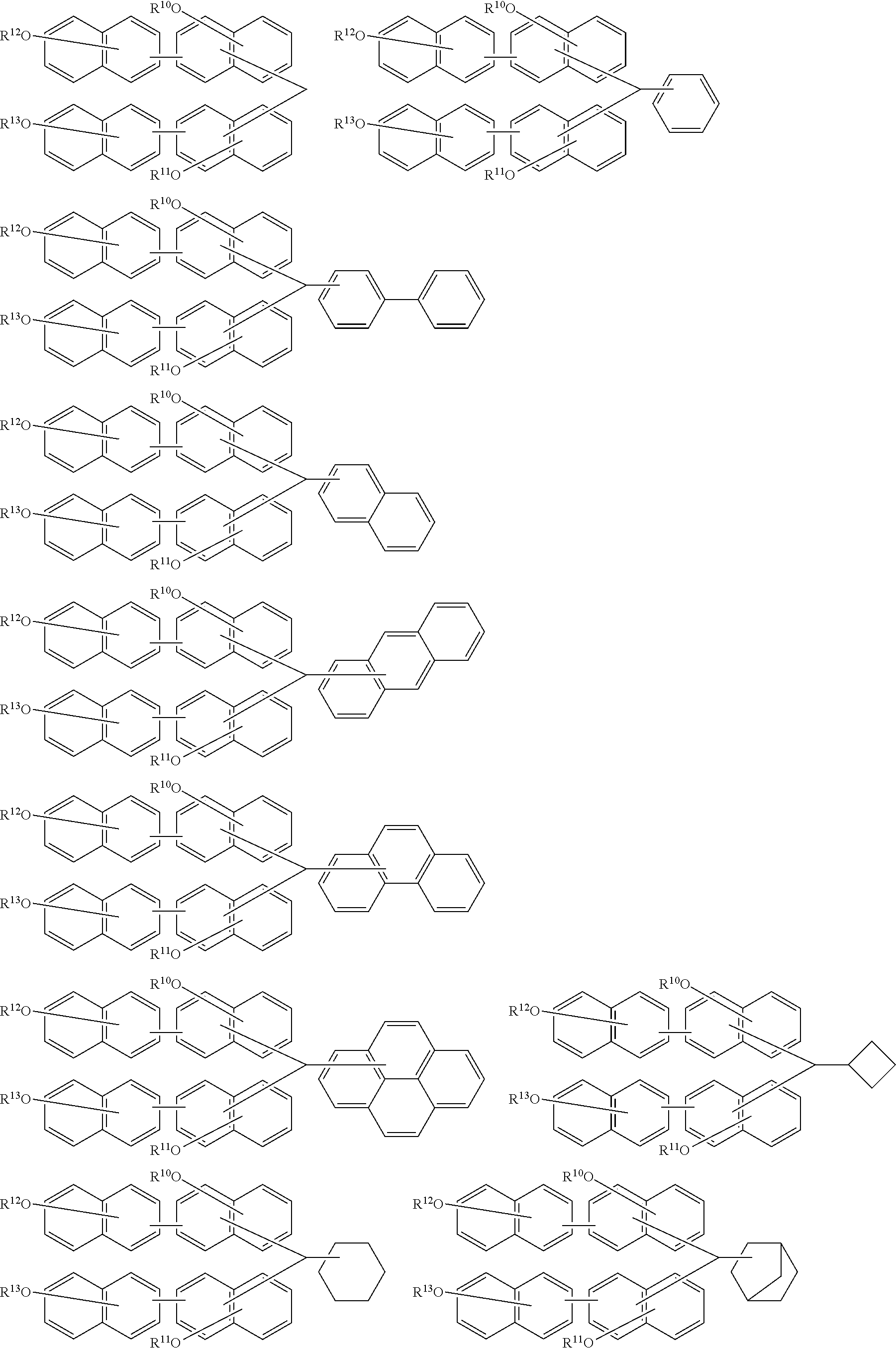

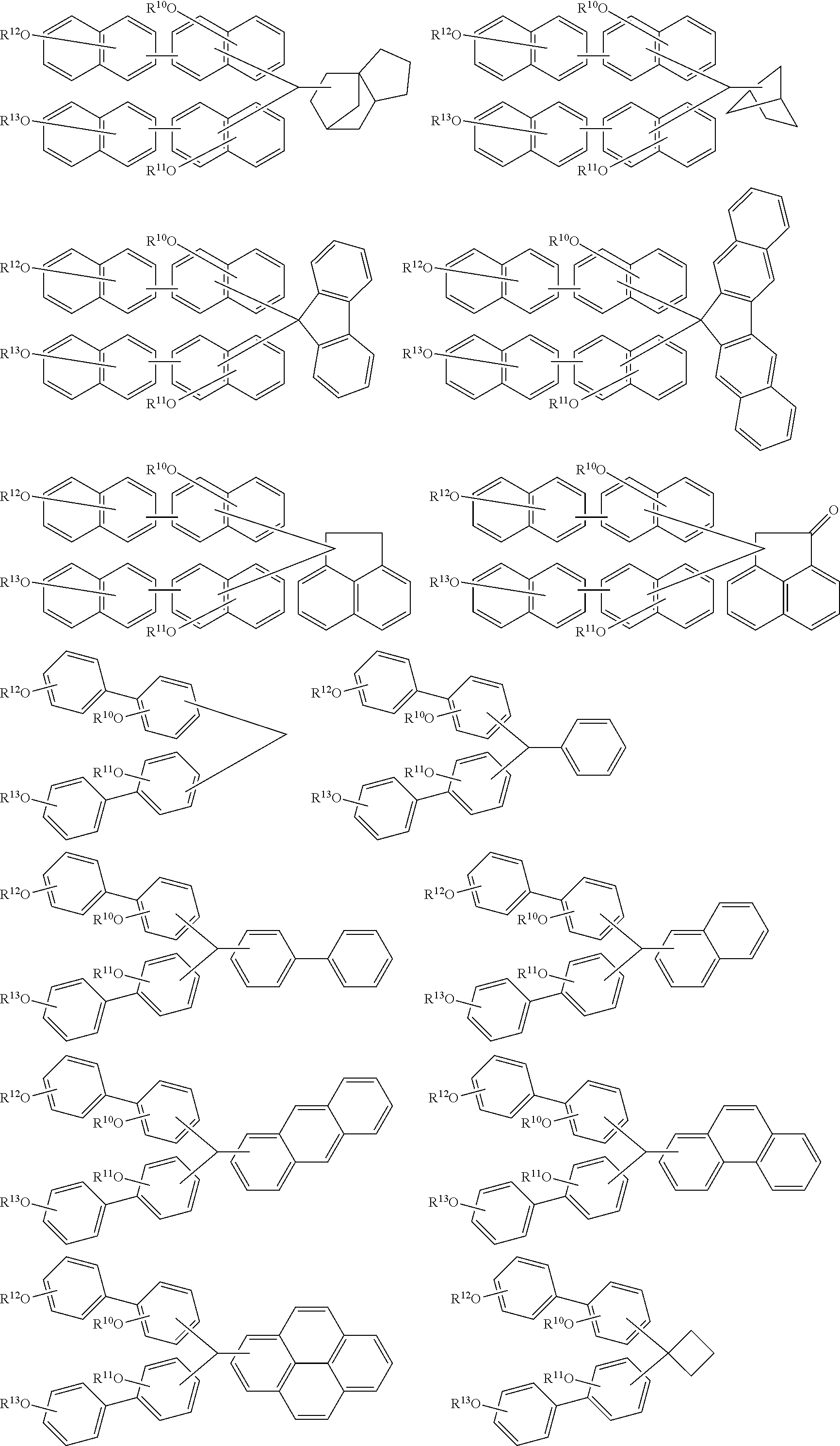

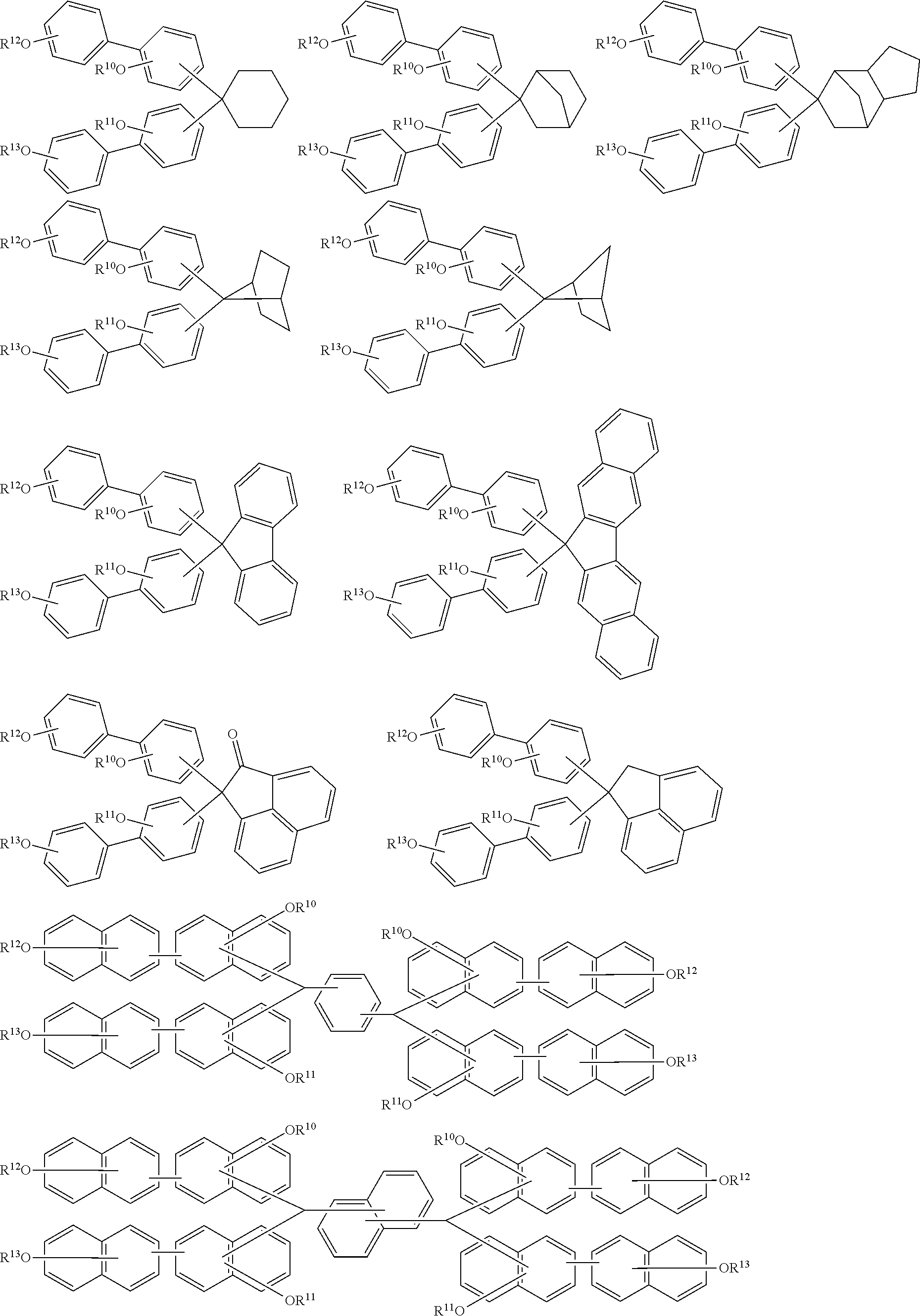

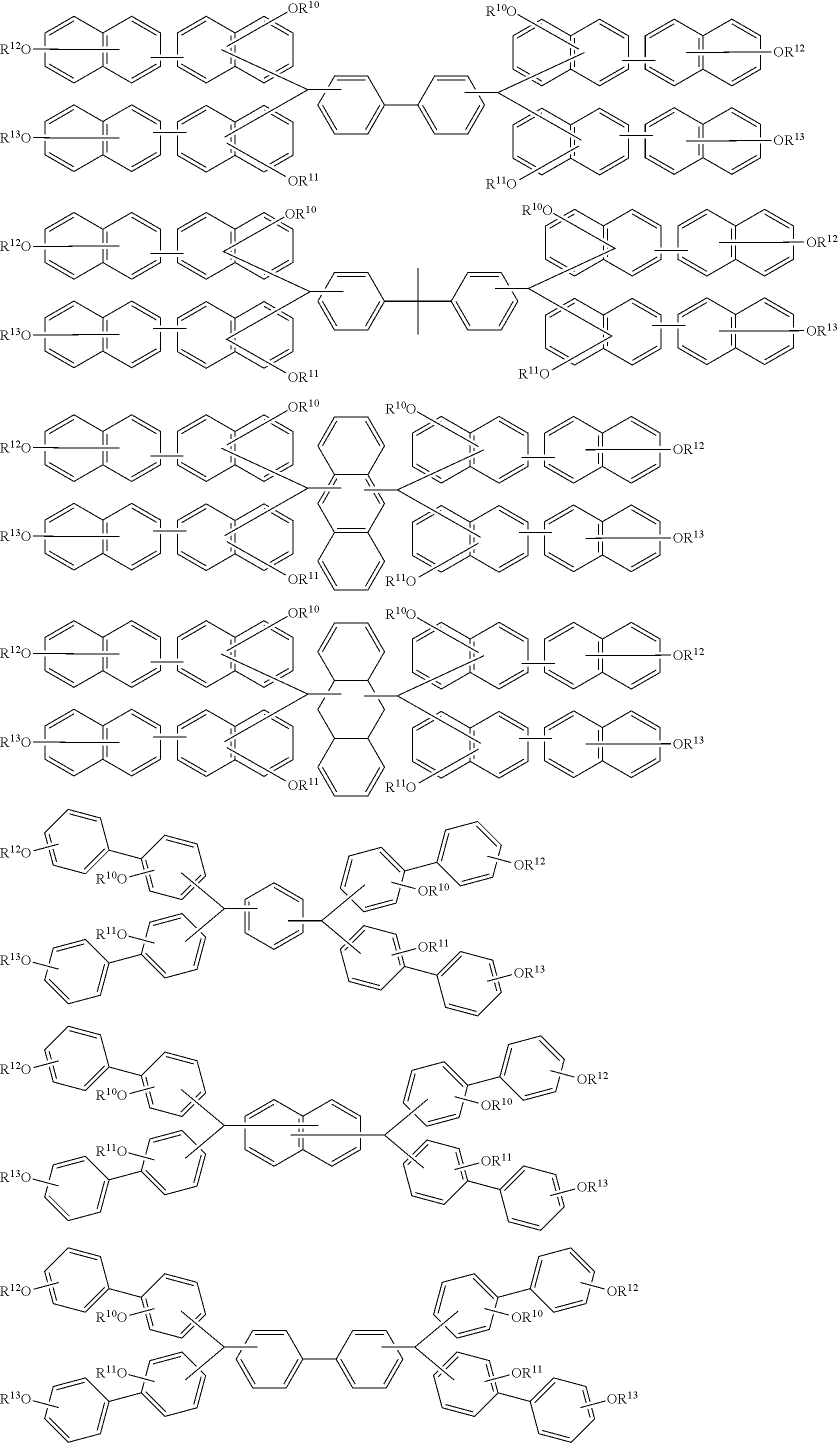

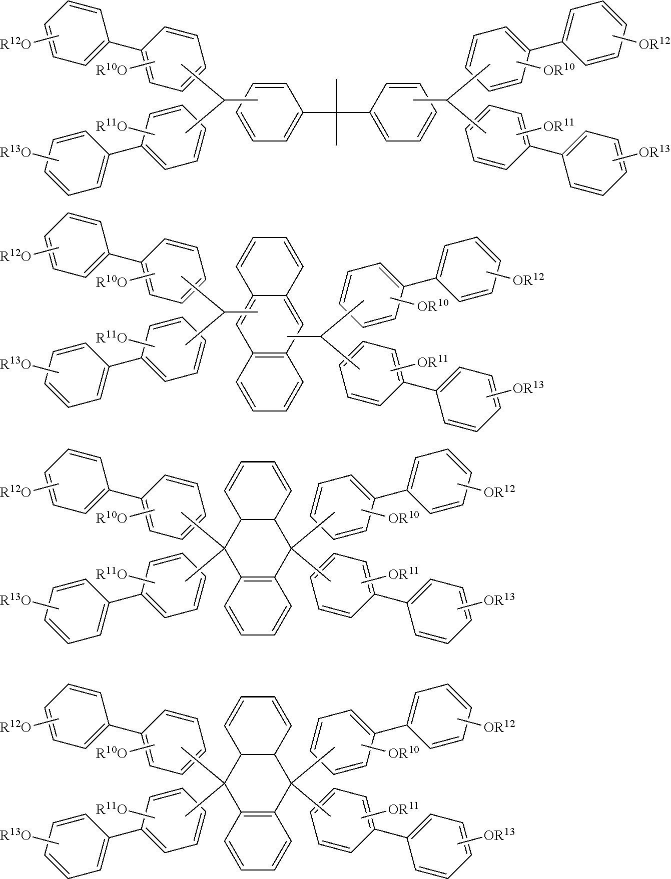

In the above compounds, R¹⁰, R¹¹, R¹², and R¹³ are as defined in those described in the above formula (1-2).

The compound represented by the above formula (1) is particularly preferably a compound represented by any of the following formulae (BiF-1) to (BiF-10) (R¹⁰ to R¹³ in the specific examples are as defined above) from the viewpoint of further solubility in an organic solvent:

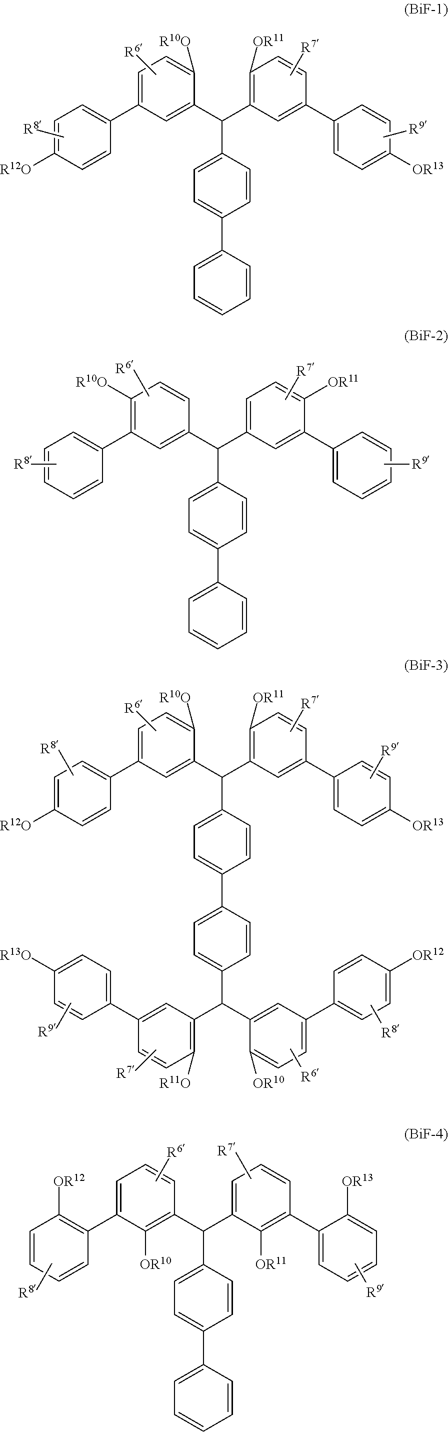

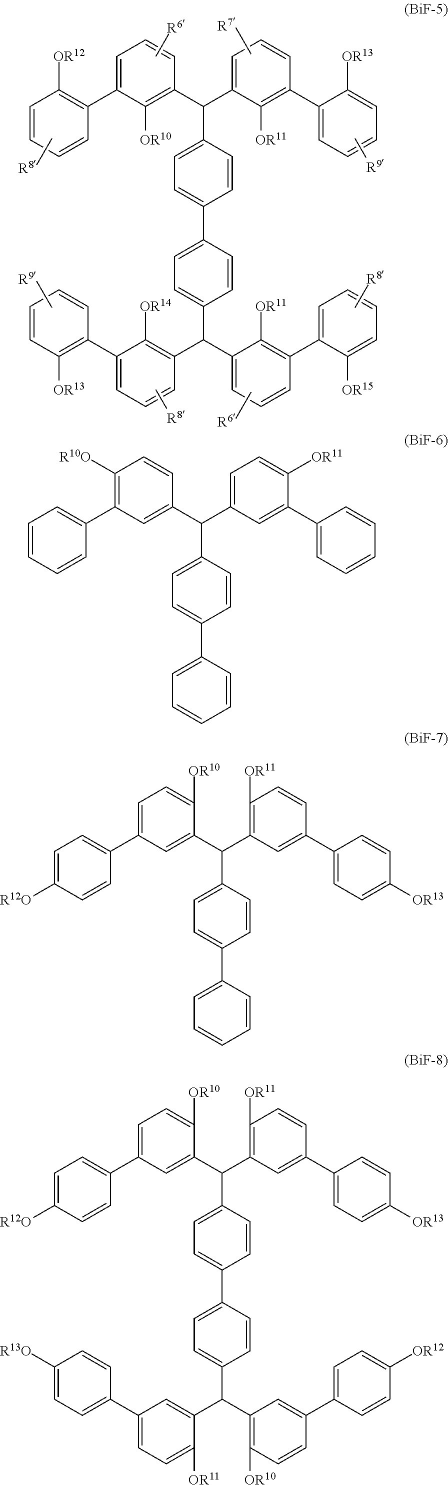

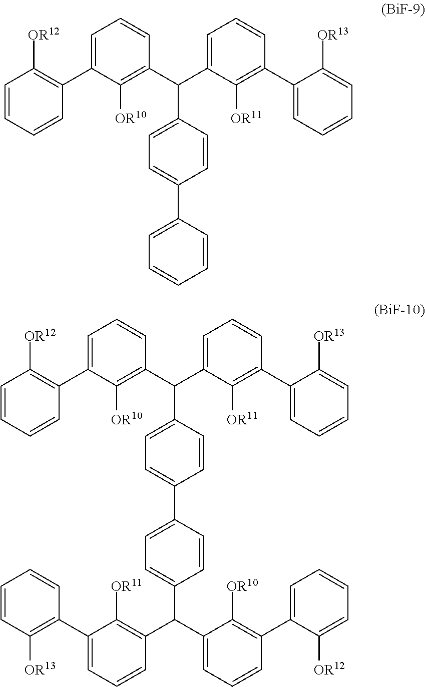

In the above formulae (BiF-1) to (BiF-10), R^{6′} to R^{9′} are each independently a hydrogen atom, an alkyl group of 1 to 30 carbon atoms optionally having a substituent, an aryl group of 6 to 30 carbon atoms optionally having a substituent, an alkenyl group of 2 to 30 carbon atoms optionally having a substituent, a halogen atom, a nitro group, an amino group, a carboxylic acid group or a thiol group, wherein at least one of R^{6′} to R^{9′} is an alkenyl group of 2 to 30 carbon atoms; and R¹⁰ to R¹³ are as defined in those described in the above formula (1c).

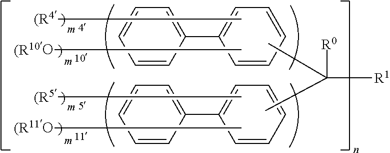

In the above formula, R⁰, R¹, and n are as defined in those described in the above formula (1-1); R^{10′} and R^{11′} are as defined in R¹⁰ and R¹¹ described in the above formula (1-1); R^{4′} and R^{5′} are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a nitro group, an amino group, a carboxylic acid group, a thiol group, a hydroxy group or a group represented by the formula (0-1), wherein the alkyl group, the aryl group, the alkenyl group, and the alkoxy group each optionally have an ether bond, a ketone bond or an ester bond, and at least one of

R^{10′} and R^{11′} contains a group represented by the formula (0-1); each of m^{4′} and m^{5′} is an integer of 0 to 8; each of m^{10′} and m^{11′} is an integer of 1 to 9; and m^{4′}+m^{10′} and m^{5′}+m^{11′} are each independently an integer of 1 to 9.

Examples of R⁰ include a methyl group, an ethyl group, a propyl group, a butyl group, a pentyl group, a hexyl group, a heptyl group, an octyl group, a nonyl group, a decyl group, an undecyl group, a dodecyl group, a triacontyl group, a phenyl group, a naphthyl group, an anthracene group, a pyrenyl group, a biphenyl group, and a heptacene group.

Examples of R^{4′} and R^{5′} include a methyl group, an ethyl group, a propyl group, a butyl group, a pentyl group, a hexyl group, a heptyl group, an octyl group, a nonyl group, a decyl group, an undecyl group, a dodecyl group, a triacontyl group, a cyclopropyl group, a cyclobutyl group, a cyclopentyl group, a cyclohexyl group, a cycloheptyl group, a cyclooctyl group, a cyclononyl group, a cyclodecyl group, a cycloundecyl group, a cyclododecyl group, a cyclotriacontyl group, a norbornyl group, an adamantyl group, a phenyl group, a naphthyl group, an anthracene group, a pyrenyl group, a biphenyl group, a heptacene group, a vinyl group, an allyl group, a triacontenyl group, a methoxy group, an ethoxy group, a triacontoxy group, a fluorine atom, a chlorine atom, a bromine atom, an iodine atom, and a thiol group.

Each of the examples of R⁰, R^{4′}, and R^{5′} includes isomers. For example, the butyl group includes a n-butyl group, an isobutyl group, a sec-butyl group, and a tert-butyl group.

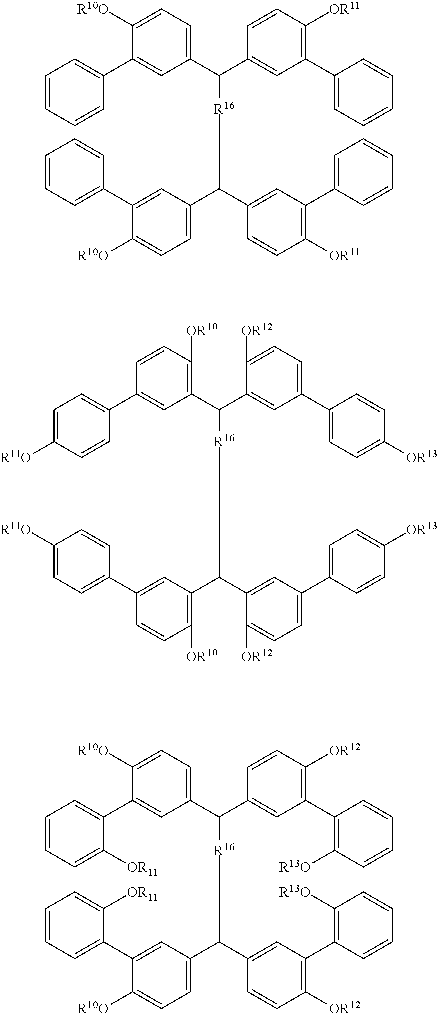

In the above formulae, R¹⁰ to R¹³ are as defined in those described in the above formula (1-2); and R¹⁶ is a linear, branched or cyclic alkylene group of 1 to 30 carbon atoms, a divalent aryl group of 6 to 30 carbon atoms, or a divalent alkenyl group of 2 to 30 carbon atoms.

Examples of R¹⁶ include a methylene group, an ethylene group, a propene group, a butene group, a pentene group, a hexene group, a heptene group, an octene group, a nonene group, a decene group, an undecene group, a dodecene group, a triacontene group, a cyclopropene group, a cyclobutene group, a cyclopentene group, a cyclohexene group, a cycloheptene group, a cyclooctene group, a cyclononene group, a cyclodecene group, a cycloundecene group, a cyclododecene group, a cyclotriacontene group, a divalent norbornyl group, a divalent adamantyl group, a divalent phenyl group, a divalent naphthyl group, a divalent anthracene group, a divalent pyrene group, a divalent biphenyl group, a divalent heptacene group, a divalent vinyl group, a divalent allyl group, and a divalent triacontenyl group.

Each of the examples of R¹⁶ includes isomers. For example, the butyl group includes a n-butyl group, an isobutyl group, a sec-butyl group, and a tert-butyl group.

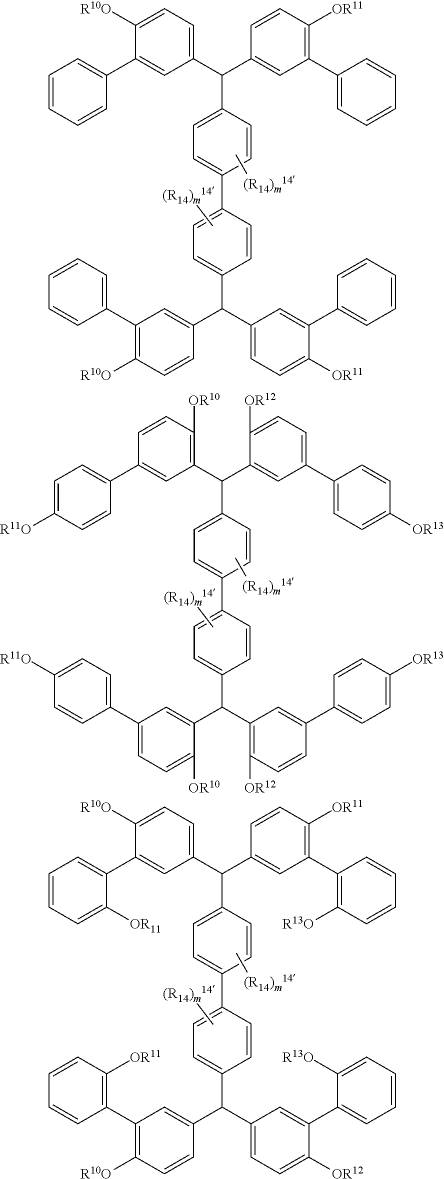

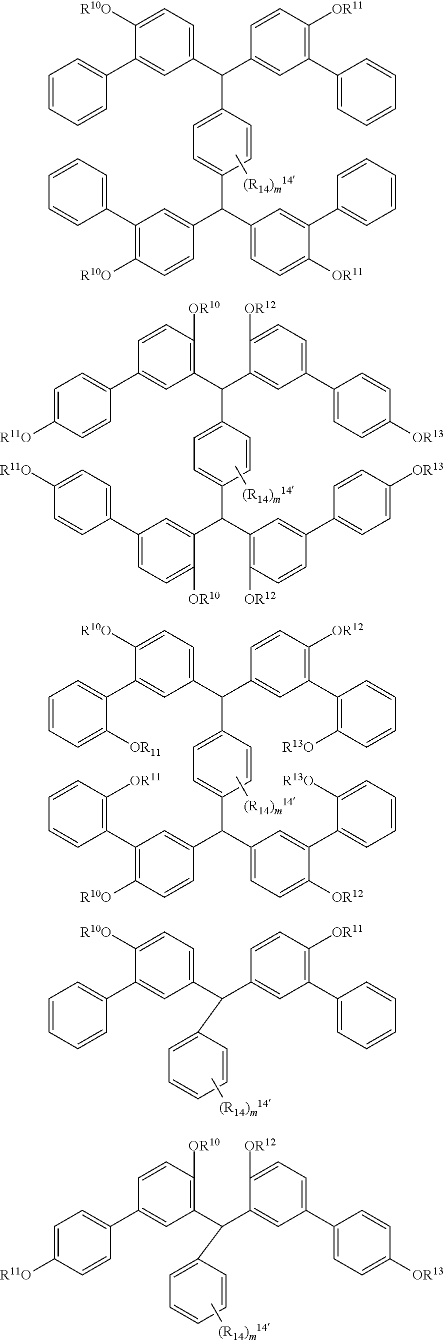

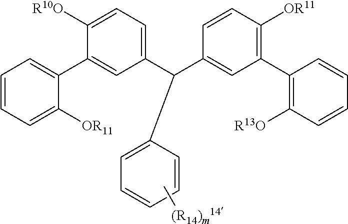

In the above formulae, R¹⁰ to R¹³ are as defined in those described in the above formula (1-2); each R⁴ is those described in the above formula (1-2); each R¹⁴ is independently a linear, branched or cyclic alkyl group of 1 to 30 carbon atoms, an aryl group of 6 to 30 carbon atoms, an alkenyl group of 2 to 30 carbon atoms, an alkoxy group of 1 to 30 carbon atoms, a halogen atom, or a thiol group; m¹⁴ is an integer of 0 to 5; m^{14′} is an integer of 0 to 4; and m¹⁴ is an integer of 0 to 5.

Examples of R¹⁴ include a methyl group, an ethyl group, a propyl group, a butyl group, a pentyl group, a hexyl group, a heptyl group, an octyl group, a nonyl group, a decyl group, an undecyl group, a dodecyl group, a triacontyl group, a cyclopropyl group, a cyclobutyl group, a cyclopentyl group, a cyclohexyl group, a cycloheptyl group, a cyclooctyl group, a cyclononyl group, a cyclodecyl group, a cycloundecyl group, a cyclododecyl group, a cyclotriacontyl group, a norbornyl group, an adamantyl group, a phenyl group, a naphthyl group, an anthracene group, a pyrenyl group, a biphenyl group, a heptacene group, a vinyl group, an allyl group, a triacontenyl group, a methoxy group, an ethoxy group, a triacontoxy group, a fluorine atom, a chlorine atom, a bromine atom, an iodine atom, and a thiol group.

Each of the examples of R¹⁴ includes isomers. For example, the butyl group includes a n-butyl group, an isobutyl group, a sec-butyl group, and a tert-butyl group.

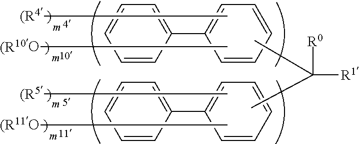

In the above formula R⁰, R^{4′}, R^{5′}, m^{4′}, m^{5′}, m^{10′}, and m^{11′} are as defined above; and R^{1′} is a group of 1 to 60 carbon atoms.

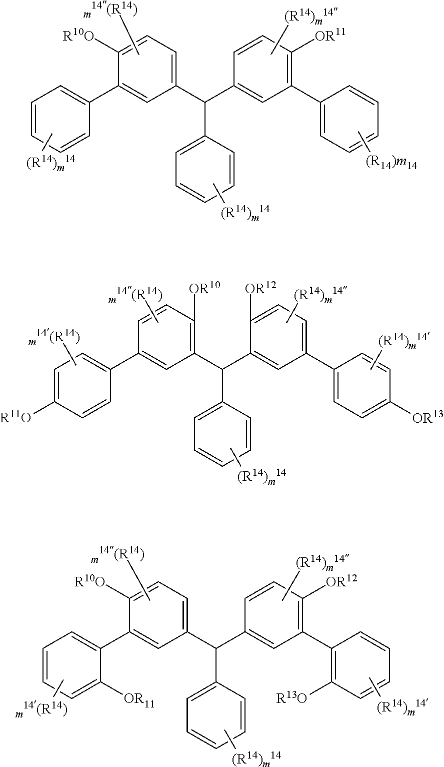

In the above formulae, R¹⁰ to R¹³ are as defined in those described in the above formula (1-2); each R¹⁴ is independently a linear, branched or cyclic alkyl group of 1 to 30 carbon atoms, an aryl group of 6 to 30 carbon atoms, an alkenyl group of 2 to 30 carbon atoms, an alkoxy group of 1 to 30 carbon atoms, a halogen atom, or a thiol group; m¹⁴ is an integer of 0 to 5; m^{14′} is an integer of 0 to 4; and m^{14″} is an integer of 0 to 3.

Examples of R¹⁴ include a methyl group, an ethyl group, a propyl group, a butyl group, a pentyl group, a hexyl group, a heptyl group, an octyl group, a nonyl group, a decyl group, an undecyl group, a dodecyl group, a triacontyl group, a cyclopropyl group, a cyclobutyl group, a cyclopentyl group, a cyclohexyl group, a cycloheptyl group, a cyclooctyl group, a cyclononyl group, a cyclodecyl group, a cycloundecyl group, a cyclododecyl group, a cyclotriacontyl group, a norbornyl group, an adamantyl group, a phenyl group, a naphthyl group, an anthracene group, a pyrenyl group, a biphenyl group, a heptacene group, a vinyl group, an allyl group, a triacontenyl group, a methoxy group, an ethoxy group, a triacontoxy group, a fluorine atom, a chlorine atom, a bromine atom, an iodine atom, and a thiol group.

Each of the examples of R¹⁴ includes isomers. For example, the butyl group includes a n-butyl group, an isobutyl group, a sec-butyl group, and a tert-butyl group.

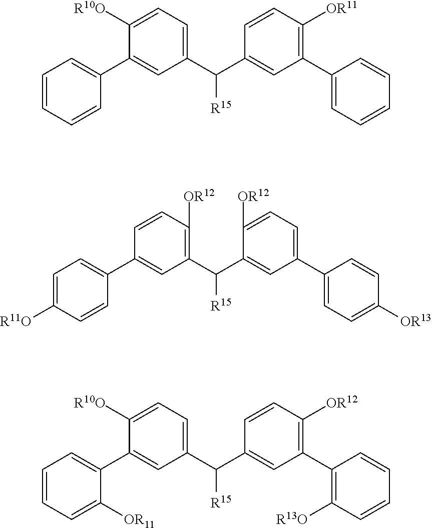

In the above formulae, R¹⁰ to R¹³ are as defined in those described in the above formula (1-2); and R¹⁵ is a linear, branched or cyclic alkyl group of 1 to 30 carbon atoms, an aryl group of 6 to 30 carbon atoms, an alkenyl group of 2 to 30 carbon atoms, an alkoxy group of 1 to 30 carbon atoms, a halogen atom, or a thiol group.

Examples of R¹⁵ include a methyl group, an ethyl group, a propyl group, a butyl group, a pentyl group, a hexyl group, a heptyl group, an octyl group, a nonyl group, a decyl group, an undecyl group, a dodecyl group, a triacontyl group, a cyclopropyl group, a cyclobutyl group, a cyclopentyl group, a cyclohexyl group, a cycloheptyl group, a cyclooctyl group, a cyclononyl group, a cyclodecyl group, a cycloundecyl group, a cyclododecyl group, a cyclotriacontyl group, a norbornyl group, an adamantyl group, a phenyl group, a naphthyl group, an anthracene group, a pyrenyl group, a biphenyl group, a heptacene group, a vinyl group, an allyl group, a triacontenyl group, a methoxy group, an ethoxy group, a triacontoxy group, a fluorine atom, a chlorine atom, a bromine atom, an iodine atom, and a thiol group.

Each of the examples of R¹⁵ includes isomers. For example, the butyl group includes a n-butyl group, an isobutyl group, a sec-butyl group, and a tert-butyl group.

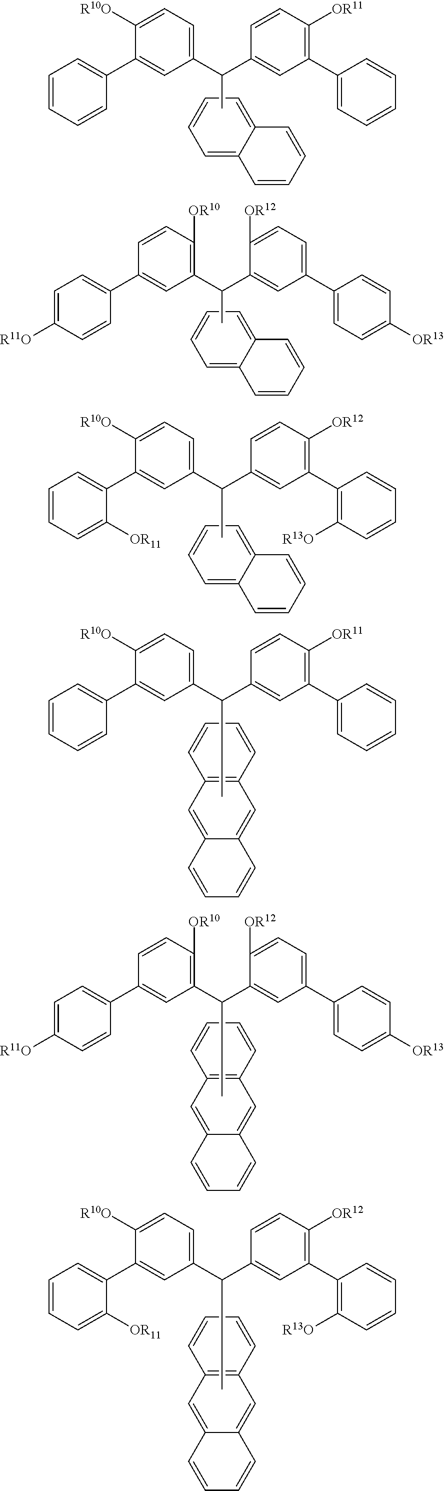

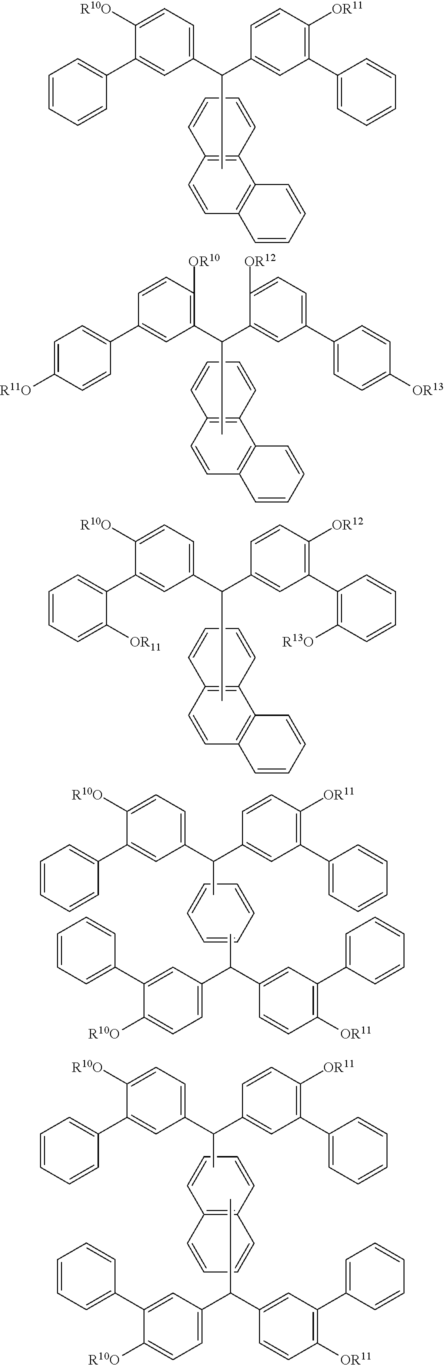

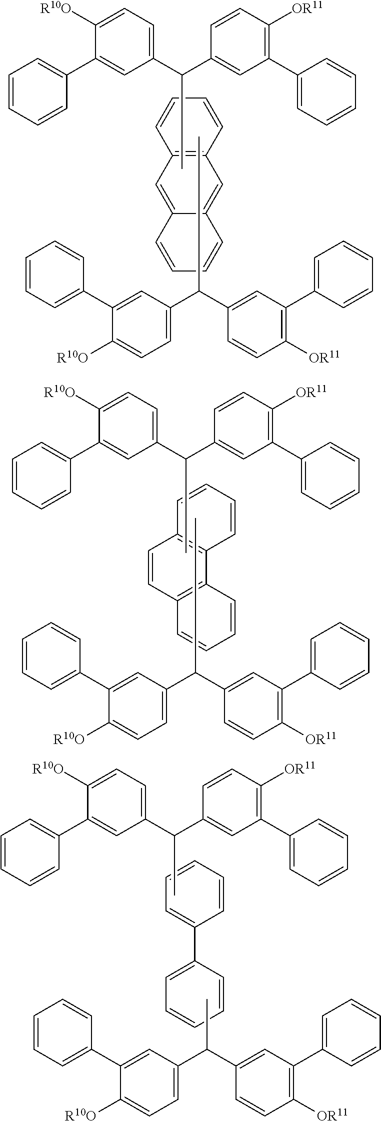

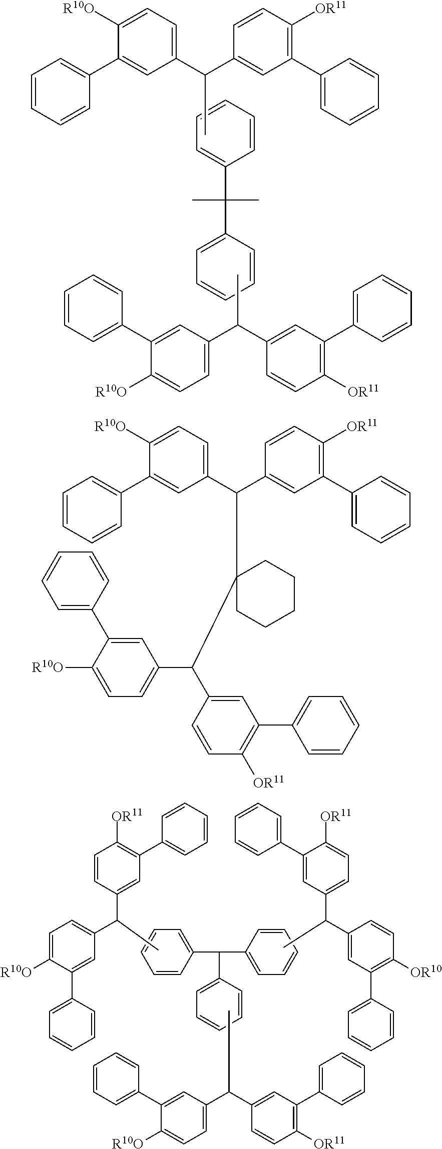

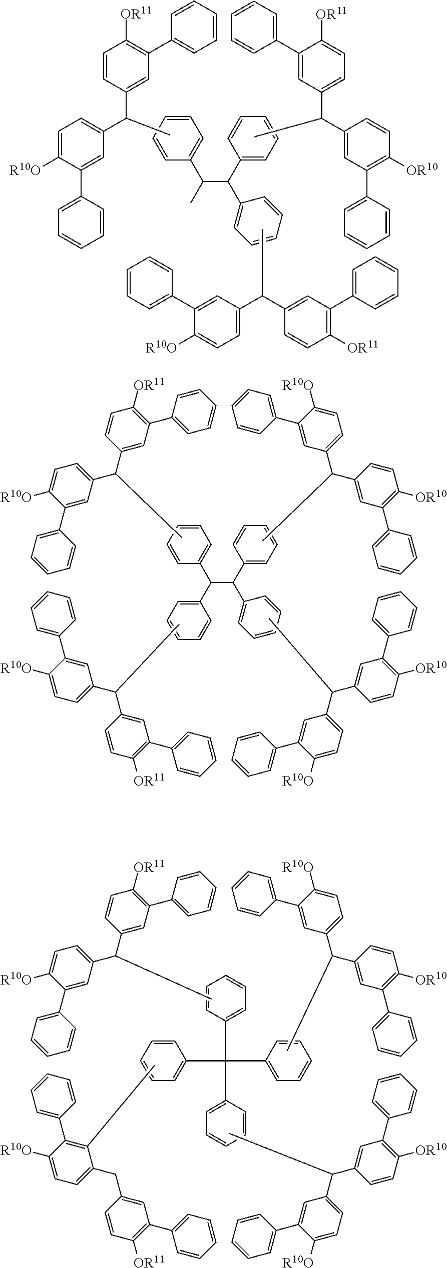

In the above formulae, R¹⁰ to R¹³ are as defined in those described in the above formula (1-2).

The following compounds are further preferable from the viewpoint of the availability of raw materials:

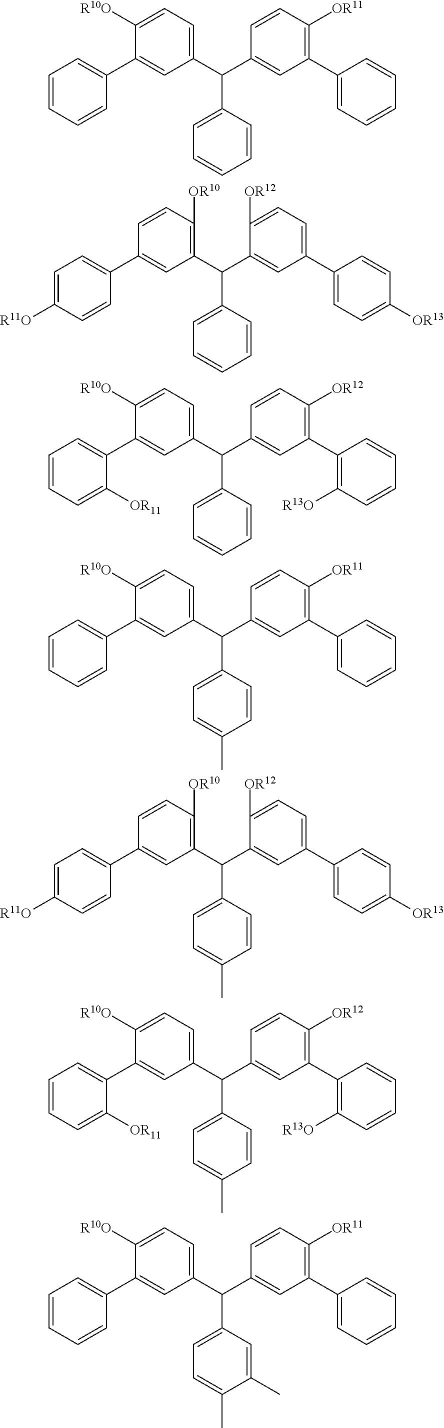

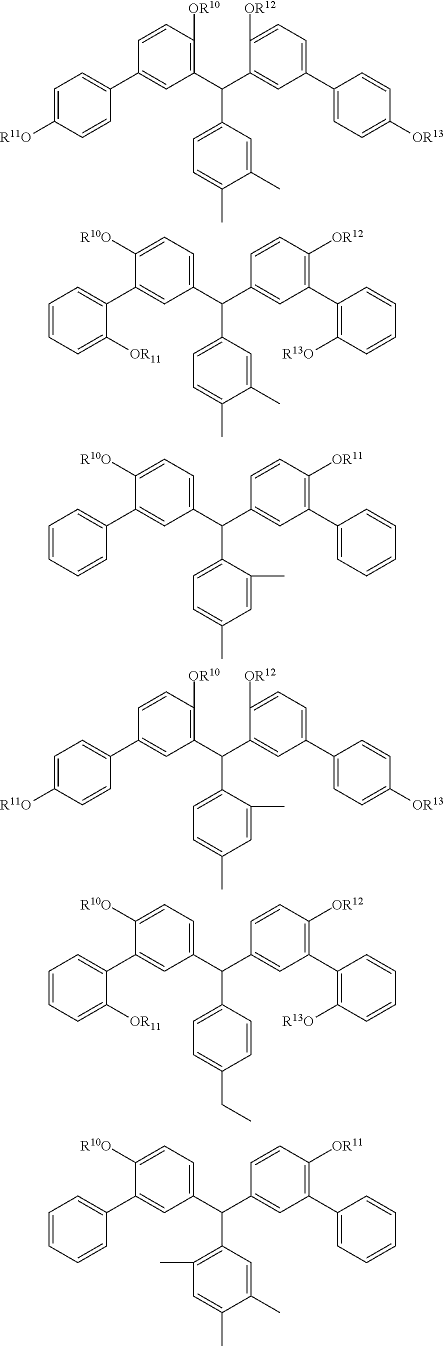

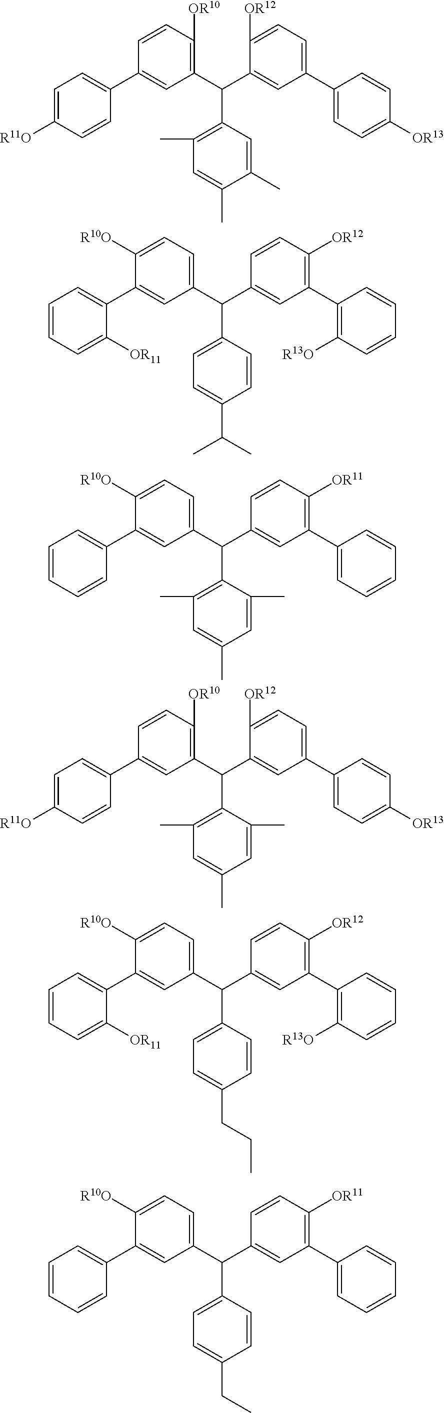

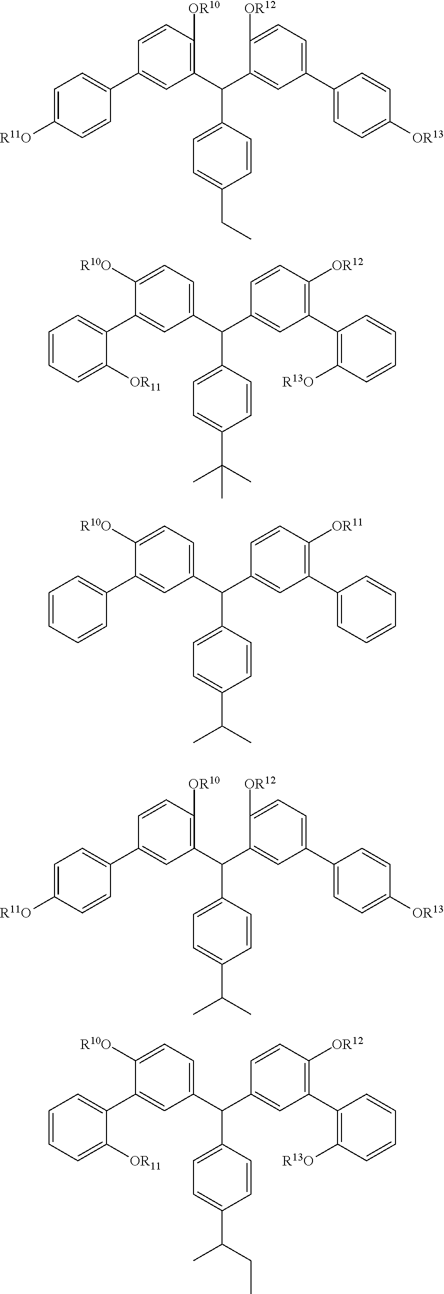

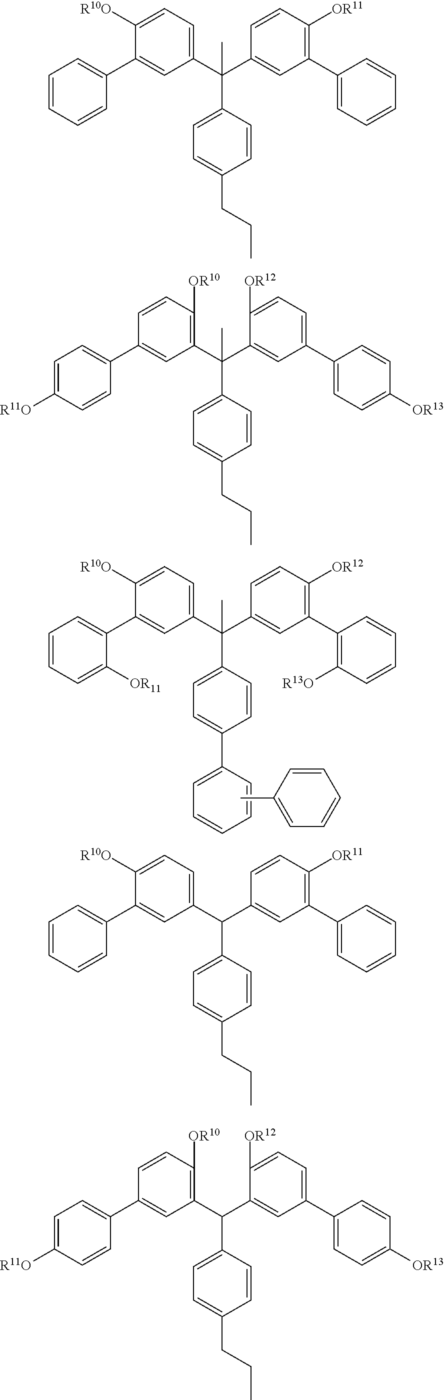

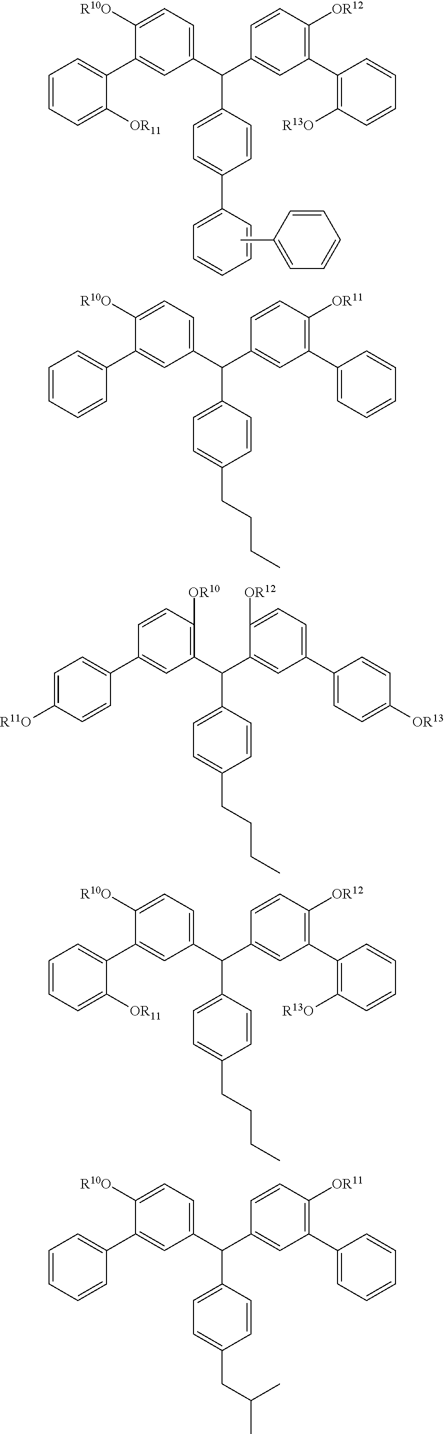

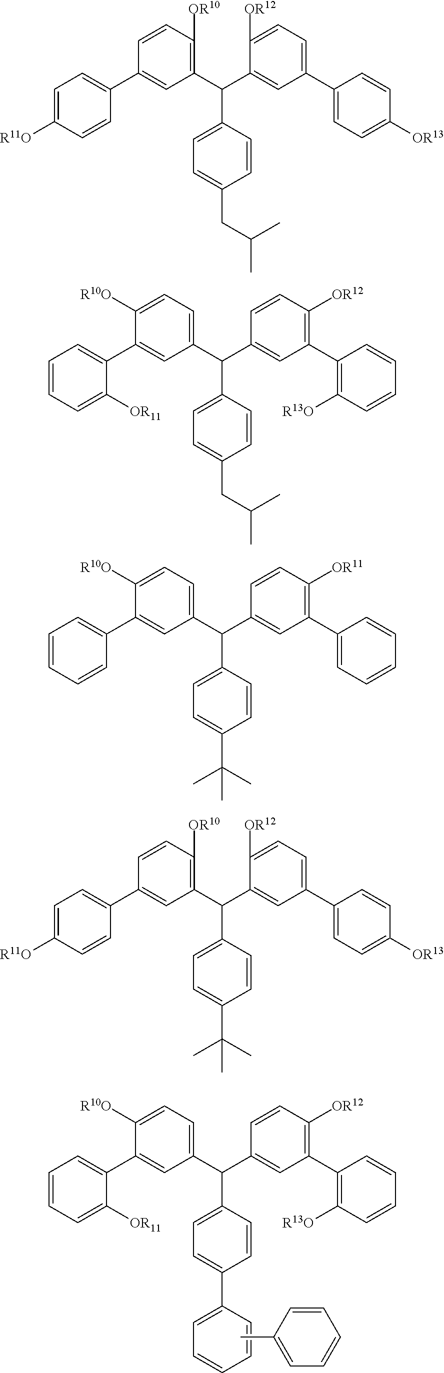

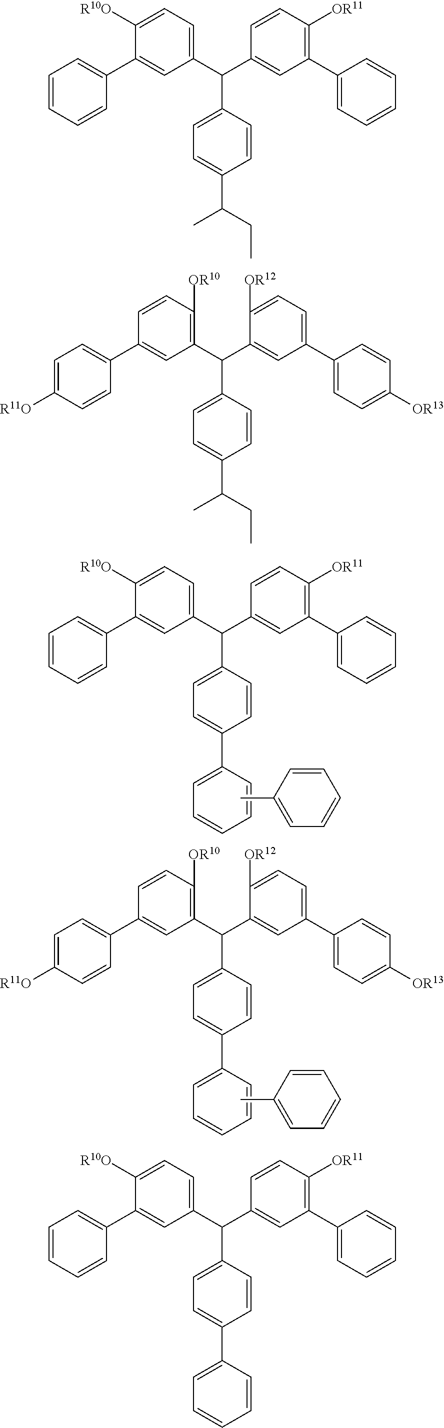

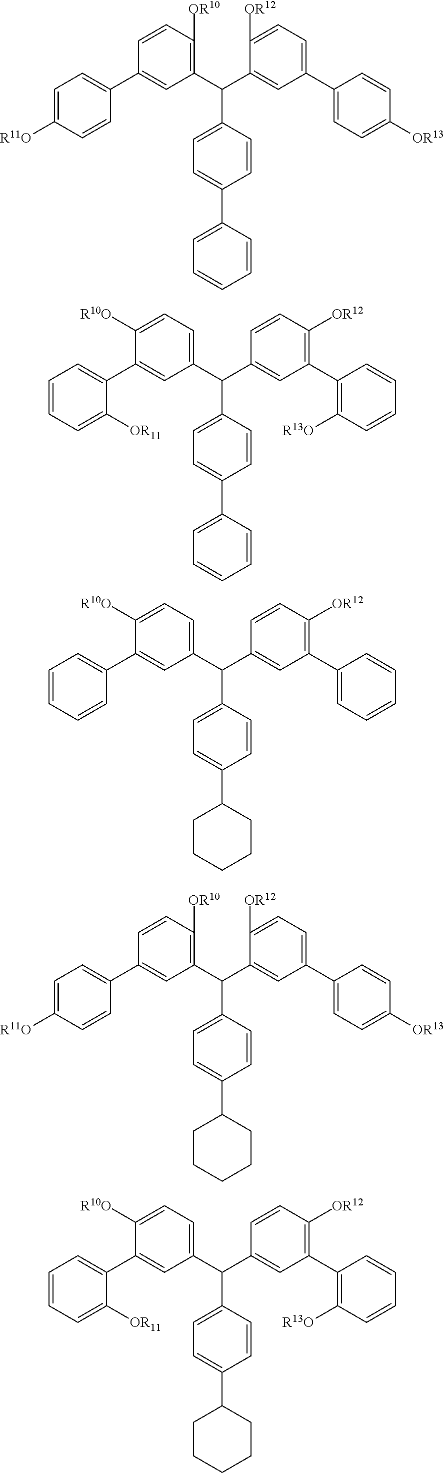

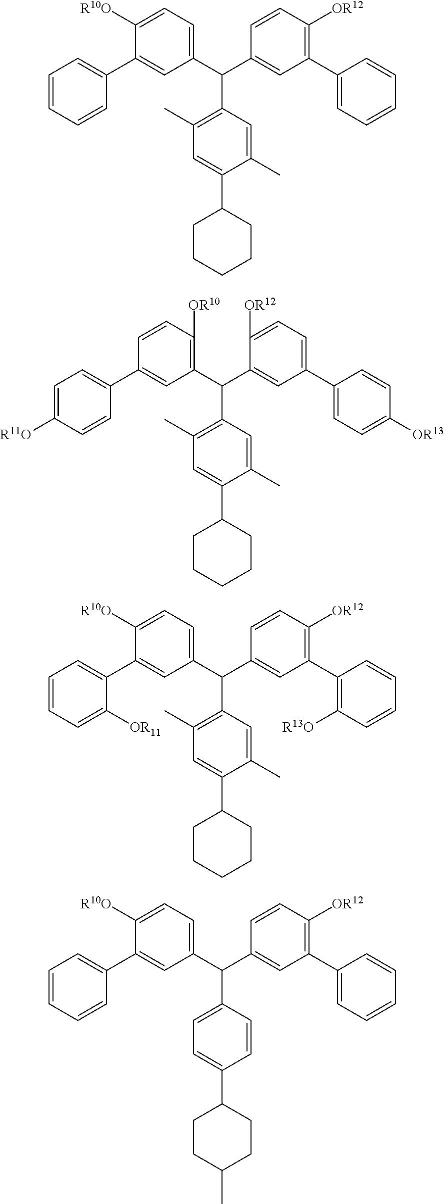

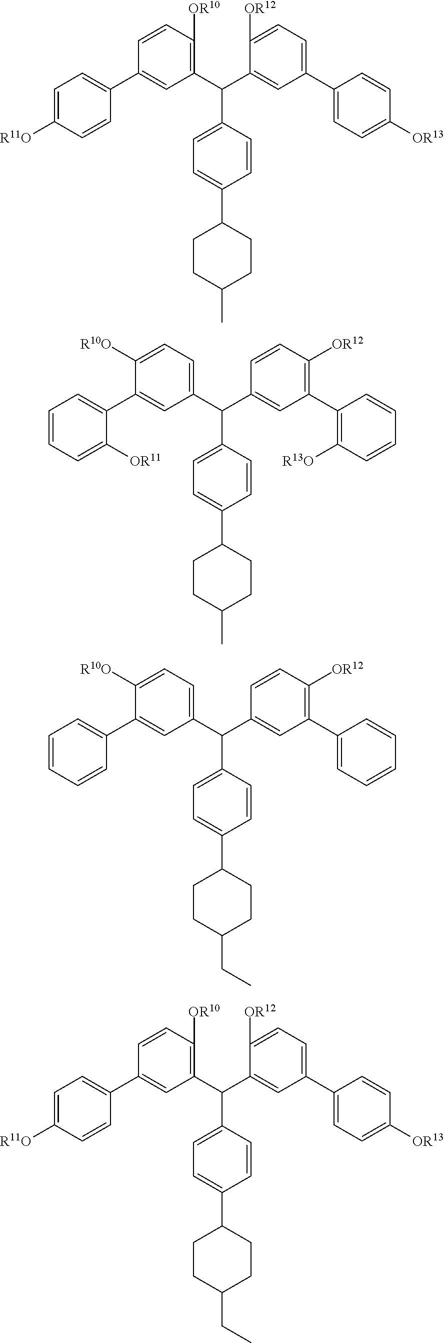

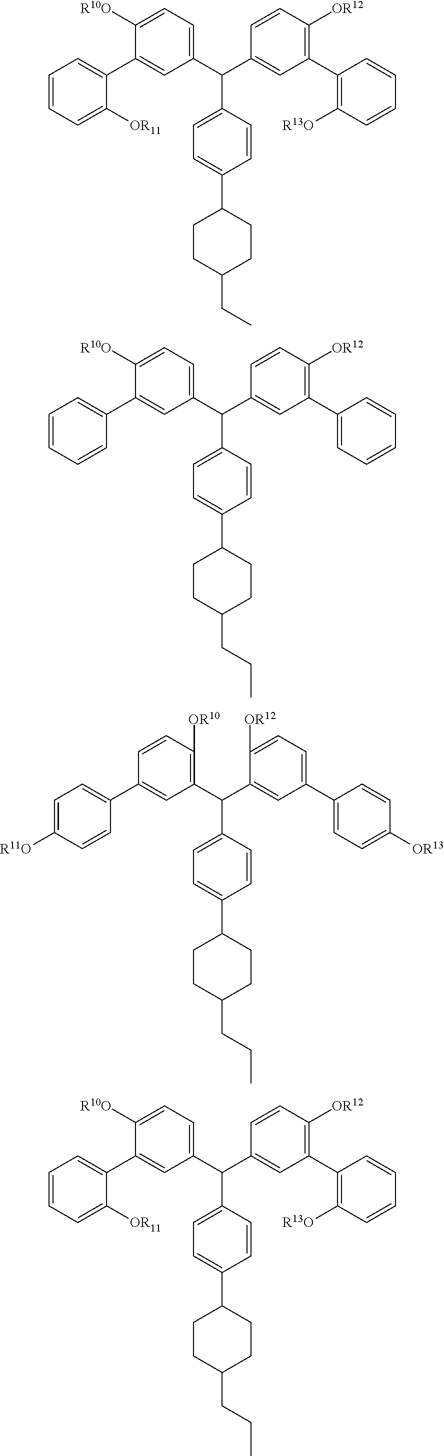

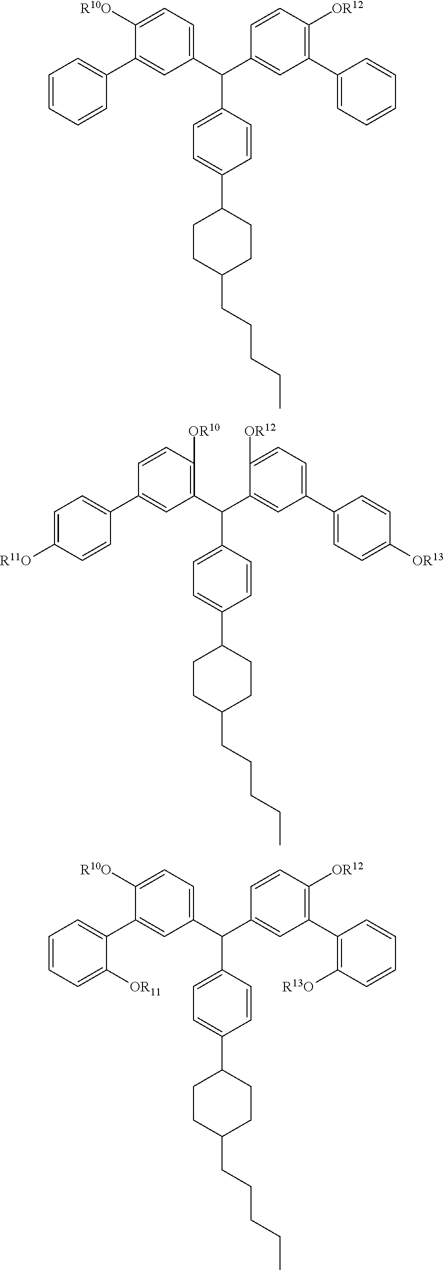

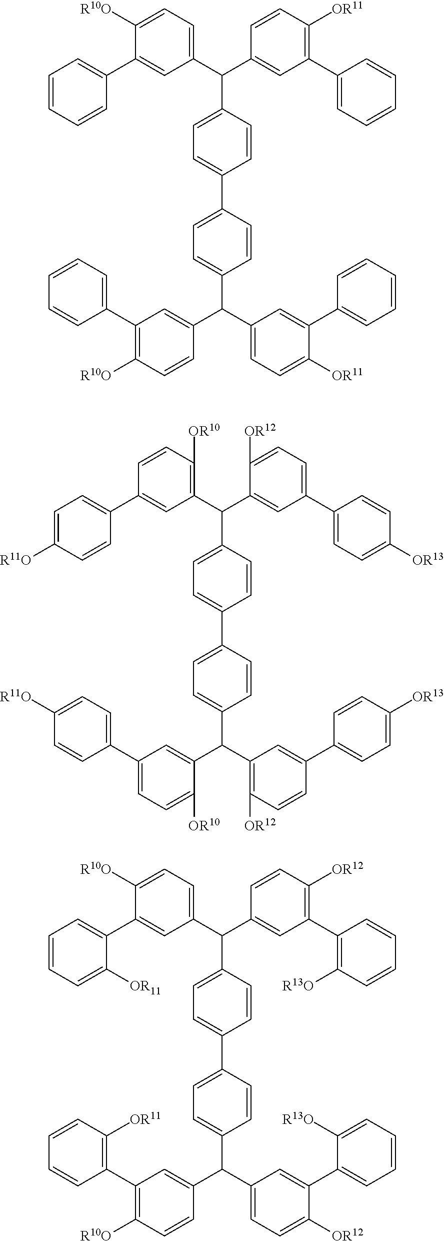

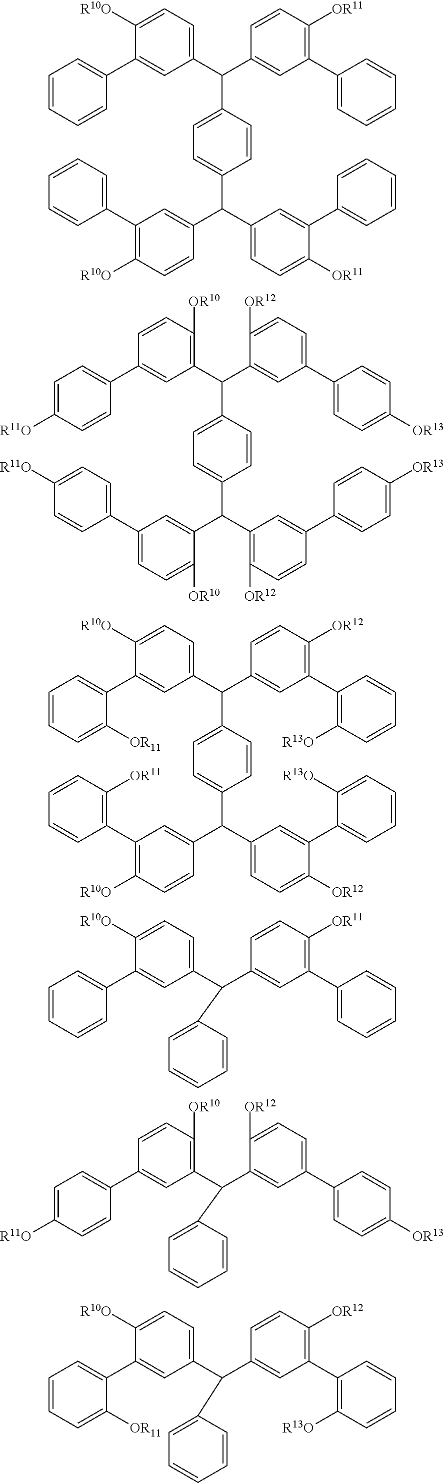

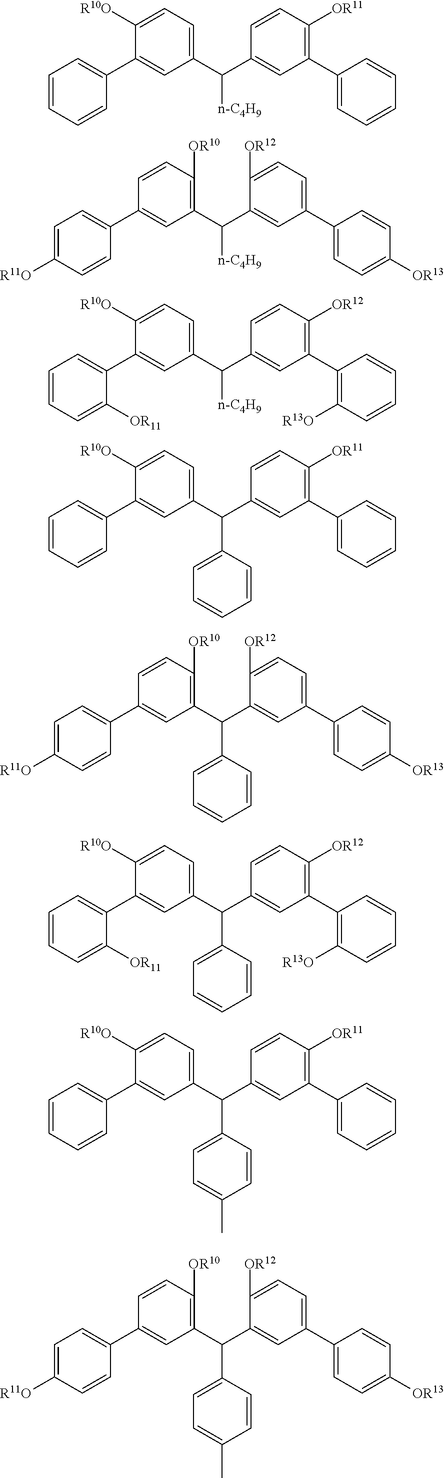

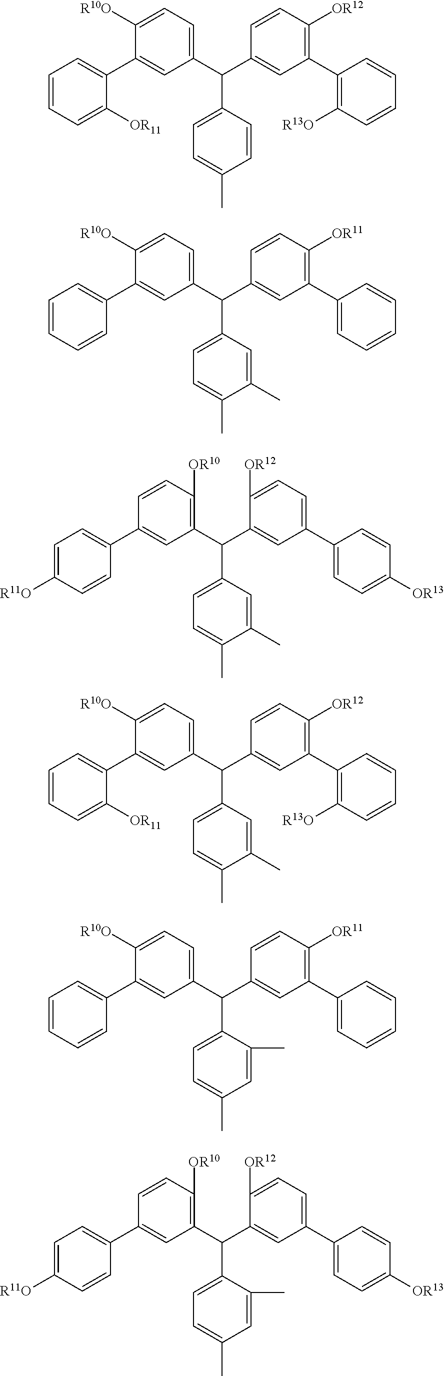

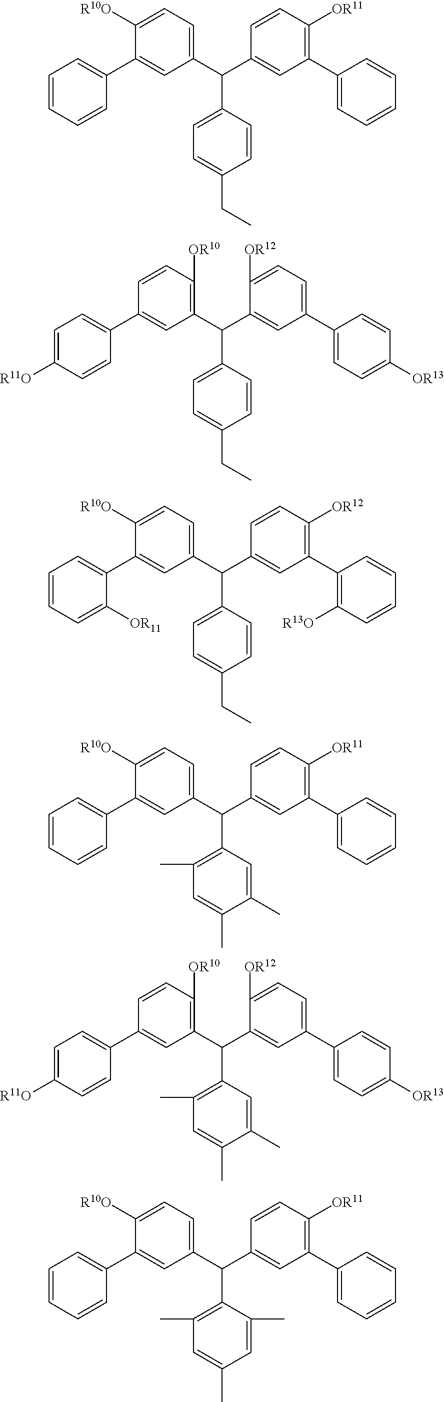

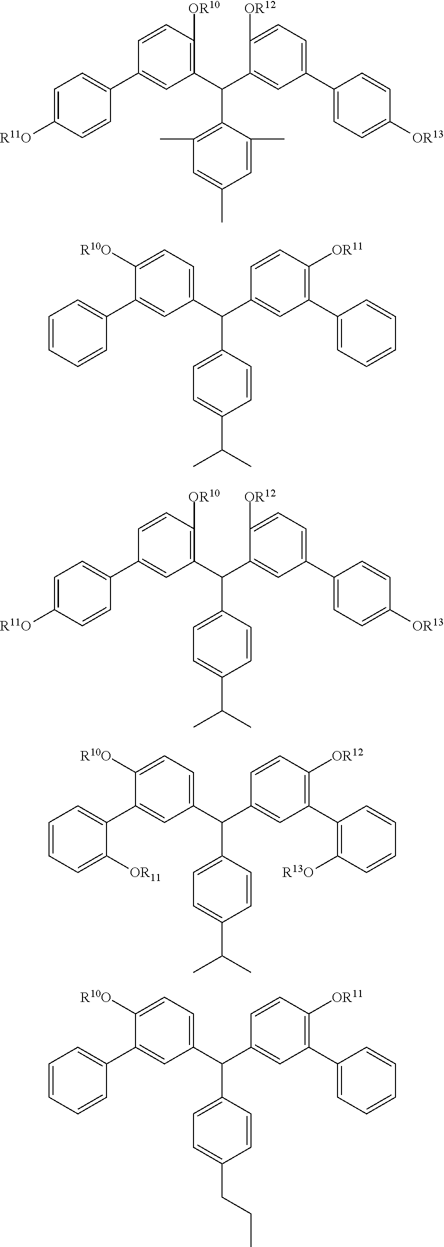

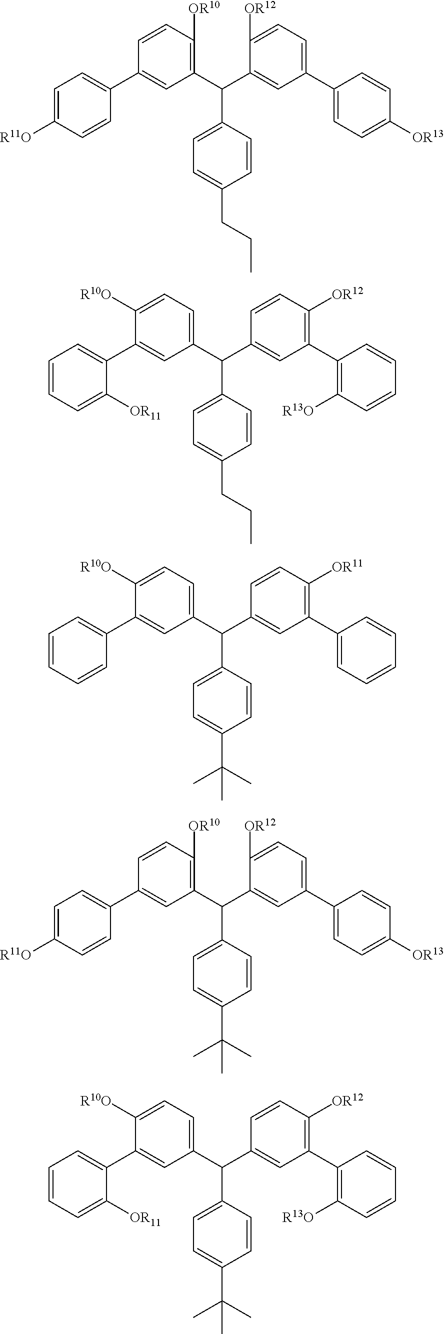

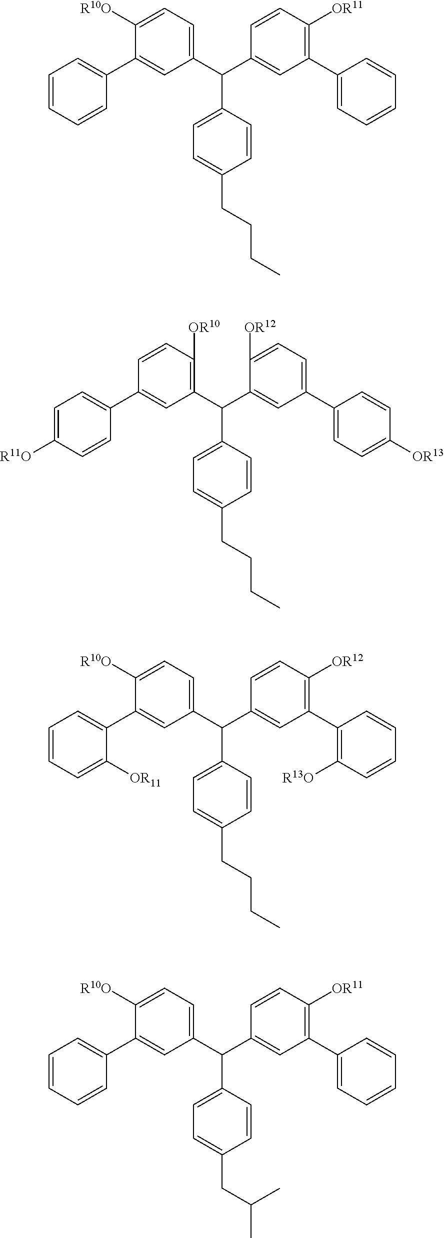

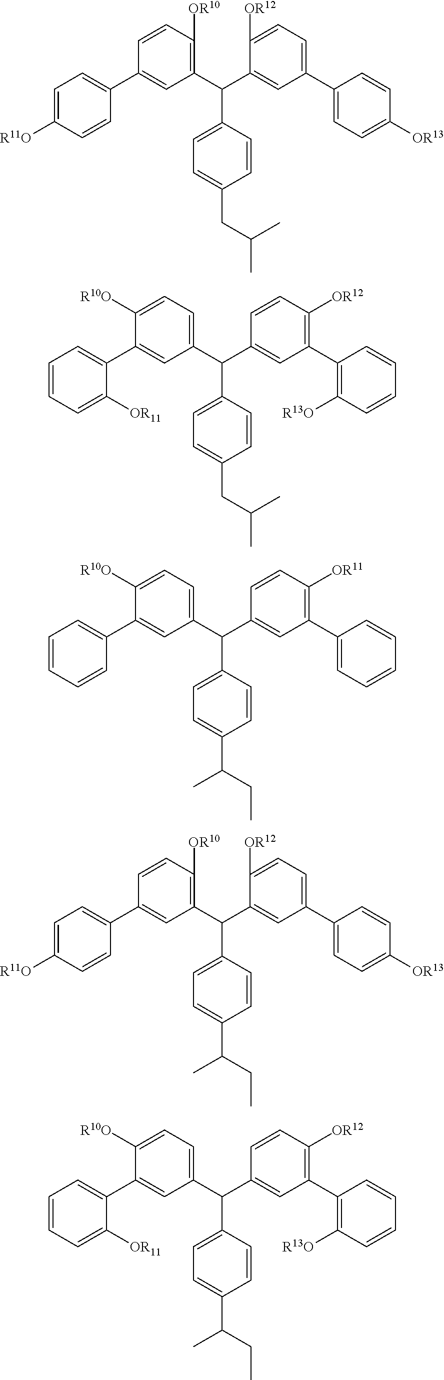

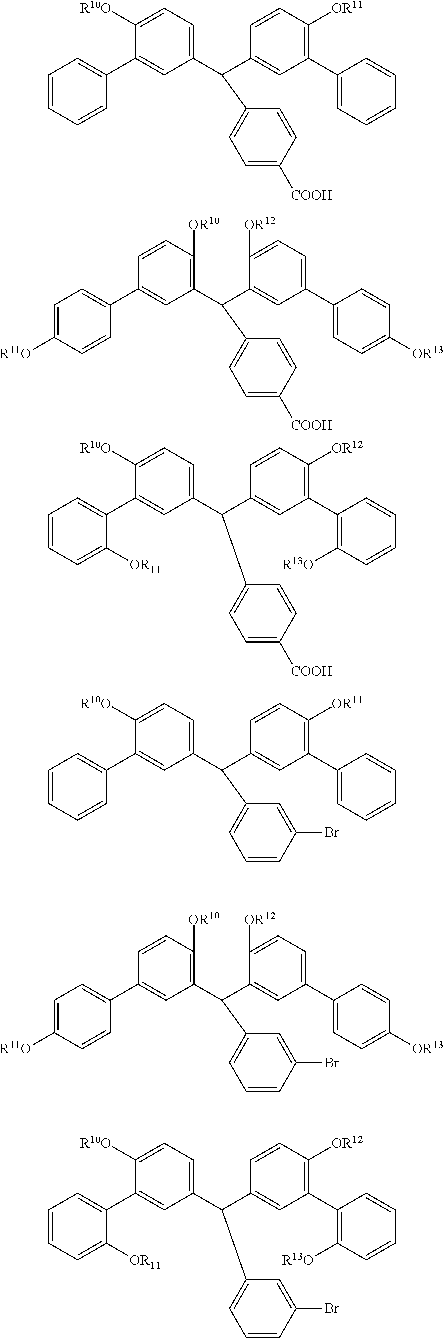

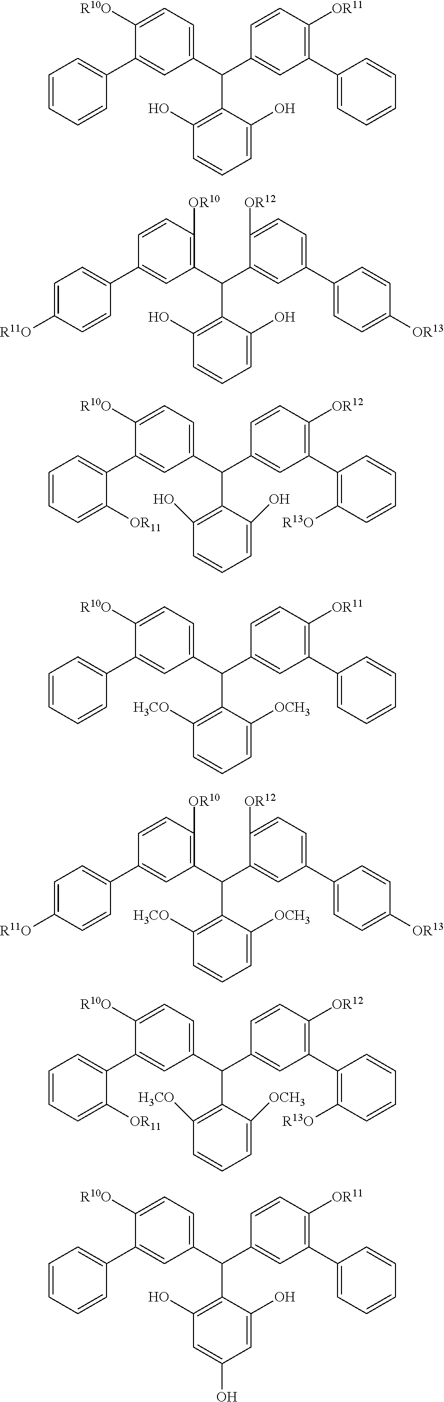

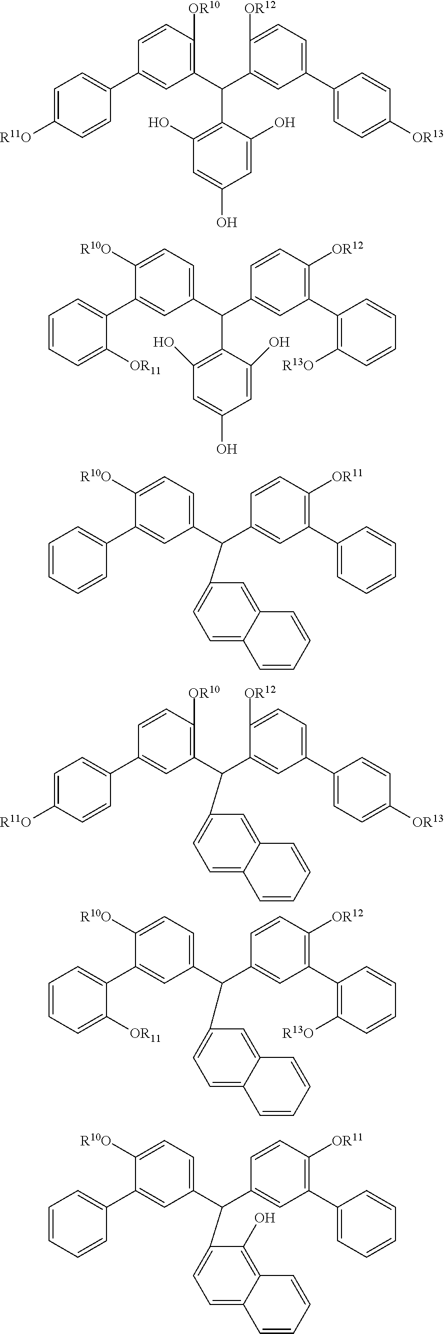

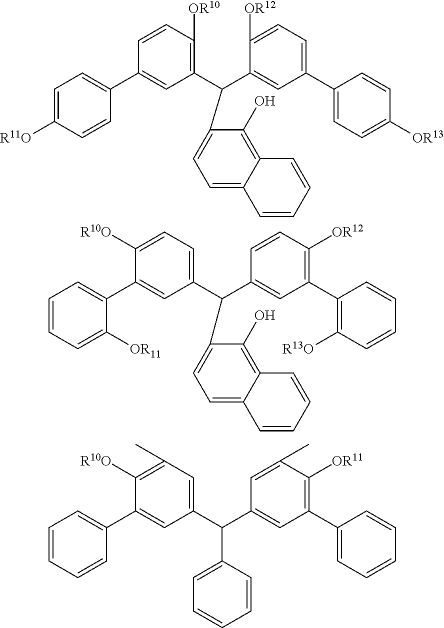

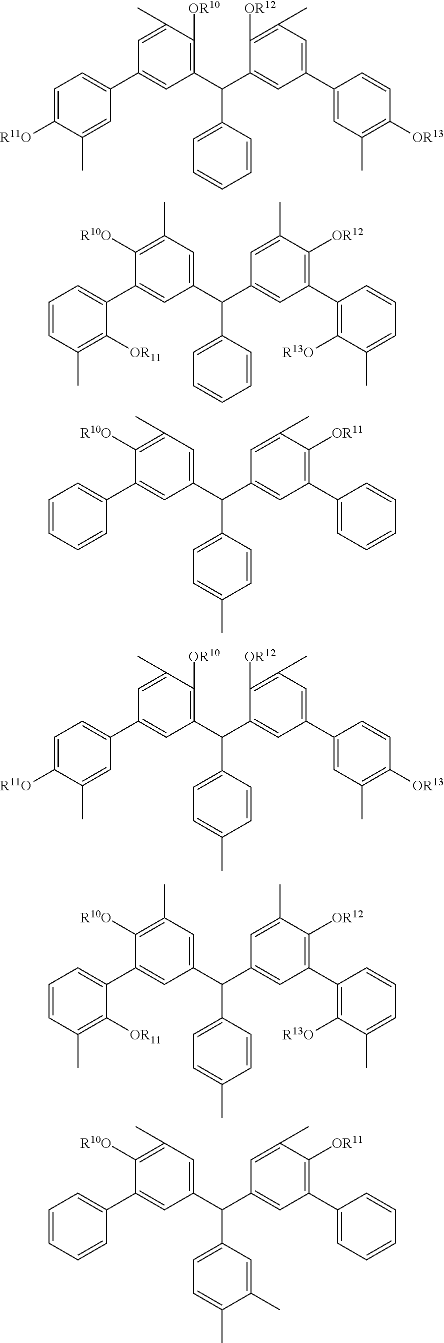

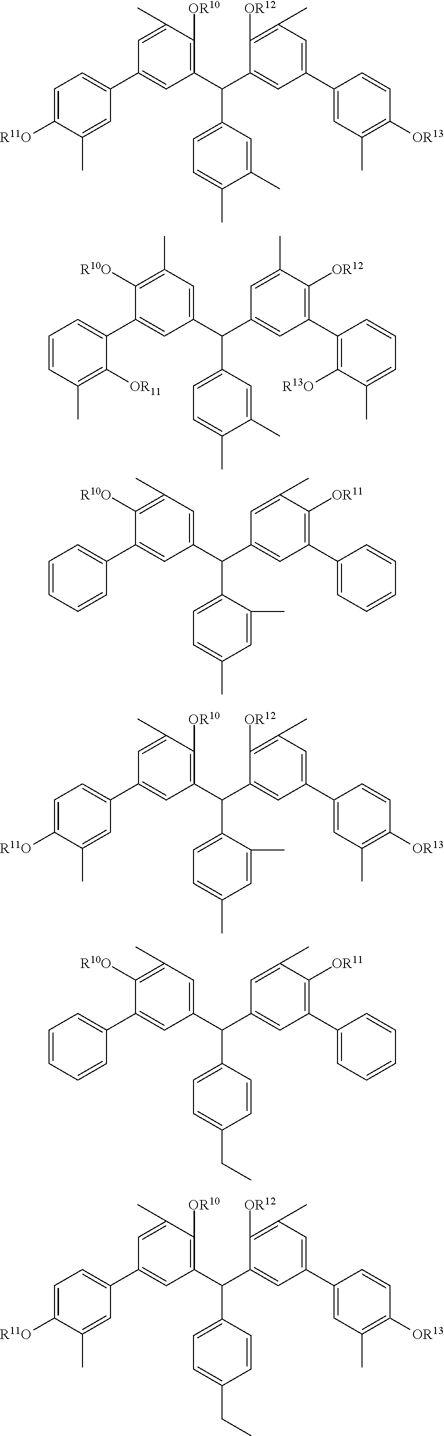

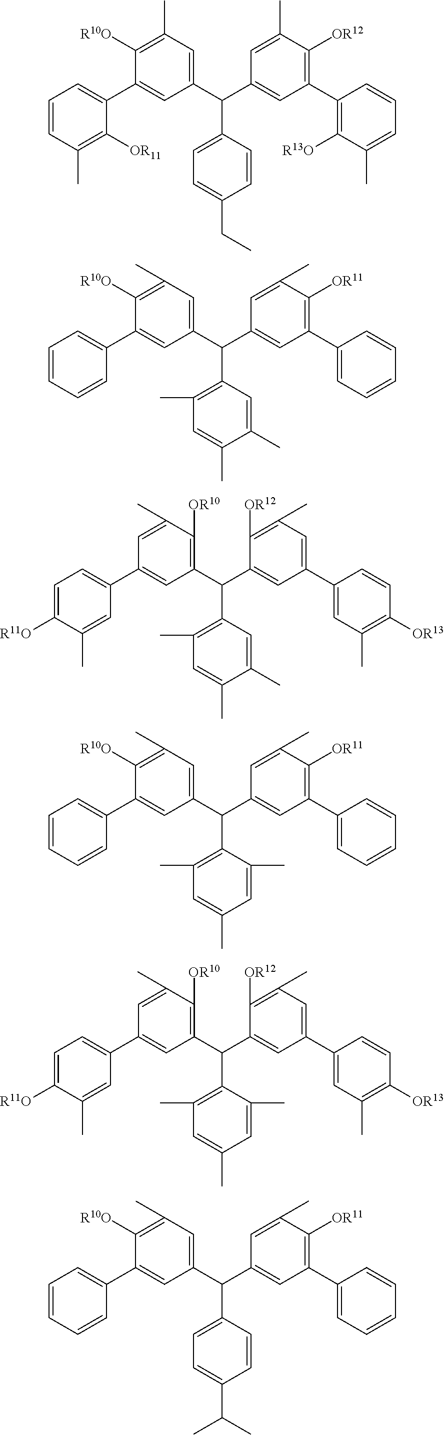

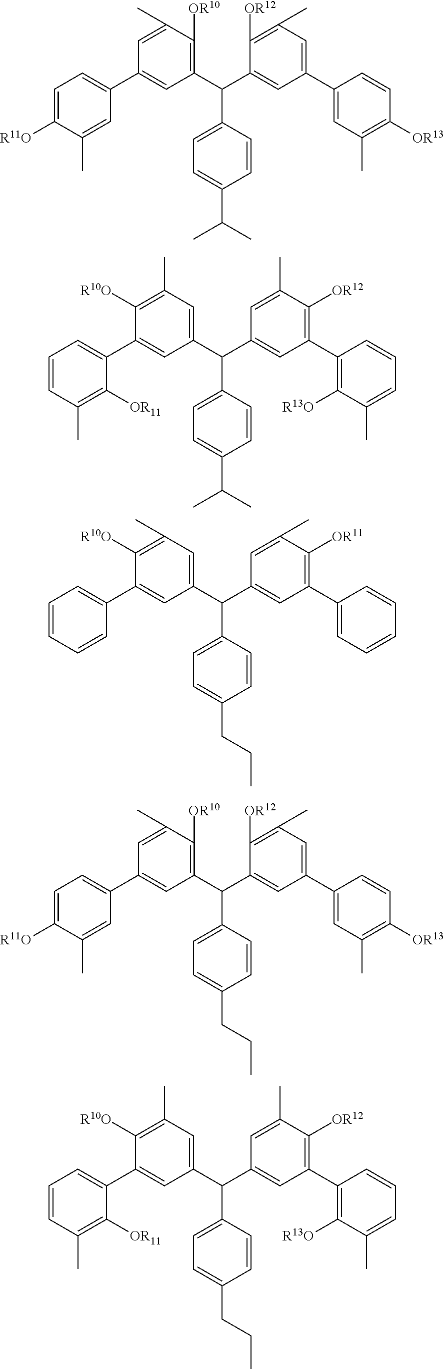

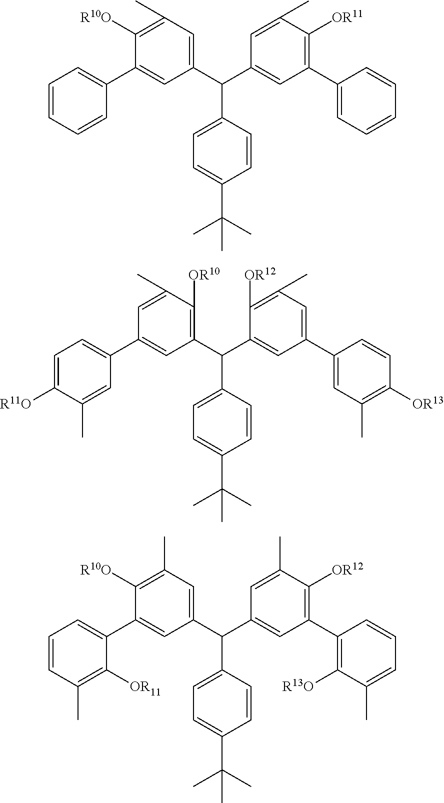

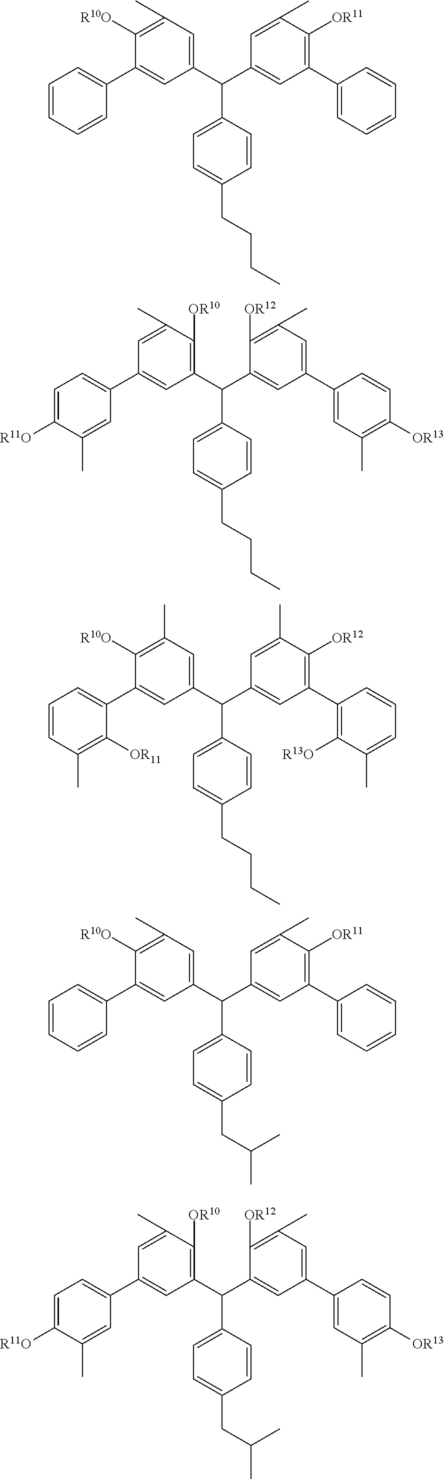

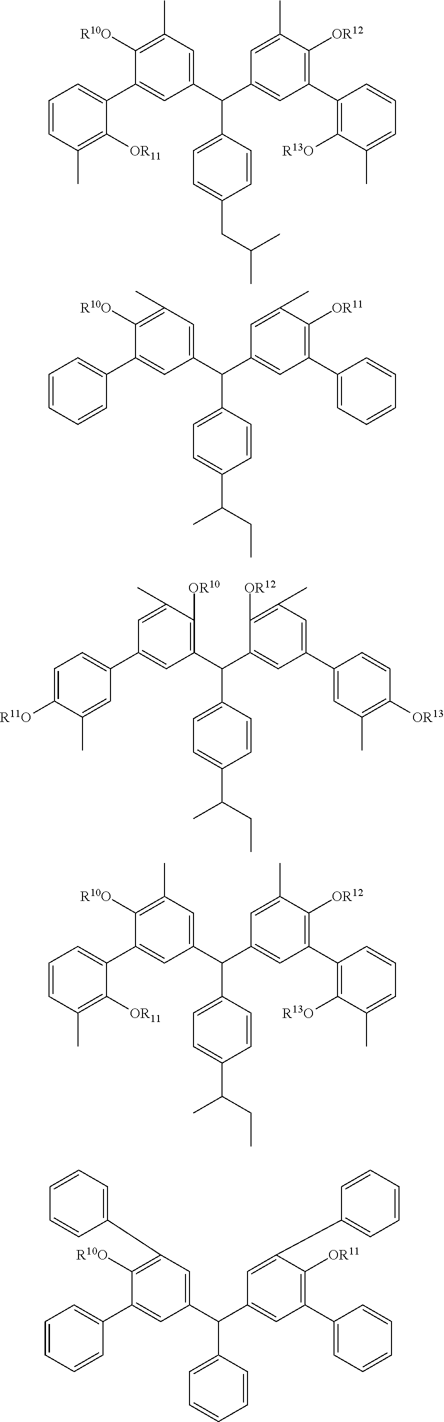

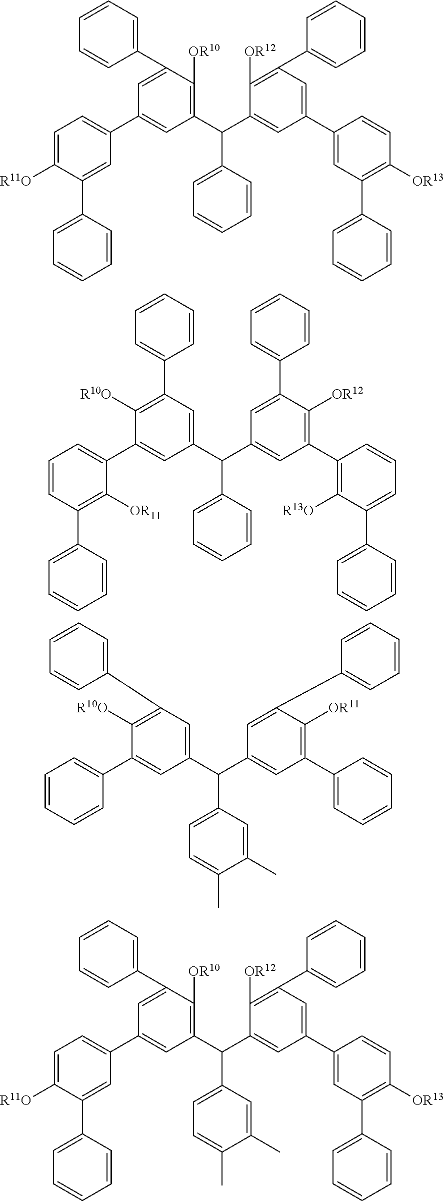

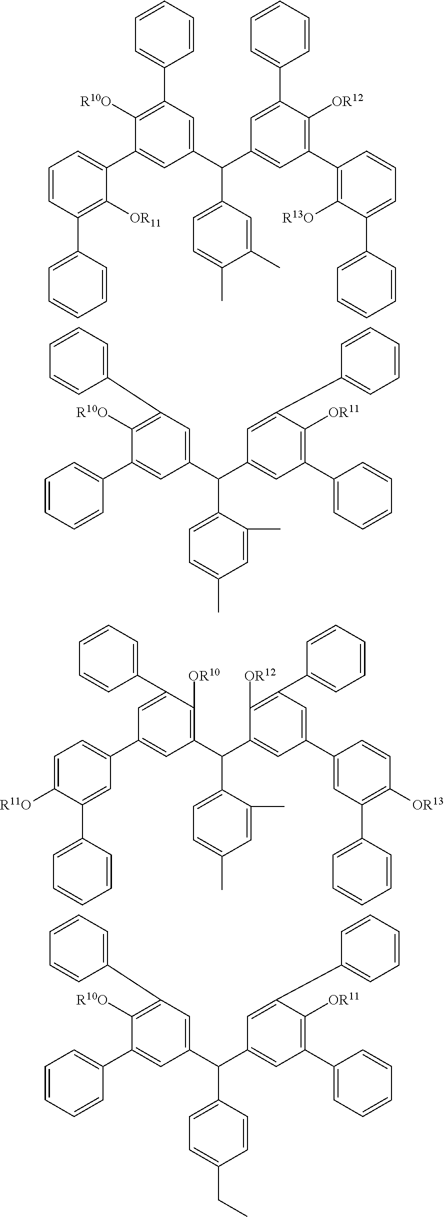

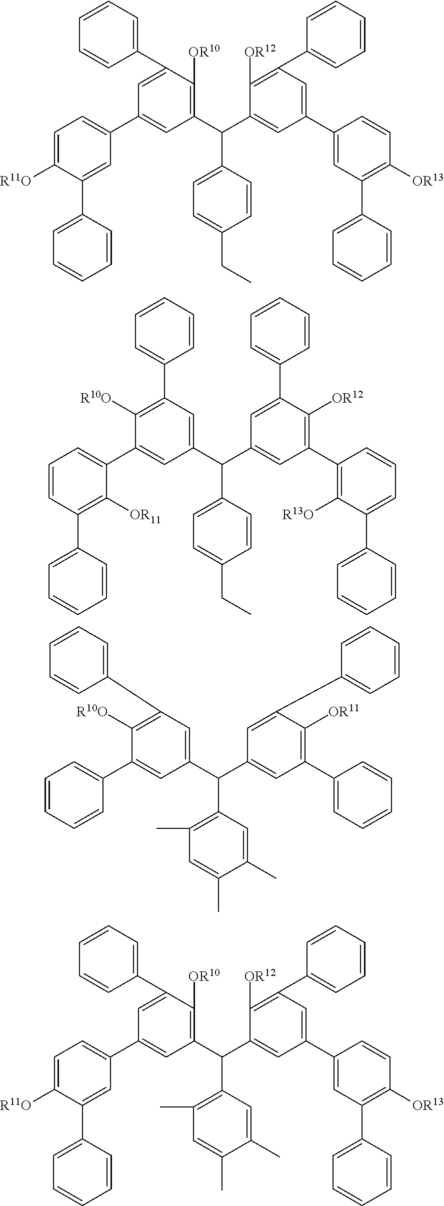

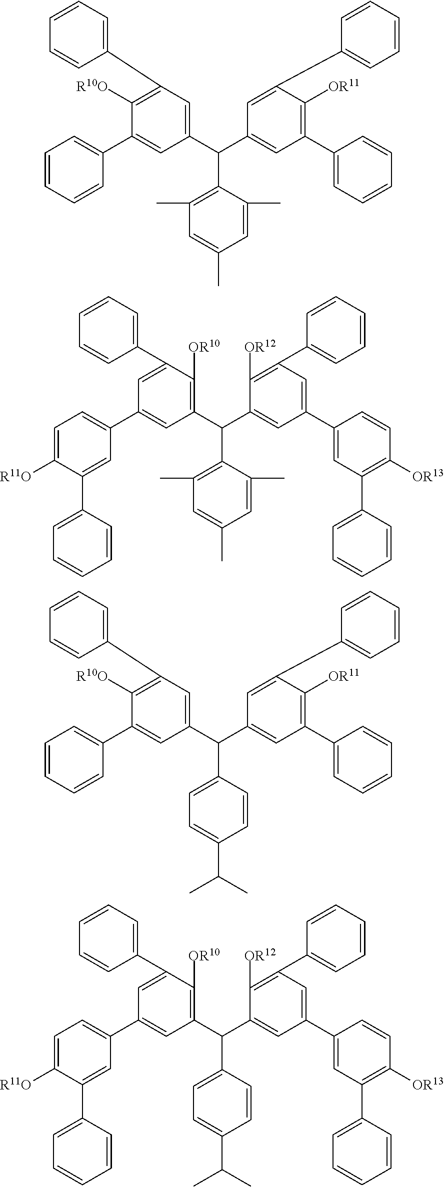

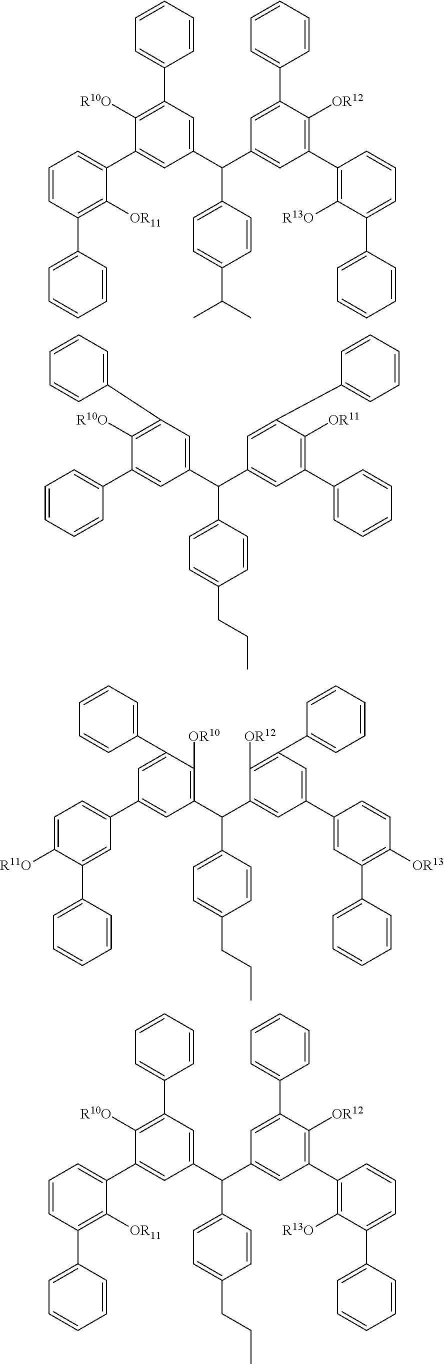

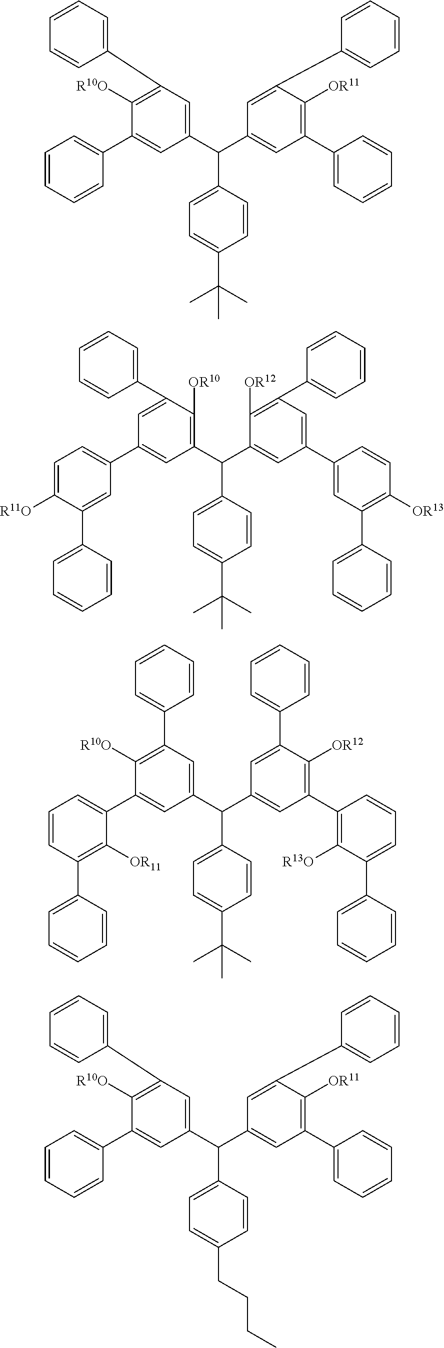

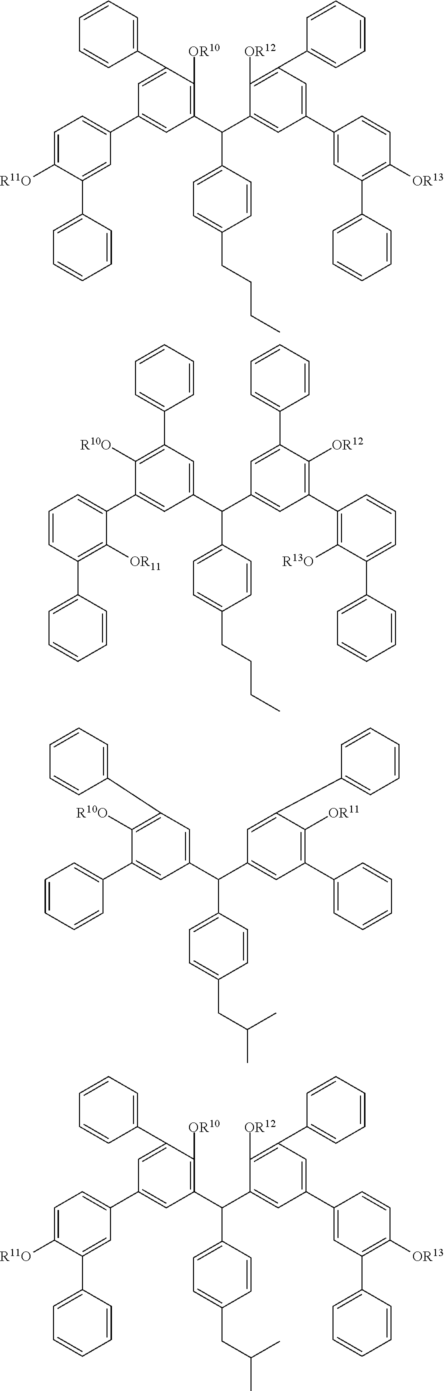

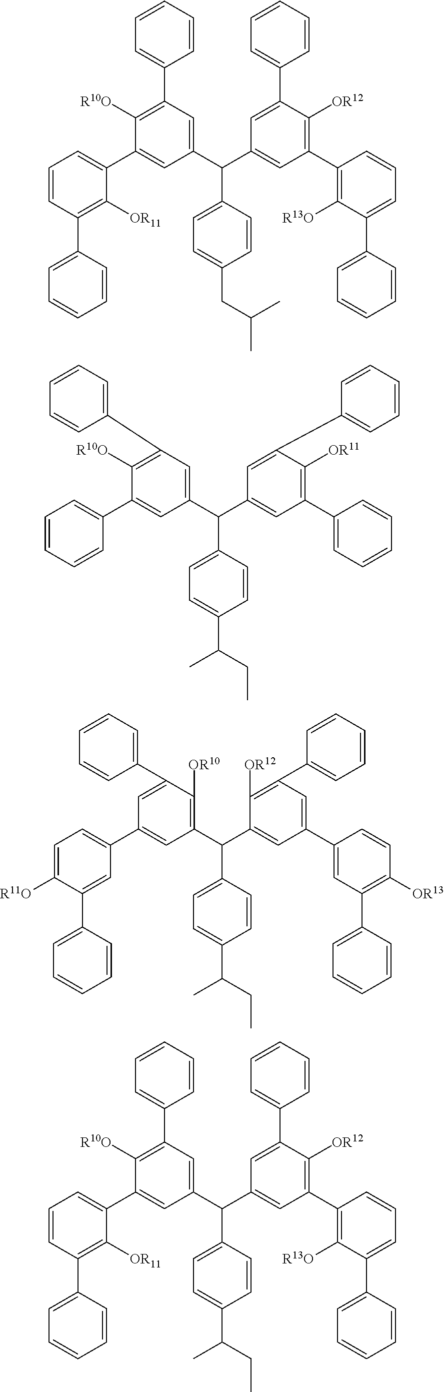

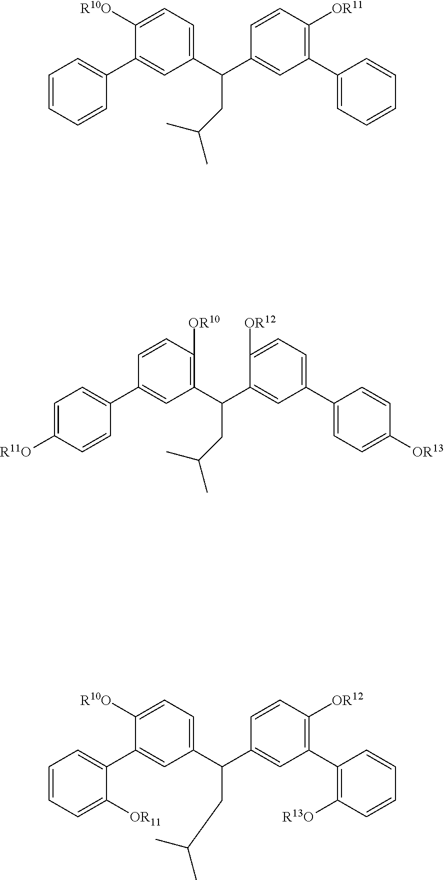

In the above formulae, R¹⁰ to R¹³ are as defined in those described in the above formula (1-2)

The compounds represented by the above formulae preferably have the following structures from the viewpoint of etching resistance:

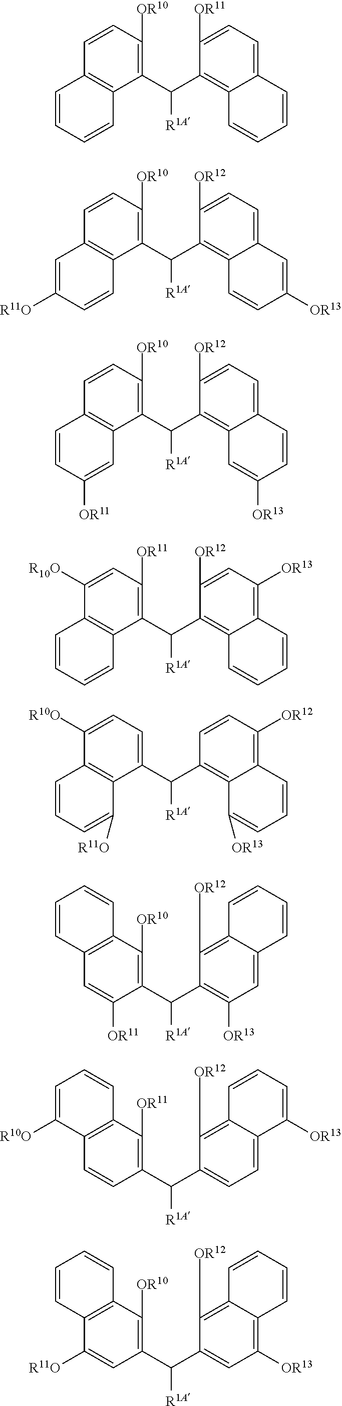

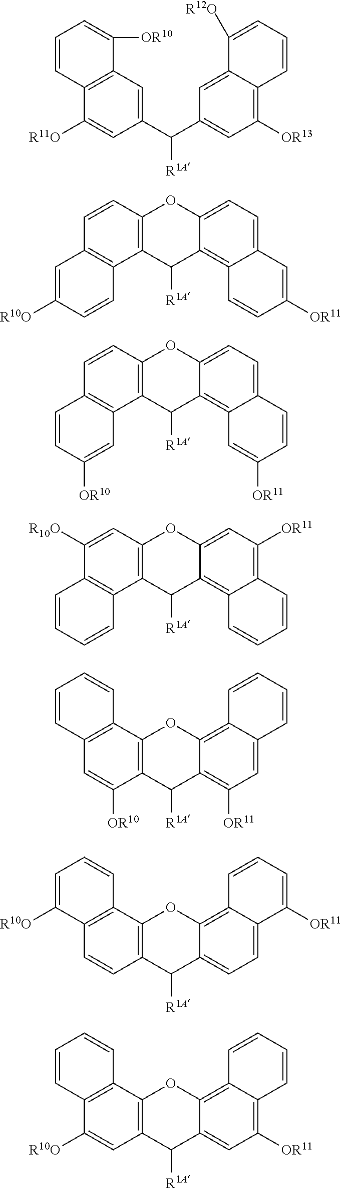

In the above formulae, R^{1A′} is as defined in R^{Z}; and R¹⁰ to R¹³ are as defined in those described in the above formula (1-2).

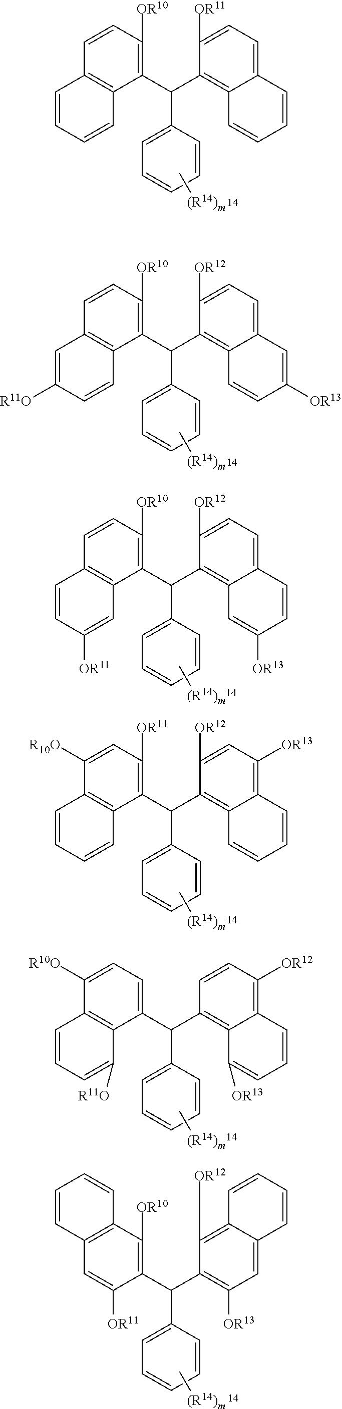

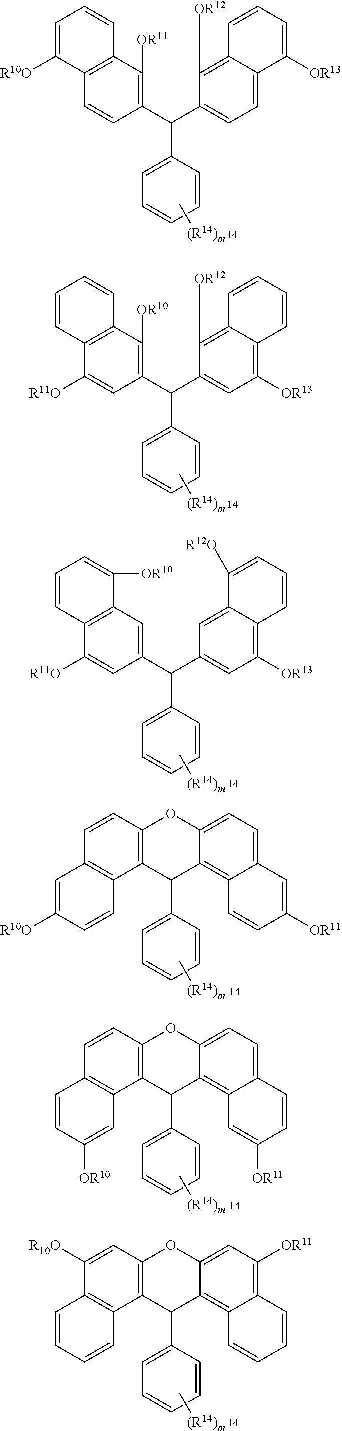

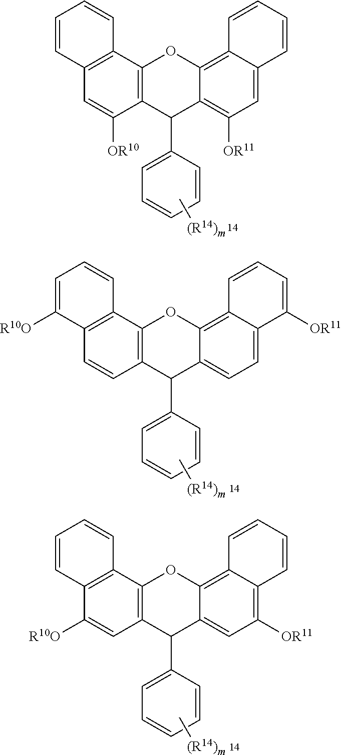

In the above formulae, R¹⁰ to R¹³ are as defined in those described in the above formula (1-2); each R¹⁴ is independently a linear, branched or cyclic alkyl group of 1 to 30 carbon atoms, an aryl group of 6 to 30 carbon atoms, an alkenyl group of 2 to 30 carbon atoms, an alkoxy group of 1 to 30 carbon atoms, a halogen atom, or a thiol group; and m¹⁴ is an integer of 0 to 4.

Examples of R¹⁴ include a methyl group, an ethyl group, a propyl group, a butyl group, a pentyl group, a hexyl group, a heptyl group, an octyl group, a nonyl group, a decyl group, an undecyl group, a dodecyl group, a triacontyl group, a cyclopropyl group, a cyclobutyl group, a cyclopentyl group, a cyclohexyl group, a cycloheptyl group, a cyclooctyl group, a cyclononyl group, a cyclodecyl group, a cycloundecyl group, a cyclododecyl group, a cyclotriacontyl group, a norbornyl group, an adamantyl group, a phenyl group, a naphthyl group, an anthracene group, a heptacene group, a vinyl group, an allyl group, a triacontenyl group, a methoxy group, an ethoxy group, a triacontoxy group, a fluorine atom, a chlorine atom, a bromine atom, an iodine atom, and a thiol group.

Each of the examples of R¹⁴ includes isomers. For example, the butyl group includes a n-butyl group, an isobutyl group, a sec-butyl group, and a tert-butyl group.

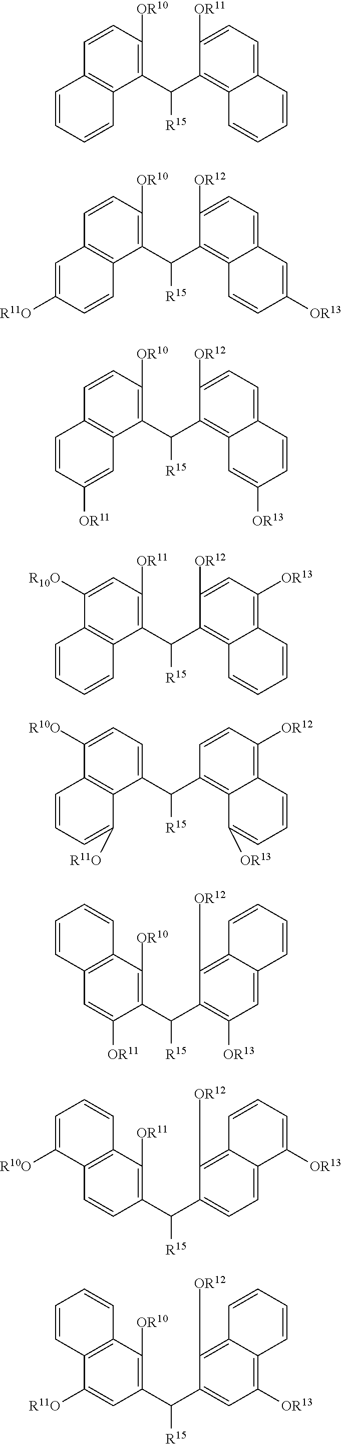

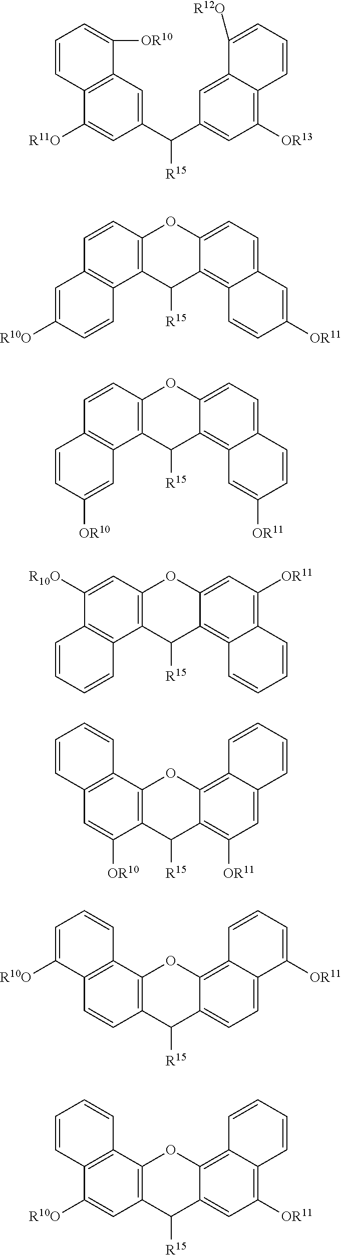

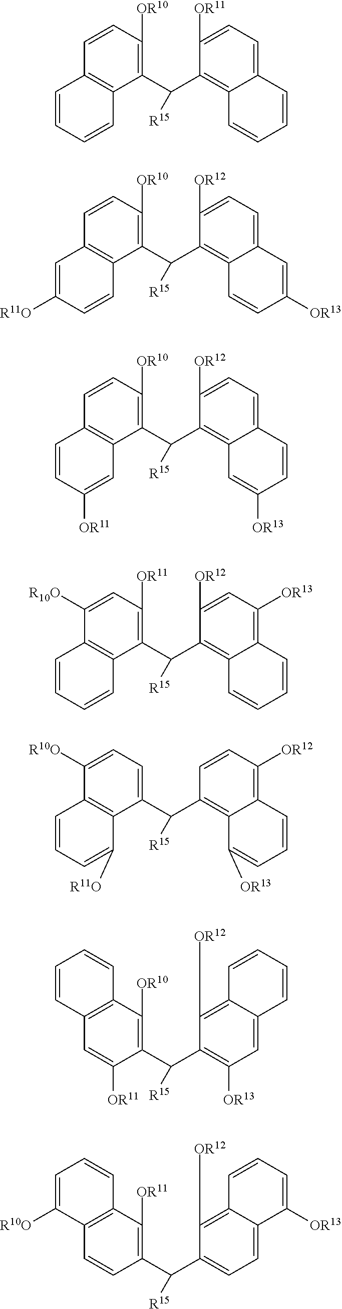

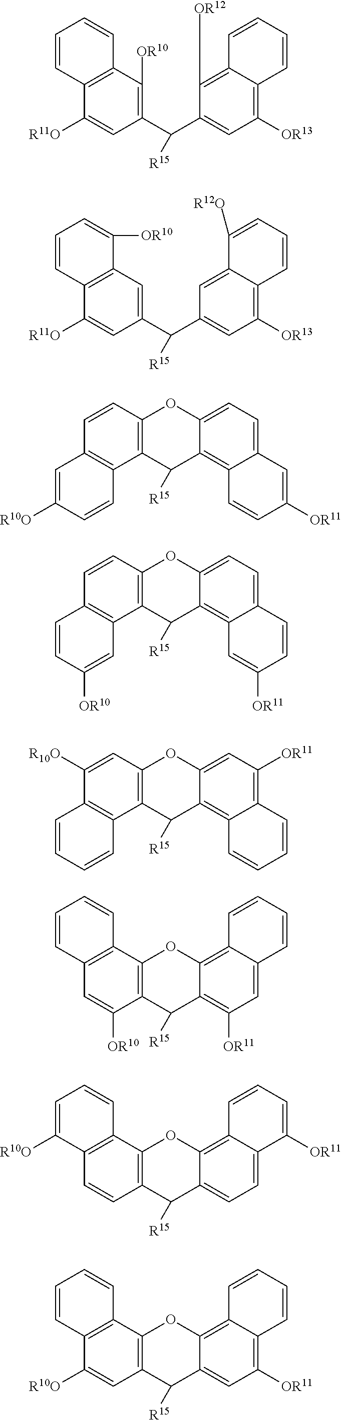

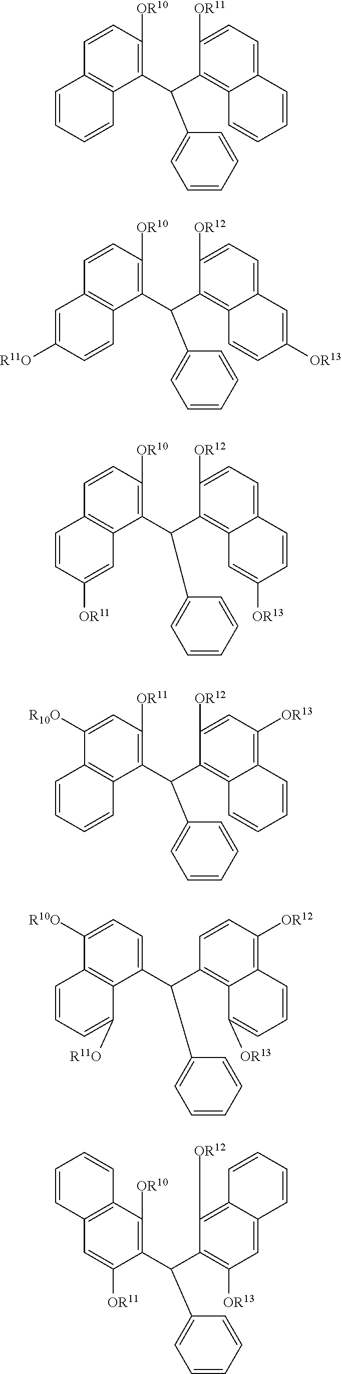

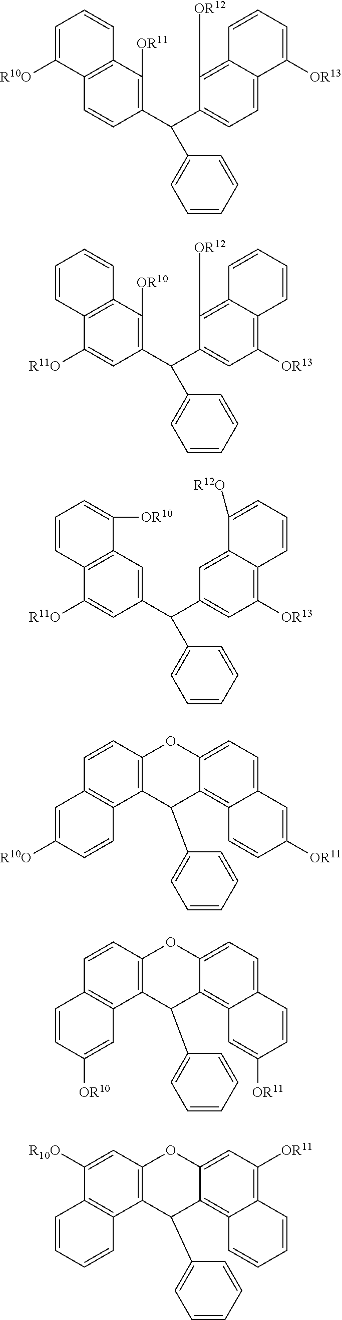

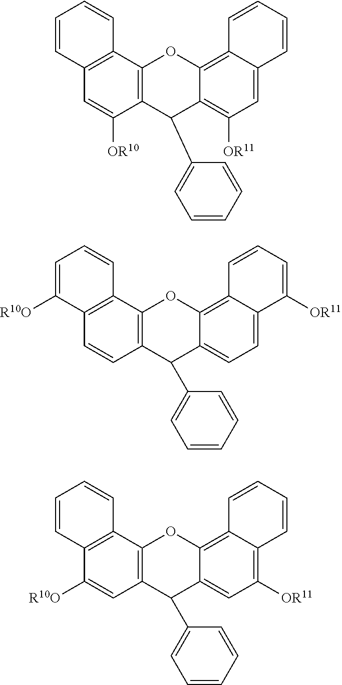

In the above formulae, R¹⁰ to R¹³ are as defined in those described in the above formula (1-2); and R¹⁵ is a linear, branched or cyclic alkyl group of 1 to 30 carbon atoms, an aryl group of 6 to 30 carbon atoms, an alkenyl group of 2 to 30 carbon atoms, an alkoxy group of 1 to 30 carbon atoms, a halogen atom, or a thiol group.

Examples of R¹⁵ include a methyl group, an ethyl group, a propyl group, a butyl group, a pentyl group, a hexyl group, a heptyl group, an octyl group, a nonyl group, a decyl group, an undecyl group, a dodecyl group, a triacontyl group, a cyclopropyl group, a cyclobutyl group, a cyclopentyl group, a cyclohexyl group, a cycloheptyl group, a cyclooctyl group, a cyclononyl group, a cyclodecyl group, a cycloundecyl group, a cyclododecyl group, a cyclotriacontyl group, a norbornyl group, an adamantyl group, a phenyl group, a naphthyl group, an anthracene group, a heptacene group, a vinyl group, an allyl group, a triacontenyl group, a methoxy group, an ethoxy group, a triacontoxy group, a fluorine atom, a chlorine atom, a bromine atom, an iodine atom, and a thiol group.

Each of the examples of R¹⁵ includes isomers. For example, the butyl group includes a n-butyl group, an isobutyl group, a sec-butyl group, and a tert-butyl group.

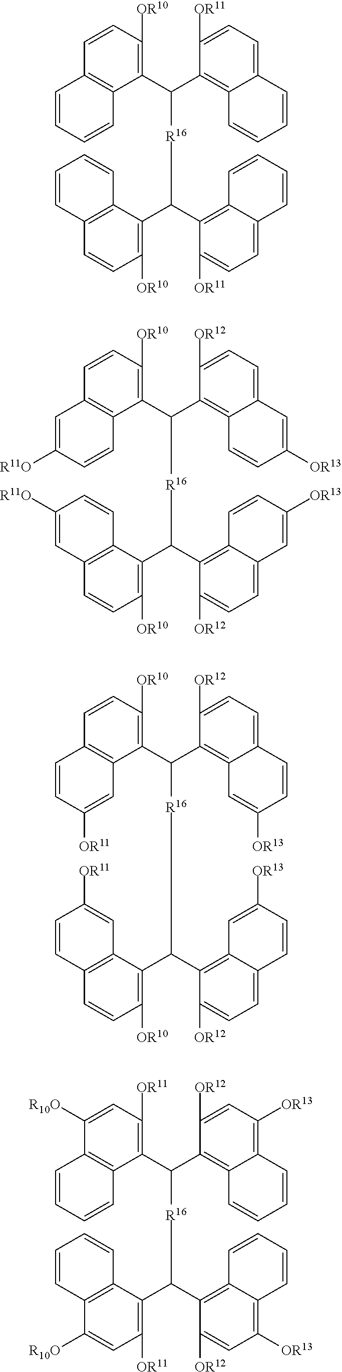

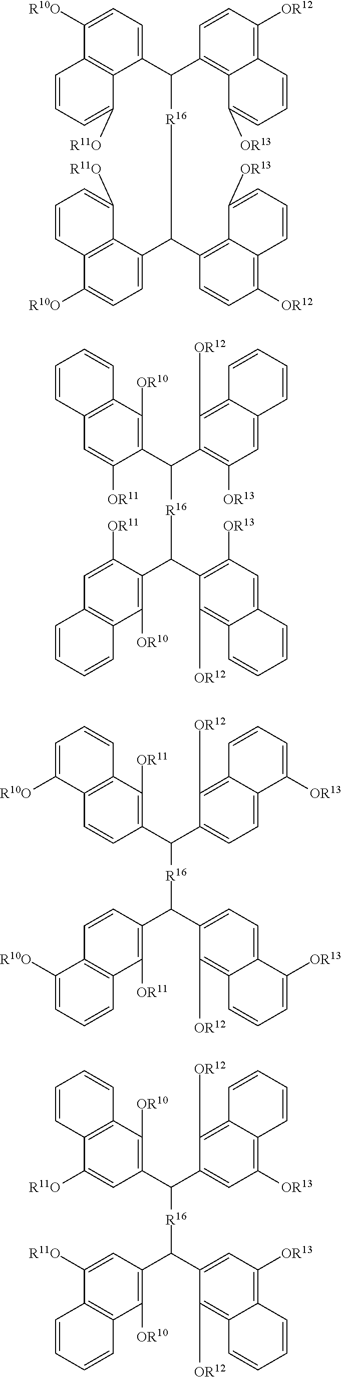

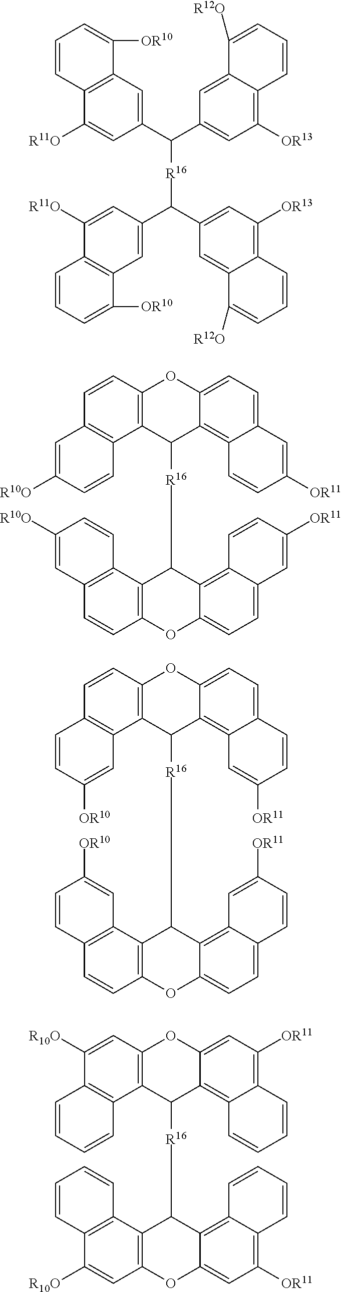

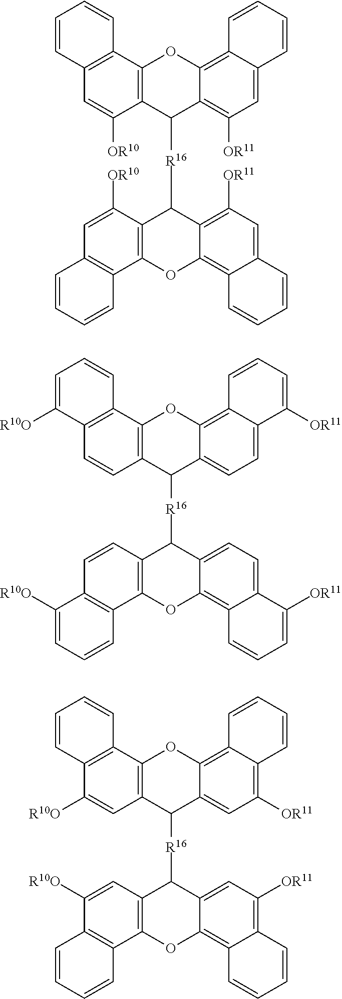

In the above formulae, R¹⁰ to R¹³ are as defined in those described in the above formula (1-2); and R¹⁶ is a linear, branched or cyclic alkylene group of 1 to 30 carbon atoms, a divalent aryl group of 6 to 30 carbon atoms, or a divalent alkenyl group of 2 to 30 carbon atoms.

Examples of R¹⁶ include a methylene group, an ethylene group, a propene group, a butene group, a pentene group, a hexene group, a heptene group, an octene group, a nonene group, a decene group, an undecene group, a dodecene group, a triacontene group, a cyclopropene group, a cyclobutene group, a cyclopentene group, a cyclohexene group, a cycloheptene group, a cyclooctene group, a cyclononene group, a cyclodecene group, a cycloundecene group, a cyclododecene group, a cyclotriacontene group, a divalent norbornyl group, a divalent adamantyl group, a divalent phenyl group, a divalent naphthyl group, a divalent anthracene group, a divalent heptacene group, a divalent vinyl group, a divalent allyl group, and a divalent triacontenyl group.

Each of the examples of R¹⁶ includes isomers. For example, the butyl group includes a n-butyl group, an isobutyl group, a sec-butyl group, and a tert-butyl group.

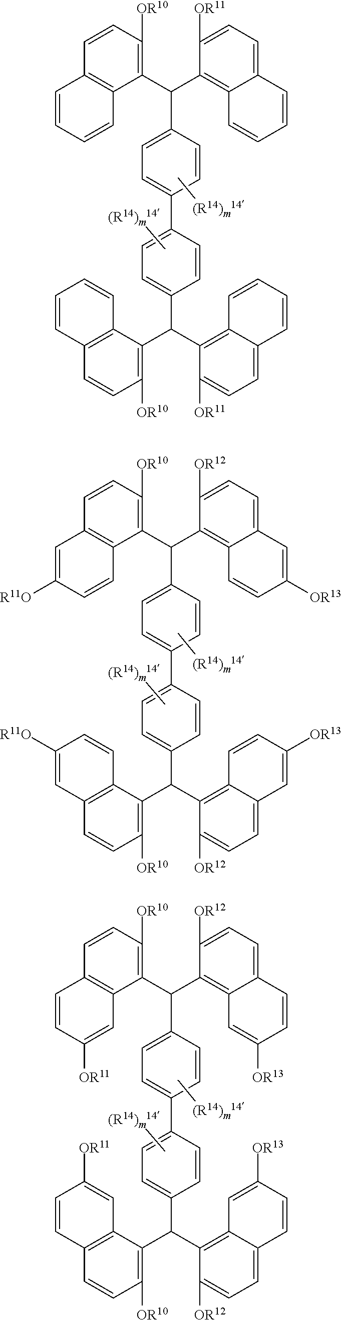

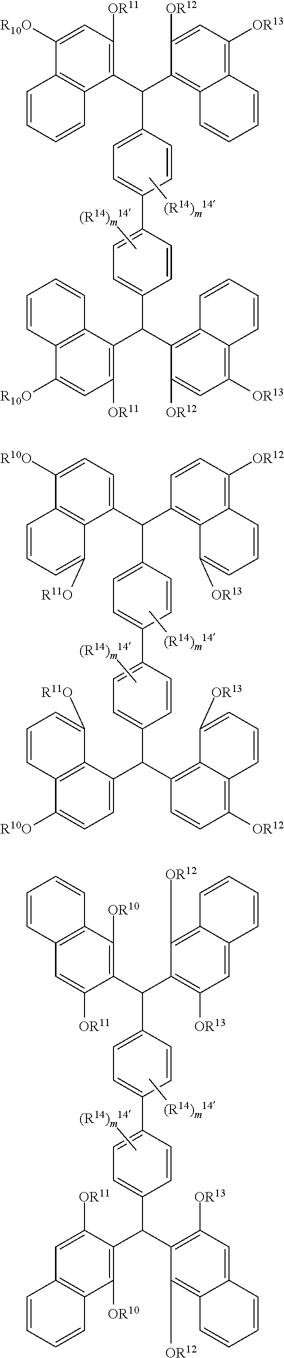

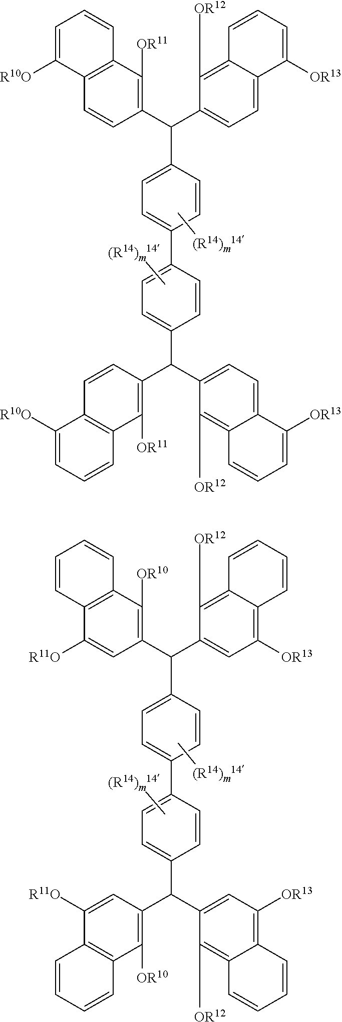

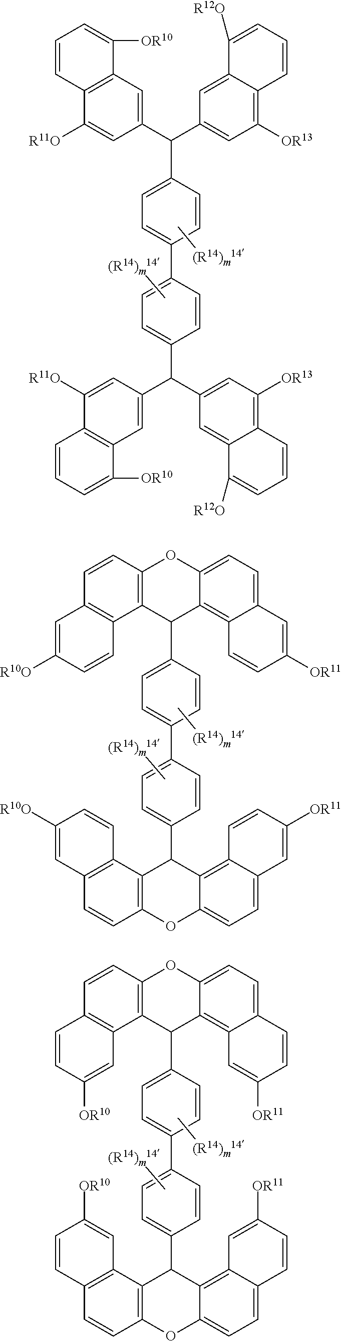

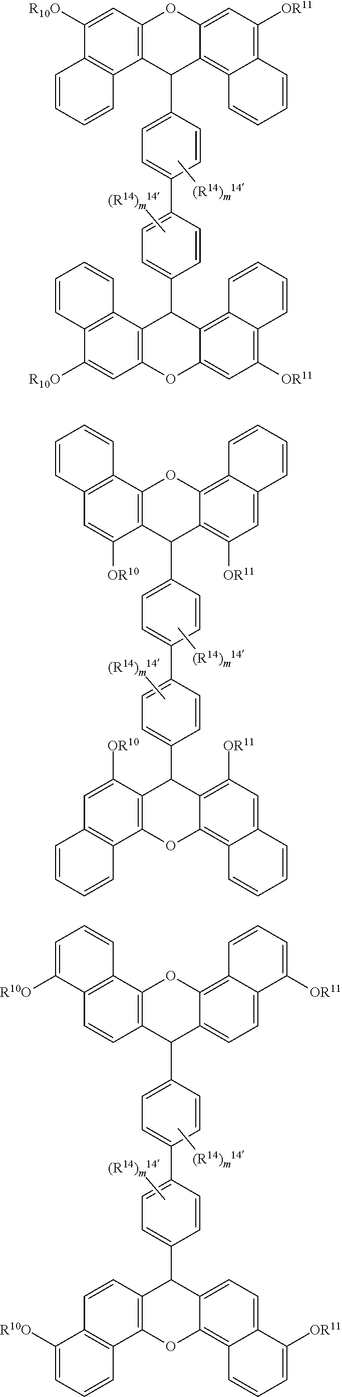

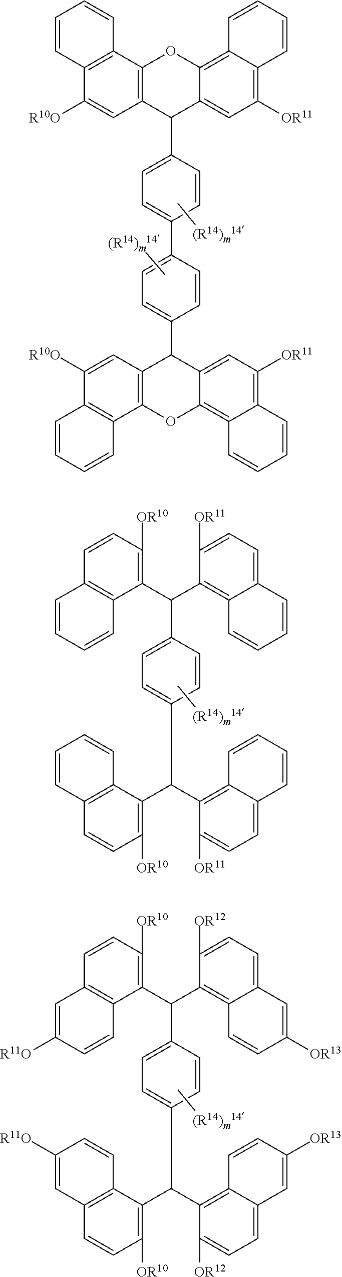

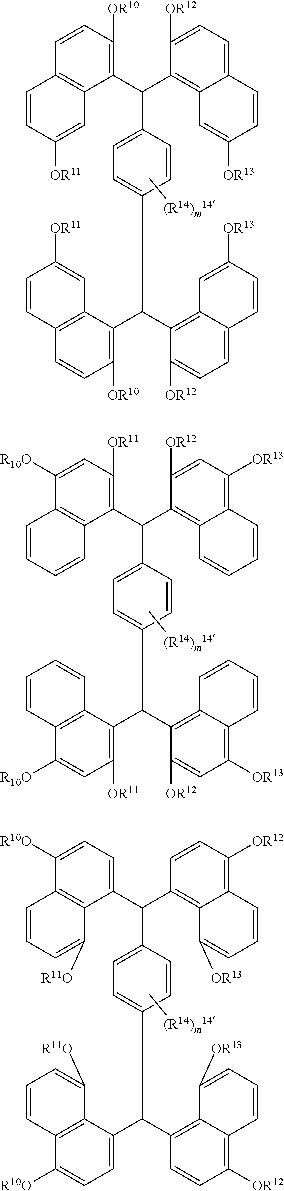

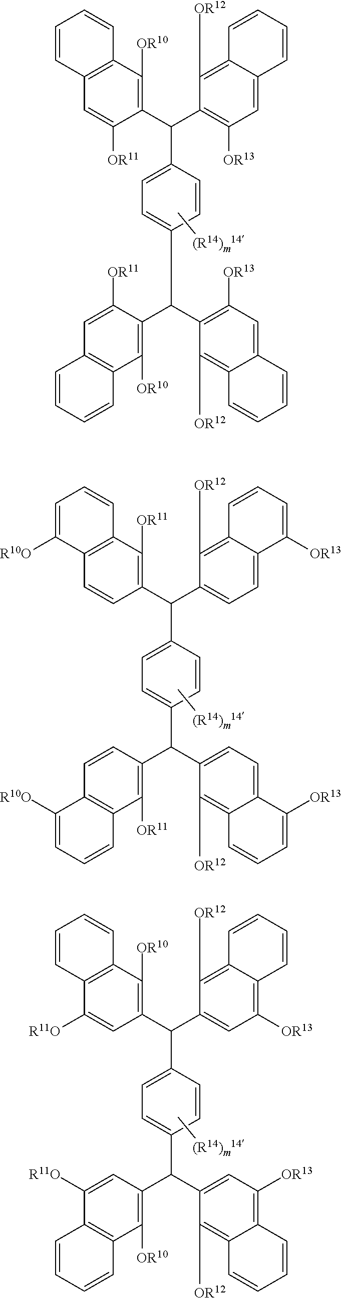

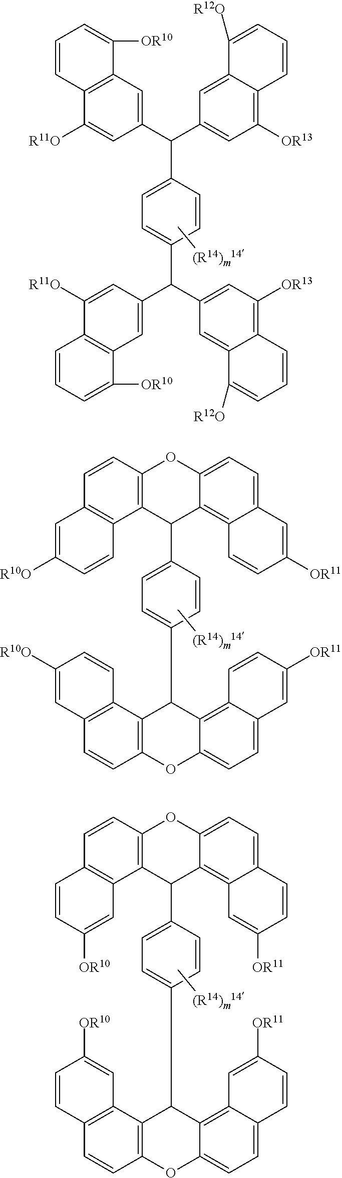

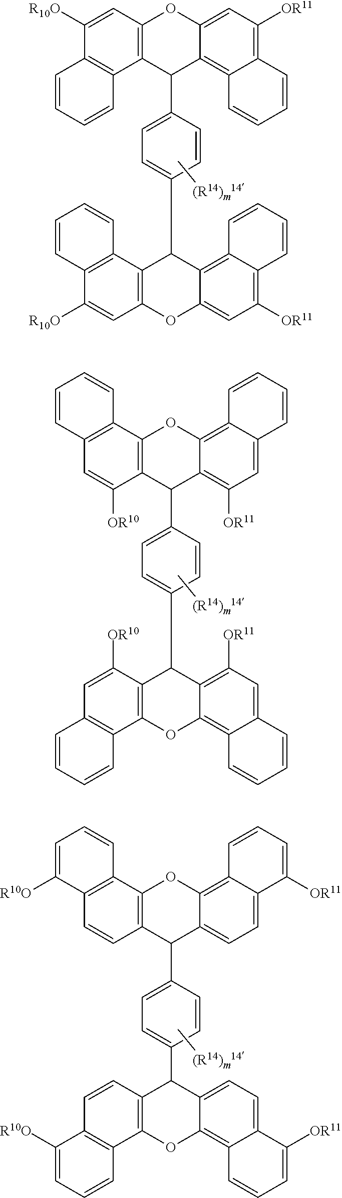

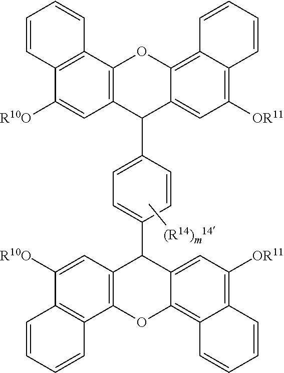

In the above formulae, R¹⁰ to R¹³ are as defined in those described in the above formula (1-2); each R¹⁴ is independently a linear, branched or cyclic alkyl group of 1 to 30 carbon atoms, an aryl group of 6 to 30 carbon atoms, an alkenyl group of 2 to 30 carbon atoms, an alkoxy group of 1 to 30 carbon atoms, a halogen atom, or a thiol group; and m^{14′} is an integer of 0 to 5.

Examples of R¹⁴ include a methyl group, an ethyl group, a propyl group, a butyl group, a pentyl group, a hexyl group, a heptyl group, an octyl group, a nonyl group, a decyl group, an undecyl group, a dodecyl group, a triacontyl group, a cyclopropyl group, a cyclobutyl group, a cyclopentyl group, a cyclohexyl group, a cycloheptyl group, a cyclooctyl group, a cyclononyl group, a cyclodecyl group, a cycloundecyl group, a cyclododecyl group, a cyclotriacontyl group, a norbornyl group, an adamantyl group, a phenyl group, a naphthyl group, an anthracene group, a heptacene group, a vinyl group, an allyl group, a triacontenyl group, a methoxy group, an ethoxy group, a triacontoxy group, a fluorine atom, a chlorine atom, a bromine atom, an iodine atom, and a thiol group.

Each of the examples of R¹⁴ includes isomers. For example, the butyl group includes a n-butyl group, an isobutyl group, a sec-butyl group, and a tert-butyl group.

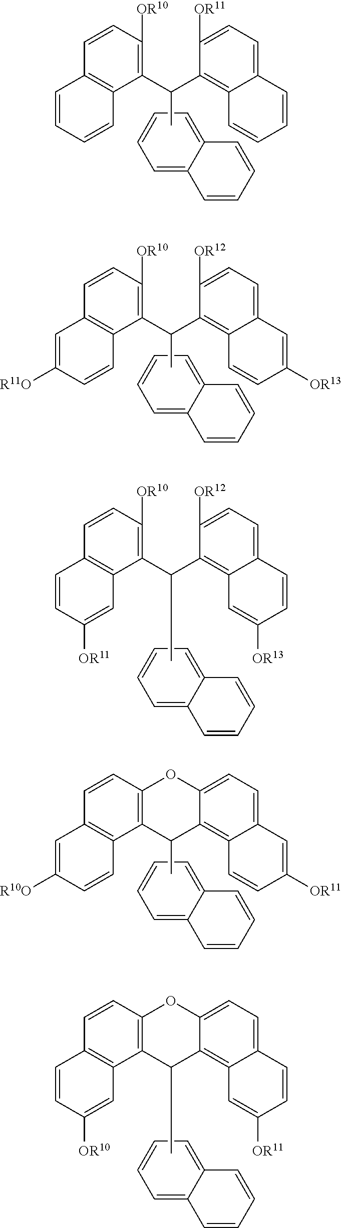

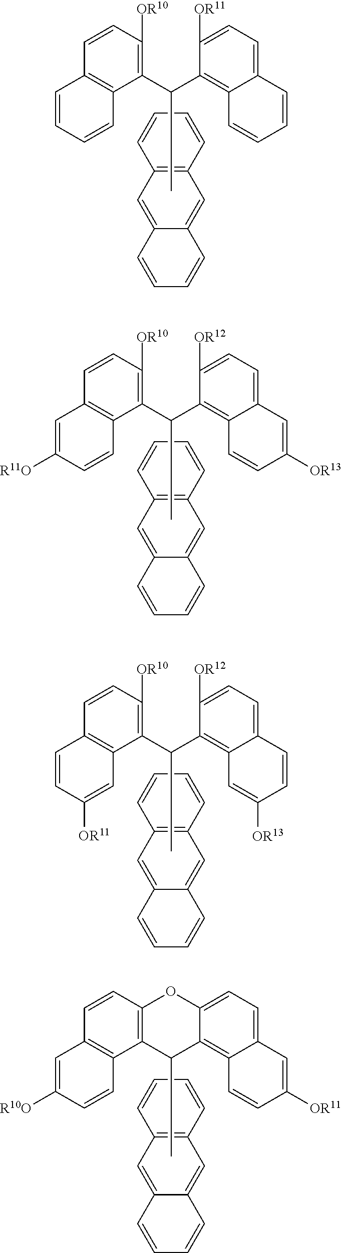

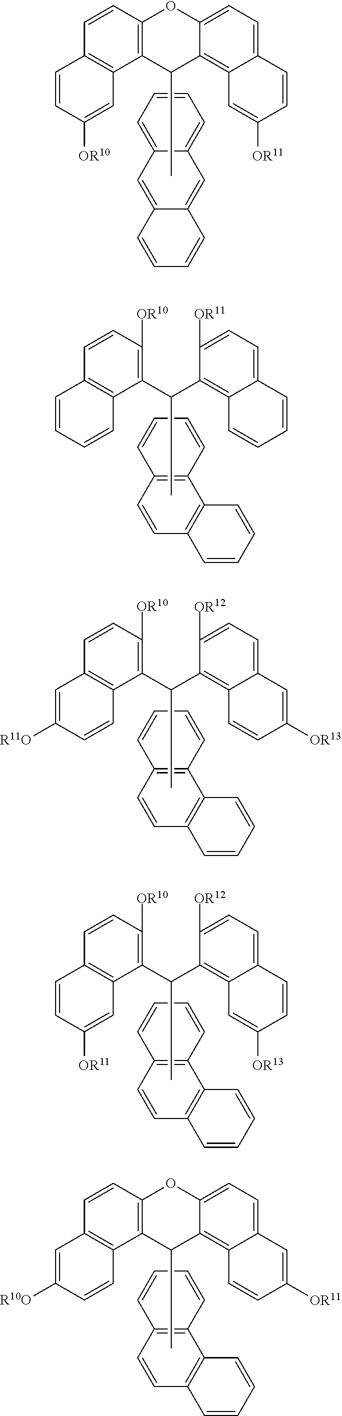

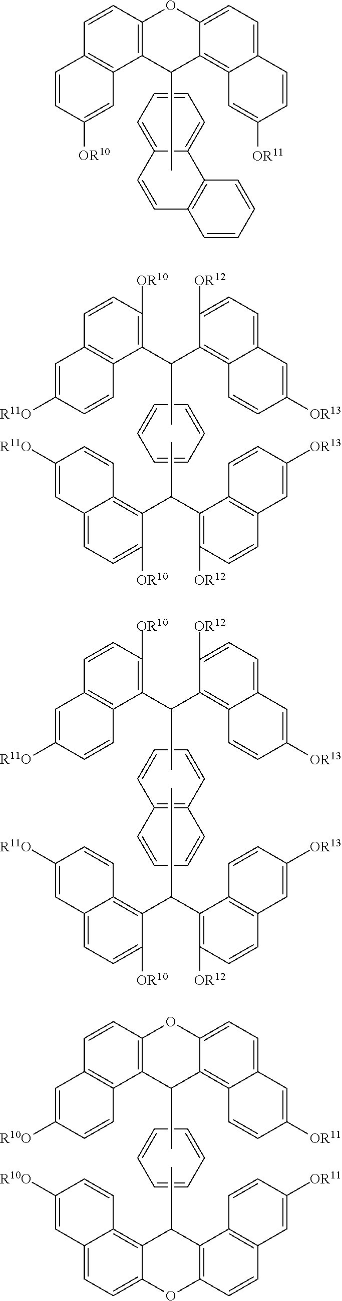

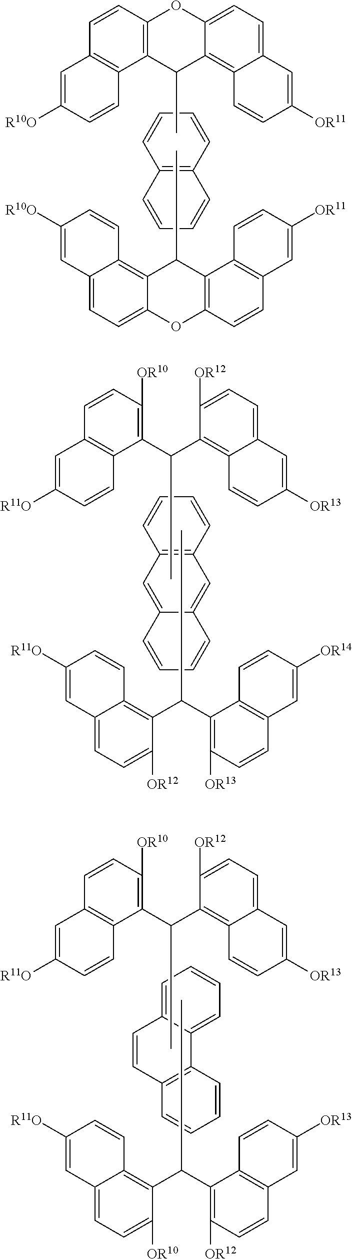

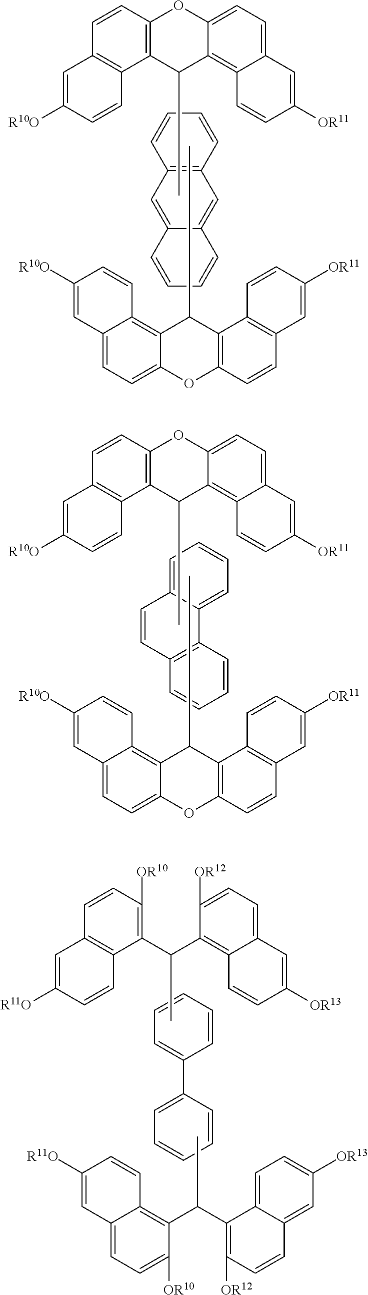

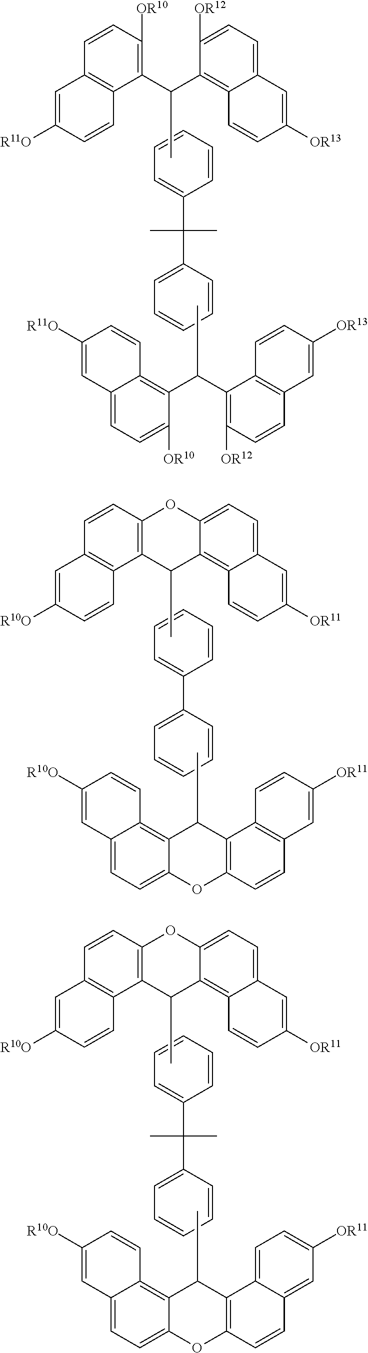

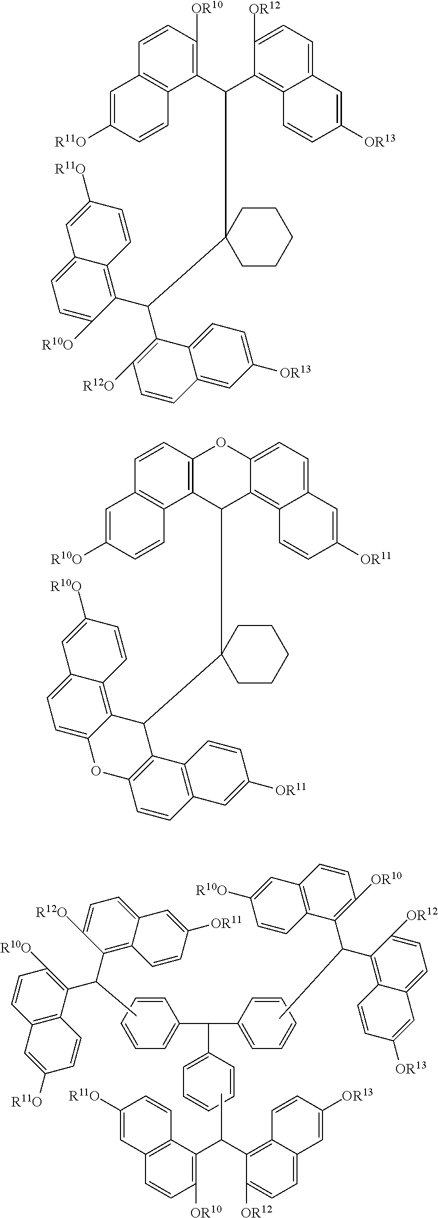

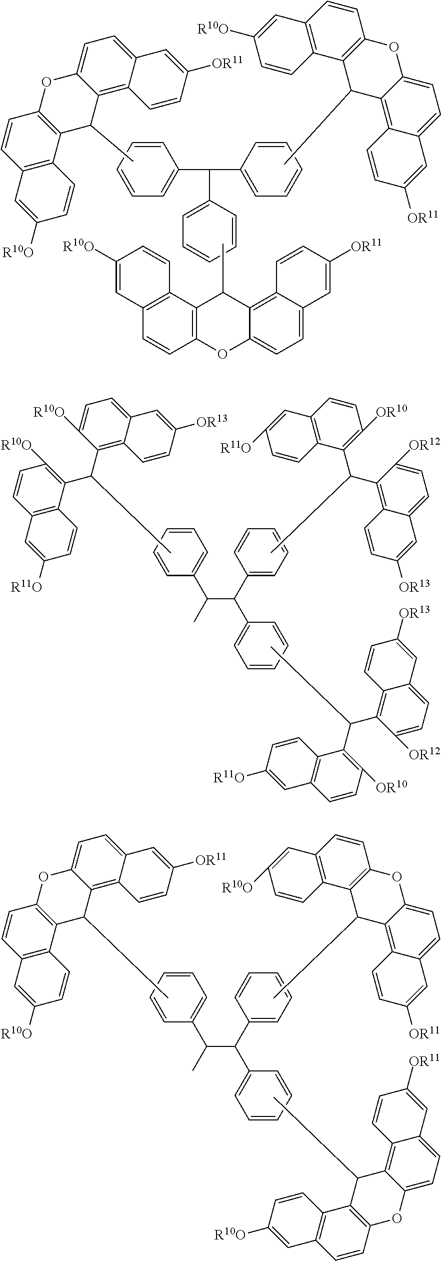

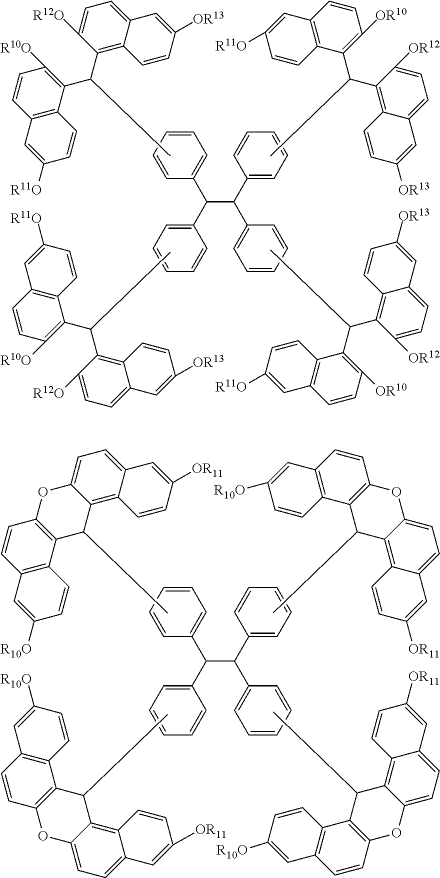

In the above formulae, R¹⁰ to R¹³ are as defined in those described in the above formula (1-2).

The compounds represented by the above formulae more preferably have a dibenzoxanthene skeleton from the viewpoint of heat resistance.

The following compounds are further preferable from the viewpoint of the availability of raw material:

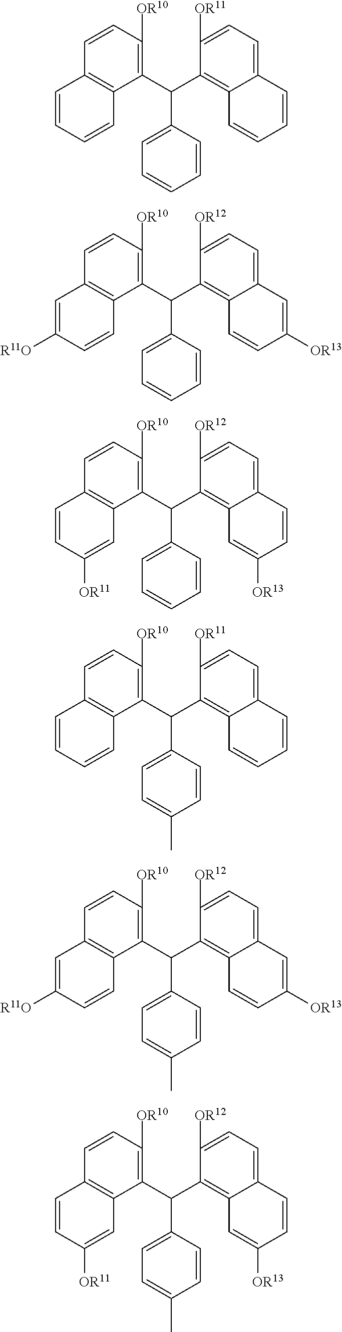

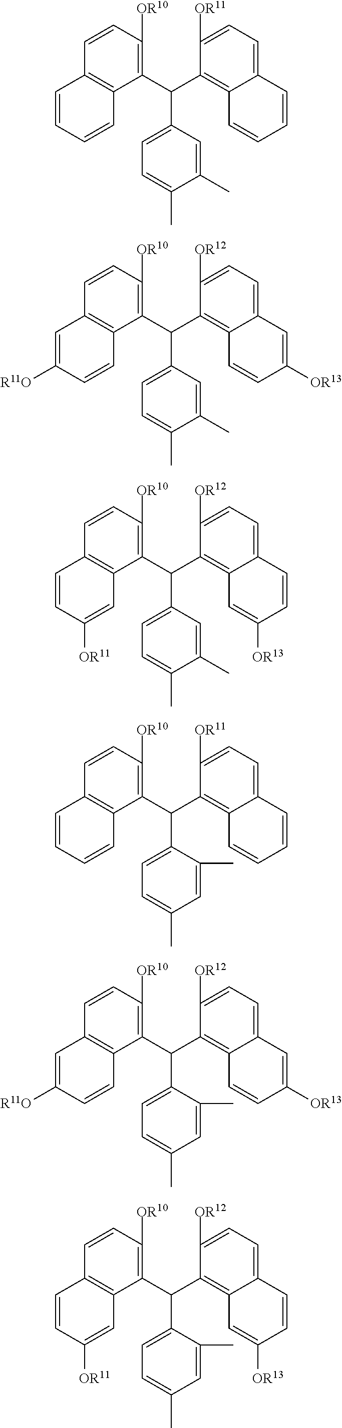

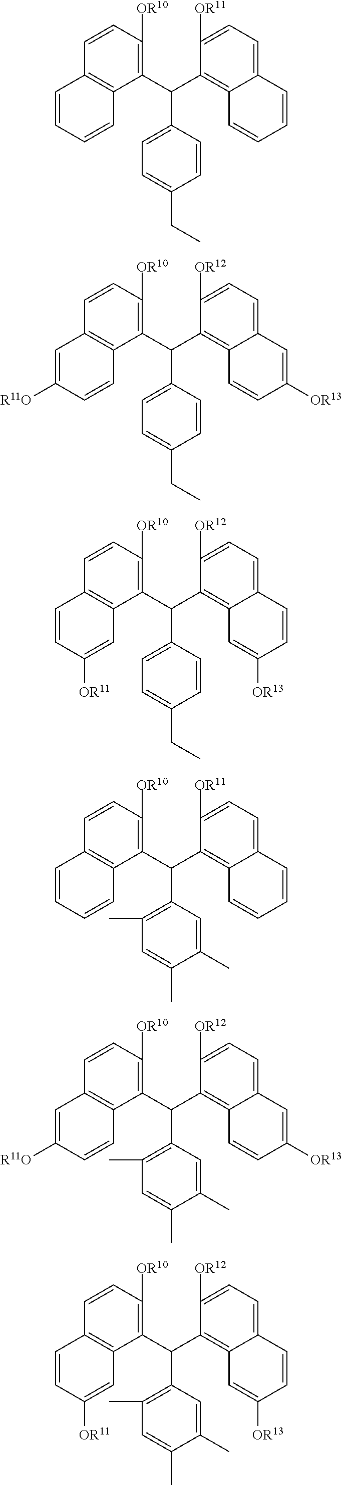

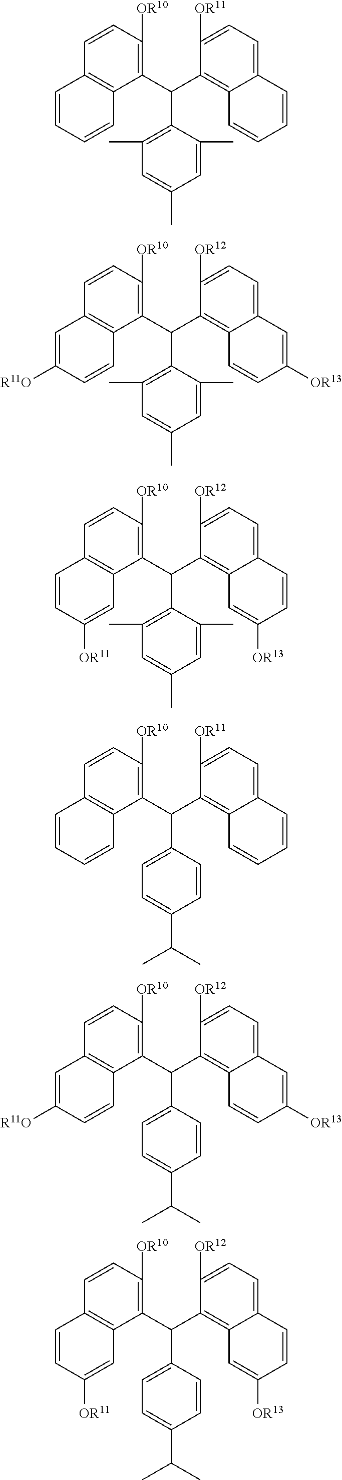

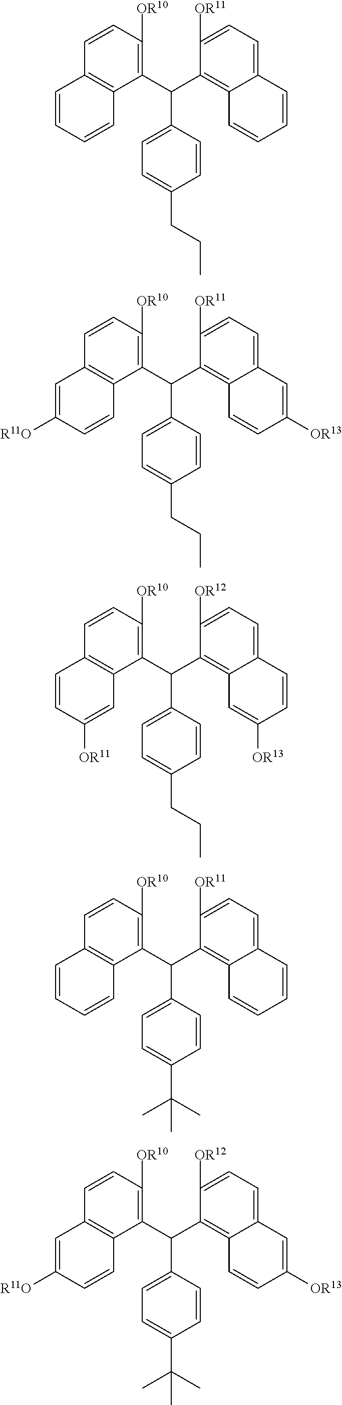

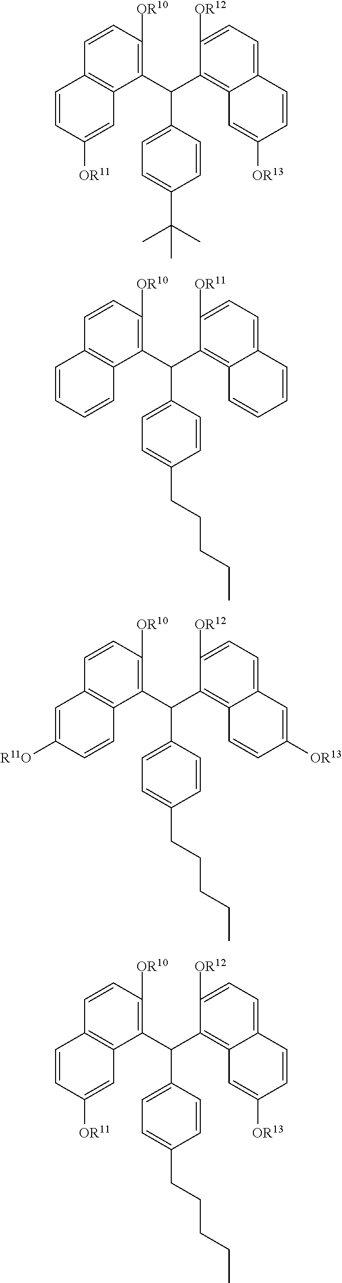

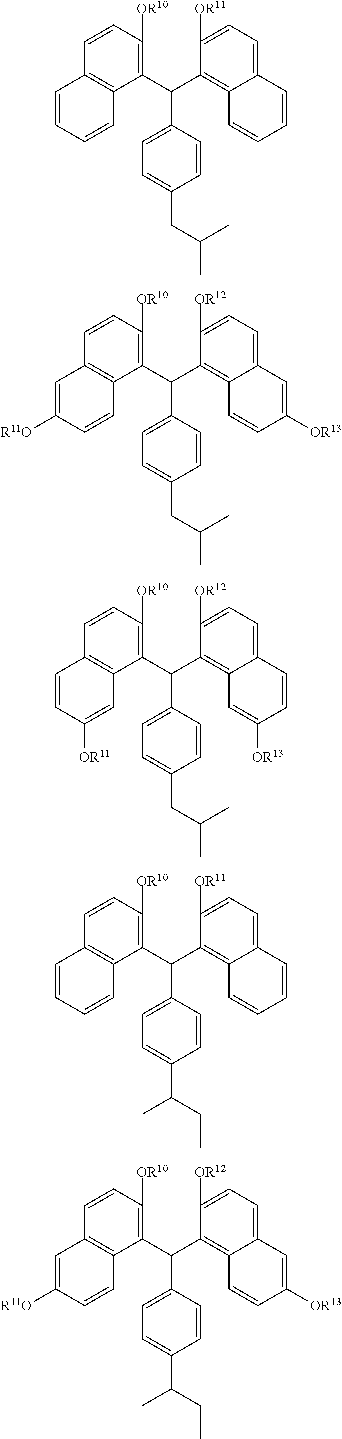

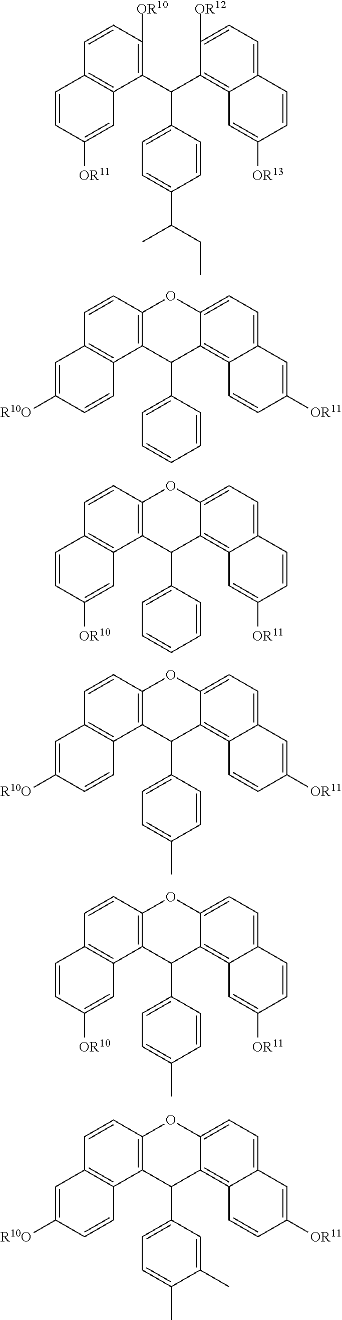

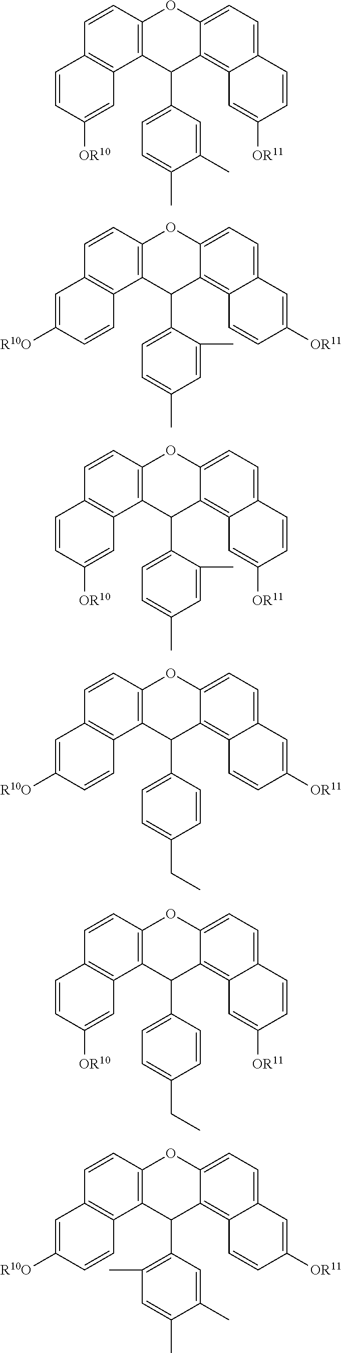

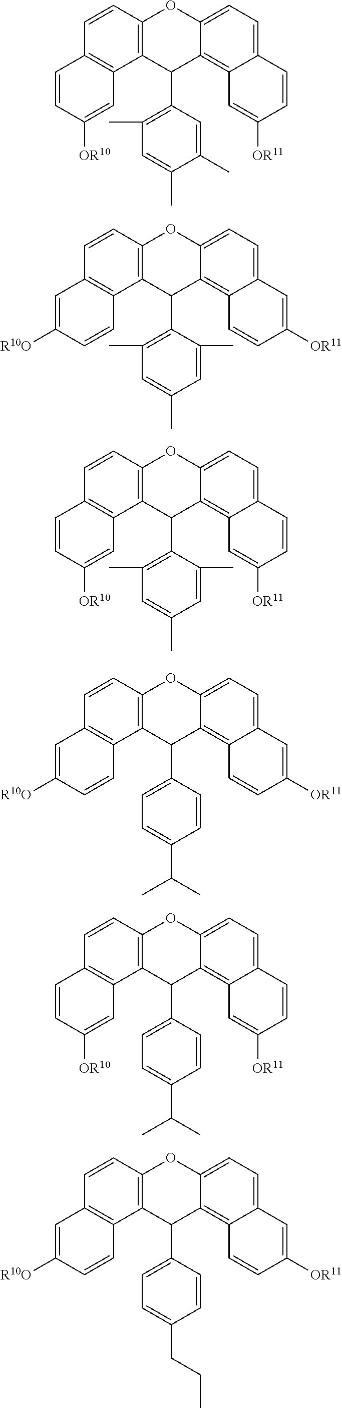

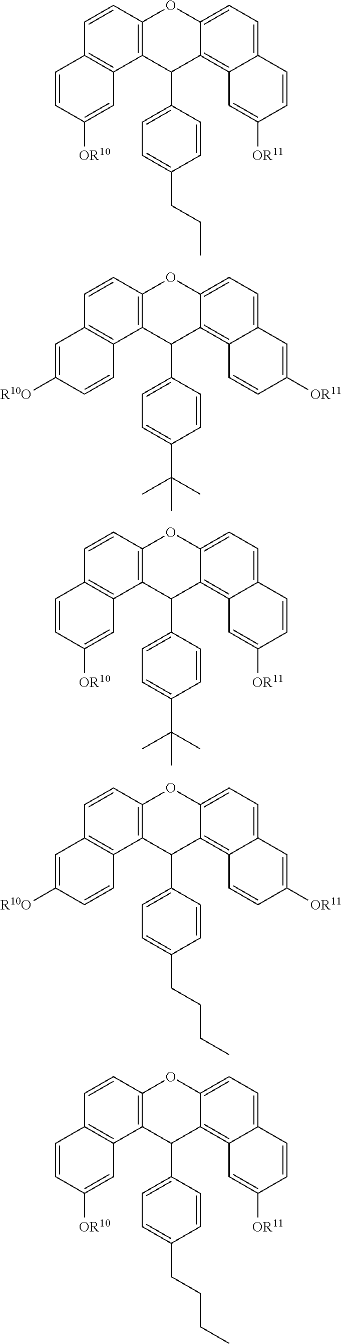

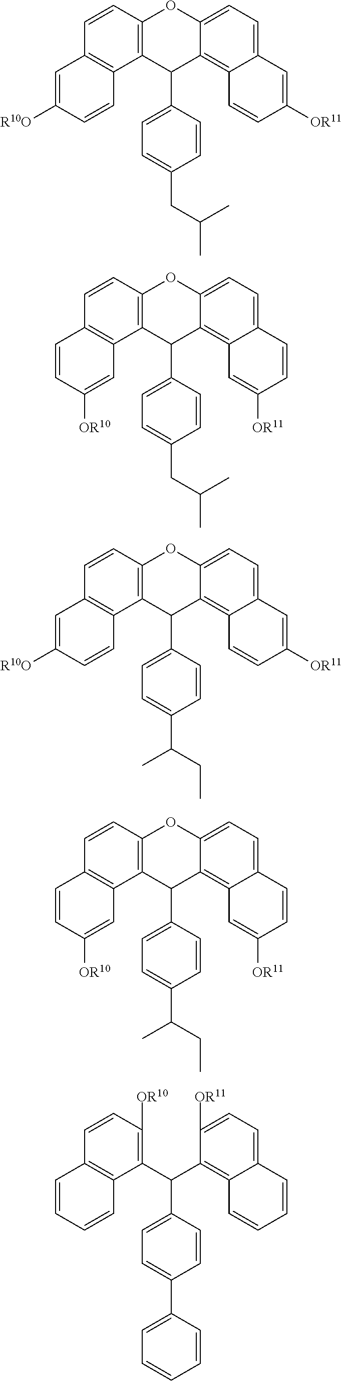

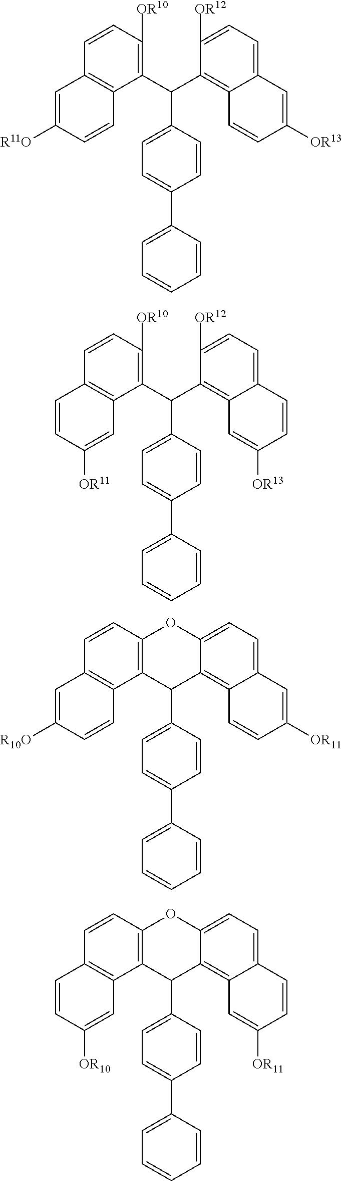

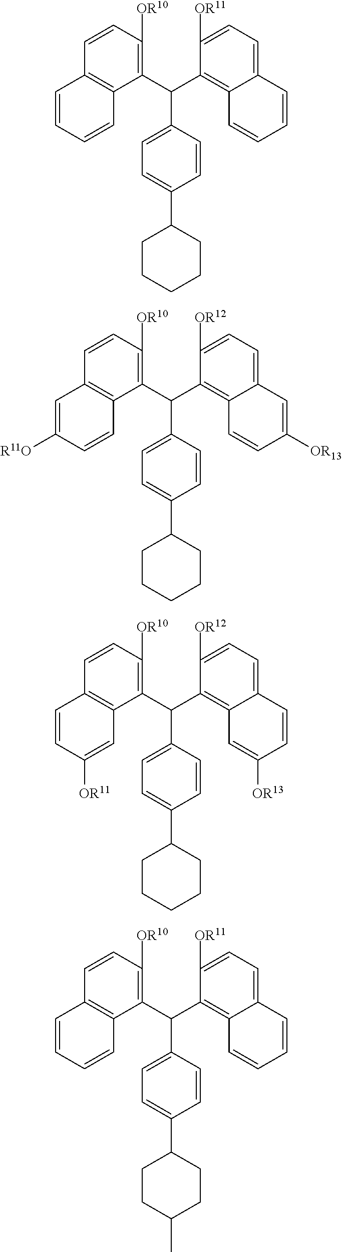

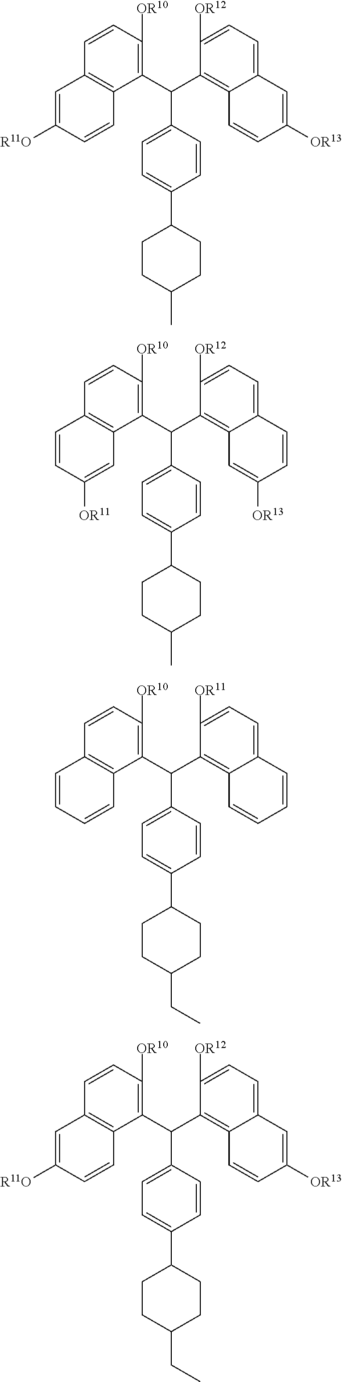

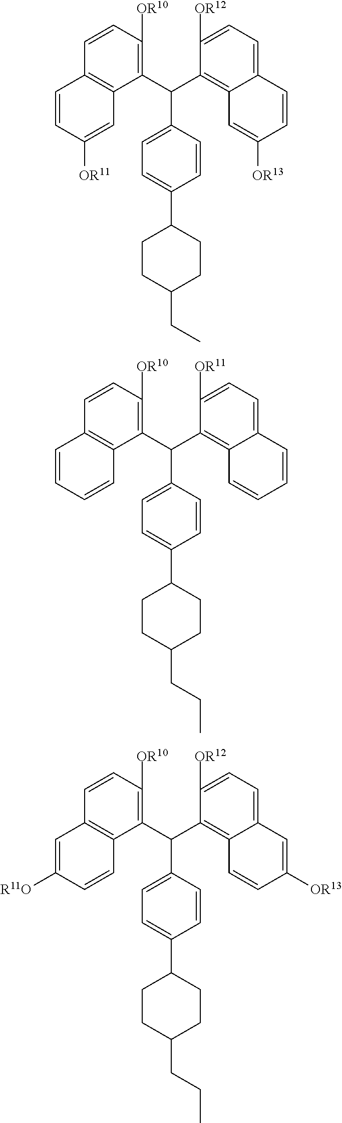

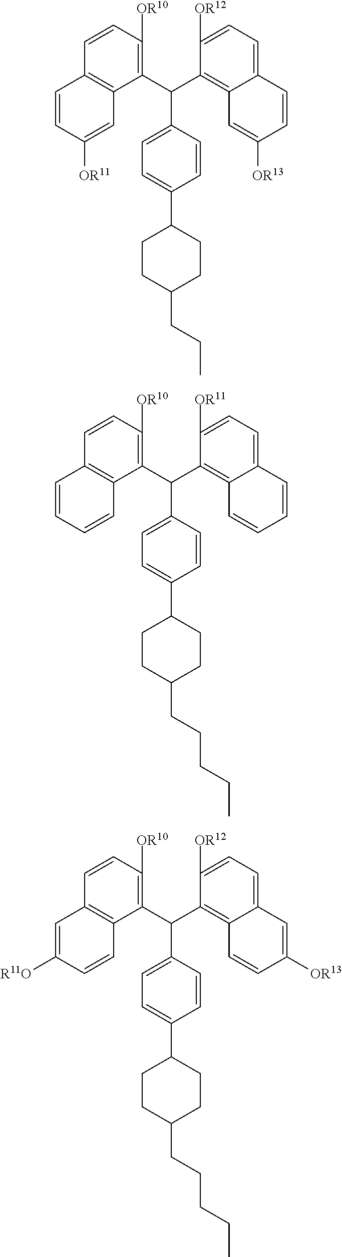

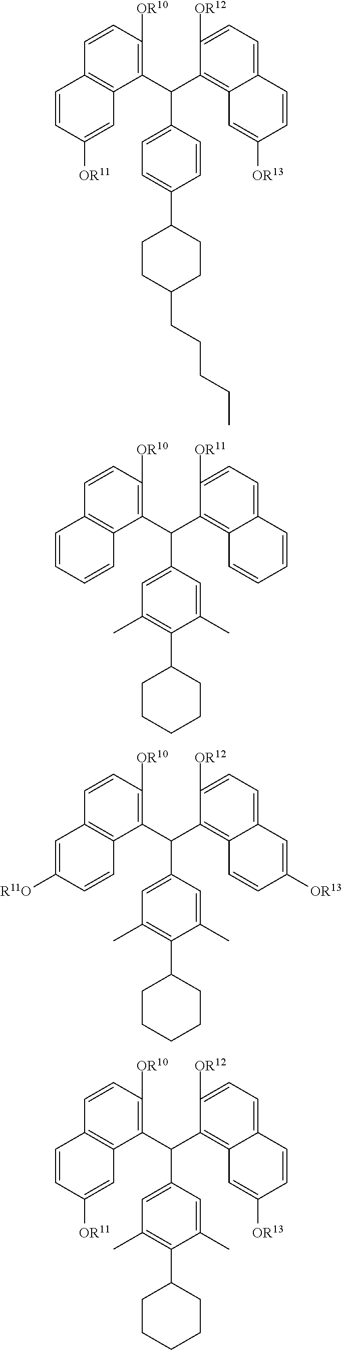

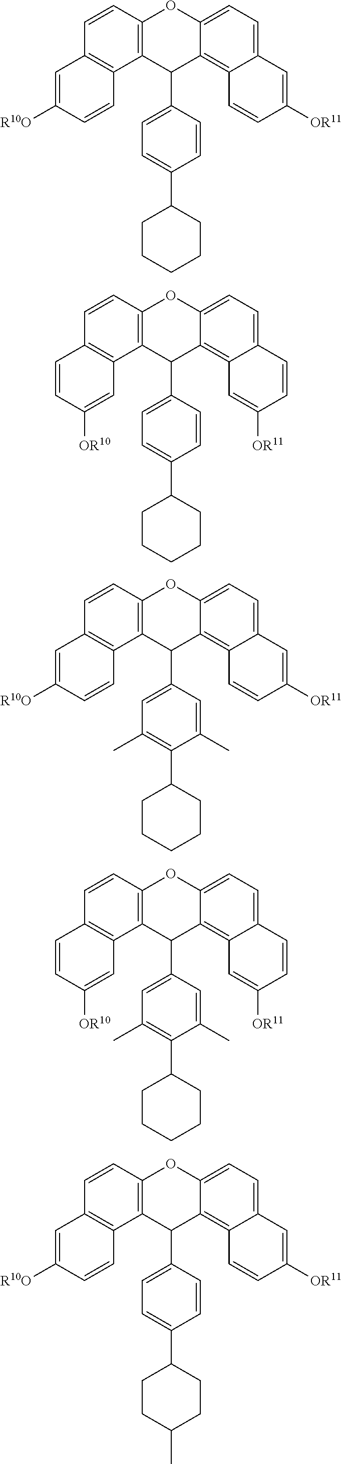

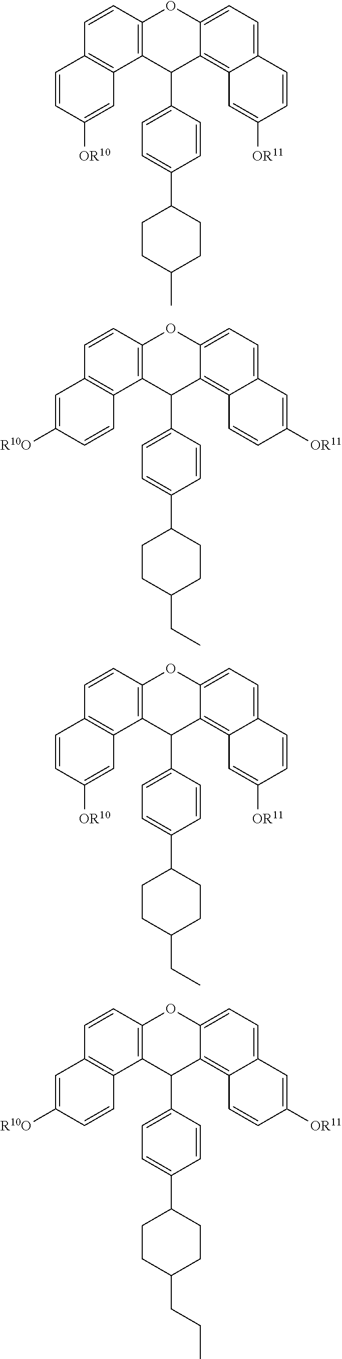

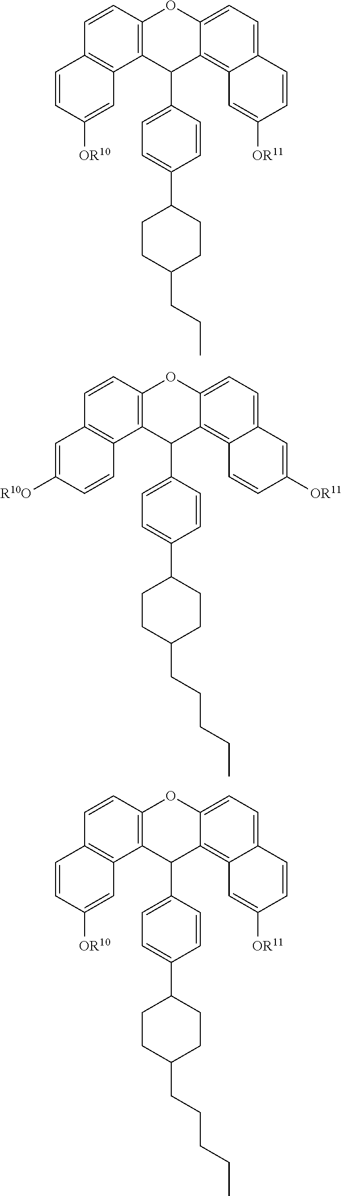

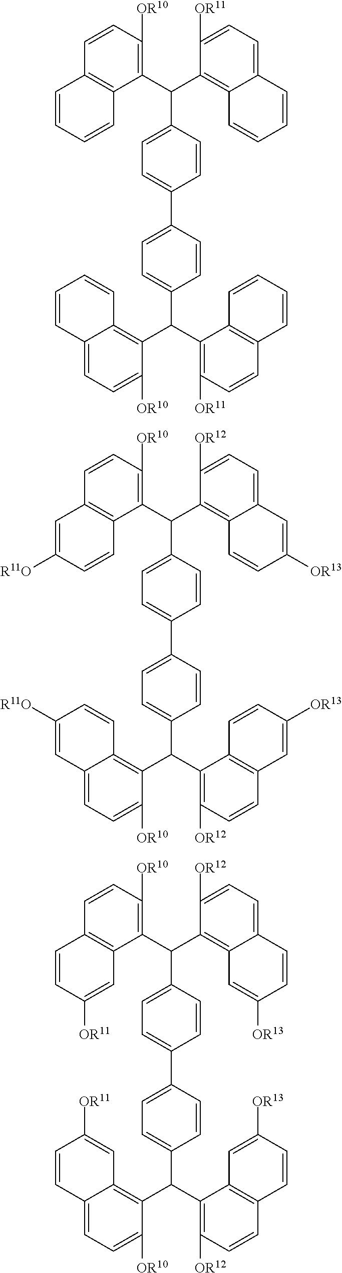

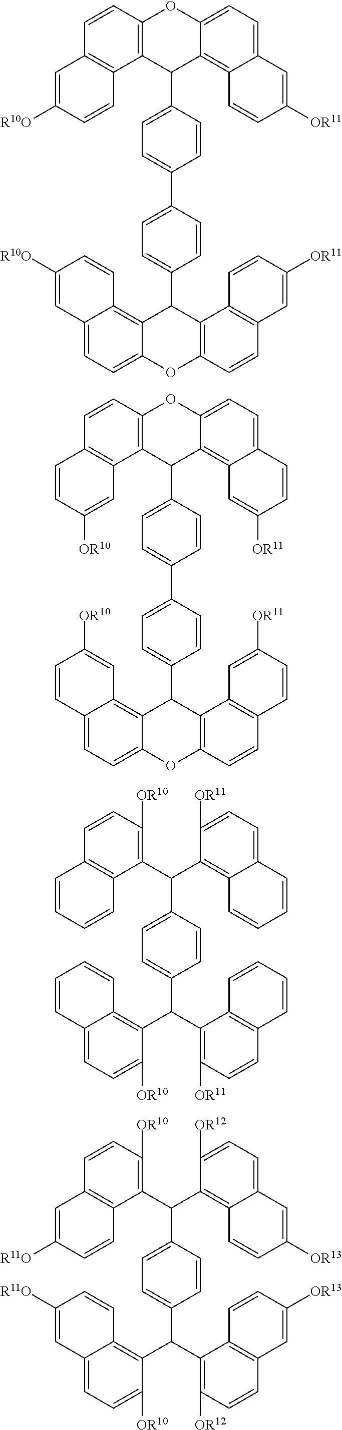

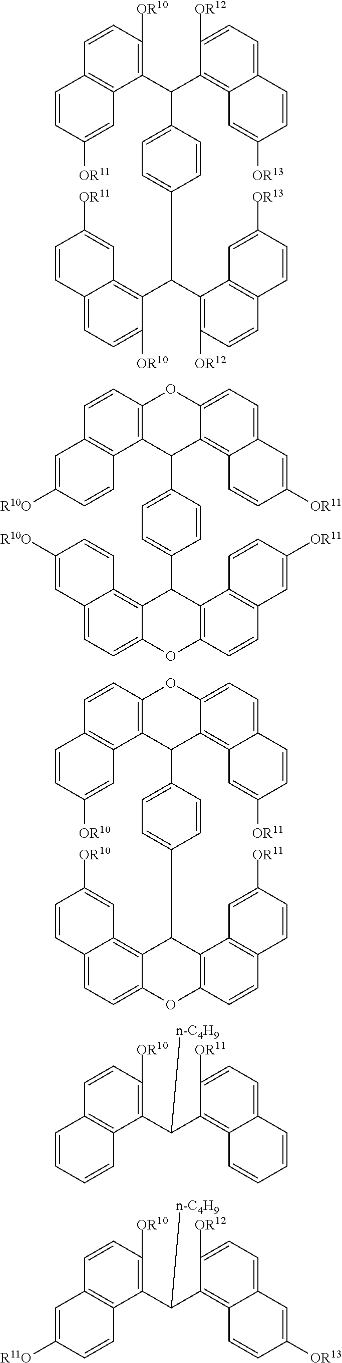

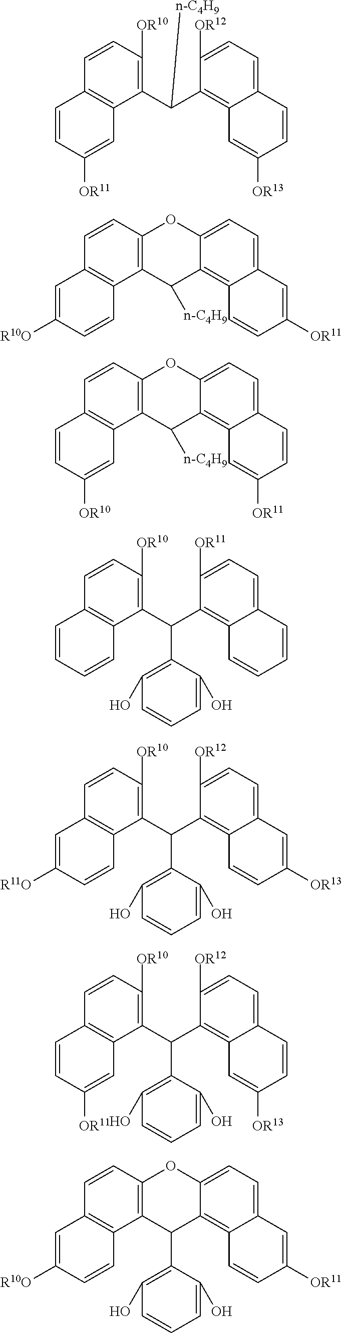

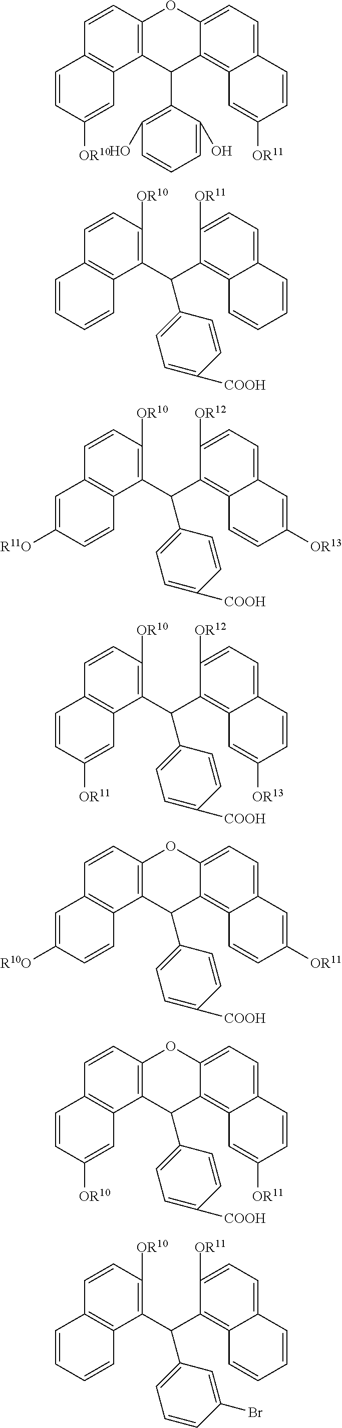

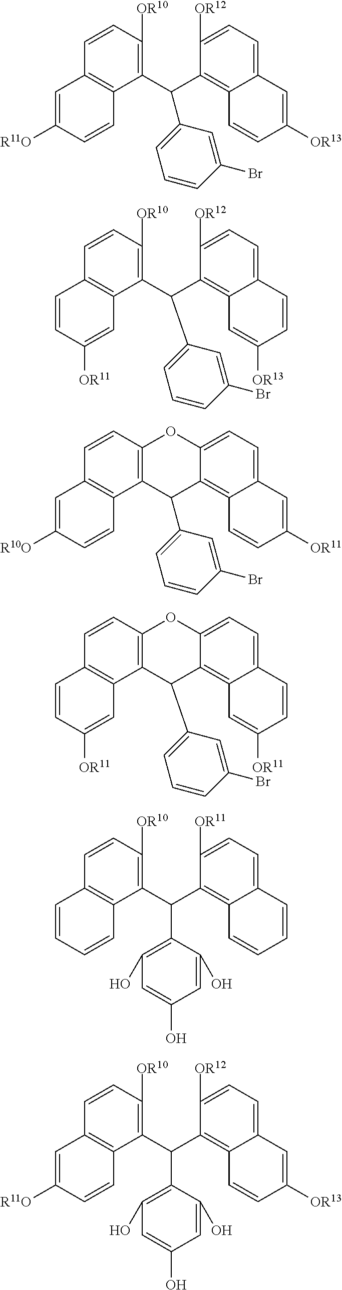

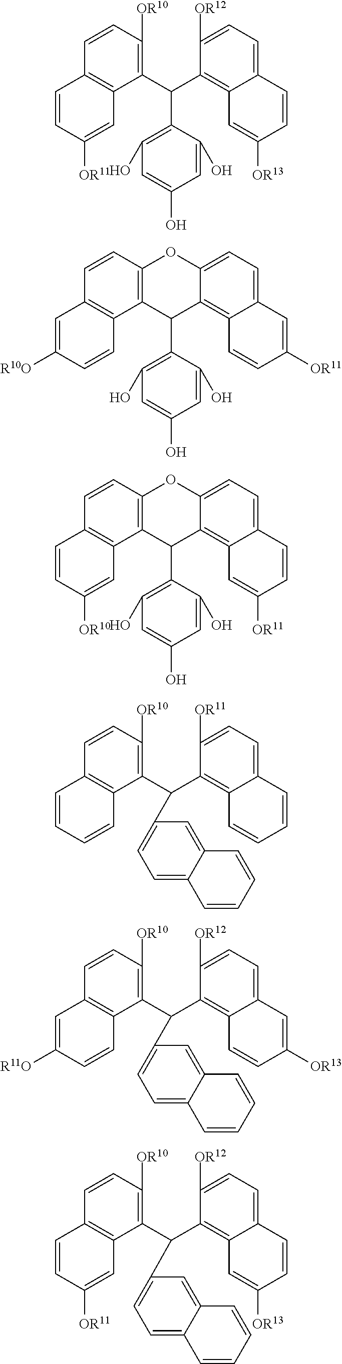

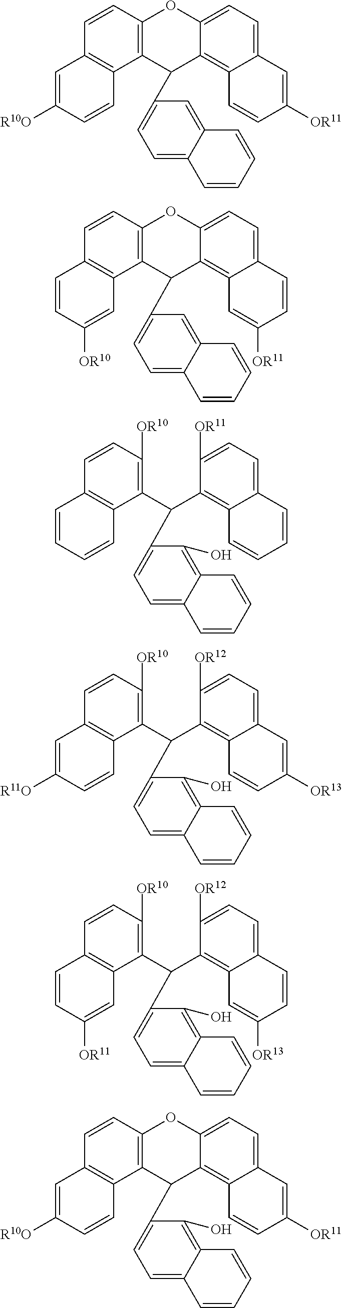

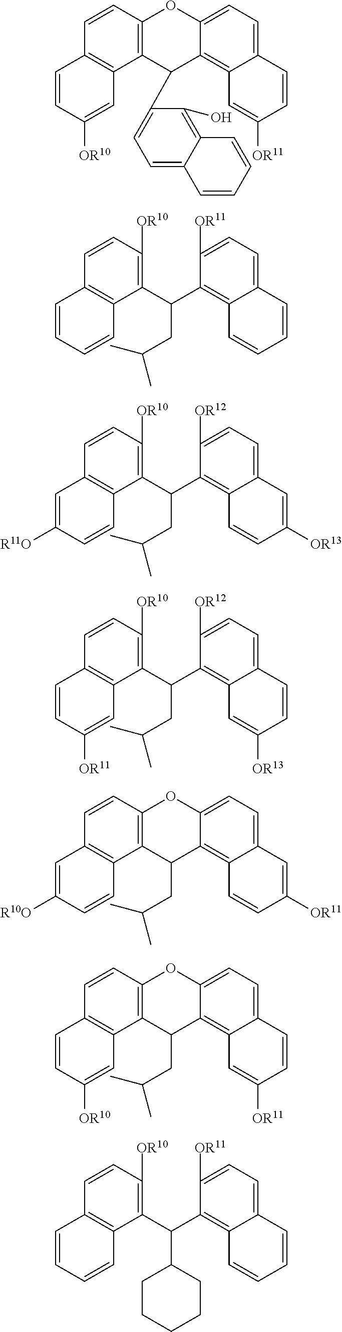

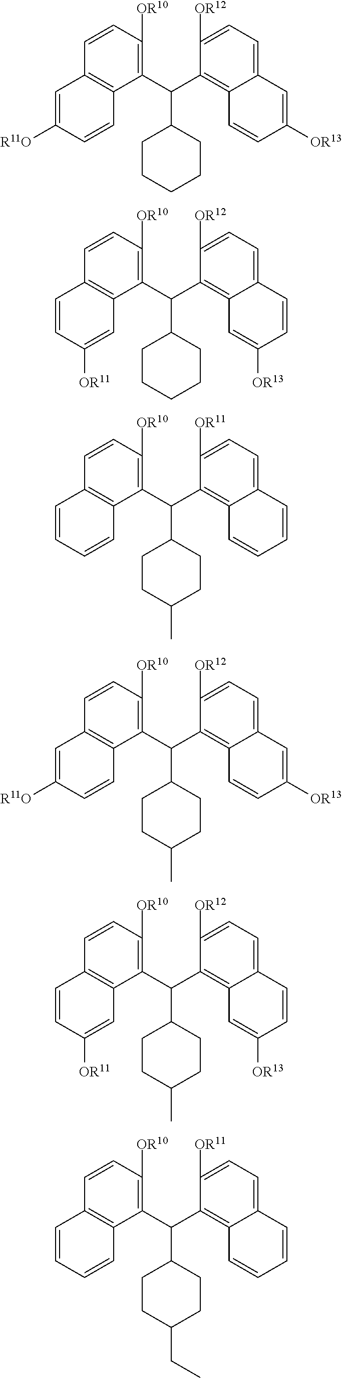

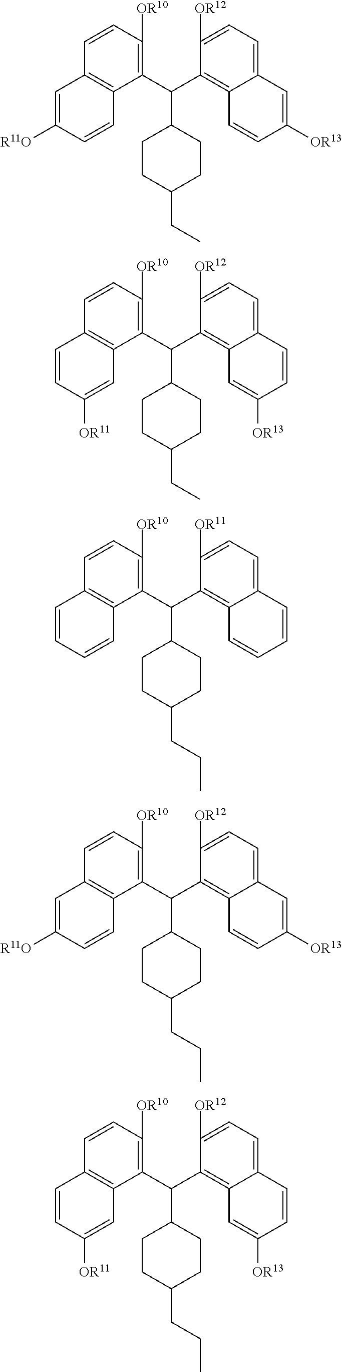

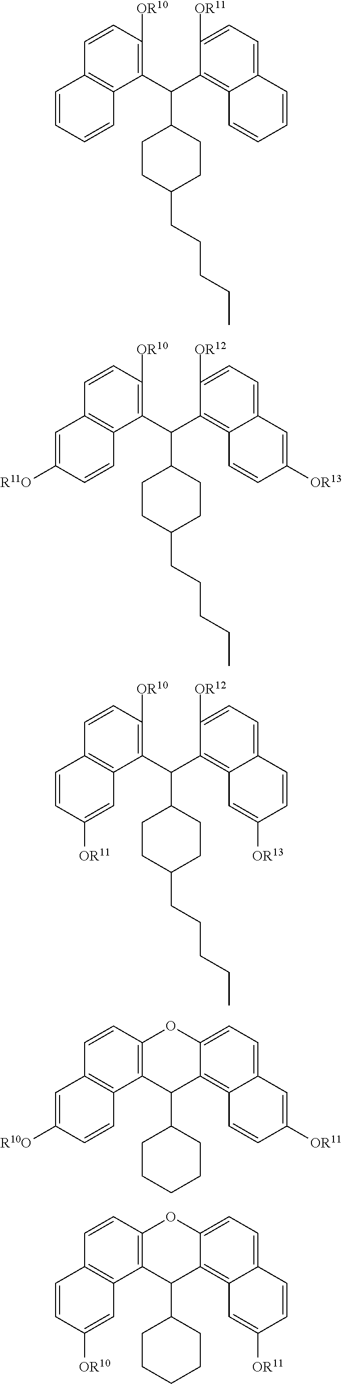

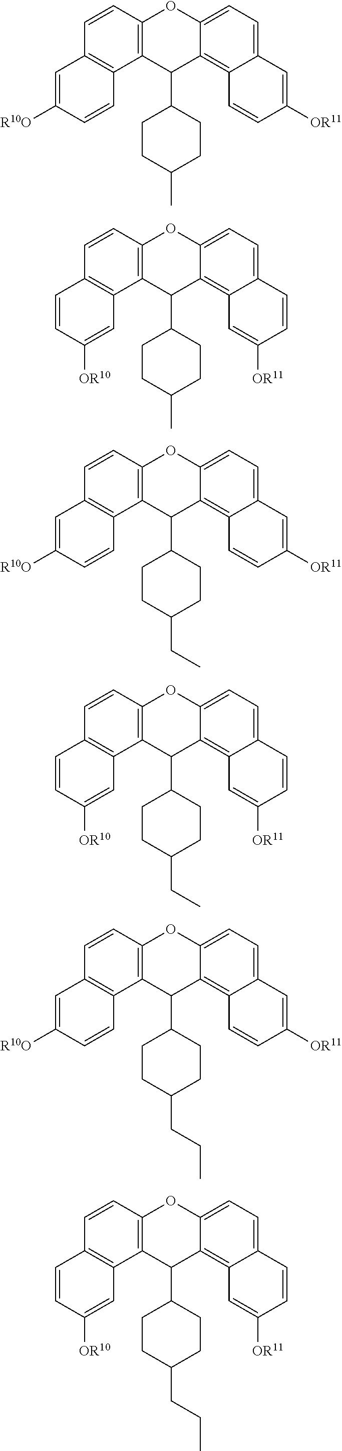

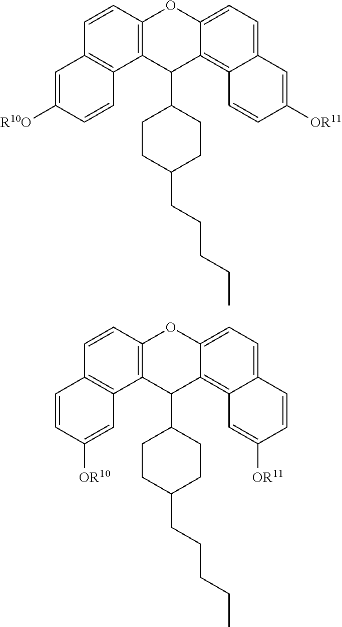

In the above formulae, R¹⁰ to R¹³ are as defined in those described in the above formula (1-2).

The compounds represented by the above formulae more preferably have a dibenzoxanthene skeleton from the viewpoint of heat resistance.

The compounds represented by the above formulae preferably have the structure given below from the viewpoint of the availability of raw materials.

These compounds more preferably have a dibenzoxanthene skeleton from the viewpoint of heat resistance.

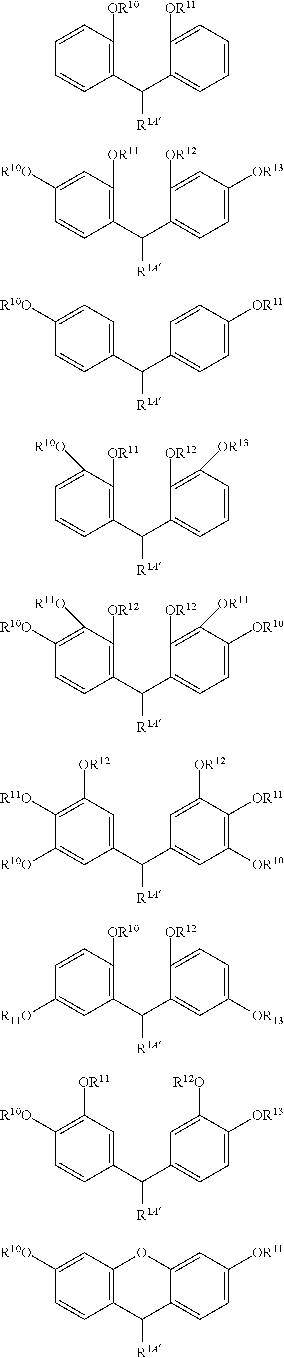

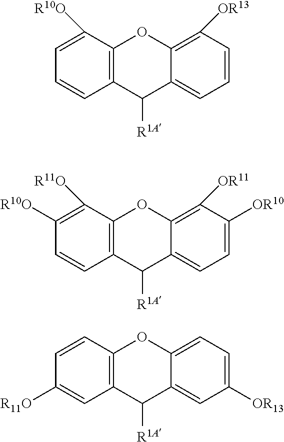

In the above formulae, R^{1A′} is as defined in R^{Z}; and R¹⁰ to R¹³ are as defined in those described in the above formula (1-2).

The compounds represented by the above formulae more preferably have a xanthene skeleton from the viewpoint of heat resistance.

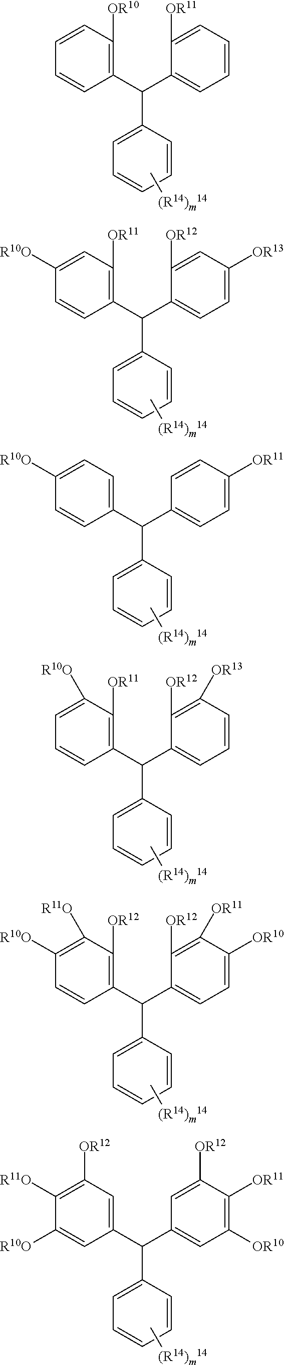

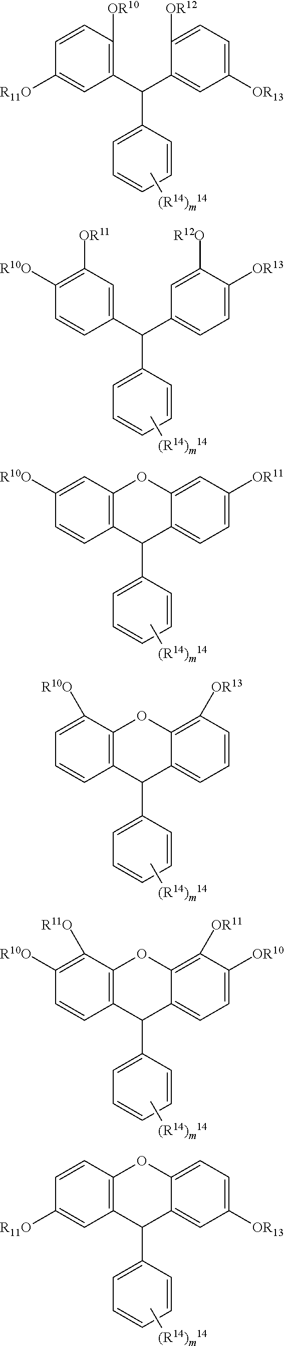

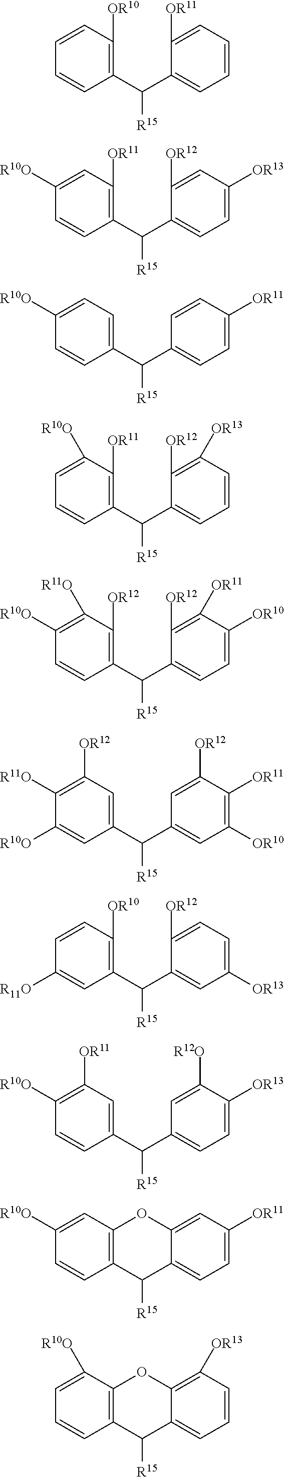

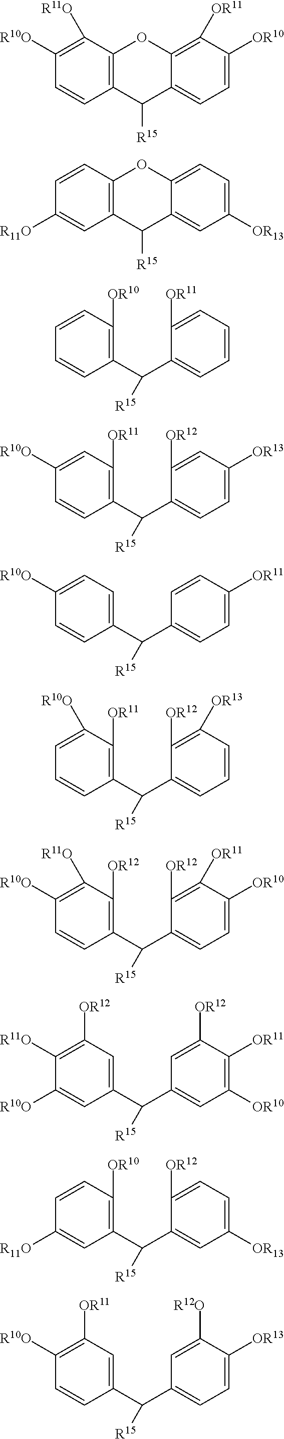

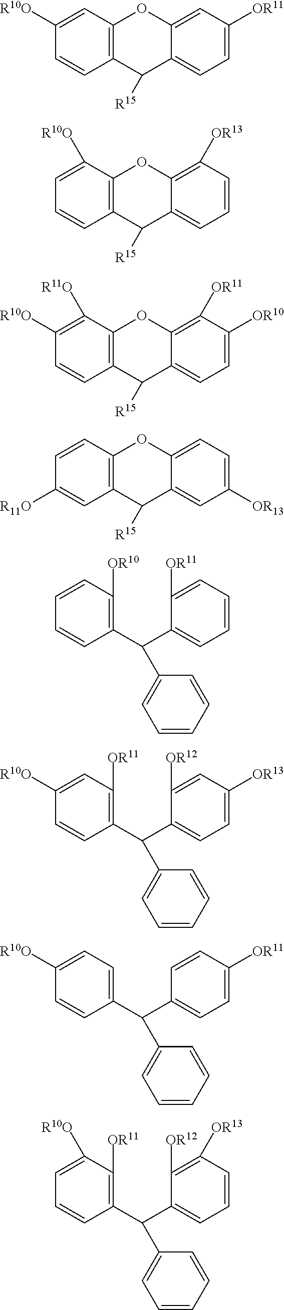

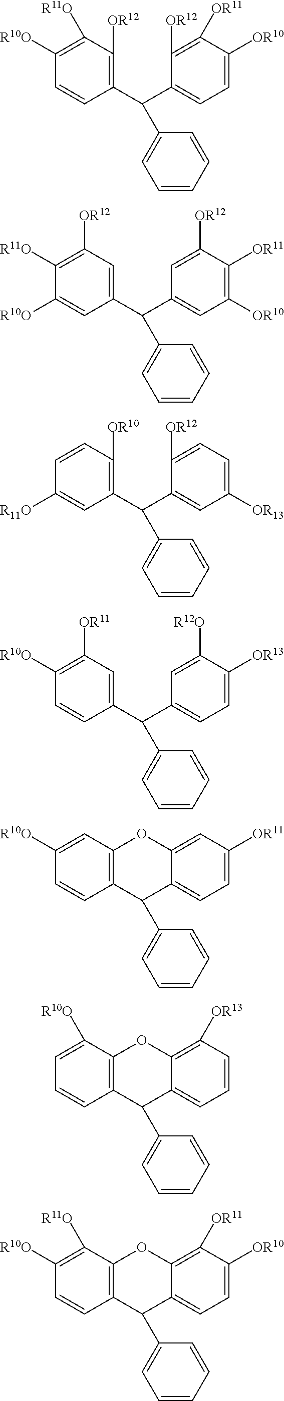

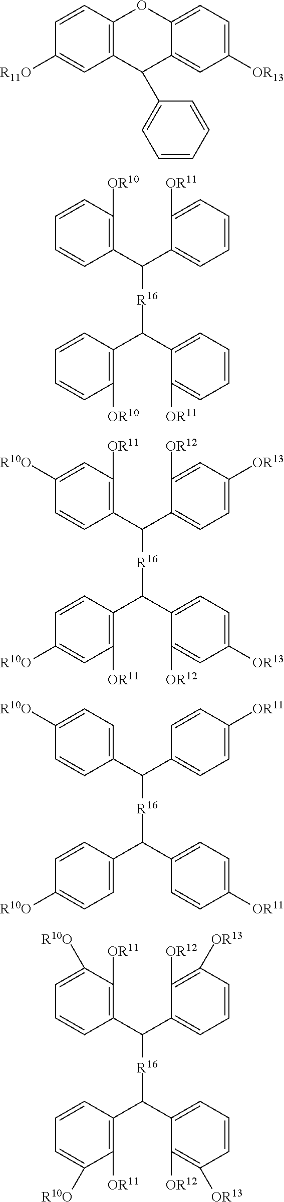

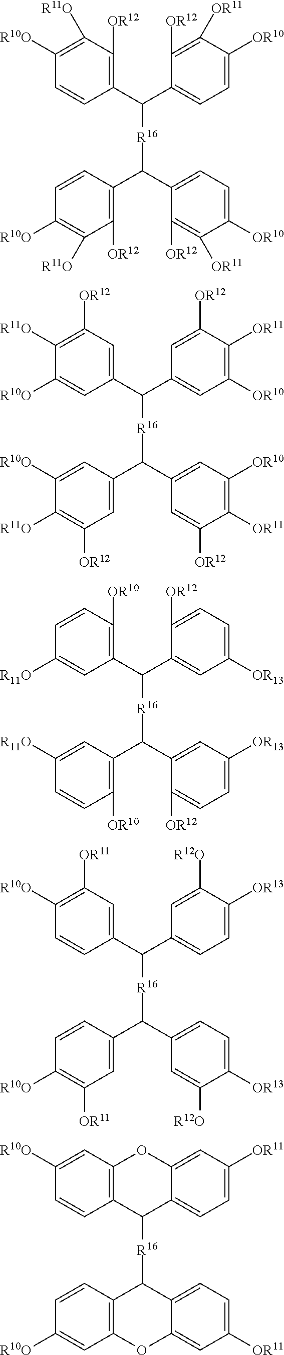

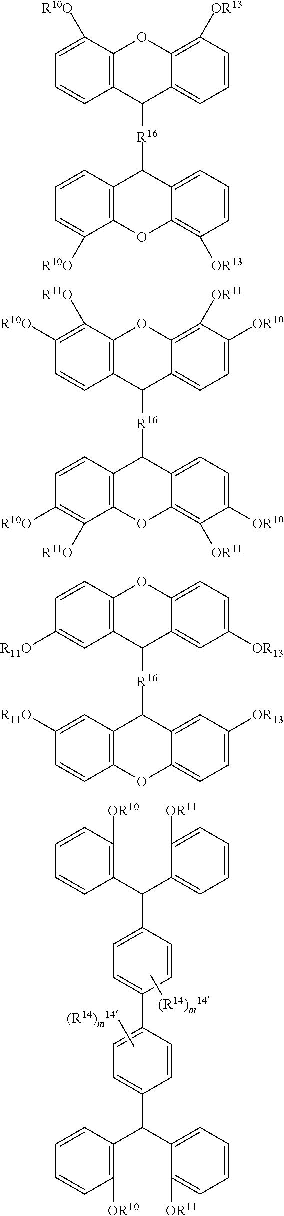

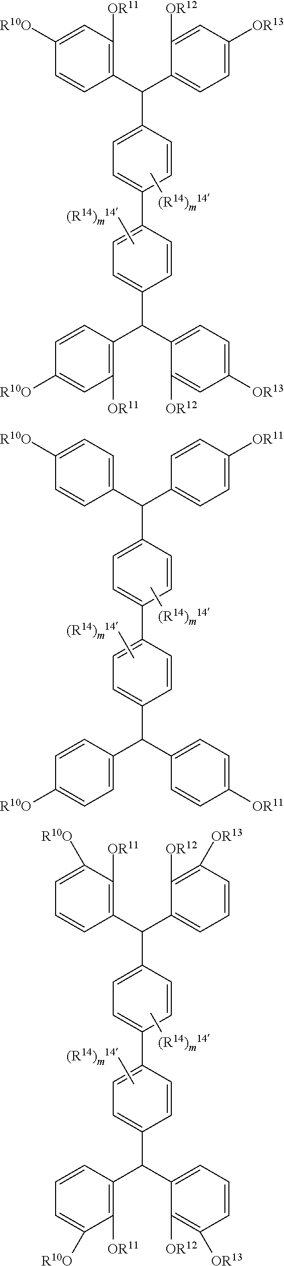

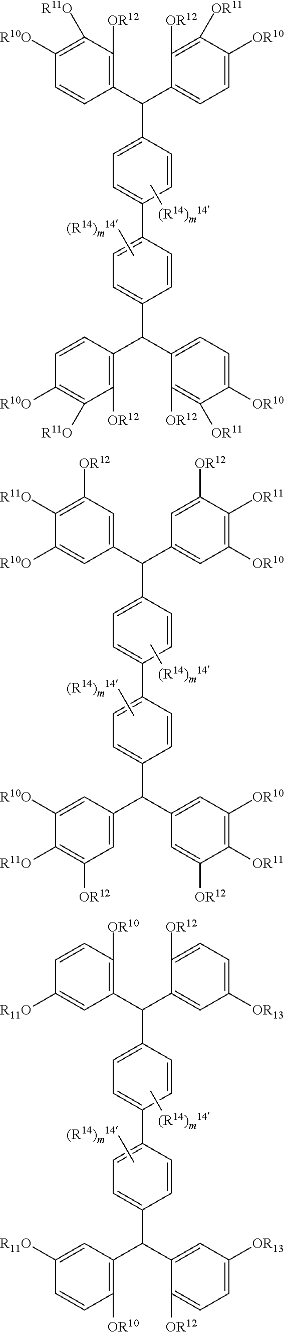

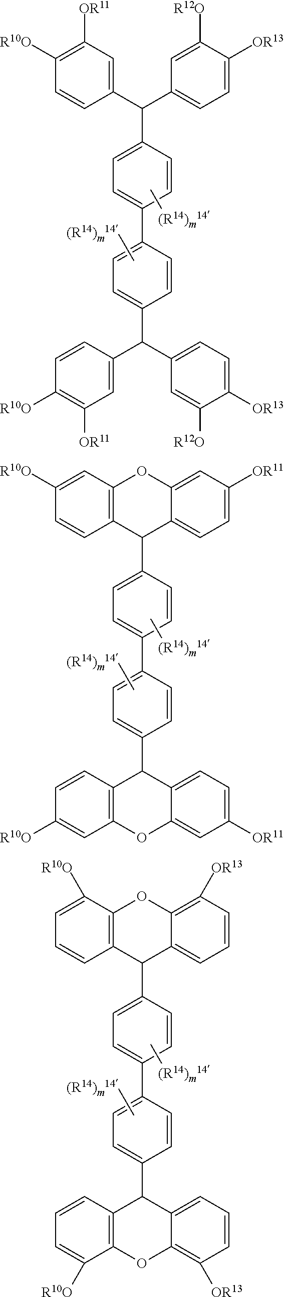

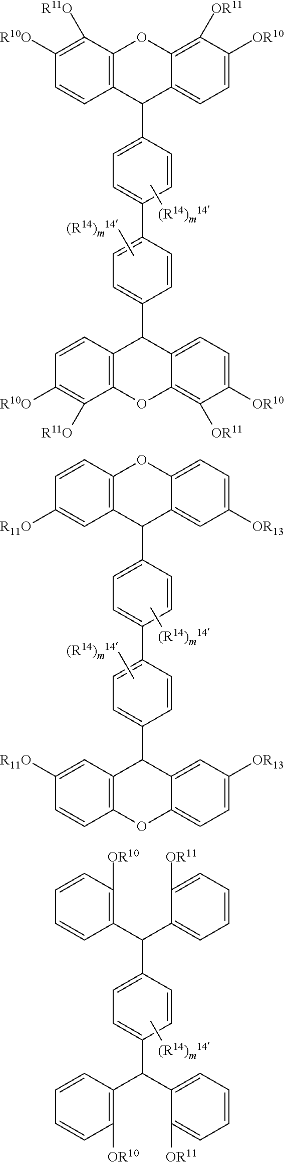

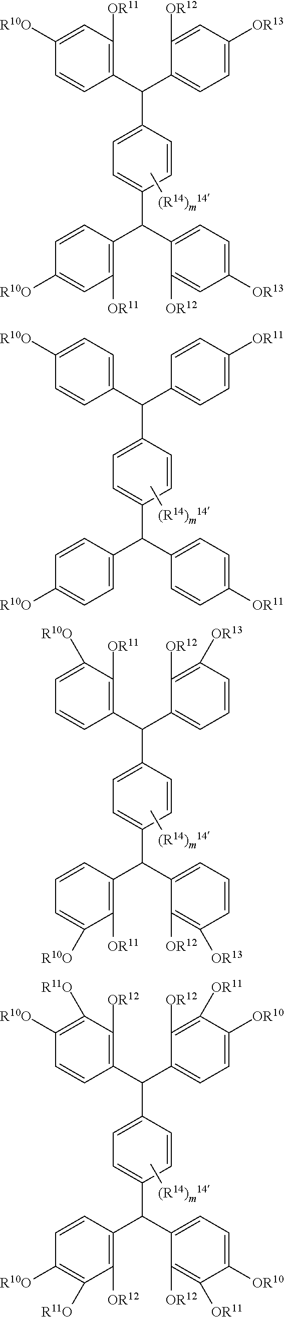

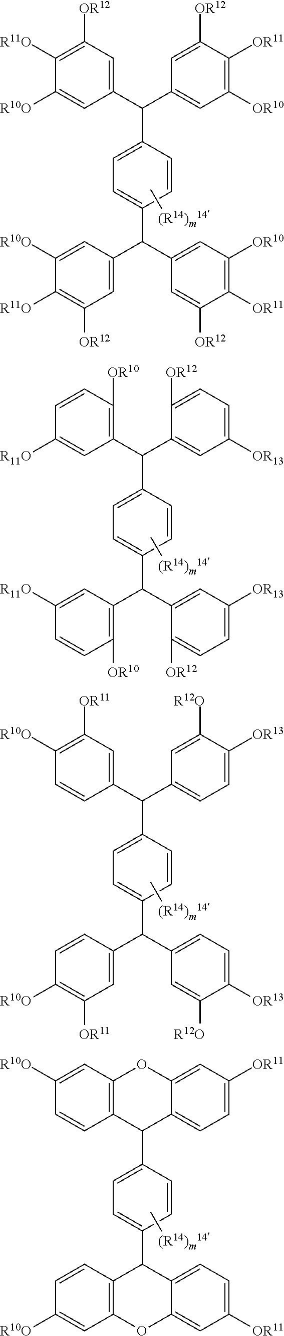

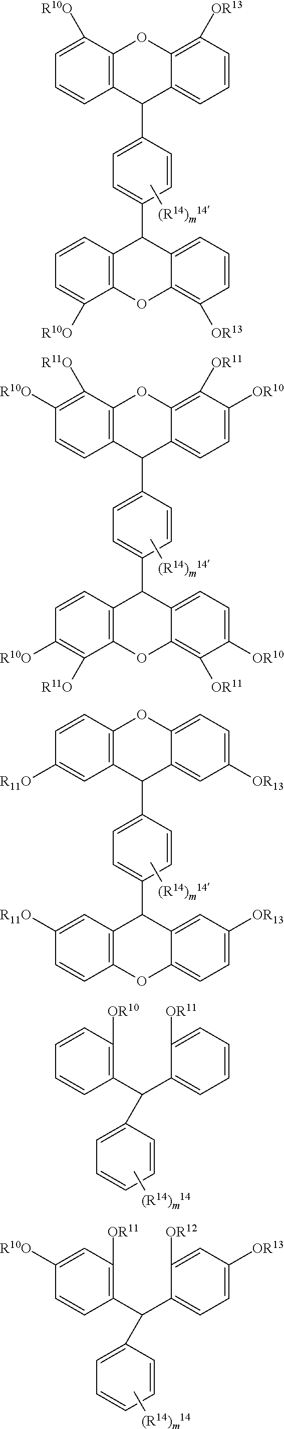

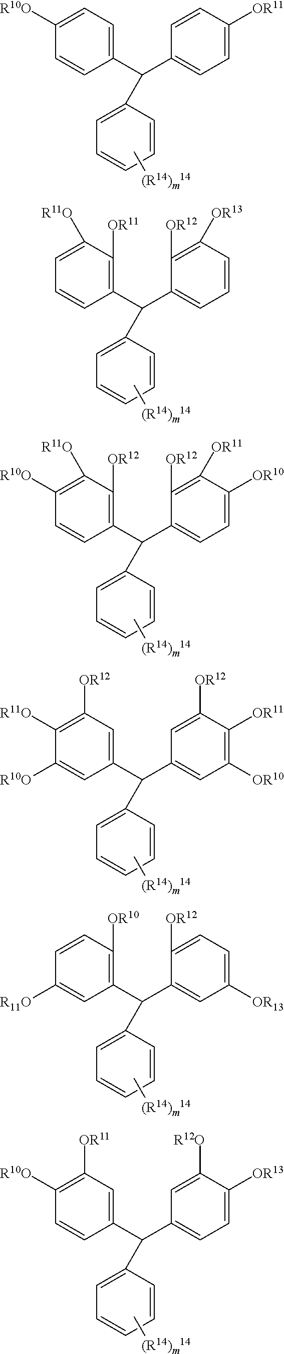

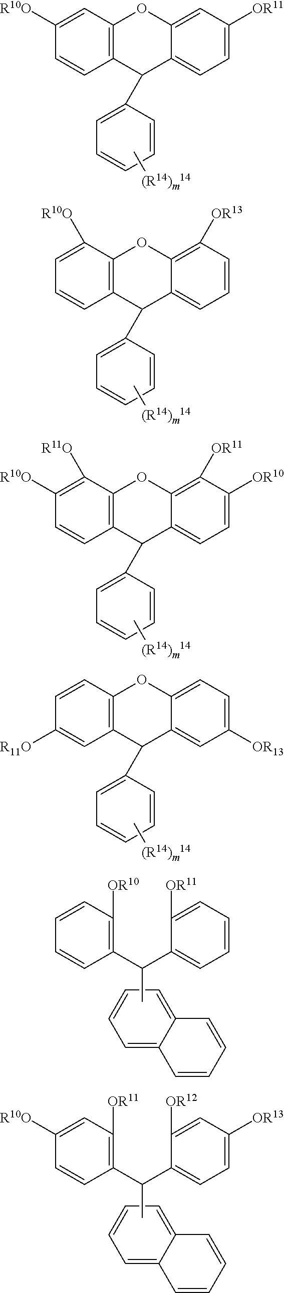

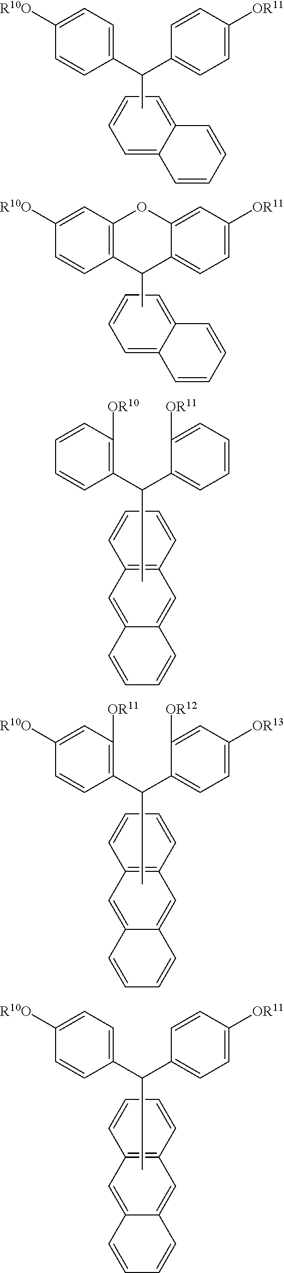

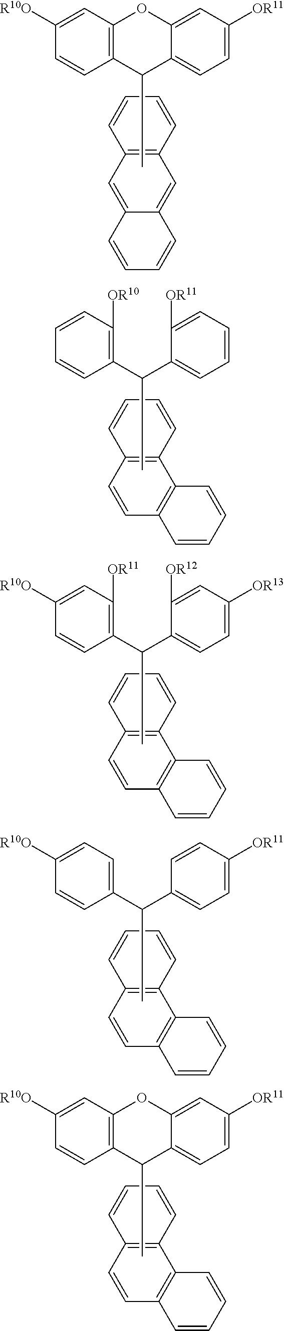

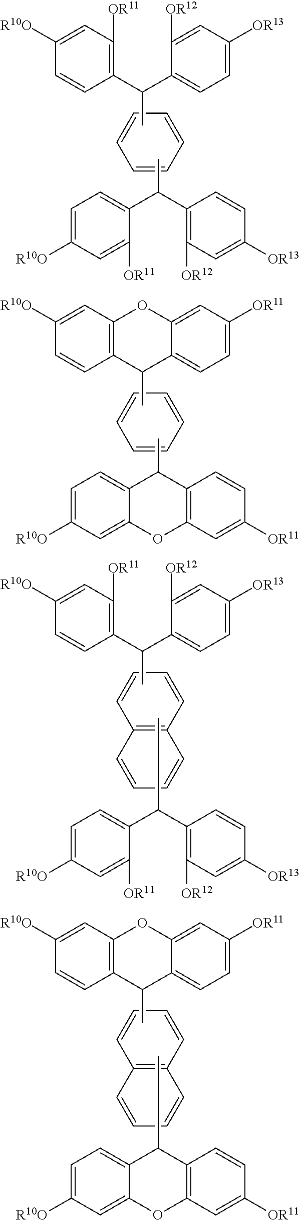

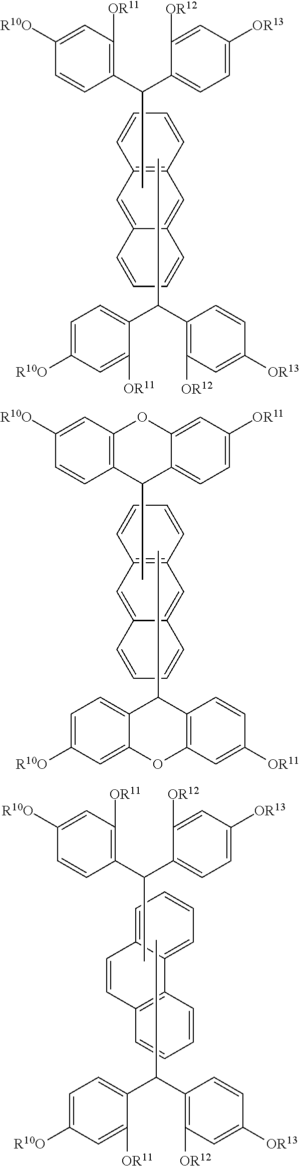

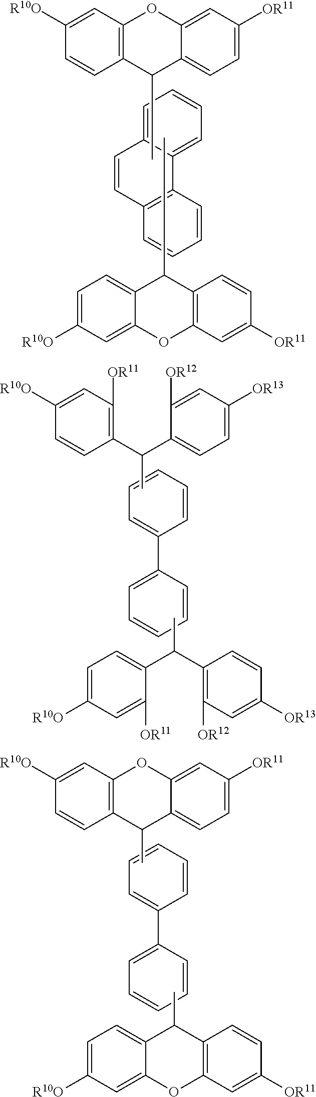

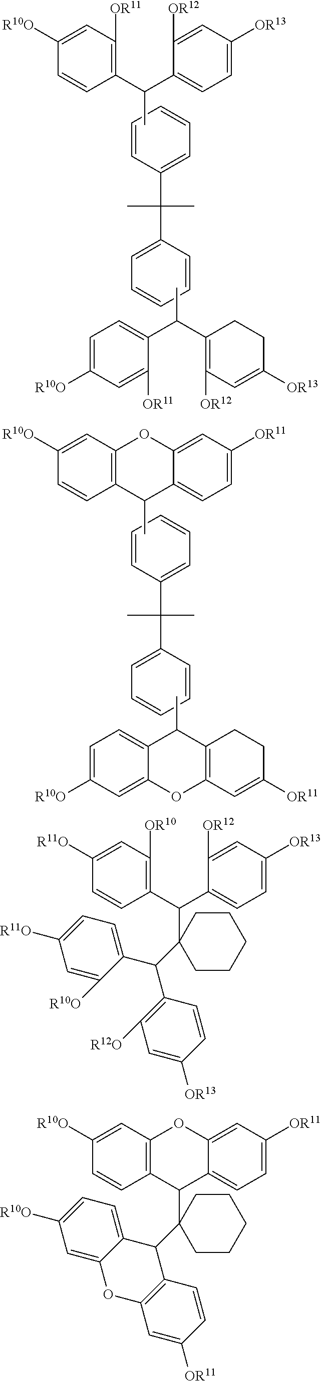

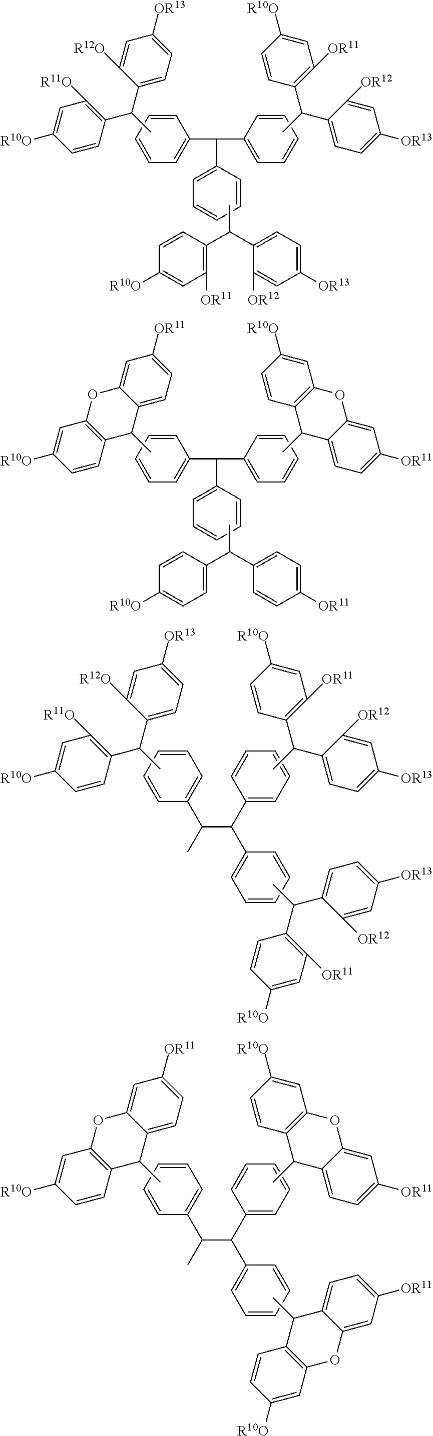

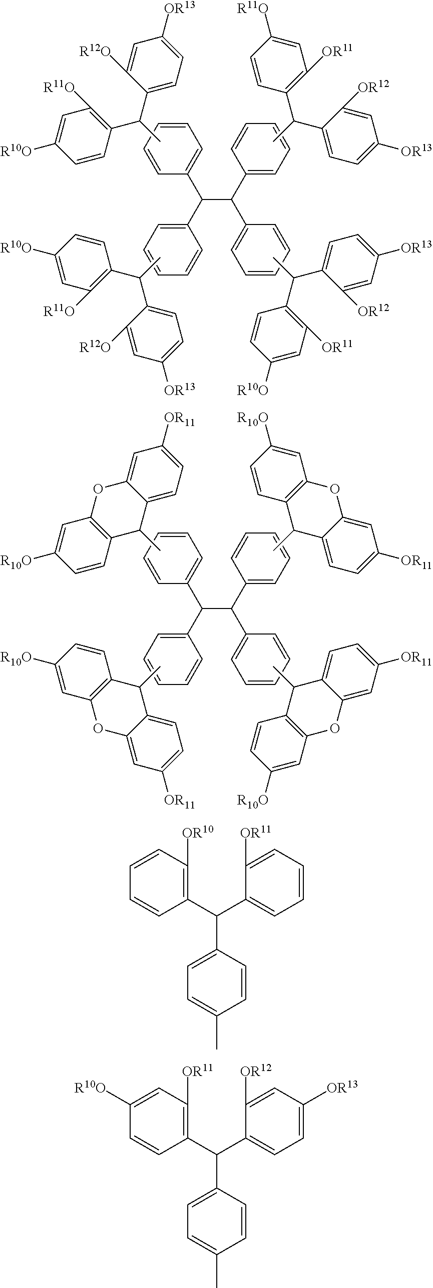

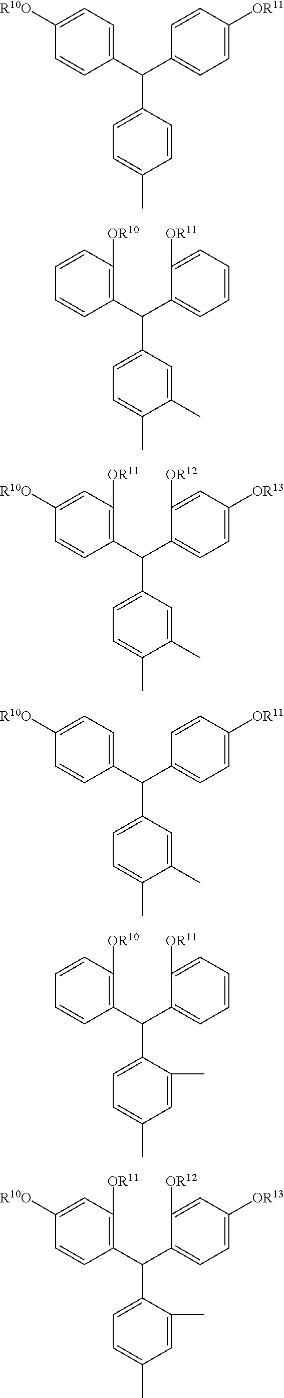

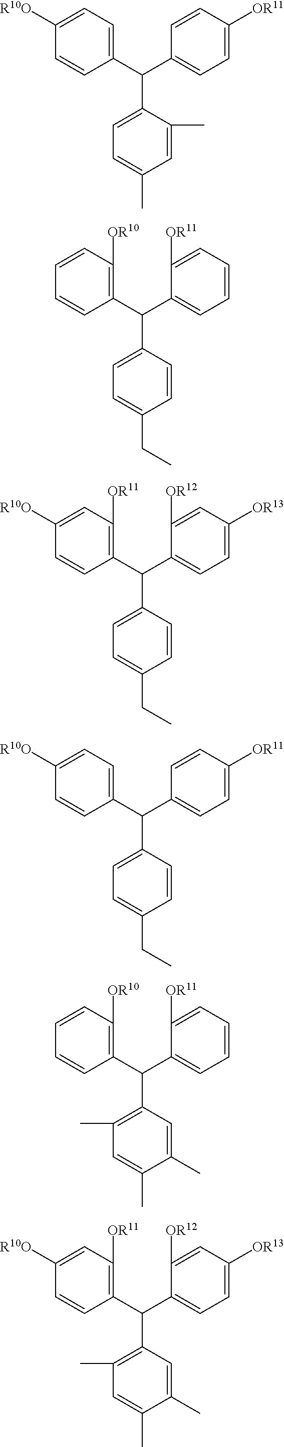

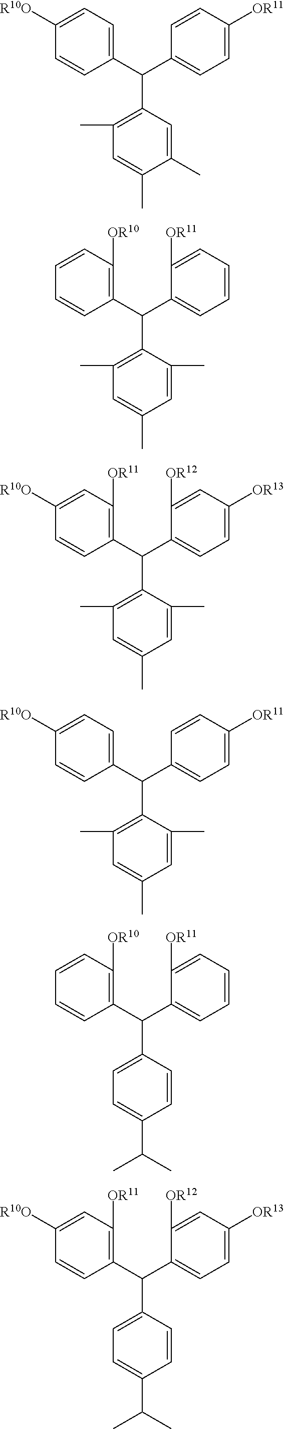

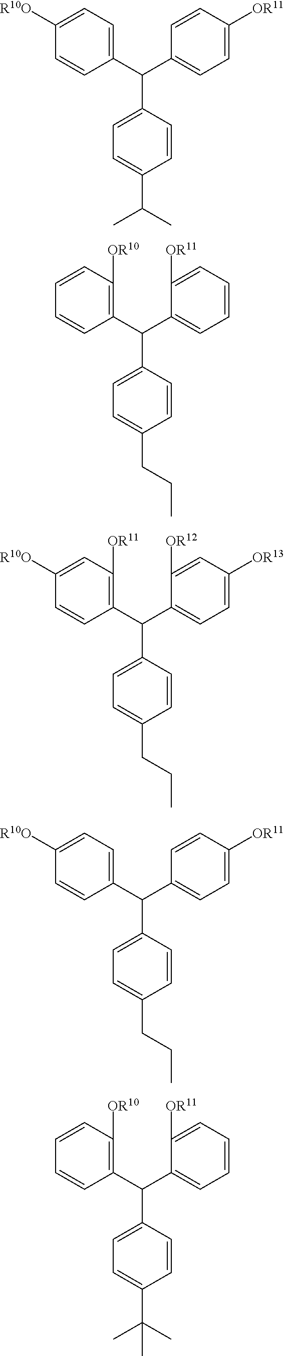

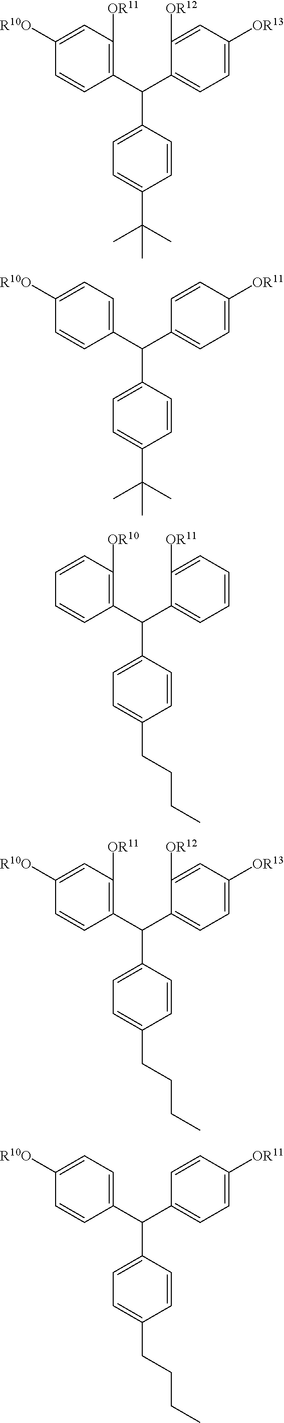

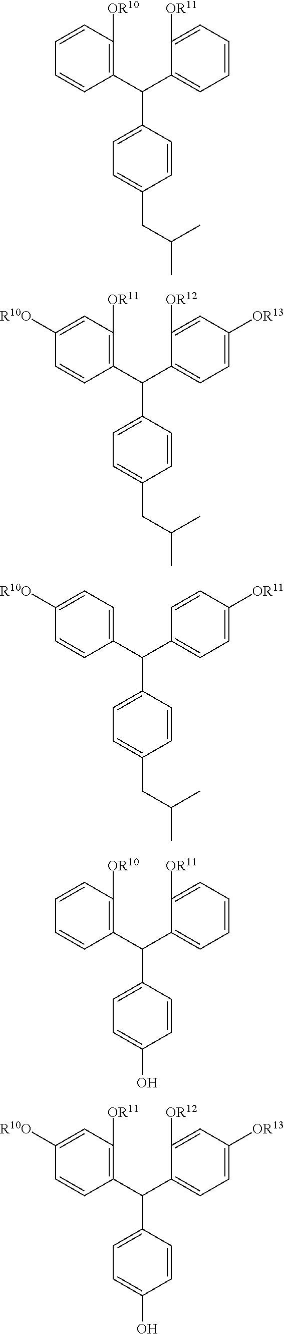

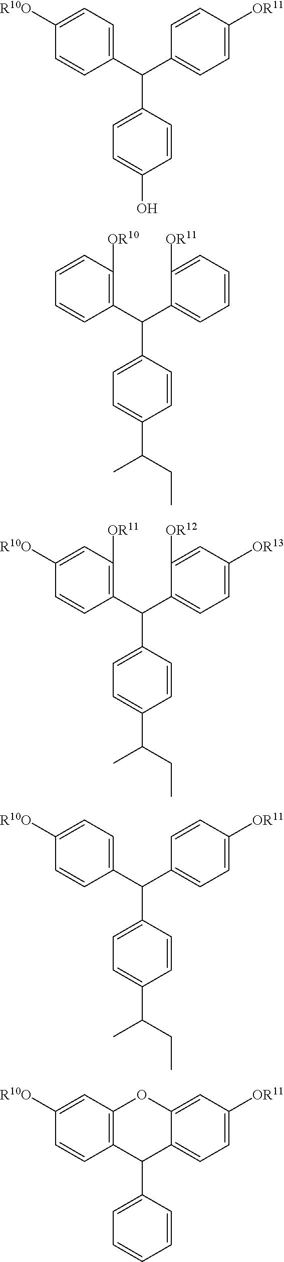

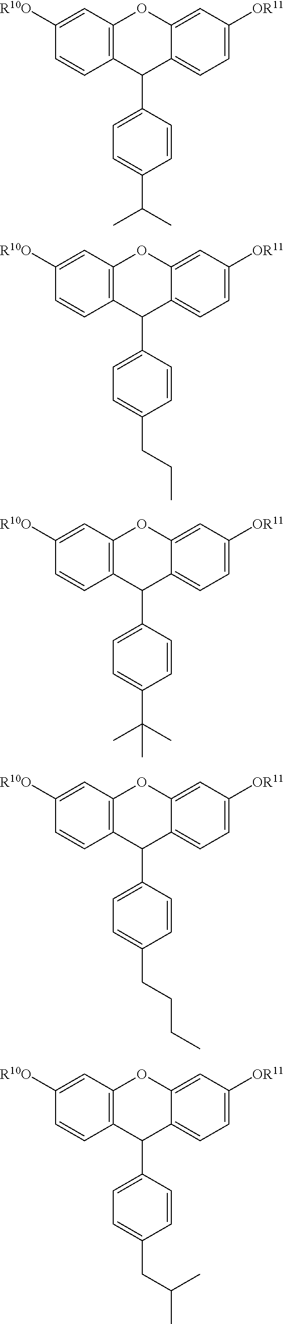

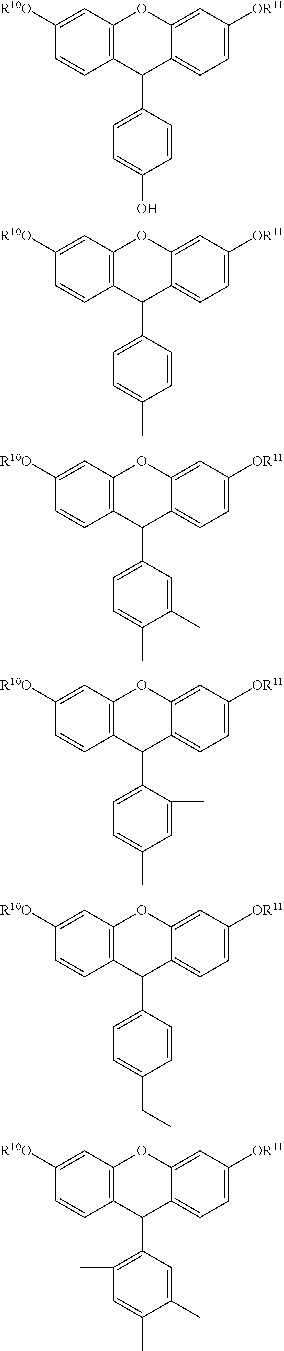

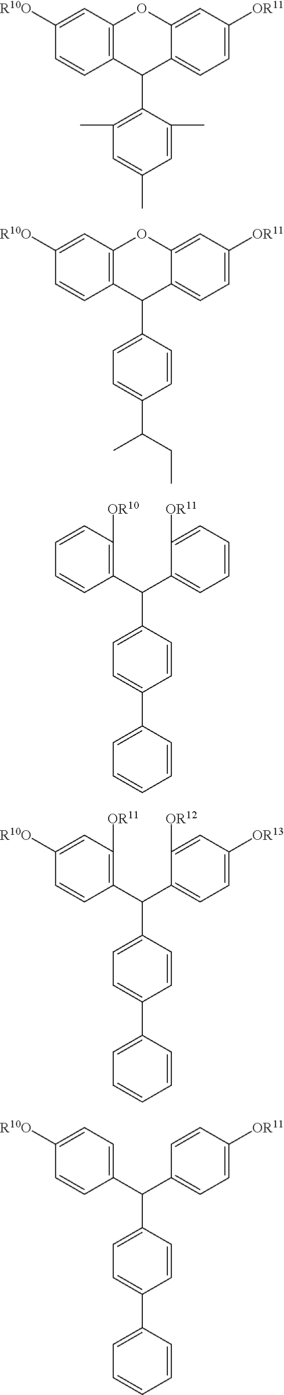

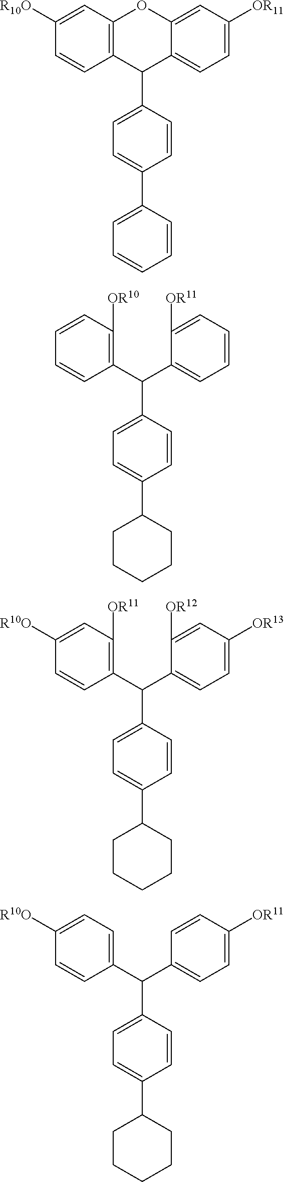

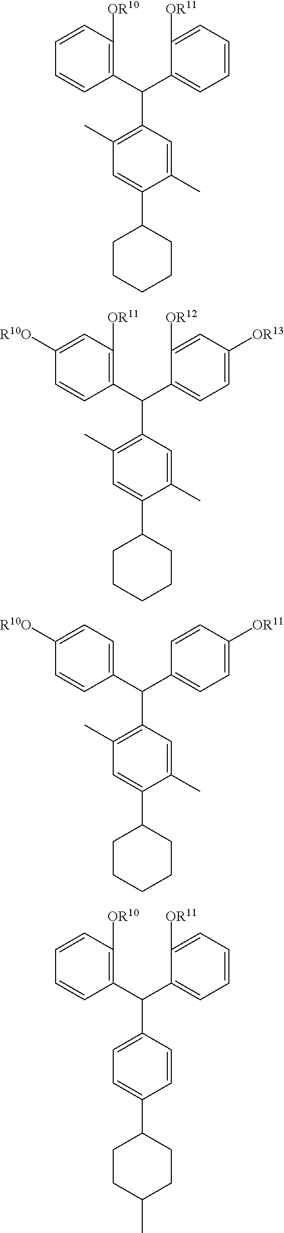

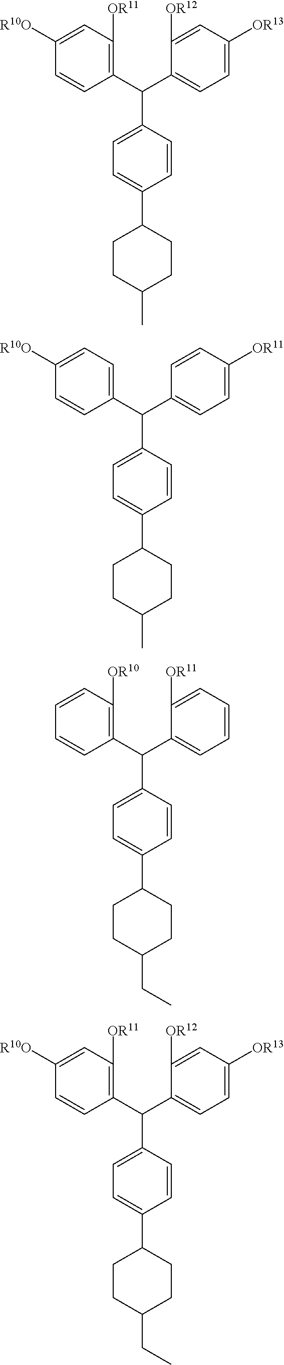

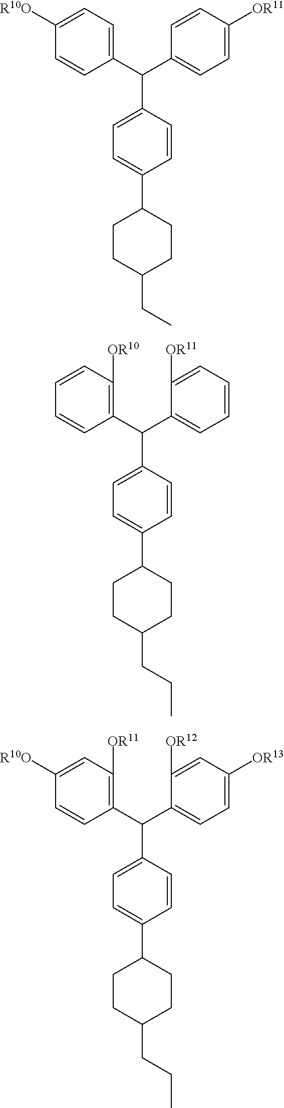

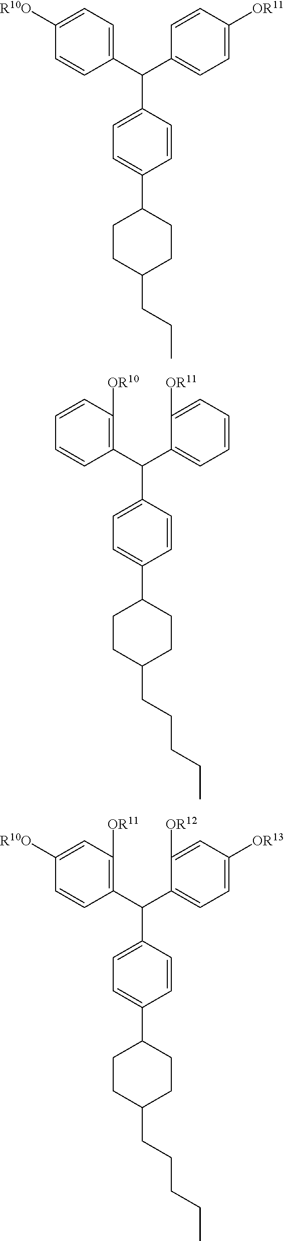

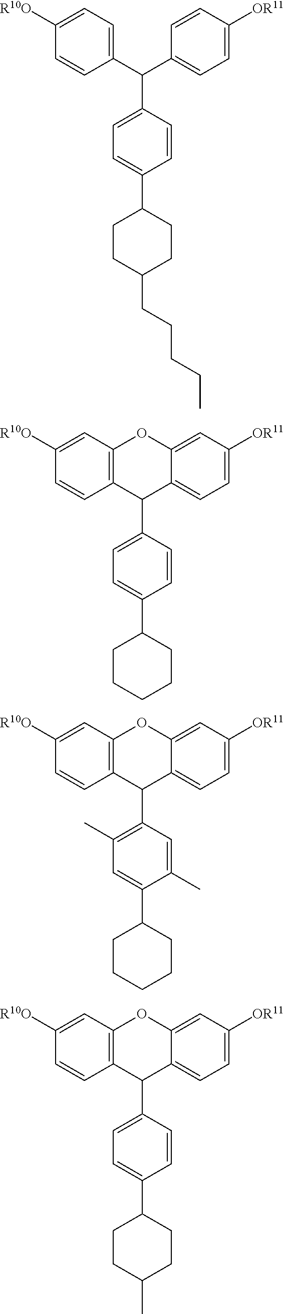

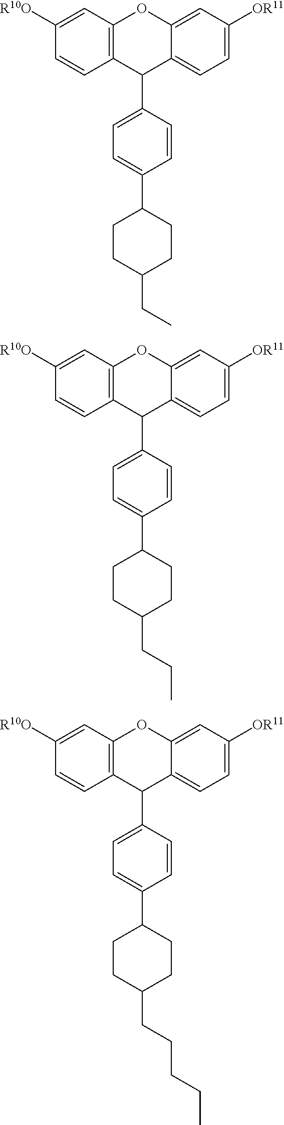

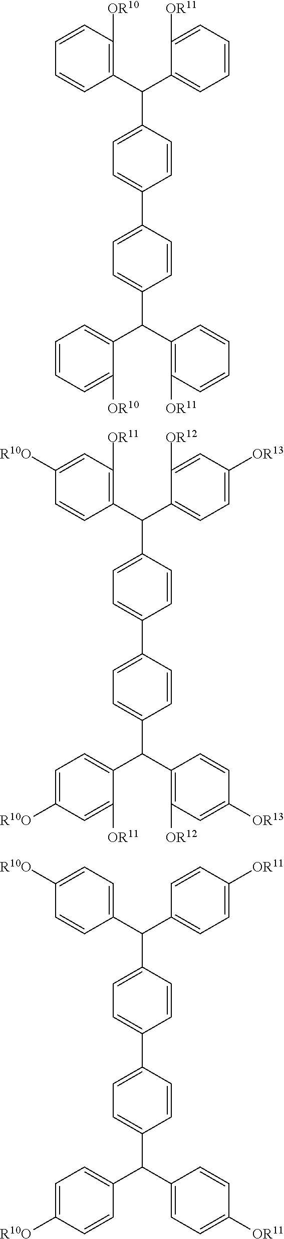

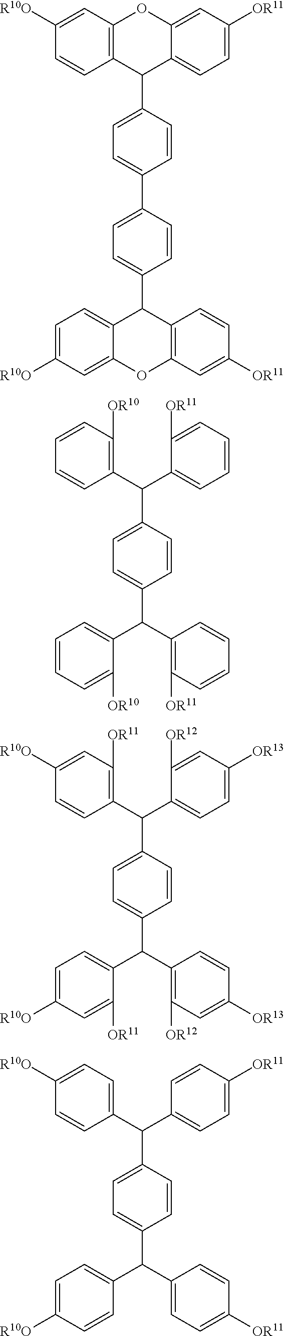

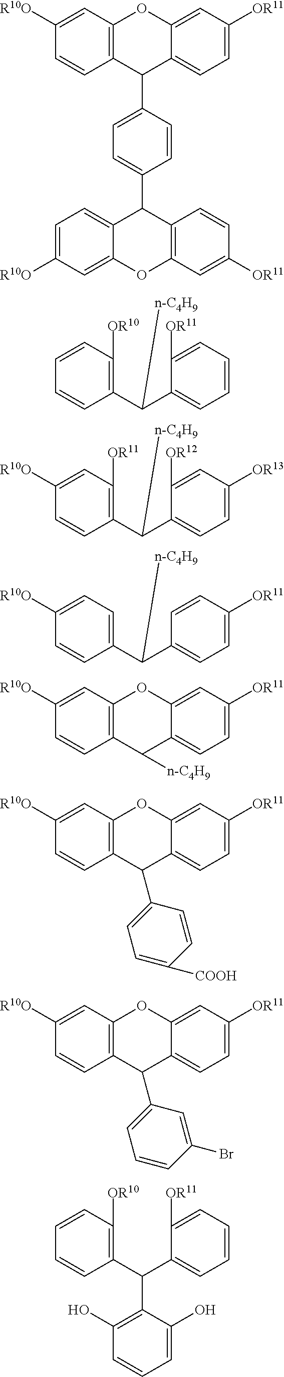

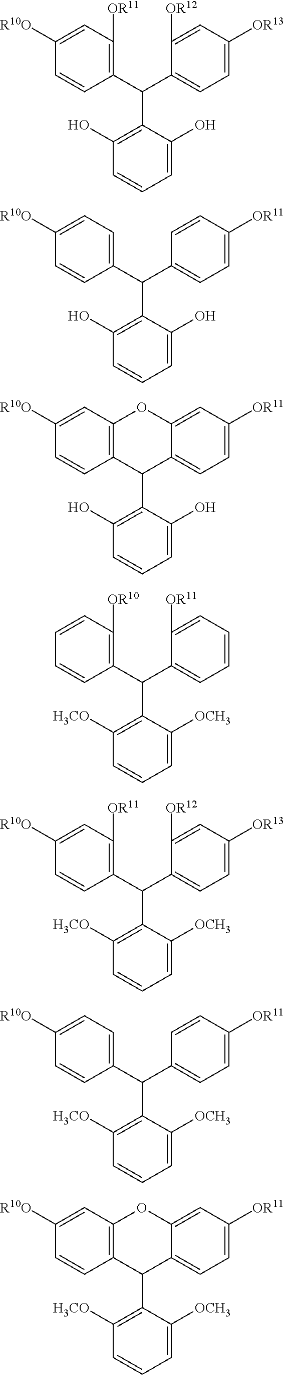

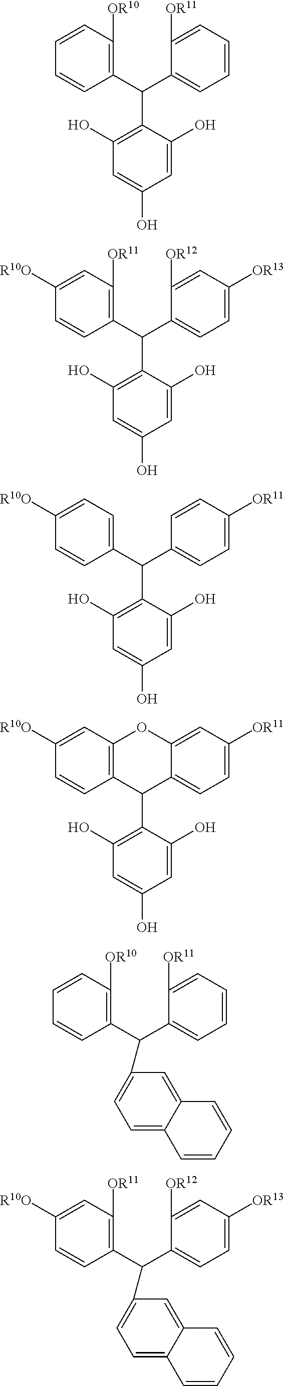

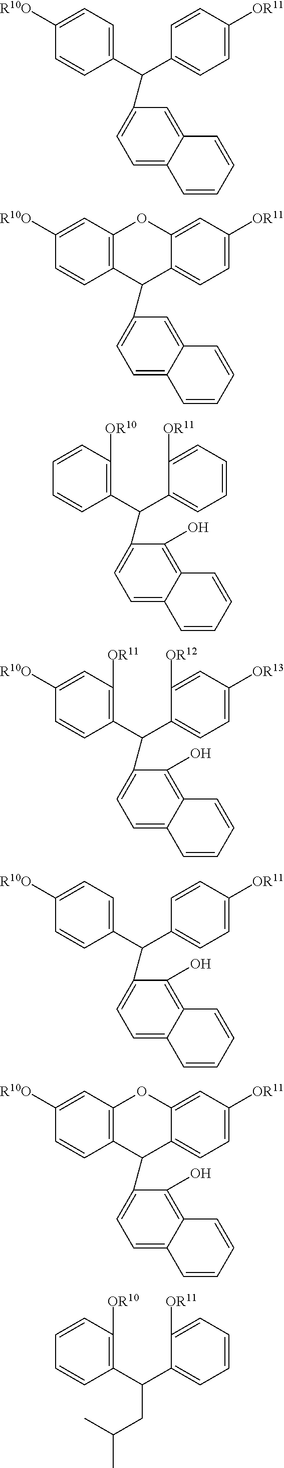

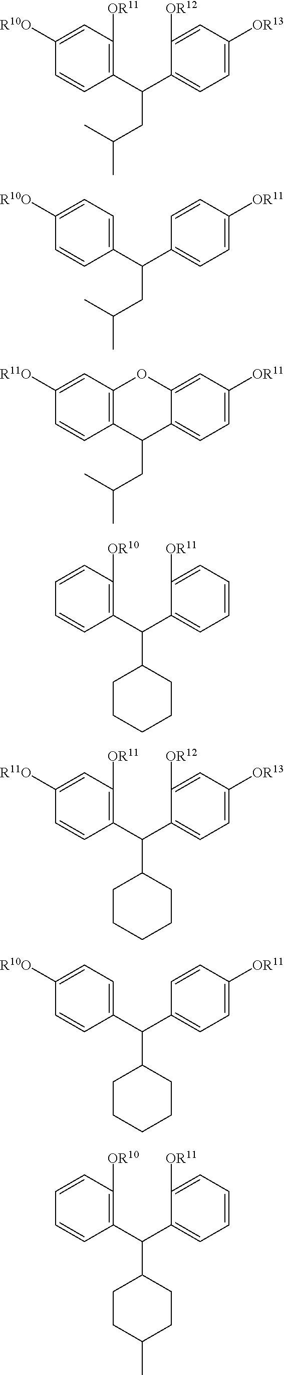

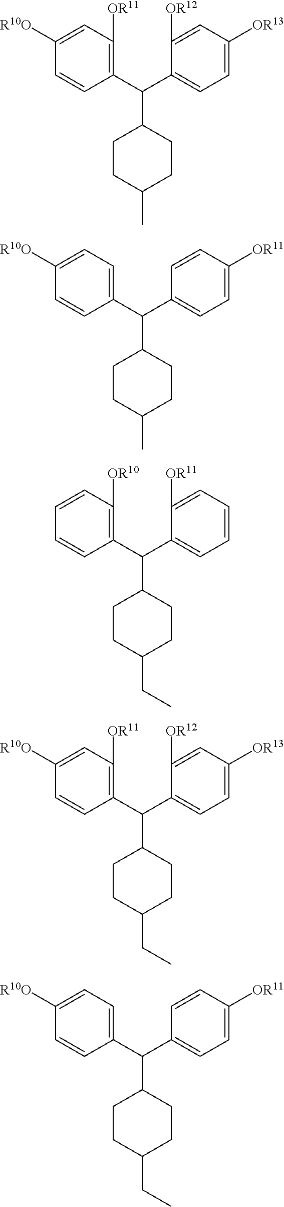

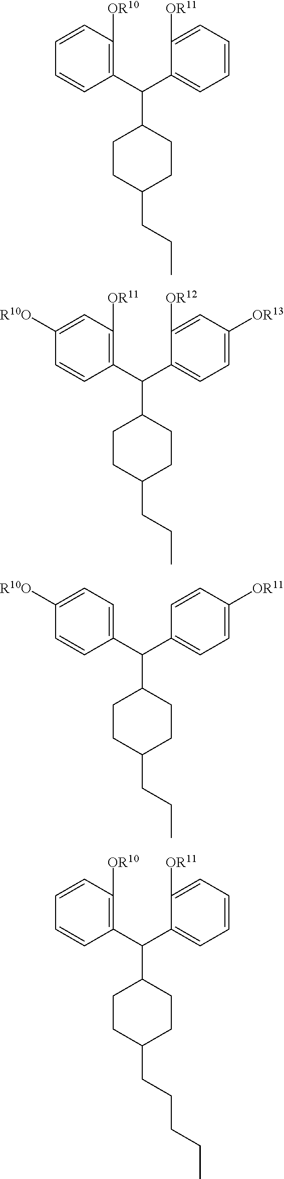

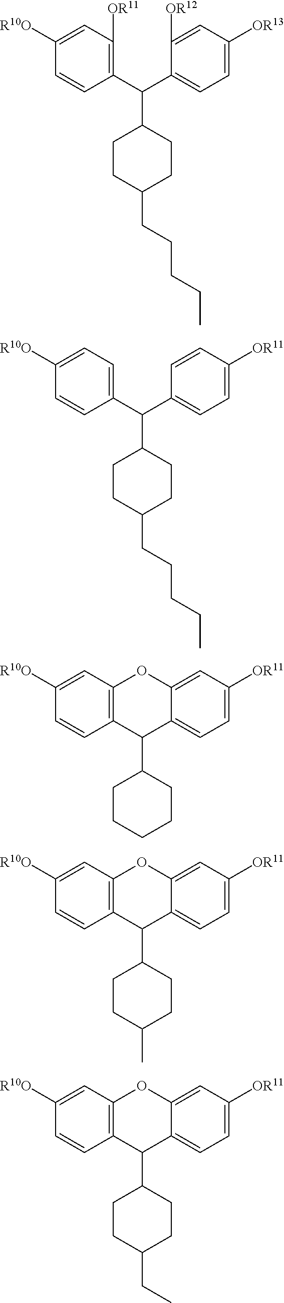

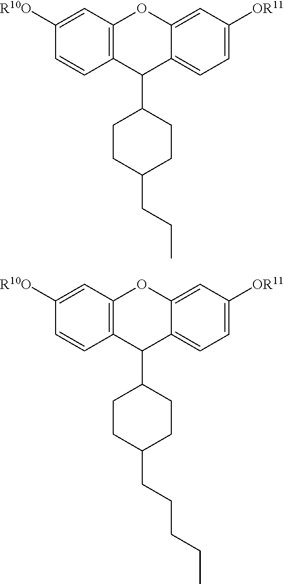

In the above formulae, R¹⁰ to R¹³ are as defined in those described in the above formula (1-2); and R¹⁴, R¹⁵, R¹⁶, m¹⁴, and m^{14′} are as defined above.

### [Method for Producing Compound Represented by Formula (1)]

The compound represented by the formula (1) according to the present embodiment can be arbitrarily synthesized by the application of a publicly known approach, and the synthesis approach is not particularly limited.

The compound represented by the above formula (1) can be obtained, for example, by subjecting a biphenol, a binaphthol or bianthracenol, and a corresponding aldehyde to polycondensation reaction in the presence of an acid catalyst at normal pressure. Also, an acid dissociation group or an acid crosslinking group can be introduced to at least one phenolic hydroxy group of the compound by a publicly known method. If necessary, this reaction can also be carried out under increased pressure.

Examples of the biphenol include, but not particularly limited to, biphenol, methylbiphenol, and methoxybinaphthol. These biphenols can be used alone as one kind or can be used in combination of two or more kinds. Among them, biphenol is more preferably used from the viewpoint of the stable supply of raw materials.

Examples of the binaphthol include, but not particularly limited to, binaphthol, methylbinaphthol, and methoxybinaphthol. These binaphthols can be used alone as one kind or can be used in combination of two or more kinds. Among them, binaphthol is more preferably used from the viewpoint of increasing a carbon atom concentration and improving heat resistance.

Examples of the aldehyde include, but not particularly limited to, formaldehyde, trioxane, paraformaldehyde, benzaldehyde, acetaldehyde, propylaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, biphenylaldehyde, naphthaldehyde, anthracenecarbaldehyde, phenanthrenecarbaldehyde, pyrenecarbaldehyde, and furfural. These aldehydes can be used alone as one kind or can be used in combination of two or more kinds. Among them, benzaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde, or furfural is preferably used from the viewpoint of conferring high heat resistance. Benzaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde, or furfural is more preferably used from the viewpoint of improving etching resistance.

An aldehyde having an aromatic ring is preferably used as the aldehyde from the viewpoint of possessing both high heat resistance and high etching resistance.

The acid catalyst used in the above reaction can be arbitrarily selected and used from publicly known catalysts and is not particularly limited. Inorganic acids and organic acids are widely known as such acid catalysts, and examples include, but not particularly limited to, inorganic acids such as hydrochloric acid, sulfuric acid, phosphoric acid, hydrobromic acid, and hydrofluoric acid; organic acids such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; Lewis acids such as zinc chloride, aluminum chloride, iron chloride, and boron trifluoride; and solid acids such as tungstosilicic acid, tungstophosphoric acid, silicomolybdic acid, and phosphomolybdic acid. Among them, organic acids and solid acids are preferable from the viewpoint of production, and hydrochloric acid or sulfuric acid is more preferably used from the viewpoint of production such as easy availability and handleability. The acid catalysts can be used alone as one kind or can be used in combination of two or more kinds. Also, the amount of the acid catalyst used can be arbitrarily set according to, for example, the kind of the raw materials used and the catalyst used and moreover the reaction conditions and is not particularly limited, but is preferably 0.01 to 100 parts by mass per 100 parts by mass of the reaction raw materials.

Upon the above reaction, a reaction solvent may be used. The reaction solvent is not particularly limited as long as the reaction of the aldehyde used with the biphenol, the binaphthol or the bianthracenediol proceeds, and can be arbitrarily selected and used from publicly known solvents. Examples of the reaction solvent include water, methanol, ethanol, propanol, butanol, tetrahydrofuran, dioxane, ethylene glycol dimethyl ether, ethylene glycol diethyl ether, and a mixed solvent thereof. The solvents can be used alone as one kind or can be used in combination of two or more kinds.

The amount of these reaction solvents used can be arbitrarily set according to, for example, the kind of the raw materials used and the catalyst and moreover the reaction conditions and is not particularly limited, but is preferably in the range of 0 to 2000 parts by mass per 100 parts by mass of the reaction raw materials. Furthermore, the reaction temperature in the above reaction can be arbitrarily selected according to the reactivity of the reaction raw materials and is not particularly limited, but is usually in the range of 10 to 200° C.

In order to obtain the compound represented by the formula (1) according to the present embodiment, a higher reaction temperature is more preferable. Specifically, the range of 60 to 200° C. is preferable. The reaction method can be arbitrarily selected and used from publicly known approaches and is not particularly limited. Examples thereof include a method of charging the biphenol, the binaphthol or the bianthracenediol, the aldehyde, and the catalyst in one portion, and a method of dropping the biphenol, the binaphthol or the bianthracenediol, and the ketone, in the presence of the catalyst. After the polycondensation reaction terminates, isolation of the obtained compound can be carried out according to a conventional method, and is not particularly limited. For example, by adopting a commonly used approach in which the temperature of the reaction vessel is elevated to 130 to 230° C. in order to remove unreacted raw materials, catalyst, etc. present in the system, and volatile portions are removed at about 1 to 50 mmHg, the compound which is the target component can be isolated.

Examples of preferable reaction conditions include conditions involving using 1 mol to an excess of the biphenol, the binaphthol or the bianthracenediol and 0.001 to 1 mol of the acid catalyst per 1 mol of the aldehyde, and reacting them at 50 to 150° C. at normal pressure for about 20 minutes to 100 hours.

The target component can be isolated by a publicly known method after the reaction terminates. The compound represented by the above formula (1) which is the target component can be obtained, for example, by concentrating the reaction solution, precipitating the reaction product by the addition of pure water, cooling the reaction solution to room temperature, then separating the precipitates by filtration, filtering and drying the obtained solid matter, then separating and purifying the solid matter from by-products by column chromatography, and distilling off the solvent, followed by filtration and drying.

A method for introducing an acid dissociation group or an acid crosslinking group to at least one phenolic hydroxy group of a polyphenol compound is publicly known. An acid dissociation group or an acid crosslinking group can be introduced to at least one phenolic hydroxy group of the compound, for example, as described below.

A compound for introducing the acid dissociation group can be synthesized by a publicly known method or easily obtained. Examples thereof include, but not particularly limited to, acid chlorides, acid anhydrides, active carboxylic acid derivative compounds such as dicarbonate, alkyl halides, vinyl alkyl ethers, dihydropyran, and halocarboxylic acid alkyl esters.

For example, the compound is dissolved or suspended in an aprotic solvent such as acetone, tetrahydrofuran (THF), or propylene glycol monomethyl ether acetate. Subsequently, a vinyl alkyl ether such as ethyl vinyl ether, or dihydropyran is added to the solution or the suspension, and the mixture is reacted at 20 to 60° C. at normal pressure for 6 to 72 hours in the presence of an acid catalyst such as pyridinium p-toluenesulfonate. The reaction solution is neutralized with an alkali compound and added to distilled water to precipitate a white solid. Then, the separated white solid can be washed with distilled water and dried to obtain the compound in which a hydrogen atom of the hydroxy group is substituted with the acid dissociation group.

Also, for example, the compound having a hydroxy group is dissolved or suspended in an aprotic solvent such as acetone, THF, or propylene glycol monomethyl ether acetate. Subsequently, an alkyl halide such as ethyl chloromethyl ether or a halocarboxylic acid alkyl ester such as methyladamantyl bromoacetate is added to the solution or the suspension, and the mixture is reacted at 20 to 110° C. at normal pressure for 6 to 72 hours in the presence of an alkali catalyst such as potassium carbonate. The reaction solution is neutralized with an acid such as hydrochloric acid and added to distilled water to precipitate a white solid. Then, the separated white solid can be washed with distilled water and dried to obtain the compound in which a hydrogen atom of the hydroxy group is substituted with the acid dissociation group.

The timing of introducing the acid dissociation group is not only after the condensation reaction of the binaphthol with the ketone, but may be prior to the condensation reaction. Alternatively, this introduction may be carried out after production of a resin mentioned later.

In the present embodiment, the acid dissociation group refers to a characteristic group that is cleaved in the presence of an acid to form a functional group that changes solubility, such as an alkali soluble group. Examples of the alkali soluble group include a phenolic hydroxy group, a carboxyl group, a sulfonic acid group, and a hexafluoroisopropanol group. Among them, a phenolic hydroxy group and a carboxyl group are preferable, and a phenolic hydroxy group is particularly preferable, from the viewpoint of alkali developability. The acid dissociation group preferably has the property of causing chained cleavage reaction in the presence of an acid, from the viewpoint of improvement in productivity of optical member formation.

A compound for introducing the acid crosslinking group can be synthesized by a publicly known method or easily obtained. Examples thereof include, but not particularly limited to, allyl halides, acrylic acid, methacrylic acid, acrylic acid halides, methacrylic acid halides, vinyl benzyl halides, and epihalohydrins.

For example, the compound is dissolved or suspended in an aprotic solvent such as acetone, tetrahydrofuran (THF), or propylene glycol monomethyl ether acetate. Subsequently, an epihalohydrin such as epichlorohydrin or epibromohydrin is added to the solution or the suspension, and the mixture is reacted at 0 to 60° C. at normal pressure for 6 to 72 hours in the presence of an acid catalyst such as hydrochloric acid. The reaction solution is neutralized with an alkali compound and added to distilled water to precipitate a white solid. Then, the separated white solid can be washed with distilled water and dried to obtain the compound in which a hydrogen atom of the hydroxy group is substituted with the acid crosslinking group.

Also, for example, the compound having a hydroxy group is dissolved or suspended in an aprotic solvent such as acetone, THF, or propylene glycol monomethyl ether acetate. Subsequently, an allyl halide such as allyl chloride or allyl bromide, acrylic acid, methacrylic acid, an acrylic acid halide such as acrylic acid chloride or acrylic acid bromide, a methacrylic acid halide such as methacrylic acid chloride or methacrylic acid bromide, or a vinyl benzyl halide such as vinyl benzyl chloride or vinyl benzyl bromide is added to the solution or the suspension, and the mixture is reacted at 0 to 110° C. at normal pressure for 6 to 72 hours in the presence of an alkali catalyst such as sodium hydroxide, triethylamine, or potassium carbonate. The reaction solution is neutralized with an acid such as hydrochloric acid and added to distilled water to precipitate a white solid. Then, the separated white solid can be washed with distilled water and dried to obtain the compound in which a hydrogen atom of the hydroxy group is substituted with the acid crosslinking group.

The timing of introducing the acid crosslinking group is not only after the condensation reaction of the binaphthol with the ketone, but may be prior to the condensation reaction. Alternatively, this introduction may be carried out after production of a resin mentioned later.

In the present embodiment, the acid crosslinking group refers to a characteristic group that reacts in the presence of a radical or an acid or an alkali and varies in solubility in an acid, an alkali, or an organic solvent for use in a coating solvent or a developing solution.

Examples of the acid crosslinking group include allyl groups, (meth)acryloyl groups, a vinyl group, an epoxy group, alkoxymethyl groups, and a cyanato group. The acid crosslinking group is not limited thereto as long as it reacts in the presence of a radical or an acid or an alkali.

The acid crosslinking group preferably has the property of causing chained cleavage reaction in the presence of an acid, from the viewpoint of improvement in productivity of optical member formation.

[Resin Obtained with Compound Represented by Formula (1) as Monomer]

The compound represented by the above formula (1) can be contained, for use, in the optical member forming composition. Alternatively, a resin obtained with the compound represented by the above formula (1) as a monomer may be contained, for use, in the optical member forming composition. The resin is obtained, for example, by reacting the compound represented by the above formula (1) with a crosslinking compound.

Examples of the resin obtained with the compound represented by the above formula (1) as a monomer include resins having a structure represented by the formula (3) given below. In other words, the optical member forming composition according to the present embodiment may contain a resin having a structure represented by the following formula (3):

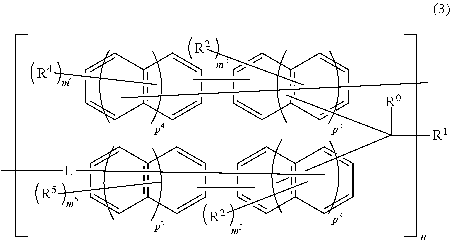

In the formula (3), L is a linear or branched alkylene group of 1 to 30 carbon atoms optionally having a substituent or a single bond.

R⁰, R¹, R² to R⁵, m² and m³, m⁴ and m⁵, p² to p⁵, and n are as defined in the above formula (1).

However, m², m³, m⁴ and m⁵ are not 0 at the same time, and at least one of R² to R⁵ is a hydroxy group or a group in which a hydrogen atom of a hydroxy group is substituted with an acid crosslinking group or an acid dissociation group.

[Method for Producing Resin Obtained with Compound Represented by Formula (1) as Monomer]

The resin according to the present embodiment is obtained by reacting the compound represented by the above formula (1) with a crosslinking compound. As the crosslinking compound, a publicly known compound can be used without particular limitations as long as it can oligomerize or polymerize the compound represented by the above formula (1). Specific examples thereof include, but not particularly limited to, aldehydes, ketones, carboxylic acids, carboxylic acid halides, halogen-containing compounds, amino compounds, imino compounds, isocyanates, and unsaturated hydrocarbon group-containing compounds.

Specific examples of the resin having a structure represented by the above formula (3) include resins that are made novolac by, for example, condensation reaction between the compound represented by the above formula (1) with an aldehyde and/or a ketone which is a crosslinking compound.

Herein, examples of the aldehyde used upon making the compound represented by the above formula (1) novolac include, but not particularly limited to, formaldehyde, trioxane, paraformaldehyde, benzaldehyde, acetaldehyde, propylaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, biphenylaldehyde, naphthaldehyde, anthracenecarbaldehyde, phenanthrenecarbaldehyde, pyrenecarbaldehyde, and furfural. Examples of the ketone include the ketones described above. Among them, formaldehyde is more preferable. These aldehydes and/or ketones can be used alone as one kind or can be used in combination of two or more kinds. The amount of the aldehyde and/or the ketone used is not particularly limited, but is preferably 0.2 to 5 mol and more preferably 0.5 to 2 mol per 1 mol of the compound represented by the above formula (1).

An acid catalyst can also be used in the condensation reaction between the compound represented by the above formula (1) and the aldehyde and/or the ketone. The acid catalyst used herein can be arbitrarily selected and used from publicly known catalysts and is not particularly limited. Inorganic acids and organic acids are widely known as such acid catalysts, and examples include, but not particularly limited to, inorganic acids such as hydrochloric acid, sulfuric acid, phosphoric acid, hydrobromic acid, and hydrofluoric acid; organic acids such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; Lewis acids such as zinc chloride, aluminum chloride, iron chloride, and boron trifluoride; and solid acids such as tungstosilicic acid, tungstophosphoric acid, silicomolybdic acid, and phosphomolybdic acid. Among them, organic acids and solid acids are preferable from the viewpoint of production, and hydrochloric acid or sulfuric acid is preferable from the viewpoint of production such as easy availability and handleability. The acid catalysts can be used alone as one kind or can be used in combination of two or more kinds.

Also, the amount of the acid catalyst used can be arbitrarily set according to, for example, the kind of the raw materials used and the catalyst and moreover the reaction conditions and is not particularly limited, but is preferably 0.01 to 100 parts by mass per 100 parts by mass of the reaction raw materials. However, the aldehyde is not necessarily needed in the case of copolymerization reaction with a compound having a non-conjugated double bond, such as indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, 5-vinylnorborn-2-ene, α-pinene, β-pinene, and limonene.

A reaction solvent can also be used in the condensation reaction between the compound represented by the above formula (1) and the aldehyde and/or the ketone. The reaction solvent in the polycondensation can be arbitrarily selected and used from publicly known solvents and is not particularly limited, and examples include water, methanol, ethanol, propanol, butanol, tetrahydrofuran, dioxane, and a mixed solvent thereof. The solvents can be used alone as one kind or can be used in combination of two or more kinds.

Also, the amount of these solvents used can be arbitrarily set according to, for example, the kind of the raw materials used and the catalyst and moreover the reaction conditions and is not particularly limited, but is preferably in the range of 0 to 2000 parts by mass based on 100 parts by mass of the reaction raw materials. Furthermore, the reaction temperature can be arbitrarily selected according to the reactivity of the reaction raw materials and is not particularly limited, but is usually in the range of 10 to 200° C. The reaction method can be arbitrarily selected and used from publicly known approaches and is not particularly limited. Examples thereof include a method of charging the compound represented by the above formula (1), the aldehyde and/or the ketone, and the catalyst in one portion, and a method of dropping the compound represented by the above formula (1) and the aldehyde and/or the ketone in the presence of the catalyst.

After the polycondensation reaction terminates, isolation of the obtained compound can be carried out according to a conventional method, and is not particularly limited. For example, by adopting a commonly used approach in which the temperature of the reaction vessel is elevated to 130 to 230° C. in order to remove unreacted raw materials, catalyst, etc. present in the system, and volatile portions are removed at about 1 to 50 mmHg, the resin made novolac which is the target component can be isolated.

Herein, the resin having a structure represented by the above formula (3) may be a homopolymer of the compound represented by the above formula (1), but may be a copolymer with an additional phenol. Herein, examples of the copolymerizable phenol include, but not particularly limited to, phenol, cresol, dimethylphenol, trimethylphenol, butylphenol, phenylphenol, diphenylphenol, naphthylphenol, resorcinol, methylresorcinol, catechol, butylcatechol, methoxyphenol, propylphenol, pyrogallol, and thymol.

Alternatively, the resin having a structure represented by the above formula (3) may be a copolymer with a polymerizable monomer, instead of the additional phenol mentioned above. Examples of such a monomer for copolymerization include, but not particularly limited to, naphthol, methylnaphthol, methoxynaphthol, dihydroxynaphthalene, indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, vinylnorbornene, pinene, and limonene. The resin having a structure represented by the above formula (3) may be a binary or higher (e.g., binary to quaternary) copolymer of the compound represented by the above formula (1) and the phenol mentioned above, may be a binary or higher (e.g., binary to quaternary) copolymer of the compound represented by the above formula (1) and the monomer for copolymerization mentioned above, or may be a ternary or higher (e.g., ternary to quaternary) copolymer of the compound represented by the above formula (1), the phenol mentioned above, and the monomer for copolymerization mentioned above.

The molecular weight of the resin having a structure represented by the above formula (3) is not particularly limited, and the weight average molecular weight (Mw) in terms of polystyrene is preferably 500 to 30,000 and more preferably 750 to 20,000. The resin having a structure represented by the above formula (3) preferably has a dispersibility (weight average molecular weight Mw/number average molecular weight Mn) within the range of 1.2 to 7 from the viewpoint of enhancing crosslinking efficiency and suppressing volatile components during baking. The Mw and the Mn refer to a weight average molecular weight (Mw) and a number average molecular weight (Mn) in terms of polystyrene determined by gel permeation chromatography (GPC) analysis.

The resin having a structure represented by the above formula (3) preferably has high solubility in a solvent from the viewpoint of easier application to a wet process, etc. More specifically, in the case of using 1-methoxy-2-propanol (PGME) and/or propylene glycol monomethyl ether acetate (PGMEA) as a solvent, the solubility of the resin in the solvent is preferably 10% by mass or more. Herein, the solubility in PGME and/or PGMEA is defined as “Mass of the resin/(Mass of the resin+Mass of the solvent)×100 (% by mass)”. For example, the solubility of the resin in PGMEA is “10% by mass or more” when 10 g of the resin is dissolved in 90 g of PGMEA, and is “less than 10% by mass” when 10 g of the resin is not dissolved in 90 g of PGMEA.

The compound represented by the above formula (0), the compound represented by the formula (0-1), and a resin obtained with the compound as a monomer can be produced in accordance with the aforementioned methods for producing compound represented by the formula (1) and the resin obtained with the compound as a monomer.

### [Compound Represented by Formula (2)]

The compound (0-1) according to the present embodiment is preferably a compound represented by the following formula (2) from the viewpoint of heat resistance and solvent solubility:

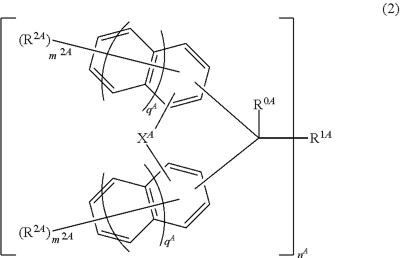

In the formula (2), R^{0A} is a hydrogen atom.

R^{1A} is an n^{A}-valent group of 1 to 60 carbon atoms or a single bond.

Each R^{2A} is independently an alkyl group of 1 to 30 carbon atoms optionally having a substituent, an aryl group of 6 to 30 carbon atoms optionally having a substituent, an alkenyl group of 2 to 10 carbon atoms optionally having a substituent, a halogen atom, a hydroxy group or a group in which a hydrogen atom of a hydroxy group is substituted with an acid dissociation group, and may be the same or different on the same naphthalene ring or benzene ring. Herein, the alkyl group, the alkenyl group and the aryl group each optionally have an ether bond, a ketone bond or an ester bond. However, in the formula (2), at least one of R^{2A} is a hydroxy group or a group in which a hydrogen atom of a hydroxy group is substituted with an acid crosslinking group or an acid dissociation group.

n^{A} is an integer of 1 to 4. Herein, in the formula (2), when n^{A} is an integer of 2 or larger, structural formulae indicated within n^{A} parentheses are the same or different.

Each X^{A} is independently an oxygen atom, a sulfur atom, a single bond or non-crosslinked state. Herein, X^{A} is preferably an oxygen atom or a sulfur atom and more preferably an oxygen atom because there is a tendency to exert excellent heat resistance. X^{A} is preferably non-crosslinked state from the viewpoint of solubility.

Each m^{2A} is independently an integer of 0 to 7. However, at least one of m^{2A} is an integer of 1 to 7.

Each q^{A} is independently 0 or 1.

The n-valent group refers to an alkyl group of 1 to 60 carbon atoms (n=1), an alkylene group of 1 to 30 carbon atoms (n=2), an alkanepropayl group of 2 to 60 carbon atoms (n=3), and an alkanetetrayl group of 3 to 60 carbon atoms (n=4). Examples of the n-valent group include groups having a linear hydrocarbon group, a branched hydrocarbon group or an alicyclic hydrocarbon group. Herein, the alicyclic hydrocarbon group also includes bridged alicyclic hydrocarbon groups. Also, the n-valent group may have an aromatic group of 6 to 60 carbon atoms.

The n-valent hydrocarbon group may have an alicyclic hydrocarbon group, a double bond, a heteroatom or an aromatic group of 6 to 60 carbon atoms. Herein, the alicyclic hydrocarbon group also includes bridged alicyclic hydrocarbon groups.

The n-valent hydrocarbon group may have an alicyclic hydrocarbon group, a double bond, a heteroatom or an aromatic group of 6 to 30 carbon atoms. Herein, the alicyclic hydrocarbon group also includes bridged alicyclic hydrocarbon groups.

The compound represented by the above formula (2) has high refractive index. Also, the compound represented by the above formula (2) has high heat resistance attributed to its rigidity of the structure despite a relatively low molecular weight and can therefore be used even under high temperature baking conditions. Furthermore, the compound represented by the above formula (2) has high solubility in a safe solvent, exhibits suppressed crystallinity, and has good heat resistance and etching resistance. Hence, the optical member forming composition comprising the compound represented by the above formula (2) can impart a good shape to an optical member. The optical member forming composition is relatively prevented from being stained by heat treatment in a wide range from a low temperature to a high temperature and is therefore useful as various optical member forming compositions. The optical member forming composition is useful for an optical component in a film form or a sheet form as well as a plastic lens (prism lens, lenticular lens, microlens, Fresnel lens, viewing angle control lens, contrast improving lens, etc.), a phase difference film, a film for electromagnetic wave shielding, a prism, an optical fiber, a solder resist for flexible printed wiring, a plating resist, an interlayer insulating film for multilayer printed circuit boards, and a photosensitive optical waveguide.

The compound represented by the above formula (2) is preferably a compound represented by the following formula (2-1) from the viewpoint of easy crosslinking and solubility in an organic solvent:

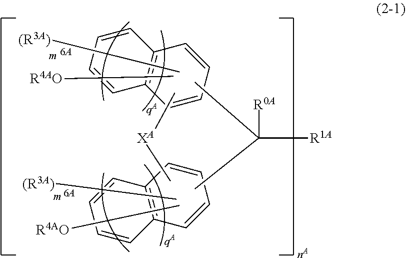

In the formula (2-1), R^{0A}, R^{1A}, n^{A}, q^{A} and X^{A} are as defined in those described in the above formula (2).

Each R^{3A} is independently a halogen atom, an alkyl group of 1 to 30 carbon atoms optionally having a substituent, an aryl group of 6 to 30 carbon atoms optionally having a substituent, or an alkenyl group of 2 to 30 carbon atoms optionally having a substituent, and may be the same or difference on the same naphthalene ring or benzene ring. Herein, the alkyl group, the alkenyl group and the aryl group each optionally have an ether bond, a ketone bond or an ester bond.

Each R^{4A} is independently a hydrogen atom, an acid crosslinking group or an acid dissociation group.

Each m^{6A} is independently an integer of 0 to 5.

The compound represented by the above formula (2-1) is preferably a compound represented by the following formula (2a) from the viewpoint of the supply of raw materials.

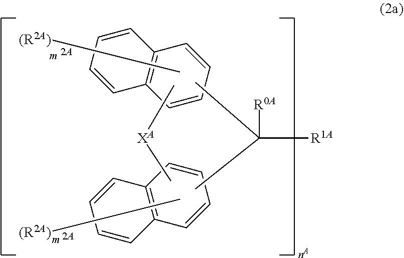

In the above formula (2a), X^{A}, R^{0A} to R^{2A}, m^{2A} and n^{A} are as defined in those described in the above formula (2).

The compound represented by the above formula (2-1) is more preferably a compound represented by the following formula (2b) from the viewpoint of solubility in an organic solvent:

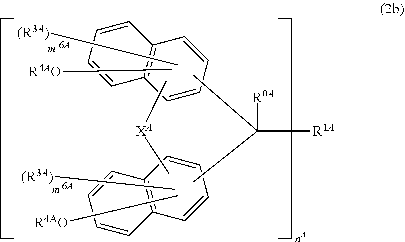

In the above formula (2b), X^{A}, R^{0A}, R^{1A}, R^{3A}, R^{4A}, m^{6A} and n^{A} are as defined in those described in the above formula (2-1).

The compound represented by the above formula (2-1) is still more preferably a compound represented by the following formula (2c) from the viewpoint of solubility in an organic solvent:

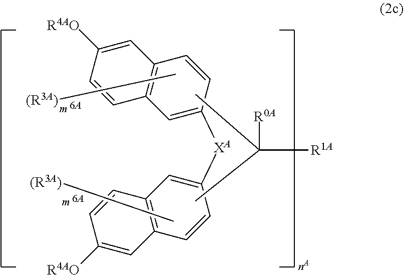

In the above formula (2c), X^{A}, R^{0A}, R^{1A}, R^{3A}, R^{4A}, m^{6A} and n^{A} are as defined in those described in the above formula (2-1).

The compound represented by the above formula (2) is particularly preferably a compound represented by the following formulae (BiN-1) to (BiN-4) or (XiN-1) to (XiN-3) from the viewpoint of further solubility in an organic solvent.

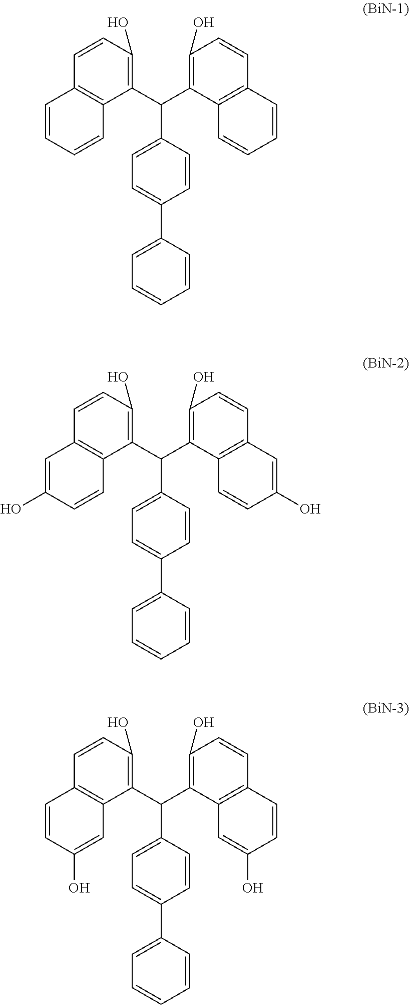 [SMILES: Oc1ccc2ccccc2c1C(c1ccc(-c2ccccc2)cc1)c1c(O)ccc2ccccc12 ; Oc1ccc2c(C(c3ccc(-c4ccccc4)cc3)c3c(O)ccc4cc(O)ccc34)c(O)ccc2c1 ; Oc1ccc2ccc(O)c(C(c3ccc(-c4ccccc4)cc3)c3c(O)ccc4ccc(O)cc34)c2c1]

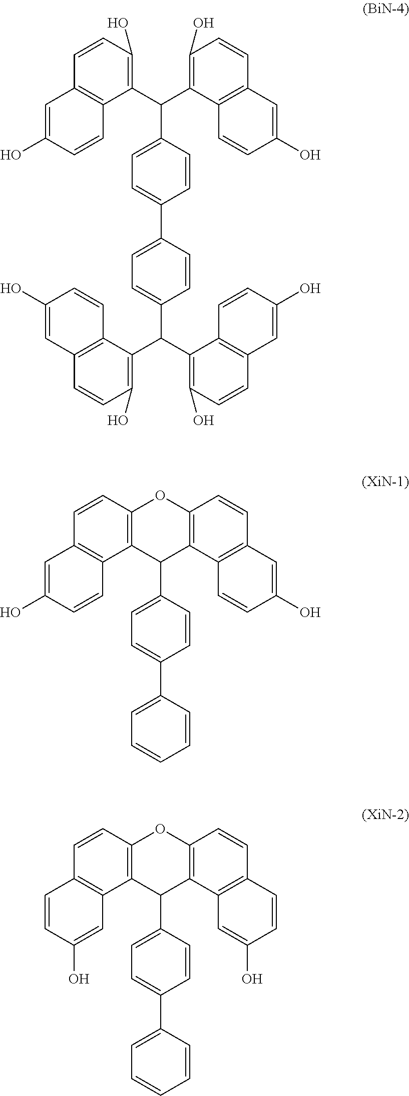 [SMILES: Oc1ccc2c(C(c3ccc(-c4ccc(C(c5c(O)ccc6cc(O)ccc56)c5c(O)ccc6cc(O)ccc56)cc4)cc3)c3c(O)ccc4cc(O)ccc34)c(O)ccc2c1 ; Oc1ccc2c3c(ccc2c1)Oc1ccc2cc(O)ccc2c1C3c1ccc(-c2ccccc2)cc1 ; Oc1ccc2ccc3c(c2c1)C(c1ccc(-c2ccccc2)cc1)c1c(ccc2ccc(O)cc12)O3]

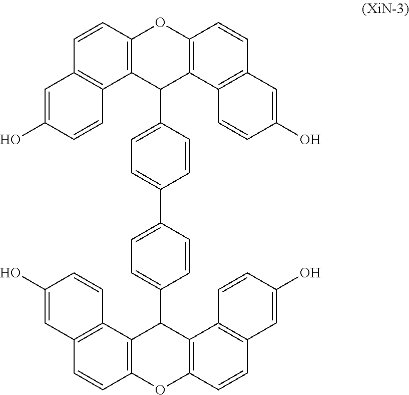 [SMILES: Oc1ccc2c3c(ccc2c1)Oc1ccc2cc(O)ccc2c1C3c1ccc(-c2ccc(C3c4c(ccc5cc(O)ccc45)Oc4ccc5cc(O)ccc5c43)cc2)cc1]

### [Method for Producing Compound Represented by Formula (2)]

The compound represented by the formula (2) according to the present embodiment can be arbitrarily synthesized by the application of a publicly known approach, and the synthesis approach is not particularly limited.

The compound represented by the above formula (2) can be obtained, for example, by subjecting a phenol, a naphthol or anthracenol, and a corresponding aldehyde to polycondensation reaction in the presence of an acid catalyst at normal pressure. Also, an acid crosslinking group or an acid dissociation group can be introduced to at least one phenolic hydroxy group of the compound by a publicly known method. If necessary, this reaction can also be carried out under increased pressure.

Examples of the phenol include, but not particularly limited to, phenol, methylphenol, methoxybenzene, catechol, resorcinol, hydroquinone, and trimethylhydroquinone.

Examples of the naphthol include, but not particularly limited to, naphthol, methylnaphthol, methoxynaphthol, and naphthalenediol. Among them, naphthalenediol is preferably used from the viewpoint that a xanthene structure can be easily formed.

Examples of the aldehyde include, but not particularly limited to, formaldehyde, trioxane, paraformaldehyde, benzaldehyde, acetaldehyde, propylaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, biphenylaldehyde, naphthaldehyde, anthracenecarbaldehyde, phenanthrenecarbaldehyde, pyrenecarbaldehyde, and furfural. These aldehydes can be used alone as one kind or can be used in combination of two or more kinds. Among them, benzaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde, or furfural is preferably used from the viewpoint of conferring high heat resistance. Benzaldehyde, hydroxybenzaldehyde, chlorobenzaldehyde, nitrobenzaldehyde, methylbenzaldehyde, ethylbenzaldehyde, butylbenzaldehyde, cyclohexylbenzaldehyde, biphenylaldehyde, naphthaldehyde, anthracenecarbaldehyde, phenanthrenecarbaldehyde, pyrenecarbaldehyde, or furfural is more preferably used from the viewpoint of improving etching resistance. An aldehyde having an aromatic ring is preferably used as the aldehyde from the viewpoint of possessing both high heat resistance and high etching resistance.

The acid catalyst used in the above reaction can be arbitrarily selected and used from publicly known catalysts and is not particularly limited. The acid catalyst can be arbitrarily selected from well-known inorganic acids and organic acids, and examples include inorganic acids such as hydrochloric acid, sulfuric acid, phosphoric acid, hydrobromic acid, and hydrofluoric acid; organic acids such as oxalic acid, formic acid, p-toluenesulfonic acid, methanesulfonic acid, trifluoroacetic acid, trifluoromethanesulfonic acid, benzenesulfonic acid, naphthalenesulfonic acid, and naphthalenedisulfonic acid; Lewis acids such as zinc chloride, aluminum chloride, iron chloride, and boron trifluoride; and solid acids such as tungstosilicic acid, tungstophosphoric acid, silicomolybdic acid, and phosphomolybdic acid. Among them, hydrochloric acid or sulfuric acid is preferably used from the viewpoint of production such as easy availability and handleability. The acid catalysts can be used alone as one kind or can be used in combination of two or more kinds.

Upon producing the compound represented by the above formula (2), a reaction solvent may be used. The reaction solvent is not particularly limited as long as the reaction of the aldehyde used with the naphthol or the like proceeds. For example, water, methanol, ethanol, propanol, butanol, tetrahydrofuran, dioxane, or a mixed solvent thereof can be used. The amount of the reaction solvent is not particularly limited and is, for example, in the range of 0 to 2000 parts by mass per 100 parts by mass of the reaction raw materials.

The reaction temperature is not particularly limited and can be arbitrarily selected according to the reactivity of the reaction raw materials, but is preferably in the range of 10 to 200° C. A lower temperature is preferable, and the range of 10 to 60° C. is more preferable, from the viewpoint of selectively synthesizing the compound represented by the formula (2) according to the present embodiment.

Examples of the reaction method include, but not particularly limited to, a method of charging the naphthol or the like, the aldehyde, and the catalyst in one portion, and a method of dropping the naphthol and the aldehyde in the presence of the catalyst. After the polycondensation reaction terminates, the temperature of the reaction vessel may be elevated to 130 to 230° C. in order to remove unreacted raw materials, catalyst, etc. present in the system, and volatile portions can be removed at about 1 to 50 mmHg.

The amount of the raw material is not particularly limited, but it is preferable, for example, to use 2 mol to an excess of the naphthol or the like and 0.001 to 1 mol of the acid catalyst per 1 mol of the aldehyde, and react them at 20 to 60° C. at normal pressure for about 20 minutes to 100 hours.

The target component is isolated by a publicly known method after the reaction terminates. Examples of the method for isolating the target component include, but not particularly limited to, a method which involves concentrating the reaction solution, precipitating the reaction product by the addition of pure water, cooling the reaction solution to room temperature, then separating the precipitates by filtration, filtering and drying the obtained solid matter, then separating and purifying the solid matter from by-products by column chromatography, and distilling off the solvent, followed by filtration and drying to isolate the target compound.

A method for introducing an acid dissociation group or an acid crosslinking group to at least one phenolic hydroxy group of a polyphenol compound is publicly known. An acid dissociation group or an acid crosslinking group can be introduced to at least one phenolic hydroxy group of the compound, for example, as described below. A compound for introducing the acid dissociation group or the acid crosslinking group can be synthesized by a publicly known method or easily obtained. Examples thereof include, but not particularly limited to, acid chlorides, acid anhydrides, active carboxylic acid derivative compounds such as dicarbonate, alkyl halides, vinyl alkyl ethers, dihydropyran, and halocarboxylic acid alkyl esters.

For example, the compound is dissolved or suspended in an aprotic solvent such as acetone, tetrahydrofuran (THF), or propylene glycol monomethyl ether acetate. Subsequently, a vinyl alkyl ether such as ethyl vinyl ether, or dihydropyran is added to the solution or the suspension, and the mixture is reacted at 20 to 60° C. at normal pressure for 6 to 72 hours in the presence of an acid catalyst such as pyridinium p-toluenesulfonate. The reaction solution is neutralized with an alkali compound and added to distilled water to precipitate a white solid. Then, the separated white solid can be washed with distilled water and dried to obtain the compound in which a hydrogen atom of the hydroxy group is substituted with the acid dissociation group.

Also, for example, the compound having a hydroxy group is dissolved or suspended in an aprotic solvent such as acetone, THF, or propylene glycol monomethyl ether acetate. Subsequently, an alkyl halide such as ethyl chloromethyl ether or a halocarboxylic acid alkyl ester such as methyladamantyl bromoacetate is added to the solution or the suspension, and the mixture is reacted at 20 to 110° C. at normal pressure for 6 to 72 hours in the presence of an alkali catalyst such as potassium carbonate. The reaction solution is neutralized with an acid such as hydrochloric acid and added to distilled water to precipitate a white solid. Then, the separated white solid can be washed with distilled water and dried to obtain the compound in which a hydrogen atom of the hydroxy group is substituted with the acid dissociation group.

The timing of introducing the acid dissociation group is not only after the condensation reaction of the binaphthol with the ketone, but may be prior to the condensation reaction. This introduction may be carried out after production of a resin mentioned later.

The acid dissociation group and the acid crosslinking group, and the compound for introducing these groups are the same as described in the compound represented by the formula (1).

[Resin Obtained with Compound Represented by Formula (2) as Monomer]

The optical member forming composition according to the present embodiment may contain the compound represented by the above formula (2) and may contain a resin obtained with the compound represented by the above formula (2) as a monomer. The resin is obtained, for example, by reacting the compound represented by the above formula (2) with a crosslinking compound.

Examples of the resin obtained with the compound represented by the above formula (2) as a monomer include resins having a structure represented by the formula (4) given below. In other words, the optical member forming composition according to the present embodiment may contain a resin having a structure represented by the following formula (4):

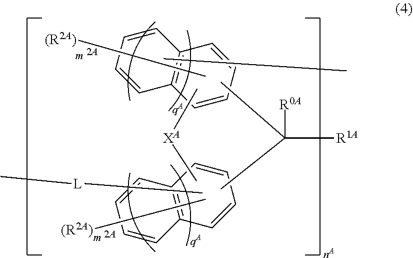

In the formula (4), L is a linear or branched alkylene group of 1 to 30 carbon atoms optionally having a substituent or a single bond.

R^{0A}, R^{1A}, R^{2A}, m^{2A}, n^{A}, q^{A} and X^{A} are as defined in the above formula (2).

When n^{A} is an integer of 2 or larger, structural formulae indicated within n^{A} parentheses are the same or different. At least one of m^{2A} is an integer of 1 to 6, and at least one of R^{2A} is a hydroxy group or a group in which a hydrogen atom of a hydroxy group is substituted with an acid crosslinking group or an acid dissociation group.

[Method for Producing Resin Obtained with Compound Represented by Formula (2) as Monomer]

The resin according to the present embodiment is obtained by reacting the compound represented by the above formula (2) with a crosslinking compound. As the crosslinking compound, a publicly known compound can be used without particular limitations as long as it can oligomerize or polymerize the compound represented by the above formula (2). Specific examples thereof include, but not particularly limited to, aldehydes, ketones, carboxylic acids, carboxylic acid halides, halogen-containing compounds, amino compounds, imino compounds, isocyanates, and unsaturated hydrocarbon group-containing compounds.

Specific examples of the resin having a structure represented by the above formula (4) include resins that are made novolac by, for example, condensation reaction between the compound represented by the above formula (2) with an aldehyde and/or a ketone which is a crosslinking compound.

Herein, examples of the aldehyde used upon making the compound represented by the above formula (2) novolac include, but not particularly limited to, formaldehyde, trioxane, paraformaldehyde, benzaldehyde, acetaldehyde, propylaldehyde, phenylacetaldehyde, phenylpropylaldehyde, hydroxybenzaldehyde, chlorobenzaldehyde, nitrobenzaldehyde, methylbenzaldehyde, ethylbenzaldehyde, butylbenzaldehyde, biphenylaldehyde, naphthaldehyde, anthracenecarbaldehyde, phenanthrenecarbaldehyde, pyrenecarbaldehyde, and furfural. Examples of the ketone include the ketones described above. Among them, formaldehyde is more preferable. These aldehydes and/or ketones can be used alone as one kind or can be used in combination of two or more kinds. The amount of the aldehyde and/or the ketone used is not particularly limited, but is preferably 0.2 to 5 mol and more preferably 0.5 to 2 mol per 1 mol of the compound represented by the above formula (2).

An acid catalyst can also be used in the condensation reaction between the compound represented by the above formula (2) and the aldehyde and/or the ketone. The acid catalyst used herein can be arbitrarily selected and used from publicly known catalysts and is not particularly limited. Inorganic acids and organic acids are widely known as such acid catalysts, and examples include, but not particularly limited to, inorganic acids such as hydrochloric acid, sulfuric acid, phosphoric acid, hydrobromic acid, and hydrofluoric acid; organic acids such as oxalic acid, malonic acid, succinic acid, adipic acid, sebacic acid, citric acid, fumaric acid, maleic acid, formic acid, p-toluenesulfonic acid, methanesulfonic acid, trifluoroacetic acid, dichloroacetic acid, trichloroacetic acid, trifluoromethanesulfonic acid, benzenesulfonic acid, naphthalenesulfonic acid, and naphthalenedisulfonic acid; Lewis acids such as zinc chloride, aluminum chloride, iron chloride, and boron trifluoride; and solid acids such as tungstosilicic acid, tungstophosphoric acid, silicomolybdic acid, and phosphomolybdic acid. Among them, organic acids or solid acids are preferable from the viewpoint of production, and hydrochloric acid or sulfuric acid is preferable from the viewpoint of production such as easy availability and handleability. The acid catalysts can be used alone as one kind or can be used in combination of two or more kinds.

Also, the amount of the acid catalyst used can be arbitrarily set according to, for example, the kind of the raw materials used and the catalyst used and moreover the reaction conditions and is not particularly limited, but is preferably 0.01 to 100 parts by mass per 100 parts by mass of the reaction raw materials. However, the aldehyde is not necessarily needed in the case of copolymerization reaction with a compound having a non-conjugated double bond, such as indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, 5-vinylnorborn-2-ene, α-pinene, β-pinene, and limonene.

A reaction solvent can also be used in the condensation reaction between the compound represented by the above formula (2) and the aldehyde and/or the ketone. The reaction solvent in the polycondensation can be arbitrarily selected and used from publicly known solvents and is not particularly limited, and examples include water, methanol, ethanol, propanol, butanol, tetrahydrofuran, dioxane, and a mixed solvent thereof. The solvents can be used alone as one kind or can be used in combination of two or more kinds.

Also, the amount of these solvents used can be arbitrarily set according to, for example, the kind of the raw materials used and the catalyst used and moreover the reaction conditions and is not particularly limited, but is preferably in the range of 0 to 2000 parts by mass per 100 parts by mass of the reaction raw materials. Furthermore, the reaction temperature can be arbitrarily selected according to the reactivity of the reaction raw materials and is not particularly limited, but is usually in the range of 10 to 200° C. The reaction method can be arbitrarily selected and used from publicly known approaches and is not particularly limited. Examples thereof include a method of charging the compound represented by the above formula (2), the aldehyde and/or the ketone, and the catalyst in one portion, and a method of dropping the compound represented by the above formula (2) and the aldehyde and/or the ketone in the presence of the catalyst.

After the polycondensation reaction terminates, isolation of the obtained compound can be carried out according to a conventional method, and is not particularly limited. For example, by adopting a commonly used approach in which the temperature of the reaction vessel is elevated to 130 to 230° C. in order to remove unreacted raw materials, catalyst, etc. present in the system, and volatile portions are removed at about 1 to 50 mmHg, the resin made novolac which is the target component can be isolated.

Herein, the resin having a structure represented by the above formula (4) may be a homopolymer of the compound represented by the above formula (2), but may be a copolymer with an additional phenol. Herein, examples of the copolymerizable phenol include, but not particularly limited to, phenol, cresol, dimethylphenol, trimethylphenol, butylphenol, phenylphenol, diphenylphenol, naphthylphenol, resorcinol, methylresorcinol, catechol, butylcatechol, methoxyphenol, methoxyphenol, propylphenol, pyrogallol, and thymol.

Alternatively, the resin having a structure represented by the above formula (4) may be a copolymer with a polymerizable monomer, instead of the additional phenol mentioned above. Examples of such a monomer for copolymerization include, but not particularly limited to, naphthol, methylnaphthol, methoxynaphthol, dihydroxynaphthalene, indene, hydroxyindene, benzofuran, hydroxyanthracene, acenaphthylene, biphenyl, bisphenol, trisphenol, dicyclopentadiene, tetrahydroindene, 4-vinylcyclohexene, norbornadiene, vinylnorbornene, pinene, and limonene. The resin having a structure represented by the above formula (4) may be a binary or higher (e.g., binary to quaternary) copolymer of the compound represented by the above formula (2) and the phenol mentioned above, may be a binary or higher (e.g., binary to quaternary) copolymer of the compound represented by the above formula (2) and the monomer for copolymerization mentioned above, or may be a ternary or higher (e.g., ternary to quaternary) copolymer of the compound represented by the above formula (2), the phenol mentioned above, and the monomer for copolymerization mentioned above.

The molecular weight of the resin having a structure represented by the above formula (4) is not particularly limited, and the weight average molecular weight (Mw) in terms of polystyrene is preferably 500 to 30,000 and more preferably 750 to 20,000. The resin having a structure represented by the above formula (4) preferably has a dispersibility (weight average molecular weight Mw/number average molecular weight Mn) within the range of 1.2 to 7 from the viewpoint of enhancing crosslinking efficiency and suppressing volatile components during baking.

The resin having a structure represented by the above formula (4) preferably has high solubility in a solvent from the viewpoint of easier application to a wet process, etc. More specifically, in the case of using 1-methoxy-2-propanol (PGME) and/or propylene glycol monomethyl ether acetate (PGMEA) as a solvent, the solubility of the resin in the solvent is preferably 10% by mass or more. Herein, the solubility in PGME and/or PGMEA is defined as “Mass of the resin/(Mass of the resin+Mass of the solvent)×100 (% by mass)”. For example, the solubility of the resin in PGMEA is “10% by mass or more” when 10 g of the resin is dissolved in 90 g of PGMEA, and is “less than 10% by mass” when 10 g of the resin is not dissolved in 90 g of PGMEA.

The optical member forming composition according to the present embodiment contains at least one selected from the group consisting of the compound represented by the above formula (0), the compound represented by the formula (0-1), the compound represented by the formula (1), the compound represented by the formula (2), and the resin obtained with each of these compounds as a monomer (hereinafter, also collectively referred to as “component (A)”).

### (Other Components of Optical Member Forming Composition)

The optical member forming composition of the present embodiment may contain components described below, in addition to containing any one or more components (A) as a solid component.

It is preferable that the optical member forming composition according to the present embodiment contains a solvent. Examples of the solvent can include, but not particularly limited to, ethylene glycol monoalkyl ether acetates such as ethylene glycol monomethyl ether acetate, ethylene glycol monoethyl ether acetate, ethylene glycol mono-n-propyl ether acetate, and ethylene glycol mono-n-butyl ether acetate; ethylene glycol monoalkyl ethers such as ethylene glycol monomethyl ether and ethylene glycol monoethyl ether; propylene glycol monoalkyl ether acetates such as propylene glycol monomethyl ether acetate, propylene glycol monoethyl ether acetate (PGMEA), propylene glycol mono-n-propyl ether acetate, and propylene glycol mono-n-butyl ether acetate; propylene glycol monoalkyl ethers such as propylene glycol monomethyl ether (PGME) and propylene glycol monoethyl ether; ester lactates such as methyl lactate, ethyl lactate, n-propyl lactate, n-butyl lactate, and n-amyl lactate; aliphatic carboxylic acid esters such as methyl acetate, ethyl acetate, n-propyl acetate, n-butyl acetate, n-amyl acetate, n-hexyl acetate, methyl propionate, and ethyl propionate; other esters such as methyl 3-methoxypropionate, ethyl 3-methoxypropionate, methyl 3-ethoxypropionate, ethyl 3-ethoxypropionate, methyl 3-methoxy-2-methylpropionate, 3-methoxybutylacetate, 3-methyl-3-methoxybutylacetate, butyl 3-methoxy-3-methylpropionate, butyl 3-methoxy-3-methylbutyrate, methyl acetoacetate, methyl pyruvate, and ethyl pyruvate; aromatic hydrocarbons such as toluene and xylene; ketones such as methyl ethyl ketone, 2-heptanone, 3-heptanone, 4-heptanone, cyclopentanone (CPN), and cyclohexanone (CHN); amides such as N,N-dimethylformamide, N-methylacetamide, N,N-dimethylacetamide, and N-methylpyrrolidone; and lactones such as γ-lactone. These solvents may be used alone or in combination of two or more kinds.

The solvent is preferably a safe solvent, more preferably at least one selected from PGMEA, PGME, CHN, CPN, 2-heptanone, anisole, butyl acetate, ethyl propionate, and ethyl lactate, and still more preferably at least one selected from PGMEA, PGME, and CHN.

In the optical member forming composition of the present embodiment, the amount of the solid component and the amount of the solvent are not particularly limited, but are preferably 1 to 80% by mass of the solid component and 20 to 99% by mass of the solvent, more preferably 1 to 50% by mass of the solid component and 50 to 99% by mass of the solvent, still more preferably 2 to 40% by mass of the solid component and 60 to 98% by mass of the solvent, and particularly preferably 2 to 10% by mass of the solid component and 90 to 98% by mass of the solvent, per 100% by mass in total of the solid component and the solvent.

The optical member forming composition of the present embodiment may contain at least one selected from the group consisting of an acid generating agent (C), an acid crosslinking agent (G), an acid diffusion controlling agent (E), and a further component (F), as other solid components. In the present specification, the “solid component” refers to a component other than the solvent.

In the optical member forming composition of the present embodiment, the content of the component (A) is not particularly limited, but is preferably 50 to 99.4% by mass of the solid components (summation of the component (A), and optionally used solid components such as acid generating agent (C), acid crosslinking agent (G), acid diffusion controlling agent (E), and further component (F), hereinafter the same), more preferably 55 to 90% by mass, still more preferably 60 to 80% by mass, and particularly preferably 60 to 70% by mass. The content of the component (A) is 50% by mass or more, whereby good refractive index tends to be obtained. The content is 99.4% by mass or less, whereby the resulting member tends to have a good shape.

When both the compound and the resin derived from the compound are contained, the content refers to the total amount of the compound and the resin derived from the compound.

### (Acid Generating Agent (C))

The optical member forming composition of the present embodiment preferably contains one or more acid generating agents (C) generating an acid directly or indirectly by heat. The content of the acid generating agent (C) is preferably 0.001 to 49% by mass of the total mass of the solid components, more preferably 1 to 40% by mass, still more preferably 3 to 30% by mass, and particularly preferably 10 to 25% by mass. When the content of the acid generating agent (C) is within the above range, higher refractive index tends to be obtained.

Concerning the optical member forming composition of the present embodiment, the acid generation method is not particularly limited as long as an acid is generated in the system. By using excimer laser instead of ultraviolet such as g-ray and i-ray, finer processing is possible, and also by using electron beam, extreme ultraviolet, X-ray or ion beam as a high energy ray, further finer processing is possible.

The acid generating agent (C) is not particularly limited, and is preferably at least one kind selected from the group consisting of compounds represented by the following formulae (8-1) to (8-8) from the viewpoint of the good amount of the acid generated:

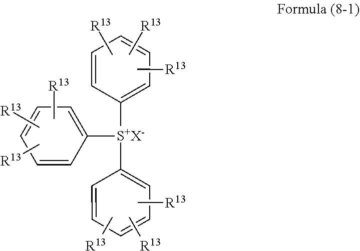

In the formula (8-1), R¹³ may be each the same or different, and are each independently a hydrogen atom, a linear, branched or cyclic alkyl group, a linear, branched or cyclic alkoxy group, a hydroxyl group, or a halogen atom, X⁻ is an alkyl group, an aryl group, a sulfonic acid ion having a halogen substituted alkyl group or a halogen substituted aryl group, or a halide ion.

The compound represented by the above formula (8-1) is preferably at least one kind selected from the group consisting of triphenylsulfonium trifluoromethanesulfonate, triphenylsulfonium nonafluoro-n-butanesulfonate, diphenyltolylsulfonium nonafluoro-n-butanesulfonate, triphenylsulfonium perfluoro-n-octanesulfonate, diphenyl-4-methylphenylsulfonium trifluoromethanesulfonate, di-2,4,6-trimethylphenylsulfonium trifluoromethanesulfonate, diphenyl-4-t-butoxyphenylsulfonium trifluoromethanesulfonate, diphenyl-4-t-butoxyphenylsulfonium nonafluoro-n-butanesulfonate, diphenyl-4-hydroxyphenylsulfonium trifluoromethanesulfonate, bis(4-fluorophenyl)-4-hydroxyphenylsulfonium trifluoromethanesulfonate, diphenyl-4-hydroxyphenylsulfonium nonafluoro-n-butanesulfonate, bis(4-hydroxyphenyl)-phenylsulfonium trifluoromethanesulfonate, tri(4-methoxyphenyl)sulfonium trifluoromethanesulfonate, tri(4-fluorophenyl)sulfonium trifluoromethanesulfonate, triphenylsulfonium p-toluenesulfonate, triphenylsulfonium benzenesulfonate, diphenyl-2,4,6-trimethylphenyl-p-toluenesulfonate, diphenyl-2,4,6-trimethylphenylsulfonium-2-trifluoromethylbenzenesulfonate, diphenyl-2,4,6-trimethylphenylsulfonium-4-trifluoromethylbenzenesulfonate, diphenyl-2,4,6-trimethylphenylsulfonium-2,4-difluorobenzenesulfonate, diphenyl-2,4,6-trimethylphenylsulfonium hexafluorobenzenesulfonate, diphenylnaphthylsulfonium trifluoromethanesulfonate, diphenyl-4-hydroxyphenylsulfonium-p-toluenesulfonate, triphenylsulfonium 10-camphorsulfonate, diphenyl-4-hydroxyphenylsulfonium 10-camphorsulfonate, and cyclo(1,3-perfluoropropanedisulfone)imidate.

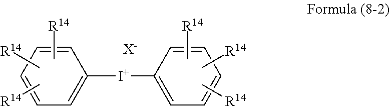

In the formula (8-2), R¹⁴ may be each the same or different, and are each independently a hydrogen atom, a linear, branched or cyclic alkyl group, a linear, branched or cyclic alkoxy group, a hydroxyl group, or a halogen atom. X⁻ is as defined in the formula (8-1)

The compound represented by the above formula (8-2) is preferably at least one kind selected from the group consisting of bis(4-t-butylphenyl)iodonium trifluoromethanesulfonate, bis(4-t-butylphenyl)iodonium nonafluoro-n-butanesulfonate, bis(4-t-butylphenyl)iodonium perfluoro-n-octanesulfonate, bis(4-t-butylphenyl)iodonium-p-toluenesulfonate, bis(4-t-butylphenyl)iodonium benzenesulfonate, bis(4-t-butylphenyl)iodonium-2-trifluoromethylbenzenesulfonate, bis(4-t-butylphenyl)iodonium-4-trifluoromethylbenzenesulfonate, bis(4-t-butylphenyl)iodonium-2,4-difluorobenzenesulfonate, bis(4-t-butylphenyl)iodonium hexafluorobenzenesulfonate, bis(4-t-butylphenyl)iodonium 10-camphorsulfonate, diphenyliodonium trifluoromethanesulfonate, diphenyliodonium nonafluoro-n-butanesulfonate, diphenyliodonium perfluoro-n-octanesulfonate, diphenyliodonium p-toluenesulfonate, diphenyliodonium benzenesulfonate, diphenyliodonium 10-camphorsulfonate, diphenyliodonium-2-trifluoromethylbenzenesulfonate, diphenyliodonium-4-trifluoromethylbenzenesulfonate, diphenyliodonium-2,4-difluorobenzenesulfonate, diphenyliodonium hexafluorobenzenesulfonate, di(4-trifluoromethylphenyl)iodonium trifluoromethanesulfonate, di(4-trifluoromethylphenyl)iodonium nonafluoro-n-butanesulfonate, di(4-trifluoromethylphenyl)iodonium perfluoro-n-octanesulfonate, di(4-trifluoromethylphenyl)iodonium-p-toluenesulfonate, di(4-trifluoromethylphenyl)iodonium benzenesulfonate, and di(4-trifluoromethylphenyl)iodonium 10-camphersulfonate.

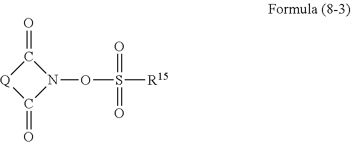

In the formula (8-3), Q is an alkylene group, an arylene group, or an alkoxylene group, and R¹⁵ is an alkyl group, an aryl group, a halogen substituted alkyl group, or a halogen substituted aryl group.

The compound represented by the above formula (8-3) is preferably at least one kind selected from the group consisting of N-(trifluoromethylsulfonyloxy)succinimide, N-(trifluoromethylsulfonyloxy)phthalimide, N-(trifluoromethylsulfonyloxy)diphenylmaleimide, N-(trifluoromethylsulfonyloxy)bicyclo[2.2.1]hept-5-en-2,3-dicarboxyimide, N-(trifluoromethylsulfonyloxy)naphthylimide, N-(10-camphorsulfonyloxy)succinimide, N-(10-camphorsulfonyloxy)phthalimide, N-(10-camphorsulfonyloxy)diphenylmaleimide, N-(10-camphorsulfonyloxy)bicyclo[2.2.1]hept-5-en-2,3-dicarboxyimide, N-(10-camphorsulfonyloxy)naphthylimide, N-(n-octanesulfonyloxy)bicyclo[2.2.1]hept-5-en-2,3-dicarboxyimide, N-(n-octanesulfonyloxy)naphthylimide, N-(p-toluenesulfonyloxy)bicyclo[2.2.1]hept-5-en-2,3-dicarboxyimide, N-(p-toluenesulfonyloxy)naphthylimide, N-(2-trifluoromethylbenzenesulfonyloxy)bicyclo[2.2.1]hept-5-en-2,3-dicarboxyimide, N-(2-trifluoromethylbenzenesulfonyloxy) naphthylimide, N-(4-trifluoromethylbenzenesulfonyloxy)bicyclo[2.2.1]hept-5-en-2,3-dicarboxyimide, N-(4-trifluoromethylbenzenesulfonyloxy)naphthylimide, N-(perfluorobenzenesulfonyloxy)bicyclo[2.2.1]hept-5-en-2,3-dicarboxyimide, N-(perfluorobenzenesulfonyloxy)naphthylimide, N-(1-naphthalenesulfonyloxy)bicyclo[2.2.1]hept-5-en-2,3-dicarboxyimide, N-(1-naphthalenesulfonyloxy)naphthylimide, N-(nonafluoro-n-butanesulfonyloxy)bicyclo[2.2.1]hept-5-en-2,3-dicarboxyimide, N-(nonafluoro-n-butanesulfonyloxy)naphthylimide, N-(perfluoro-n-octanesulfonyloxy)bicyclo[2.2.1]hept-5-en-2,3-dicarboxyimide, and N-(perfluoro-n-octanesulfonyloxy)naphthylimide.

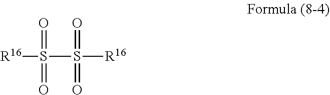

In the formula (8-4), R¹⁶ may be each the same or different, and are each independently an optionally substituted linear, branched or cyclic alkyl group, an optionally substituted aryl group, an optionally substituted heteroaryl group, or an optionally substituted aralkyl group.

The compound represented by the above formula (8-4) is preferably at least one kind selected from the group consisting of diphenyldisulfone, di(4-methylphenyl)disulfone, dinaphthyldisulfone, di(4-tert-butylphenyl)disulfone, di(4-hydroxyphenyl)disulfone, di(3-hydroxynaphthyl)disulfone, di(4-fluorophenyl)disulfone, di(2-fluorophenyl)disulfone, and di(4-trifluoromethylphenyl)disulfone.

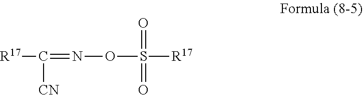

In the formula (8-5), R¹⁷ may be the same or different, and are each independently an optionally substituted linear, branched or cyclic alkyl group, an optionally substituted aryl group, an optionally substituted heteroaryl group, or an optionally substituted aralkyl group.

The compound represented by the above formula (8-5) is preferably at least one kind selected from the group consisting of α-(methylsulfonyloxyimino)-phenylacetonitrile, α-(methylsulfonyloxyimino)-4-methoxyphenylacetonitrile, α-(trifluoromethylsulfonyloxyimino)-phenylacetonitrile, α-(trifluoromethylsulfonyloxyimino)-4-methoxyphenylacetonitrile, α-(ethylsulfonyloxyimino)-4-methoxyphenylacetonitrile, α-(propylsulfonyloxyimino)-4-methylphenylacetonitrile, and α-(methylsulfonyloxyimino)-4-bromophenylacetonitrile.

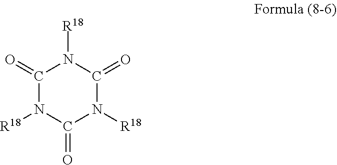

In the formula (8-6), R¹⁸ may be each the same or different, and are each independently a halogenated alkyl group having one or more chlorine atoms and one or more bromine atoms. The number of carbons in the halogenated alkyl group is preferably 1 to 5.

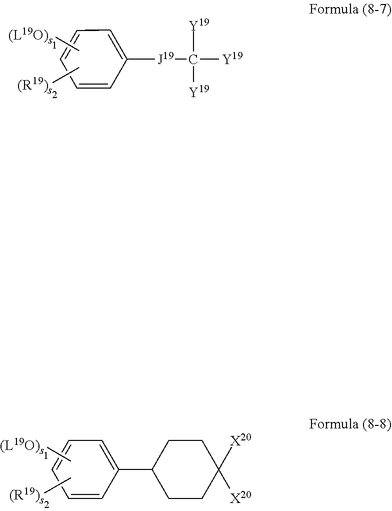

In the formulae (8-7) and (8-8), R¹⁹ and R²⁰ are each independently an alkyl group of 1 to 3 carbon atoms such as a methyl group, an ethyl group, an n-propyl group, and an isopropyl group; a cycloalkyl group such as a cyclopentyl group and a cyclohexyl group; an alkoxyl group of 1 to 3 carbon atoms such as a methoxy group, an ethoxy group, and a propoxy group; or an aryl group such as a phenyl group, a toluoyl group, and a naphthyl group, and preferably an aryl group of 6 to 10 carbon atoms. L¹⁹ and L²⁰ are each independently an organic group having a 1,2-naphthoquinonediazide group. Specifically, preferable examples of the organic group having a 1,2-naphthoquinonediazide group can include a 1,2-quinonediazidesulfonyl group such as a 1,2-naphthoquinonediazide-4-sulfonyl group, a 1,2-naphthoquinonediazide-5-sulfonyl group, and a 1,2-naphthoquinonediazide-6-sulfonyl group. Particularly, a 1,2-naphthoquinonediazide-4-sulfonyl group and a 1,2-naphthoquinonediazide-5-sulfonyl group are preferable. Each s₁ is independently an integer of 1 to 3; each s₂ is independently an integer of 0 to 4; and 1<s₁+s₂<5. J¹⁹ is a single bond, a polymethylene group of 1 to 4 carbon atoms, a cycloalkylene group, a phenylene group, a group represented by the following formula (8-7-1), a carbonyl group, an ester group, an amide group, or an ether group. Y¹⁹ is a hydrogen atom, an alkyl group, or an aryl group, and X²⁰ are each independently a group represented by the following formula (8-8-1):

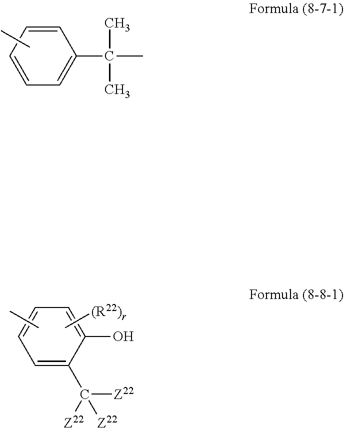

In the above formula (8-8-1), Z²² are each independently an alkyl group, a cycloalkyl group, or an aryl group; R²² is an alkyl group, a cycloalkyl group, or an alkoxyl group; and r is an integer of 0 to 3.

Examples of the other acid generating agent include bissulfonyldiazomethanes such as bis(p-toluenesulfonyl)diazomethane, bis(2,4-dimethylphenylsulfonyl)diazomethane, bis(tert-butylsulfonyl)diazomethane, bis(n-butylsulfonyl)diazomethane, bis(isobutylsulfonyl)diazomethane, bis(isopropylsulfonyl)diazomethane, bis(n-propylsulfonyl)diazomethane, bis(cyclohexylsulfonyl)diazomethane, bis(isopropylsulfonyl)diazomethane, 1,3-bis(cyclohexylsulfonylazomethylsulfonyl)propane, 1,4-bis(phenylsulfonylazomethylsulfonyl)butane, 1,6-bis(phenylsulfonylazomethylsulfonyl)hexane, and 1,10-bis(cyclohexylsulfonylazomethylsulfonyl)decane; and

halogen-containing triazine derivatives such as 2-(4-methoxyphenyl)-4,6-(bistrichloromethyl)-1,3,5-triazine, 2-(4-methoxynaphthyl)-4,6-(bistrichloromethyl)-1,3,5-triazine, tris(2,3-dibromopropyl)-1,3,5-triazine, and tris(2,3-dibromopropyl)isocyanurate.

The acid generating agent (C) is preferably an acid generating agent having an aromatic ring, and more preferably an acid generating agent represented by the formula (8-1) or (8-2), from the viewpoint of heat resistance. Among them, an acid generating agent having a sulfonate ion wherein X⁻ of the formula (8-1) or (8-2) has an aryl group or a halogen substituted aryl group is further preferable; an acid generating agent having a sulfonate ion wherein X⁻ of the formula (8-1) or (8-2) has an aryl group is still further preferable; and diphenyltrimethylphenylsulfonium p-toluenesulfonate, triphenylsulfonium p-toluenesulfonate, triphenylsulfonium trifluoromethanesulfonate, and triphenylsulfonium nonafluoromethanesulfonate are particularly preferable, from the viewpoint of a better shape of an optical member. By using the acid generating agent, line edge roughness can be reduced.

The acid generating agent (C) may be used alone or in combination of two or more kinds.

### (Acid Crosslinking Agent (G))

The optical member forming composition of the present embodiment preferably contains one or more acid crosslinking agents (G), as an additive agent for enhancing the strength of a structure. The acid crosslinking agent (G) is a compound capable of intramolecular or intermolecular crosslinking the component (A) in the presence of the acid generated from the acid generating agent (C). Examples of such an acid crosslinking agent (G) can include, but not particularly limited to, a compound having one or more groups (hereinafter, referred to as “crosslinkable group”) capable of crosslinking the component (A).

Specific examples of such a crosslinkable group are not particularly limited, and examples include (i) a hydroxyalkyl group or a group derived therefrom, such as a hydroxy (alkyl of 1 to 6 carbon atoms) group, an alkoxy of 1 to 6 carbon atoms (alkyl of 1 to 6 carbon atoms) group, and an acetoxy (alkyl of 1 to 6 carbon atoms) group; (ii) a carbonyl group or a group derived therefrom, such as a formyl group and a carboxy (alkyl of 1 to 6 carbon atoms) group; (iii) a nitrogenous group-containing group such as a dimethylaminomethyl group, a diethylaminomethyl group, a dimethylolaminomethyl group, a diethylolaminomethyl group, and a morpholinomethyl group; (iv) a glycidyl group-containing group such as a glycidyl ether group, a glycidyl ester group, and a glycidylamino group; (v) a group derived from an aromatic group such as an allyloxy of 1 to 6 carbon atoms (alkyl of 1 to 6 carbon atoms) group and an aralkyloxy of 1 to 6 carbon atoms (alkyl of 1 to 6 carbon atoms) group such as a benzyloxymethyl group and a benzoyloxymethyl group; and (vi) a polymerizable multiple bond-containing group such as a vinyl group and an isopropenyl group. As the crosslinkable group of the acid crosslinking agent (G), a hydroxyalkyl group and an alkoxyalkyl group or the like are preferable, and an alkoxymethyl group is particularly preferable.

Examples of the acid crosslinking agent (G) having the crosslinkable group include, but not particularly limited to, (i) a methylol group-containing compound such as a methylol group-containing melamine compound, a methylol group-containing benzoguanamine compound, a methylol group-containing urea compound, a methylol group-containing glycoluril compound, and a methylol group-containing phenolic compound; (ii) an alkoxyalkyl group-containing compound such as an alkoxyalkyl group-containing melamine compound, an alkoxyalkyl group-containing benzoguanamine compound, an alkoxyalkyl group-containing urea compound, an alkoxyalkyl group-containing glycoluril compound, and an alkoxyalkyl group-containing phenolic compound; (iii) a carboxymethyl group-containing compound such as a carboxymethyl group-containing melamine compound, a carboxymethyl group-containing benzoguanamine compound, a carboxymethyl group-containing urea compound, a carboxymethyl group-containing glycoluril compound, and a carboxymethyl group-containing phenolic compound; (iv) an epoxy compound such as a bisphenol A based epoxy compound, a bisphenol F based epoxy compound, a bisphenol S based epoxy compound, a novolac resin based epoxy compound, a resol resin based epoxy compound, and a poly(hydroxystyrene) based epoxy compound.

As the acid crosslinking agent (G), a compound having a phenolic hydroxyl group, and a compound and resin where the above crosslinkable group is introduced into an acid functional group in an alkali soluble resin to impart crosslinkability can be further used. The introduction rate of the crosslinkable group in that case is not particularly limited, and is adjusted to be, for example, 5 to 100 mol %, preferably 10 to 60 mol %, and more preferably 15 to 40 mol % based on the total acid functional groups in the compound having a phenolic hydroxy group, and the alkali soluble resin. When the introduction rate of the crosslinkable group is within the above range, there is a tendency that the crosslinking reaction occurs sufficiently, and a decrease in the film remaining rate, and swelling phenomena and meandering or the like of a pattern are avoided, which is preferable.

The acid crosslinking agent (G) is preferably an alkoxyalkylated urea compound or resin thereof, or an alkoxyalkylated glycoluril compound or resin thereof. The acid crosslinking agent (G) is particularly preferably a compound represented by any of the following formulae (11-1) to (11-3) or an alkoxymethylated melamine compound (hereinafter, also collectively referred to as “acid crosslinking agent (G1)”).

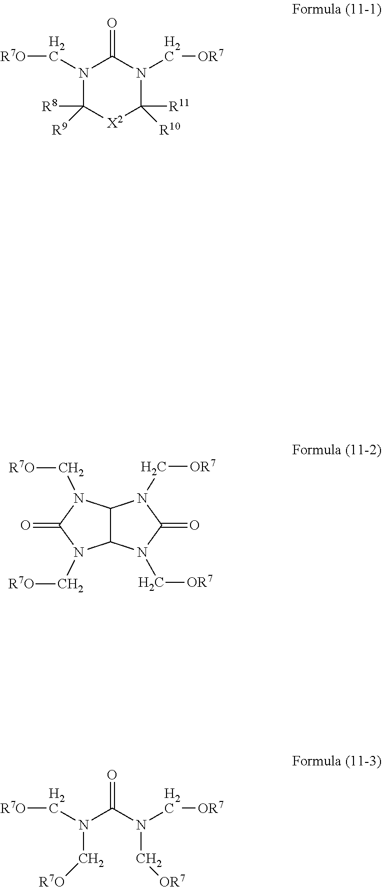

In the above formulae (11-1) to (11-3), R⁷ each independently represents a hydrogen atom, an alkyl group, or an acyl group; R⁸ to R¹¹ each independently represents a hydrogen atom, a hydroxyl group, an alkyl group, or an alkoxyl group; and X² represents a single bond, a methylene group, or an oxygen atom.

The alkyl group represented by R⁷ is not particularly limited, and is preferably of 1 to 6 carbon atoms, and more preferably of 1 to 3 carbon atoms. Examples thereof include a methyl group, an ethyl group, and a propyl group. The acyl group represented by R⁷ is not particularly limited, and is preferably of 2 to 6 carbon atoms, and more preferably of 2 to 4 carbon atoms. Examples thereof include an acetyl group and a propionyl group. The alkyl group represented by R⁸ to R¹¹ is not particularly limited, and is preferably of 1 to 6 carbon atoms, and more preferably of 1 to 3 carbon atoms. Examples thereof include a methyl group, an ethyl group, and a propyl group. The alkoxy group represented by R⁸ to R¹¹ is not particularly limited, and is preferably of 1 to 6 carbon atoms, and more preferably of 1 to 3 carbon atoms. Examples thereof include a methoxy group, an ethoxy group, and a propoxy group. X² is preferably a single bond or a methylene group. R⁷ to R¹¹ and X² may be substituted with an alkyl group such as a methyl group and an ethyl group, an alkoxy group such as a methoxy group and an ethoxy group, a hydroxyl group, and a halogen atom or the like. A plurality of R⁷ and R⁸ to R¹¹ may be each the same or different.

Specific examples of the compound represented by the formula (11-1) can include compounds represented below.

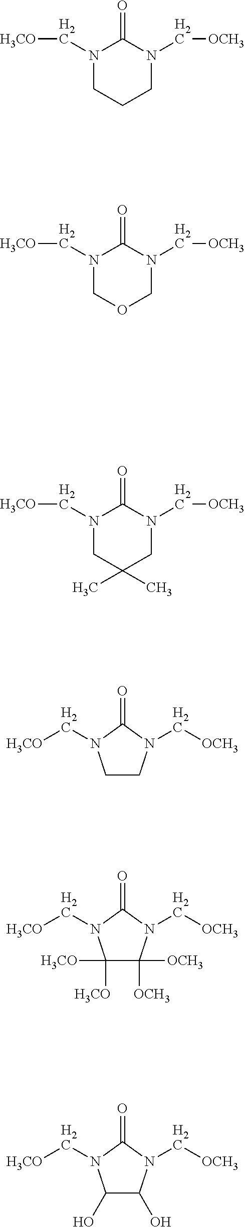 [SMILES: COCN1CCCN(COC)C1=O ; COCN1COCN(COC)C1=O ; COCN1CC(C)(C)CN(COC)C1=O ; COCN1CCN(COC)C1=O ; COCN1C(=O)N(COC)C(OC)(OC)C1(OC)OC ; COCN1C(=O)N(COC)C(O)C1O]

The compound represented by the formula (11-2) is not particularly limited, and specific examples include N,N,N,N-tetra(methoxymethyl)glycoluril, N,N,N,N-tetra(ethoxymethyl)glycoluril, N,N,N,N-tetra(n-propoxymethyl)glycoluril, N,N,N,N-tetra(isopropoxymethyl)glycoluril, N,N,N,N-tetra(n-butoxymethyl)glycoluril, and N,N,N,N-tetra(t-butoxymethyl)glycoluril. Among these, N,N,N,N-tetra(methoxymethyl)glycoluril is particularly preferable.

The compound represented by the formula (11-3) is not particularly limited, and specific examples include compounds represented below.

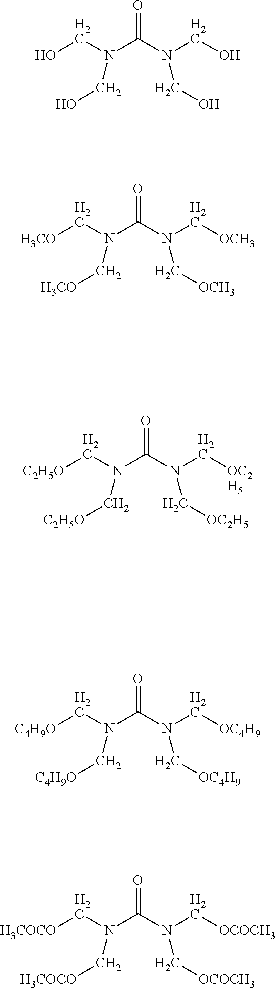

The alkoxymethylated melamine compound is not particularly limited, and specific examples include N,N,N,N,N,N-hexa(methoxymethyl)melamine, N,N,N,N,N,N-hexa(ethoxymethyl)melamine, N,N,N,N,N,N-hexa(n-propoxymethyl)melamine, N,N,N,N,N,N-hexa(isopropoxymethyl)melamine, N,N,N,N,N,N-hexa(n-butoxymethyl)melamine, and N,N,N,N,N,N-hexa(t-butoxymethyl)melamine. Among these, N,N,N,N,N,N-hexa(methoxymethyl)melamine is particularly preferable.

The acid crosslinking agent (G1) can be obtained by, for example, conducting a condensation reaction of a urea compound or a glycoluril compound with formalin to introduce a methylol group, etherifying the product with lower alcohols such as methyl alcohol, ethyl alcohol, propyl alcohol, and butyl alcohol, and then cooling the reaction solution to collect a precipitated compound or resin thereof. The acid crosslinking agent (G1) can be obtained as a commercially available product such as CYMEL (trade name, manufactured by MT AquaPolymer) and NIKALAC (manufactured by Sanwa Chemical).

Other examples of the acid crosslinking agent (G) can include a phenol derivative having 1 to 6 benzene rings within a molecule and two or more hydroxyalkyl groups and/or alkoxyalkyl groups within the entire molecule, the hydroxyalkyl groups and/or alkoxyalkyl groups being bonded to any of the above benzene rings (hereinafter, also referred to as “acid crosslinking agent (G2)”). Among them, a phenol derivative having a molecular weight of 1500 or less, 1 to 6 benzene rings and a total of two or more hydroxyalkyl groups and/or alkoxyalkyl groups within a molecule, the hydroxyalkyl groups and/or alkoxyalkyl groups being bonded to any one of the above benzene rings, or a plurality of benzene rings, is preferable.

The hydroxyalkyl group bonded to a benzene ring is not particularly limited to, and the one of 1 to 6 carbon atoms such as a hydroxymethyl group, a 2-hydroxyethyl group, and a 2-hydroxy-1-propyl group is preferable. As the alkoxyalkyl group bonded to a benzene ring, the one of 2 to 6 carbon atoms is preferable. Specific examples thereof include a methoxymethyl group, an ethoxymethyl group, an n-propoxymethyl group, an isopropoxymethyl group, an n-butoxymethyl group, an isobutoxymethyl group, a sec-butoxymethyl group, a t-butoxymethyl group, a 2-methoxyethyl group, and a 2-methoxy-1-propyl group.

Among these phenol derivatives, particularly preferable ones are shown below:

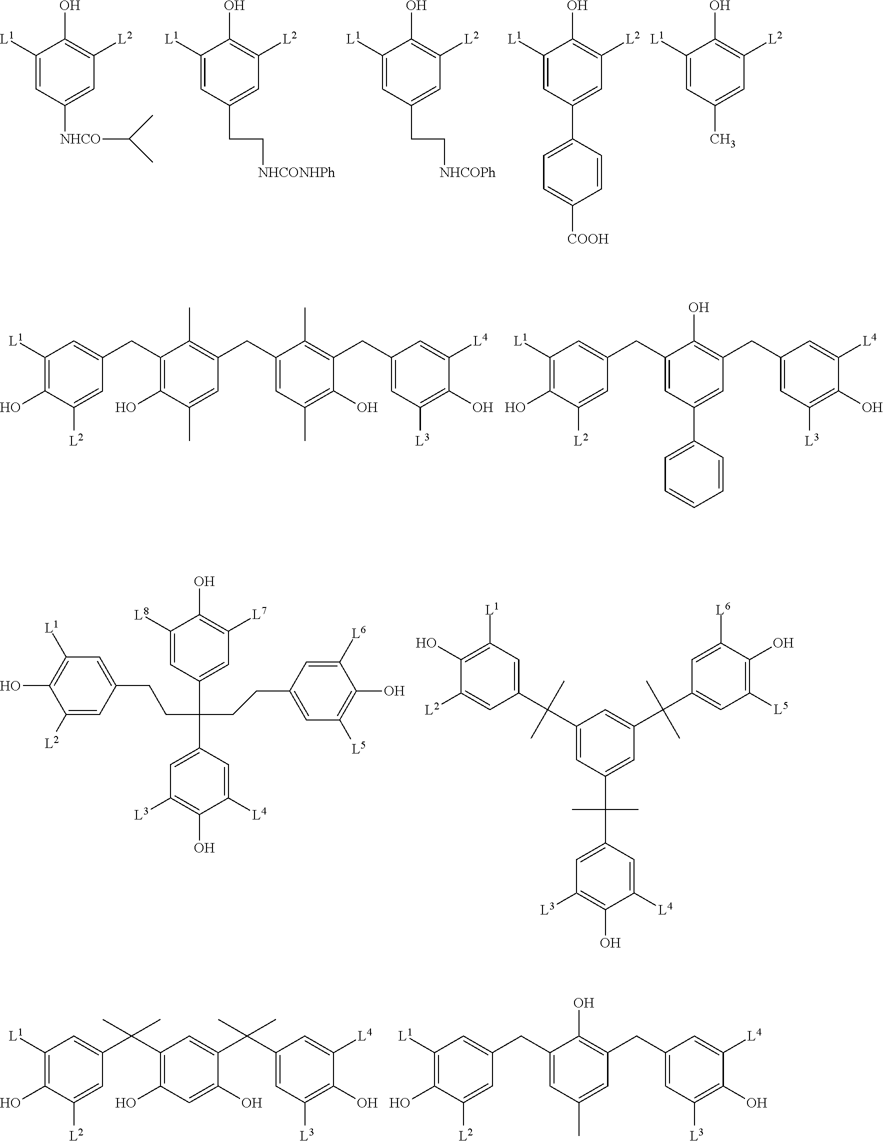

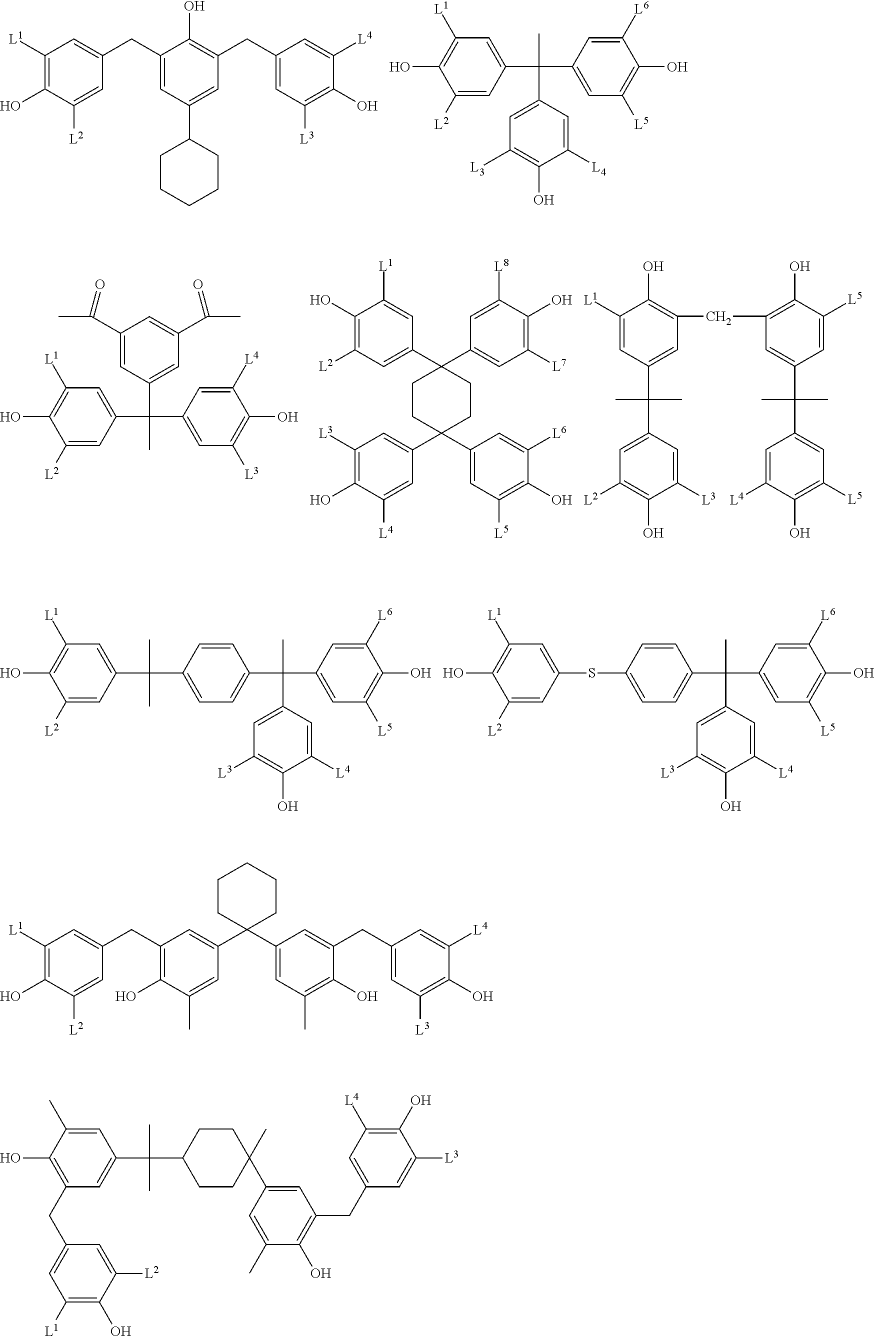

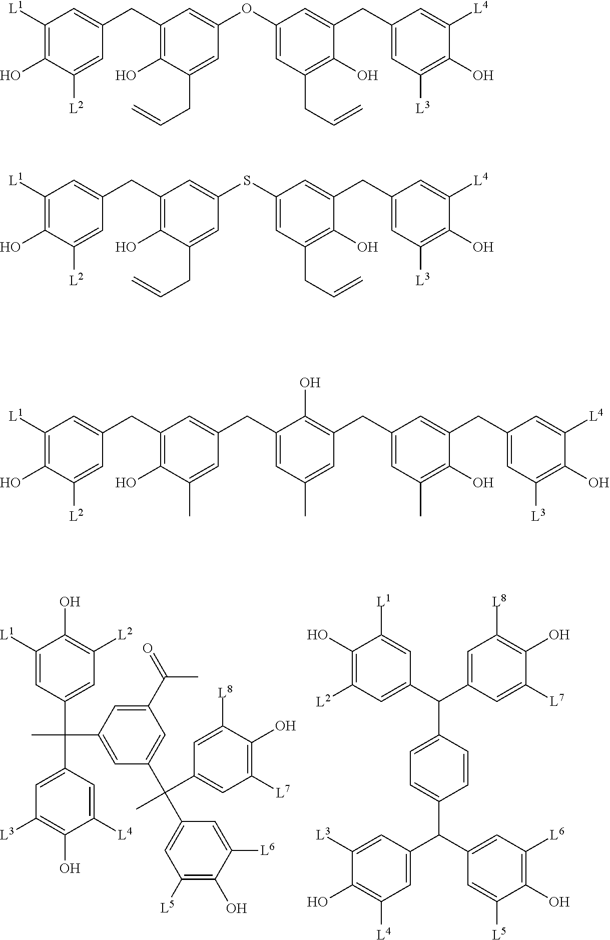

In the above formulae, L¹ to L⁸ may be the same or different, and each independently represents a hydroxymethyl group, a methoxymethyl group, or an ethoxymethyl group. A phenol derivative having a hydroxymethyl group can be obtained by reacting the corresponding phenolic compound having no hydroxymethyl group (a compound where L to L⁸ in the above formulae are a hydrogen atom) with formaldehyde in the presence of a basic catalyst. In this case, in order to prevent resinification and gelation, the reaction temperature is preferably 60° C. or less. Specifically, it can be synthesized according to methods described in Japanese Patent Application Laid-Open Nos. 6-282067 and 7-64285 or the like.

A phenol derivative having an alkoxymethyl group can be obtained by reacting the corresponding phenol derivative having a hydroxymethyl group with an alcohol in the presence of an acid catalyst. In this case, in order to prevent resinification and gelation, the reaction temperature is preferably 100° C. or less. Specifically, it can be synthesized according to methods described in EP632003A1 or the like.

While the phenol derivative having a hydroxymethyl group and/or an alkoxymethyl group thus synthesized is preferable in terms of stability upon storage, the phenol derivative having an alkoxymethyl group is particularly preferable. The acid crosslinking agent (G2) may be used alone, or may be used in combination of two or more kinds.

Other examples of the acid crosslinking agent (G) can include a compound having at least one α-hydroxyisopropyl group (hereinafter, also referred to as “acid crosslinking agent (G3)”). The compound is not particularly limited in the structure, as long as it has an α-hydroxyisopropyl group. A hydrogen atom of a hydroxyl group in the above α-hydroxyisopropyl group may be substituted with one or more acid dissociation reactive groups (R—COO— group, R—SO₂— group or the like, wherein R represents a substituent group selected from the group consisting of a linear hydrocarbon group of 1 to 12 carbon atoms, a cyclic hydrocarbon group of 3 to 12 carbon atoms, an alkoxy group of 1 to 12 carbon atoms, a 1-branched alkyl group of 3 to 12 carbon atoms, and an aromatic hydrocarbon group of 6 to 12 carbon atoms). Examples of a compound having the above α-hydroxyisopropyl group include one kind or two kinds or more of a substituted or non-substituted aromatic based compound, a diphenyl compound, a naphthalene compound, a furan compound or the like containing at least one α-hydroxyisopropyl group. Specific examples thereof include a compound represented by the following formula (12-1) (hereinafter, referred to as “benzene based compound (1)”), a compound represented by the following formula (12-2) (hereinafter, referred to as “diphenyl based compound (2)”), a compound represented by the following formula (12-3) (hereinafter, referred to as “naphthalene based compound (3)”), and a compound represented by the following formula (12-4) (hereinafter, referred to as “furan based compound (4)”).

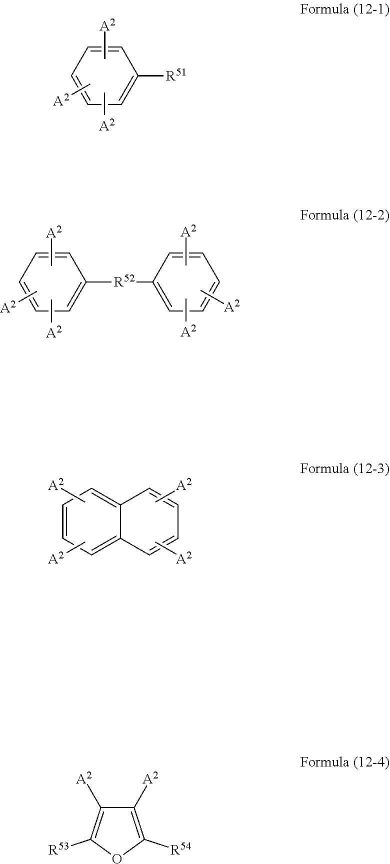

In the above formulae (12-1) to (12-4), each A² independently represents an α-hydroxyisopropyl group or a hydrogen atom, and at least one A² is an α-hydroxyisopropyl group. In the formula (12-1), R⁵¹ represents a hydrogen atom, a hydroxyl group, a linear or branched alkylcarbonyl group of 2 to 6 carbon atoms, or a linear or branched alkoxycarbonyl group of 2 to 6 carbon atoms. Furthermore, in the formula (12-2), R⁵² represents a single bond, a linear or branched alkylene group of 1 to 5 carbon atoms, —O—, —CO—, or —COO—. Also, in the formula (12-4), R⁵³ and R⁵⁴ represent a hydrogen atom or a linear or branched alkyl group of 1 to 6 carbon atoms independently from each other.

Examples of the benzene based compound (1) are not particularly limited, and examples include α-hydroxyisopropylbenzenes such as α-hydroxyisopropylbenzene, 1,3-bis(α-hydroxyisopropyl)benzene, 1,4-bis(α-hydroxyisopropyl)benzene, 1,2,4-tris(α-hydroxyisopropyl)benzene, and 1,3,5-tris(α-hydroxyisopropyl)benzene; α-hydroxyisopropylphenols such as 3-α-hydroxyisopropylphenol, 4-α-hydroxyisopropylphenol, 3,5-bis(α-hydroxyisopropyl)phenol, and 2,4,6-tris(α-hydroxyisopropyl)phenol; α-hydroxyisopropylphenyl alkyl ketones such as 3-α-hydroxyisopropylphenyl methyl ketone, 4-α-hydroxyisopropylphenyl methyl ketone, 4-α-hydroxyisopropylphenyl ethyl ketone, 4-α-hydroxyisopropylphenyl-n-propyl ketone, 4-α-hydroxyisopropylphenyl isopropyl ketone, 4-α-hydroxyisopropylphenyl-n-butyl ketone, 4-α-hydroxyisopropylphenyl-t-butyl ketone, 4-α-hydroxyisopropylphenyl-n-pentyl ketone, 3,5-bis(α-hydroxyisopropyl)phenyl methyl ketone, 3,5-bis(α-hydroxyisopropyl)phenyl ethyl ketone, and 2,4,6-tris(α-hydroxyisopropyl)phenyl methyl ketone; alkyl 4-α-hydroxyisopropylbenzoates such as methyl 3-α-hydroxyisopropylbenzoate, methyl 4-α-hydroxyisopropylbenzoate, ethyl 4-α-hydroxyisopropylbenzoate, n-propyl 4-α-hydroxyisopropylbenzoate, isopropyl 4-α-hydroxyisopropylbenzoate, n-butyl 4-α-hydroxyisopropylbenzoate, t-butyl 4-α-hydroxyisopropylbenzoate, n-pentyl 4-α-hydroxyisopropylbenzoate, methyl 3,5-bis(α-hydroxyisopropyl)benzoate, ethyl 3,5-bis(α-hydroxyisopropyl)benzoate, and methyl 2,4,6-tris(α-hydroxyisopropyl)benzoate.

Examples of the diphenyl based compound (2) are not particularly limited, and examples include α-hydroxyisopropylbiphenyls such as 3-α-hydroxyisopropylbiphenyl, 4-α-hydroxyisopropylbiphenyl, 3,5-bis(α-hydroxyisopropyl)biphenyl, 3,3′-bis(α-hydroxyisopropyl)biphenyl, 3,4′-bis(α-hydroxyisopropyl)biphenyl, 4,4′-bis(α-hydroxyisopropyl)biphenyl, 2,4,6-tris(α-hydroxyisopropyl)biphenyl, 3,3′,5-tris(α-hydroxyisopropyl)biphenyl, 3,4′,5-tris(α-hydroxyisopropyl)biphenyl, 2,3′,4,6,-tetrakis(α-hydroxyisopropyl)biphenyl, 2,4,4′,6,-tetrakis(α-hydroxyisopropyl)biphenyl, 3,3′,5,5′-tetrakis(α-hydroxyisopropyl)biphenyl, 2,3′,4,5′,6-pentakis(α-hydroxyisopropyl)biphenyl, and 2,2′,4,4′,6,6′-hexakis(α-hydroxyisopropyl)biphenyl; α-hydroxyisopropyldiphenylalkanes such as 3-α-hydroxyisopropyldiphenylmethane, 4-α-hydroxyisopropyldiphenylmethane, 1-(4-α-hydroxyisopropylphenyl)-2-phenylethane, 1-(4-α-hydroxyisopropylphenyl)-2-phenylpropane, 2-(4-α-hydroxyisopropylphenyl)-2-phenylpropane, 1-(4-α-hydroxyisopropylphenyl)-3-phenylpropane, 1-(4-α-hydroxyisopropylphenyl)-4-phenylbutane, 1-(4-α-hydroxyisopropylphenyl)-5-phenylpentane, 3,5-bis(α-hydroxyisopropyldiphenylmethane, 3,3′-bis(α-hydroxyisopropyl)diphenylmethane, 3,4′-bis(α-hydroxyisopropyl)diphenylmethane, 4,4′-bis(α-hydroxyisopropyl)diphenylmethane, 1,2-bis(4-α-hydroxyisopropylphenyl)ethane, 1,2-bis(4-α-hydroxypropylphenyl)propane, 2,2-bis(4-α-hydroxypropylphenyl)propane, 1,3-bis(4-α-hydroxypropylphenyl)propane, 2,4,6-tris(α-hydroxyisopropyl)diphenylmethane, 3,3′,5-tris(α-hydroxyisopropyl)diphenylmethane, 3,4′,5-tris(α-hydroxyisopropyl)diphenylmethane, 2,3′,4,6-tetrakis(α-hydroxyisopropyl)diphenylmethane, 2,4,4′,6-tetrakis(α-hydroxyisopropyl)diphenylmethane, 3,3′,5,5′-tetrakis(α-hydroxyisopropyl)diphenylmethane, 2,3′,4,5′,6-pentakis(α-hydroxyisopropyl)diphenylmethane, and 2,2′,4,4′,6,6′-hexakis(α-hydroxyisopropyl)diphenylmethane; α-hydroxyisopropyldiphenyl ethers such as 3-α-hydroxyisopropyldiphenyl ether, 4-α-hydroxyisopropyldiphenyl ether, 3,5-bis(α-hydroxyisopropyl)diphenyl ether, 3,3′-bis(α-hydroxyisopropyl)diphenyl ether, 3,4′-bis(α-hydroxyisopropyl)diphenyl ether, 4,4′-bis(α-hydroxyisopropyl)diphenyl ether, 2,4,6-tris(α-hydroxyisopropyl)diphenyl ether, 3,3′,5-tris(α-hydroxyisopropyl)diphenyl ether, 3,4′,5-tris(α-hydroxyisopropyl)diphenyl ether, 2,3′,4,6-tetrakis(α-hydroxyisopropyl)diphenyl ether, 2,4,4′,6-tetrakis(α-hydroxyisopropyl)diphenyl ether, 3,3′,5,5′-tetrakis(α-hydroxyisopropyl)diphenyl ether, 2,3′,4,5′,6-pentakis(α-hydroxyisopropyl)diphenyl ether, and 2,2′,4,4′,6,6′-hexakis(α-hydroxyisopropyl)diphenyl ether; α-hydroxyisopropyldiphenyl ketones such as 3-α-hydroxyisopropyldiphenyl ketone, 4-α-hydroxyisopropyldiphenyl ketone, 3,5-bis(α-hydroxyisopropyl)diphenyl ketone, 3,3′-bis(α-hydroxyisopropyl)diphenyl ketone, 3,4′-bis(α-hydroxyisopropyl)diphenyl ketone, 4,4′-bis(α-hydroxyisopropyl)diphenyl ketone, 2,4,6-tris(α-hydroxyisopropyl)diphenyl ketone, 3,3′,5-tris(α-hydroxyisopropyl)diphenyl ketone, 3,4′,5-tris(α-hydroxyisopropyl)diphenyl ketone, 2,3′,4,6-tetrakis(α-hydroxyisopropyl)diphenyl ketone, 2,4,4′,6-tetrakis(α-hydroxyisopropyl)diphenyl ketone, 3,3′,5,5′-tetrakis(α-hydroxyisopropyl)diphenyl ketone, 2,3′,4,5′,6-pentakis(α-hydroxyisopropyl)diphenyl ketone, and 2,2′,4,4′,6,6′-hexakis(α-hydroxyisopropyl)diphenyl ketone; phenyl α-hydroxyisopropylbenzoates such as phenyl 3-α-hydroxyisopropylbenzoate, phenyl 4-α-hydroxyisopropylbenzoate, 3-α-hydroxyisopropylphenyl benzoate, 4-α-hydroxyisopropylphenyl benzoate, phenyl 3,5-bis(α-hydroxyisopropyl)benzoate, 3-α-hydroxyisopropylphenyl 3-α-hydroxyisopropylbenzoate, 4-α-hydroxyisopropylphenyl 3-α-hydroxyisopropylbenzoate, 3-α-hydroxyisopropylphenyl 4-α-hydroxyisopropylbenzoate, 4-α-hydroxyisopropylphenyl 4-α-hydroxyisopropylbenzoate, 3,5-bis(α-hydroxyisopropyl)phenyl benzoate, phenyl 2,4,6-tris(α-hydroxyisopropyl)benzoate, 3-α-hydroxyisopropylphenyl 3,5-bis(α-hydroxyisopropyl)benzoate, 4-α-hydroxyisopropylphenyl 3,5-bis(α-hydroxyisopropyl)benzoate, 3,5-bis(α-hydroxyisopropyl)phenyl 3-α-hydroxyisopropylbenzoate, 3,5-bis(α-hydroxyisopropyl)phenyl 4-α-hydroxyisopropylbenzoate, 2,4,6-tris(α-hydroxyisopropyl)phenyl benzoate, 3-α-hydroxyisopropylphenyl 2,4,6-tris(α-hydroxyisopropyl)benzoate, 4-α-hydroxyisopropylphenyl 2,4,6-tris(α-hydroxyisopropyl)benzoate, 3,5-bis(α-hydroxyisopropyl)phenyl 3,5-bis(α-hydroxyisopropyl)benzoate, 2,4,6-tris(α-hydroxyisopropyl)phenyl 3-α-hydroxyisopropylbenzoate, 2,4,6-tris(α-hydroxyisopropyl)phenyl 4-α-hydroxyisopropylbenzoate, 3,5-bis(α-hydroxyisopropyl)phenyl 2,4,6-tris(α-hydroxyisopropyl)benzoate, 2,4,6-tris(α-hydroxyisopropyl)phenyl 3,5-bis(α-hydroxyisopropyl)benzoate, and 2,4,6-tris(α-hydroxyisopropyl)phenyl 2,4,6-tris(α-hydroxyisopropyl)benzoate.

Examples of the naphthalene based compound (3) are not particularly limited, and examples include 1-(α-hydroxyisopropyl)naphthalene, 2-(α-hydroxyisopropyl)naphthalene, 1,3-bis(α-hydroxyisopropyl)naphthalene, 1,4-bis(α-hydroxyisopropyl)naphthalene, 1,5-bis(α-hydroxyisopropyl)naphthalene, 1,6-bis(α-hydroxyisopropyl)naphthalene, 1,7-bis(α-hydroxyisopropyl)naphthalene, 2,6-bis(α-hydroxyisopropyl)naphthalene, 2,7-bis(α-hydroxyisopropyl)naphthalene, 1,3,5-tris(α-hydroxyisopropyl)naphthalene, 1,3,6-tris(α-hydroxyisopropyl)naphthalene, 1,3,7-tris(α-hydroxyisopropyl)naphthalene, 1,4,6-tris(α-hydroxyisopropyl)naphthalene, 1,4,7-tris(α-hydroxyisopropyl)naphthalene, and 1,3,5,7-tetrakis(α-hydroxyisopropyl)naphthalene.

Examples of the furan based compound (4) include, but not particularly limited to, 3-(α-hydroxyisopropyl)furan, 2-methyl-3-(α-hydroxyisopropyl)furan, 2-methyl-4-(α-hydroxyisopropyl)furan, 2-ethyl-4-(α-hydroxyisopropyl)furan, 2-n-propyl-4-(α-hydroxyisopropyl)furan, 2-isopropyl-4-(α-hydroxyisopropyl)furan, 2-n-butyl-4-(α-hydroxyisopropyl)furan, 2-t-butyl-4-(α-hydroxyisopropyl)furan, 2-n-pentyl-4-(α-hydroxyisopropyl)furan, 2,5-dimethyl-3-(α-hydroxyisopropyl)furan, 2,5-diethyl-3-(α-hydroxyisopropyl)furan, 3,4-bis(α-hydroxyisopropyl)furan, 2,5-dimethyl-3,4-bis(α-hydroxyisopropyl)furan, and 2,5-diethyl-3,4-bis(α-hydroxyisopropyl)furan.

As the acid crosslinking agent (G3), a compound having two or more free α-hydroxyisopropyl groups is preferable; the above benzene based compound (1) having two or more α-hydroxyisopropyl groups, the above diphenyl based compound (2) having two or more α-hydroxyisopropyl groups, and the above naphthalene based compound (3) having two or more α-hydroxyisopropyl groups are further preferable; and α-hydroxyisopropylbiphenyls having two or more α-hydroxyisopropyl groups and the above naphthalene based compound (3) having two or more α-hydroxyisopropyl groups are particularly preferable.

The acid crosslinking agent (G3) can normally be obtained by a method for reacting an acetyl group-containing compound such as 1,3-diacetylbenzene with Grignard reagent such as CH₃MgBr to methylate and then hydrolyzing, or a method for oxidizing an isopropyl group-containing compound such as 1,3-diisopropylbenzene with oxygen or the like to produce a peroxide and then reducing.

In the optical member forming composition of the present embodiment, the content of the acid crosslinking agent (G) is preferably 0.5 to 49% by mass of the total mass of the solid components, more preferably 0.5 to 40% by mass, still more preferably 1 to 30% by mass, and particularly preferably 2 to 20% by mass. When the content of the acid crosslinking agent (G) is 0.5% by mass or more, there is a tendency that the inhibiting effect of the solubility of the optical member forming composition in an organic solvent can be improved. On the other hand, when the content is 49% by mass or less, there is a tendency that a decrease in the heat resistance of the optical member forming composition can be inhibited.

The content of at least one kind of compound selected from the acid crosslinking agent (G1), acid crosslinking agent (G2), and acid crosslinking agent (G3) in the acid crosslinking agent (G) is also not particularly limited, and can be within various ranges according to the kind of substrates or the like used upon forming an optical member forming composition.

In all acid crosslinking agent components, the content of the alkoxymethylated melamine compound and/or the compounds represented by formula (12-1) to formula (12-4) is not particularly limited, but is preferably 50 to 99% by mass, more preferably 60 to 99% by mass, still more preferably 70 to 98% by mass, and particularly preferably 80 to 97% by mass. When the content of the alkoxymethylated melamine compound and/or the compounds represented by formula (12-1) to formula (12-4) is 50% by mass or more of all acid crosslinking agent components, there is a tendency that the resolution can be further improved. When the content is 99% by mass or less, there is a tendency that the structure is likely to have a good shape.

### (Acid Diffusion Controlling Agent (E))

The optical member forming composition of the present embodiment may contain an acid diffusion controlling agent (E) having a function of controlling diffusion of an acid generated from an acid generating agent in the optical member forming composition to inhibit any unpreferable chemical reaction or the like. By using the acid diffusion controlling agent (E), the storage stability of the optical member forming composition is improved. Also, along with the further improvement of the resolution, the line width change of a structure due to variation in the post exposure delay time after heating can be inhibited, and the composition has extremely excellent process stability.

The acid diffusion controlling agent (E) is not particularly limited, and examples include a radiation degradable basic compound such as a nitrogen atom-containing basic compound, a basic sulfonium compound, and a basic iodonium compound. The acid diffusion controlling agent (E) may be used alone or in combination of two or more kinds.

The acid diffusion controlling agent is not particularly limited, and examples include a nitrogen-containing organic compound, and a basic compound degradable by exposure. The nitrogen-containing organic compound is not particularly limited, and examples include a compound represented by the following formula (14):

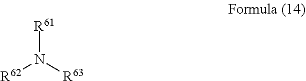

Examples of the nitrogen-containing organic compound include a compound represented by the above formula (14) (hereinafter, referred to as a “nitrogen-containing compound (I)”), a diamino compound having two nitrogen atoms within the same molecule (hereinafter, referred to as a “nitrogen-containing compound (II)”), a polyamino compound or polymer having three or more nitrogen atoms (hereinafter, referred to as a “nitrogen-containing compound (III)”), an amide group-containing compound, a urea compound, and a nitrogen-containing heterocyclic compound. The acid diffusion controlling agent (E) may be used alone as one kind or may be used in combination of two or more kinds.

In the above formula (14), R⁶¹, R⁶², and R⁶³ represent a hydrogen atom, a linear, branched or cyclic alkyl group, an aryl group, or an aralkyl group independently from each other. The above alkyl group, aryl group, or aralkyl group may be non-substituted or may be substituted with a hydroxyl group or the like. Herein, the above linear, branched or cyclic alkyl group is not particularly limited, and examples include the one of 1 to 15 carbon atoms, and preferably 1 to 10 carbon atoms. Specific examples thereof include a methyl group, an ethyl group, an n-propyl group, an isopropyl group, an n-butyl group, an isobutyl group, a sec-butyl group, a t-butyl group, an n-pentyl group, a neopentyl group, an n-hexyl group, a texyl group, an n-heptyl group, an n-octyl group, an n-ethylhexyl group, an n-nonyl group, and an n-decyl group. Examples of the above aryl group include the one of 6 to 12 carbon atoms. Specific examples thereof include a phenyl group, a tolyl group, a xylyl group, a cumenyl group, and a 1-naphthyl group. Furthermore, the above aralkyl group is not particularly limited, and examples include the one of 7 to 19 carbon atoms, and preferably 7 to 13 carbon atoms. Specific examples thereof include a benzyl group, an α-methylbenzyl group, a phenethyl group, and a naphthylmethyl group.

The nitrogen-containing compound (I) is not particularly limited, and specific examples include particularly mono(cyclo)alkylamines such as n-hexylamine, n-heptylamine, n-octylamine, n-nonylamine, n-decylamine, n-dodecylamine, and cyclohexylamine; di(cyclo)alkylamines such as di-n-butylamine, di-n-pentylamine, di-n-hexylamine, di-n-heptylamine, di-n-octylamine, di-n-nonylamine, di-n-decylamine, methyl-n-dodecylamine, di-n-dodecylmethyl, cyclohexylmethylamine, and dicyclohexylamine; tri(cyclo)alkylamines such as triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, dimethyl-n-dodecylamine, di-n-dodecylmethylamine, dicyclohexylmethylamine, and tricyclohexylamine; alkanolamines such as monoethanolamine, diethanolamine, and triethanolamine; and aromatic amines such as aniline, N-methylaniline, N,N-dimethylaniline, 2-methylaniline, 3-methylaniline, 4-methylaniline, 4-nitroaniline, diphenylamine, triphenylamine, and 1-naphthylamine.

The nitrogen-containing compound (II) is not particularly limited, and specific examples include particularly ethylenediamine, N,N,N′,N′-tetramethylethylenediamine, N,N,N′,N′-tetrakis(2-hydroxypropyl)ethylenediamine, tetramethylenediamine, hexamethylenediamine, 4,4′-diaminodiphenylmethane, 4,4′-diaminodiphenyl ether, 4,4′-diaminobenzophenone, 4,4′-diaminodiphenylamine, 2,2-bis(4-aminophenyl)propane, 2-(3-aminophenyl)-2-(4-aminophenyl)propane, 2-(4-aminophenyl)-2-(3-hydroxyphenyl)propane, 2-(4-aminophenyl)-2-(4-hydroxyphenyl)propane, 1,4-bis[1-(4-aminophenyl)-1-methylethyl]benzene, and 1,3-bis[1-(4-aminophenyl)-1-methylethyl]benzene.

The nitrogen-containing compound (III) is not particularly limited, and specific examples include particularly polymers of polyethyleneimine, polyarylamine, and N-(2-dimethylaminoethyl)acrylamide.

The amide group-containing compound is not particularly limited, and specific examples include particularly formamide, N-methylformamide, N,N-dimethylformamide, acetamide, N-methylacetamide, N,N-dimethylacetamide, propioneamide, benzamide, pyrrolidone, and N-methylpyrrolidone.

The urea compound is not particularly limited, and specific examples include particularly urea, methylurea, 1,1-dimethylurea, 1,3-dimethylurea, 1,1,3,3-tetramethylurea, 1,3-diphenylurea, and tri-n-butylthiourea.

The nitrogen-containing heterocyclic compound is not particularly limited, and specific examples include particularly imidazoles such as imidazole, benzimidazole, 4-methylimidazole, 4-methyl-2-phenylimidazole, and 2-phenylbenzimidazole; pyridines such as pyridine, 2-methylpyridine, 4-methylpyridine, 2-ethylpyridine, 4-ethylpyridine, 2-phenylpyridine, 4-phenylpyridine, 2-methyl-4-phenylpyridine, nicotine, nicotinic acid, amide nicotinate, quinoline, 8-oxyquinoline, and acridine; and

pyrazine, pyrazole, pyridazine, quinozaline, purine, pyrrolidine, piperidine, morpholine, 4-methylmorpholine, piperazine, 1,4-dimethylpiperazine, and 1,4-diazabicyclo[2.2.2]octane.

The radiation degradable basic compound is not particularly limited, and examples include a sulfonium compound represented by the following formula (15-1) and an iodonium compound represented by the following formula (15-2):

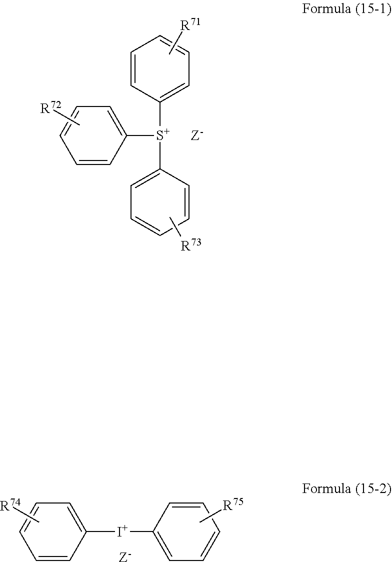

In the above formulae (15-1) and (15-2), R⁷¹, R⁷², R⁷³, R⁷⁴, and R⁷⁵ each independently represent a hydrogen atom, an alkyl group of 1 to 6 carbon atoms, an alkoxyl group of 1 to 6 carbon atoms, a hydroxyl group, or a halogen atom. Z⁻ represents HO⁻, R—COO⁻ (R represents an alkyl group of 1 to 6 carbon atoms, an aryl group of 6 to 11 carbon atoms, or an alkaryl group of 7 to 12 carbon atoms), or an anion represented by the following formula (15-3):

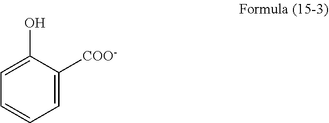

Specific examples of the radiation degradable basic compound are not particularly limited, and examples include triphenylsulfonium hydroxide, triphenylsulfonium acetate, triphenylsulfonium salicylate, diphenyl-4-hydroxyphenylsulfonium hydroxide, diphenyl-4-hydroxyphenylsulfonium acetate, diphenyl-4-hydroxyphenylsulfonium salicylate, bis(4-t-butylphenyl)iodonium hydroxide, bis(4-t-butylphenyl)iodonium acetate, bis(4-t-butylphenyl)iodonium hydroxide, bis(4-t-butylphenyl)iodonium salicylate, 4-t-butylphenyl-4-hydroxyphenyliodonium hydroxide, 4-t-butylphenyl-4-hydroxyphenyliodonium acetate, and 4-t-butylphenyl-4-hydroxyphenyliodonium salicylate.

The content of the acid diffusion controlling agent (E) is preferably 0.001 to 49% by mass of the total mass of the solid components, more preferably 0.01 to 10% by mass, still more preferably 0.01 to 5% by mass, and particularly preferably 0.01 to 3% by mass. When the content of the acid diffusion controlling agent (E) is within the above range, there is a tendency that a decrease in resolution, and deterioration of the pattern shape and the dimension fidelity or the like can be further inhibited. Moreover, even though the post exposure delay time from electron beam irradiation to heating after radiation irradiation becomes longer, the risk of deteriorating the shape of the pattern upper layer portion can be reduced. When the content of the acid diffusion controlling agent (E) is 10% by mass or less, there is a tendency that a decrease in sensitivity, and developability of the unexposed portion or the like can be prevented. By using the acid diffusion controlling agent, the storage stability of an optical member forming composition improves, also along with improvement of the resolution, the line width change of an optical member forming composition due to variation in the post exposure delay time before radiation irradiation and the post exposure delay time after radiation irradiation can be inhibited, and the composition has extremely excellent process stability.

### (Other Component (F))

To the optical member forming composition of the present embodiment, within the range of not inhibiting the purpose of the present embodiment, if required, as the other component (F), one kind or two kinds or more of various additive agents such as a dissolution promoting agent, a dissolution controlling agent, a sensitizing agent, a surfactant and an organic carboxylic acid or an oxo acid of phosphor, or derivative thereof can be added.

### [Dissolution Promoting Agent]

The dissolution promoting agent is a component having a function of increasing the solubility of the component (A) in a developing solution to moderately increase the dissolution rate of the compound upon developing, when the solubility of the component (A) is too low. The dissolution promoting agent can be used, within the range of not deteriorating the effect of the present invention. Examples of the dissolution promoting agent can include low molecular weight phenolic compounds, such as bisphenols and tris(hydroxyphenyl)methane. These dissolution promoting agents can be used alone or in mixture of two or more kinds. The content of the dissolution promoting agent, which is arbitrarily adjusted according to the kind of the component (A) to be used, is preferably 0 to 49% by mass of the total mass of the solid components, more preferably 0 to 5% by mass, still more preferably 0 to 1% by mass, and particularly preferably 0% by mass.

### [Dissolution Controlling Agent]

The dissolution controlling agent is a component having a function of controlling the solubility of the component (A) in a developing solution to moderately decrease the dissolution rate upon developing, when the solubility of the component (A) is too high. As such a dissolution controlling agent, the one which does not chemically change in steps such as calcination of an optical component, heating, and development is preferable.

The dissolution controlling agent is not particularly limited, and examples include aromatic hydrocarbons such as phenanthrene, anthracene, and acenaphthene; ketones such as acetophenone, benzophenone, and phenyl naphtyl ketone; and sulfones such as methyl phenyl sulfone, diphenyl sulfone, and dinaphthyl sulfone. These dissolution controlling agents can be used alone or in two or more kinds.

The content of the dissolution controlling agent is not particularly limited and is arbitrarily adjusted according to the kind of the component (A), but is preferably 0 to 49% by mass of the total mass of the solid components, more preferably 0 to 5% by mass, still more preferably 0 to 1% by mass, and particularly preferably 0% by mass.

### [Sensitizing Agent]

The sensitizing agent is a component having a function of absorbing irradiated radiation energy, transmitting the energy to the acid generating agent (C), and thereby increasing the acid production amount, and improving the apparent sensitivity of a resist. Such a sensitizing agent is not particularly limited, and examples include benzophenones, biacetyls, pyrenes, phenothiazines, and fluorenes. These sensitizing agents can be used alone or in two or more kinds. The content of the sensitizing agent, which is arbitrarily adjusted according to the kind of the component (A), is preferably 0 to 49% by mass of the total mass of the solid components, more preferably 0 to 5% by mass, still more preferably 0 to 1% by mass, and particularly preferably 0% by mass.

### [Surfactant]

The surfactant is a component having a function of improving coatability and striation of the optical member forming composition of the present embodiment or the like. Such a surfactant is not particularly limited, and may be any of anionic, cationic, nonionic or amphoteric. The surfactant is preferably a nonionic surfactant. The nonionic surfactant has a good affinity with a solvent used in production of optical member forming compositions and tends to have more remarkable effects. Specific examples of the nonionic surfactant include, but not particularly limited to, polyoxyethylene higher alkyl ethers, polyoxyethylene higher alkyl phenyl ethers, and higher fatty acid diesters of polyethylene glycol. Examples of commercially available products can include, hereinafter by trade name, EFTOP (manufactured by Jemco Inc.), MEGAFAC (manufactured by DIC Corporation), Fluorad (manufactured by Sumitomo 3M Limited), AsahiGuard, Surflon (hereinbefore, manufactured by Asahi Glass Co., Ltd.), Pepole (manufactured by Toho Chemical Industry Co., Ltd.), KP (manufactured by Shin-Etsu Chemical Co., Ltd.), and Polyflow (manufactured by Kyoeisha Chemical Co., Ltd.). The content of the surfactant is not particularly limited, and is arbitrarily adjusted according to the kind of the component (A), but is preferably 0 to 49% by mass of the total mass of the solid components, more preferably 0 to 5% by mass, still more preferably 0 to 1% by mass, and particularly preferably 0% by mass.

### [Organic Carboxylic Acid or Oxo Acid of Phosphor or Derivative Thereof]

For the purpose of prevention of sensitivity deterioration or improvement of a structure and post exposure delay stability or the like, and as an additional optional component, the optical member forming composition of the present embodiment may contain an organic carboxylic acid or an oxo acid of phosphor or derivative thereof. These compounds can be used in combination with the acid diffusion controlling agent, or may be used alone. The organic carboxylic acid is not particularly limited, and, for example, is suitably malonic acid, citric acid, malic acid, succinic acid, benzoic acid, salicylic acid, or the like. Examples of the oxo acid of phosphor or derivative thereof include phosphoric acid or derivative thereof such as ester including phosphoric acid, di-n-butyl ester phosphate, and diphenyl ester phosphate; phosphonic acid or derivative thereof such as ester including phosphonic acid, dimethyl ester phosphonate, di-n-butyl ester phosphonate, phenylphosphonic acid, diphenyl ester phosphonate, and dibenzyl ester phosphonate; and phosphinic acid and derivative thereof such as ester including phosphinic acid and phenylphosphinic acid. Among these, phosphonic acid is particularly preferable.

The organic carboxylic acid or the oxo acid of phosphor or derivative thereof can be used alone or in combination of two or more kinds. The content of the organic carboxylic acid or the oxo acid of phosphor or derivative thereof, which is arbitrarily adjusted according to the kind of the resin derived from the component (A), is preferably 0 to 49% by mass of the total mass of the solid components, more preferably 0 to 5% by mass, still more preferably 0 to 1% by mass, and particularly preferably 0% by mass.

### [Other Additive Agent]

The optical member forming composition of the present embodiment can contain one kind or two kinds or more of additive agents other than the above dissolution controlling agent, sensitizing agent, and surfactant, within the range of not inhibiting the purpose of the present invention, if required. Examples of such an additive agent include, but not particularly limited to, a dye, a pigment, and an adhesion aid. The composition contains the dye or the pigment, whereby there is a tendency that a latent image of the exposed portion is visualized and influence of halation upon exposure can be alleviated. The composition contains the adhesion aid, whereby there is a tendency that adhesiveness to a substrate can be improved. Furthermore, examples of other additive agents can include a halation preventing agent, a storage stabilizing agent, a defoaming agent, and a shape improving agent. Specific examples thereof can include 4-hydroxy-4′-methylchalkone.

It is preferable that the optical member forming composition of the present embodiment further contains a crosslinking agent.

The crosslinking agent is preferably at least one selected from the group consisting of a phenol compound, an epoxy compound, a cyanate compound, an amino compound, a benzoxazine compound, a melamine compound, a guanamine compound, a glycoluril compound, a urea compound, an isocyanate compound and an azide compound. The crosslinking agent more preferably has at least one allyl group.

The content of the crosslinking agent is preferably 0.1 to 50% by mass of the total mass of the solid components, more preferably 0.1 to 10% by mass, and still more preferably 0.1 to 1% by mass.

It is preferable that the optical member forming composition of the present embodiment further contains a crosslinking promoting agent.

The crosslinking promoting agent is preferably at least one selected from the group consisting of an amine, an imidazole, an organic phosphine, and a Lewis acid.

The content of the crosslinking promoting agent is preferably 0.1 to 10% by mass of the total mass of the solid components, more preferably 0.1 to 5% by mass, and still more preferably 0.1 to 1% by mass.

It is preferable that the optical member forming composition of the present embodiment further contains a radical polymerization initiator.

The radical polymerization initiator is preferably at least one selected from the group consisting of a ketone based photopolymerization initiator, an organic peroxide based polymerization initiator and an azo based polymerization initiator.

The content of the radical polymerization initiator is preferably 0.1 to 10% by mass of the total mass of the solid components, more preferably 0.1 to 5% by mass, and still more preferably 0.1 to 1% by mass.

The total content of the optional component (F) is preferably 0 to 49% by mass of the total mass of the solid components, more preferably 0 to 5% by mass, still more preferably 0 to 1% by mass, and particularly preferably 0% by mass.

In the optical member forming composition of the present embodiment, the content of the component (A), the acid generating agent (C), the acid diffusion controlling agent (E), the optional component (F) (the component (A)/the acid generating agent (C)/the acid diffusion controlling agent (E)/the optional component (F)) is preferably 50 to 99.4/0.001 to 49/0.001 to 49/0 to 49 in % by mass based on the solid content, more preferably 55 to 90/1 to 40/0.01 to 10/0 to 5, still more preferably 60 to 80/3 to 30/0.01 to 5/0 to 1, and particularly preferably 60 to 70/10 to 25/0.01 to 3/0.

The content ratio of each component is selected from each range so that the summation thereof is 100% by mass. When the content ratio of each component is within the above range, performance such as sensitivity, resolution, and developability tends to be even better.

The method for preparing the optical member forming composition of the present embodiment is not particularly limited, and, examples include a method involving dissolving each component in a solvent upon use into a homogenous solution, and then if required, filtering through a filter or the like with a pore diameter of about 0.2 m, for example.

The optical member forming composition of the present embodiment can contain other resins within the range of not inhibiting the purpose of the present invention. Examples of other resins include, but not particularly limited to, a novolac resin, polyvinyl phenols, polyacrylic acid, polyvinyl alcohol, a styrene-maleic anhydride resin, an acrylic acid, vinyl alcohol or vinylphenol as a monomeric unit, or derivative thereof. The content of other resins is not particularly limited, and is arbitrarily adjusted according to the kind of the component (A), but is preferably 30 parts by mass or less per 100 parts by mass of the component (A), more preferably 10 parts by mass or less, still more preferably 5 parts by mass or less, and particularly preferably 0 parts by mass.

A cured product can be obtained by curing the optical member forming composition of the present embodiment. The cured product can be used as various resins. Such a cured product can be used for various purposes as a highly versatile material that confers various properties such as a high melting point, high refractive index and high transparency. The cured product can be obtained by using a publicly known method appropriate for each composition of the above composition, such as light irradiation or heating.

The cured product can be used as, for example, various synthetic resins such as an epoxy resin, a polycarbonate resin, and an acrylic resin and further as an optical material such as a lens or an optical sheet, or a highly functional material such as a hologram recording material, an organic photoreceptor (e.g., a light emitting layer for phosphor elements), a photoresist material, an antireflective film, a multilayer resist material, or a semiconductor encapsulant, by exploiting functionality.

### EXAMPLES

The present embodiment will be more specifically described with reference to synthesis examples and examples below. However, the present invention is not limited to these examples by any means.

A carbon concentration and an oxygen concentration, and a molecular weight were measured as follows.

### (Carbon Concentration and Oxygen Concentration)

The carbon concentration and the oxygen concentration (% by mass) were measured by organic elemental analysis.

Apparatus: CHN Coder MT-6 (manufactured by Yaic. Yanaco)

### (Molecular Weight)

The molecular weight of the compound was measured by LC-MS analysis using Acquity UPLC/MALDI-Synapt HDMS manufactured by Waters Corp.

### (Example 1) Synthesis of BiP-1

In a container (internal capacity: 300 mL) equipped with a stirrer, a condenser tube, and a burette, 12 g (69.0 mmol) of o-phenylphenol (reagent manufactured by Sigma-Aldrich) was melted at 120° C. and then fed with 0.27 g of sulfuric acid, and 2.7 g (13.8 mmol) of 4-biphenylaldehyde (reagent manufactured by Sigma-Aldrich) was added thereto. The contents were reacted by stirring at 120° C. for 6 hours to obtain a reaction solution. Next, 100 mL of N-methyl-2-pyrrolidone (manufactured by Kanto Chemical Co., Inc.) and 50 mL of pure water were added to the reaction solution, followed by extraction with ethyl acetate. Next, the extract was separated until neutral by the addition of pure water, and then concentrated to obtain a solution.

After separation of the obtained solution by column chromatography, 5.0 g of the target compound (BiP-1) represented by the formula (BiP-1) given below was obtained.

As a result of measuring the molecular weight of the obtained compound (BiP-1) by the above method, it was 504. Also, the carbon concentration was 88.1% by mass, and the oxygen concentration was 6.3% by mass.

The following peaks were found by NMR measurement performed on the obtained compound (BiP-1) under the above measurement conditions, and the compound was confirmed to have a chemical structure of the formula (BiP-1) shown below.

δ (ppm) 9.48 (2H, O—H), 6.88-7.61 (25H, Ph-H), 5.54 (1H, C—H)

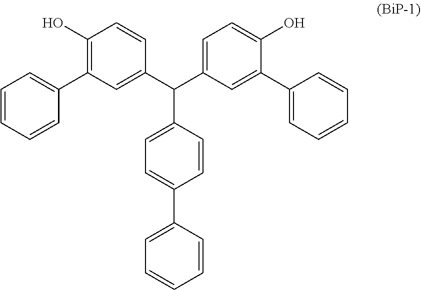 [SMILES: Oc1ccc(C(c2ccc(-c3ccccc3)cc2)c2ccc(O)c(-c3ccccc3)c2)cc1-c1ccccc1]

### (Example 2) Synthesis of BiP-2

2.0 g of the target compound (BiP-2) represented by the following formula (BiP-2) was obtained in the same way as in Example 1 except that 4,4′-biphenol was used instead of o-phenylphenol.

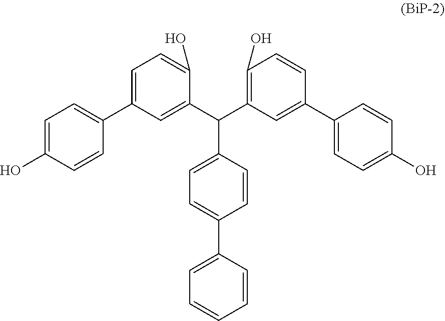 [SMILES: Oc1ccc(-c2ccc(O)c(C(c3ccc(-c4ccccc4)cc3)c3cc(-c4ccc(O)cc4)ccc3O)c2)cc1]

### (Example 3) Synthesis of XiN-1

2.0 g of the target compound (XiN-1) represented by the following formula (XiN-1) was obtained in the same way as in Example 1 except that 2,6-dihydroxynaphthalene was used instead of o-phenylphenol.

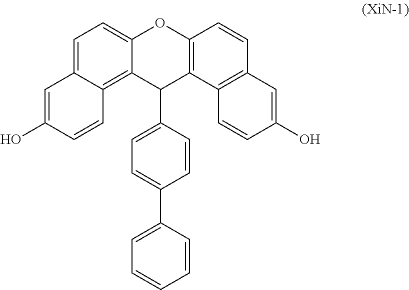 [SMILES: Oc1ccc2c3c(ccc2c1)Oc1ccc2cc(O)ccc2c1C3c1ccc(-c2ccccc2)cc1]

### (Example 4) Synthesis of XiN-3

1.5 g of the target compound (XiN-3) represented by the following formula (XiN-3) was obtained in the same way as in Example 1 except that 2,6-dihydroxynaphthalene was used instead of o-phenylphenol, and 4,4′-diformylbiphenyl was used instead of 4-biphenylaldehyde.

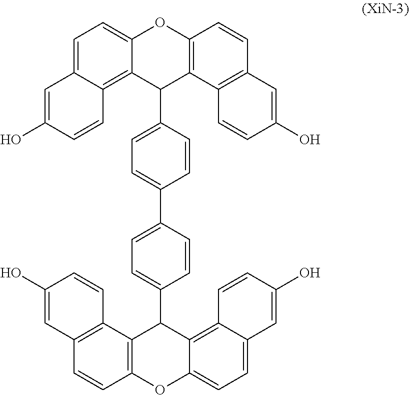 [SMILES: Oc1ccc2c3c(ccc2c1)Oc1ccc2cc(O)ccc2c1C3c1ccc(-c2ccc(C3c4c(ccc5cc(O)ccc45)Oc4ccc5cc(O)ccc5c43)cc2)cc1]

### (Example 5) Synthesis of XBiN-3

1.5 g of the target compound (XBiN-3) represented by the following formula (XBiN-3) was obtained in the same way as in Example 1 except that 2,7-dihydroxynaphthalene was used instead of o-phenylphenol, and 4,4′-diformylbiphenyl was used instead of 4-biphenylaldehyde.

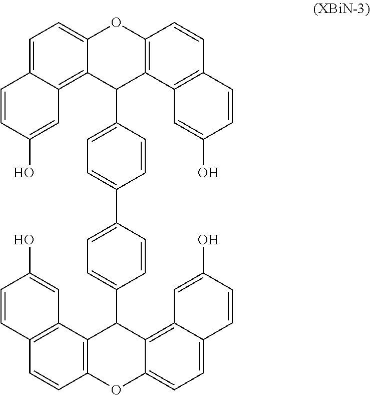 [SMILES: Oc1ccc2ccc3c(c2c1)C(c1ccc(-c2ccc(C4c5c(ccc6ccc(O)cc56)Oc5ccc6ccc(O)cc6c54)cc2)cc1)c1c(ccc2ccc(O)cc12)O3]

### Examples 6 to 13

Each target component was obtained in the same way as in Example 1 except that o-phenylphenol and 4-biphenylaldehyde used as the raw materials of Example 1 were changed as shown in Table 1.

The structure of each component was identified by 1H-NMR as shown in Table 2.

TABLE 1

| Ex- | | | |
|---|---|---|---|
| am- | | | |
| ple | Raw material 1 | Raw material 2 | Product |
| 6 | 2,6-Dihydroxynaphthalene | Isobutylbenzaldehyde | XBisN-2 |
| 7 | 2,6-Dihydroxynaphthalene | n-Propylbenzaldehyde | XBisN-3 |
| 8 | 2,6-Dihydroxynaphthalene | 4-Hydroxybenzaldehyde | XBisN-4 |
| 9 | 2,6-Dihydroxynaphthalene | 4-Cyclohexylbenzalde- | XBisN-5 |
| | | hyde | |
| 10 | 2,7-Dihydroxynaphthalene | Isobutylbenzaldehyde | XBisN-6 |
| 11 | 2,7-Dihydroxynaphthalene | n-Propylbenzaldehyde | XBisN-7 |
| 12 | 2,7-Dihydroxynaphthalene | 4-Hydroxybenzaldehyde | XBisN-8 |
| 13 | 2,7-Dihydroxynaphthalene | 4-Cyclohexylbenzalde- | XBisN-9 |
| | | hyde | |

TABLE 2

| | Compound | |
|---|---|---|
| Example | name | 1H-NMR |
| 6 | XBisN-2 | δ (ppm) 9.7(2H, O—H), 7.2~8.5(14H, |
| | | Ph—H), 6.6(1H, C—H), 2.3(6H, C—H3), |
| | | 1.4~1.9(3H, —CH2—CH) |
| 7 | XBisN-3 | δ (ppm) 9.7(2H, O—H), 7.2~8.5(14H, Ph—H), |
| | | 6.6(1H, C—H), 2.4(3H, C—H3), 1.4~1.8(3H, |
| | | —CH2—CH) |
| 8 | XBisN-4 | δ (ppm) 9.4~9.7(3H, O—H), 7.2~8.3(14H, |
| | | Ph—H), 6.6(1H, C—H) |
| 9 | XBisN-5 | δ (ppm) 9.2(2H, O—H), 6.8~7.8(14H, Ph—H), |
| | | 1.4~1.9(10H, C—H2), 2.8(1H, C—H), 2.6(1H, |
| | | C—H) |
| 10 | XBisN-6 | δ (ppm) 9.7(2H, O—H), 7.2~8.5(14H, Ph—H), |
| | | 6.6(1H, C—H), 2.3(6H, C—H3), 1.4~1.9(3H, |
| | | —CH2—CH) |
| 11 | XBisN-7 | δ (ppm) 9.7(2H, O—H), 7.2~8.5(14H, Ph—H), |
| | | 6.6(1H, C—H), 2.4(3H, C—H3), |
| | | 1.4~1.8(3H, —CH2—CH2) |
| 12 | XBisN-8 | δ (ppm) 9.4~9.7(3H, O—H), 7.2~8.3(14H, |
| | | Ph—H), 6.6(1H, C—H) |
| 13 | XBisN-9 | δ (ppm) 9.2(2H, O—H), 6.8~7.8(14H, Ph—H), |
| | | 1.4~1.9(10H, C—H2), 2.8(1H, C—H), |
| | | 12.6(1H, —C—H) |

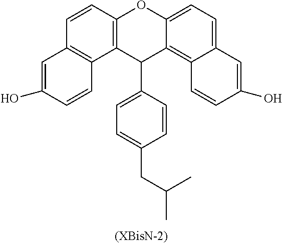 [SMILES: CC(C)Cc1ccc(C2c3c(ccc4cc(O)ccc34)Oc3ccc4cc(O)ccc4c32)cc1]

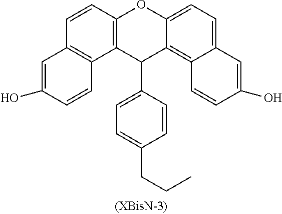 [SMILES: CCCc1ccc(C2c3c(ccc4cc(O)ccc34)Oc3ccc4cc(O)ccc4c32)cc1]

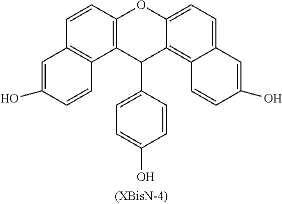 [SMILES: Oc1ccc(C2c3c(ccc4cc(O)ccc34)Oc3ccc4cc(O)ccc4c32)cc1]

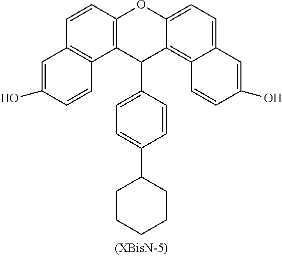 [SMILES: Oc1ccc2c3c(ccc2c1)Oc1ccc2cc(O)ccc2c1C3c1ccc(C2CCCCC2)cc1]

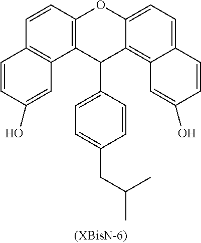 [SMILES: CC(C)Cc1ccc(C2c3c(ccc4ccc(O)cc34)Oc3ccc4ccc(O)cc4c32)cc1]

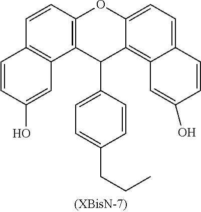 [SMILES: CCCc1ccc(C2c3c(ccc4ccc(O)cc34)Oc3ccc4ccc(O)cc4c32)cc1]

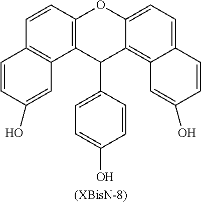 [SMILES: Oc1ccc(C2c3c(ccc4ccc(O)cc34)Oc3ccc4ccc(O)cc4c32)cc1]

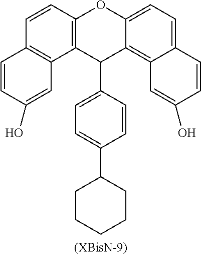 [SMILES: Oc1ccc2ccc3c(c2c1)C(c1ccc(C2CCCCC2)cc1)c1c(ccc2ccc(O)cc12)O3]

### Examples 14 to 16

Each target component was obtained in the same way as in Example 1 except that: o-phenylphenol and 4-biphenylaldehyde used as the raw materials of Example 1 were changed as shown in Table 3; 1.5 mL of water, 73 mg (0.35 mmol) of dodecylmercaptan, and 2.3 g (22 mmol) of 37% hydrochloric acid were added; and the reaction temperature was changed to 55° C.

The structure of each component was identified by ¹H-NMR as shown in Table 4.

TABLE 3

| Example | Raw material 1 | Raw material 2 | Product |
|---|---|---|---|
| 14 | Resorcinol | 4-Biphenylaldehyde | P-5 |
| 15 | Resorcinol | Benzaldehyde | P-6 |
| 16 | Resorcinol | 4-Cyclohexylbenzaldehyde | P-7 |

TABLE 4

| | Compound | |
|---|---|---|
| Example | name | 1H-NMR |
| 14 | P-5 | δ (ppm), 9.2~9.4(4H, O—H), 6.6~7.2(15H, |
| | | Ph—H), 6.3(1H, C—H) |
| 15 | P-6 | δ (ppm) 9.3~9.4(4H, 0—H), 6.6~7.2(11H, |
| | | Ph—H), 6.2(1H, C—H) |
| 16 | P-7 | δ (ppm) 9.2~9.4(4H, O—H), 6.4~7.2(10H, |
| | | Ph—H), 1.4~1.9(10H, C—H2), 2.7(1H, |
| | | C—H), 2.5(1H, C—H) |

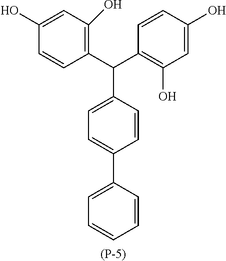

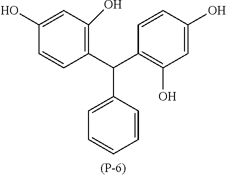

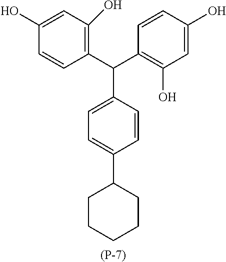

### (Example 17) Synthesis of Resin (R1-XiN-1)

A 4-neck flask (internal capacity: 1 L) equipped with a Dimroth condenser tube, a thermometer, and a stirring blade and having a detachable bottom was prepared. To this 4-neck flask, 32.6 g (70 mmol) of the compound (XiN-1) obtained in Example 3 (manufactured by Mitsubishi Gas Chemical Co., Inc.), 21.0 g (280 mmol as formaldehyde) of 40% by mass of an aqueous formalin solution (manufactured by Mitsubishi Gas Chemical Co., Inc.), and 0.97 mL of 98% by mass of sulfuric acid (manufactured by Kanto Chemical Co., Inc.) were fed in the current of nitrogen, and the mixture was reacted for 7 hours while being refluxed at 100° C. at normal pressure. Subsequently, 180.0 g of orthoxylene (a special grade reagent manufactured by Wako Pure Chemical Industries, Ltd.) was added as a diluting solvent to the reaction solution, and the mixture was left to stand still, followed by removal of an aqueous phase as a lower phase. Neutralization and washing with water were further performed, and orthoxylene was distilled off under reduced pressure to obtain 34.1 g of the resin (R1-XiN-1) as a brown solid.

The molecular weight of the obtained resin (R1-XiN-1) was Mn: 1975, Mw: 3650, Mw/Mn: 1.84.

### (Example 18) Synthesis of Resin (R2-XiN-1)

A 4-neck flask (internal capacity: 1 L) equipped with a Dimroth condenser tube, a thermometer, and a stirring blade and having a detachable bottom was prepared. To this 4-neck flask, 32.6 g (70 mmol) of the compound (XiN-1) obtained in Example 3 (manufactured by Mitsubishi Gas Chemical Co., Inc.), 50.9 g (280 mmol) of 4-biphenylaldehyde (manufactured by Mitsubishi Gas Chemical Co., Inc.), 100 mL of anisole (manufactured by Kanto Chemical Co., Inc.) and 10 mL of oxalic acid dihydrate (manufactured by Kanto Chemical Co., Inc.) were fed in the current of nitrogen, and the mixture was reacted for 7 hours while being refluxed at 100° C. at normal pressure. Subsequently, 180.0 g of orthoxylene (a special grade reagent manufactured by Wako Pure Chemical Industries, Ltd.) was added as a diluting solvent to the reaction solution, and the mixture was left to stand still, followed by removal of an aqueous phase as a lower phase. Neutralization and washing with water were further performed, and the solvent and unreacted 4-biphenylaldehyde in the organic phase were distilled off under reduced pressure to obtain 34.7 g of the resin (R2-XiN-1) as a brown solid.

The molecular weight of the obtained resin (R2-XiN-1) was Mn: 1610, Mw: 2567, Mw/Mn: 1.59.

Solubility, refractive index, transparency, and heat resistance were evaluated according to the methods given below using the obtained compounds and resins. The evaluation results are shown in Table 5.

### (Solubility)

The solubility was evaluated by precisely weighing the compound into a test tube, adding propylene glycol monomethyl ether acetate at 23° C. so as to attain a predetermined concentration, applying ultrasonic waves for 30 minutes in an ultrasonic cleaner, then visually observing the subsequent state of the fluid, determining the concentration of the amount of complete dissolution, and conducting evaluation according to the following criteria.

### [Solubility Test]

A: 5.0% by mass Amount of dissolution

B: 3.0% by mass Amount of dissolution <5.0% by mass

C: Amount of dissolution <3.0% by mass

### (Refractive Index and Transparency)

The compound was dissolved at 5% by mass in propylene glycol methyl ether to prepare an optical member forming material. Next, a silicon substrate was spin coated with the optical member forming material, and then baked at 110° C. for 60 seconds to form an optical member forming film with a film thickness of 1000 nm.

Subsequently, the refractive index and transparency tests at a wavelength of 633 nm were conducted using a vacuum ultraviolet variable angle spectroscopic ellipsometer (VUV-VASE) manufactured by J. A. Woollam Co., Inc. The refractive index and the transparency were evaluated according to the following criteria.

### [Evaluation Criteria for Refractive Index]

A: The refractive index was 1.65 or more.

C: The refractive index was less than 1.65.

### [Evaluation Criteria for Transparency]

A: The absorption constant was less than 0.03.

B: The absorption constant was 0.03 or more and less than 0.05.

C: The absorption constant was 0.05 or more.

### (Film Heat Resistance)

The optical member forming film obtained as described above was baked at 110° C. for 5 minutes, and evaluation of film heat resistance was carried out according to the following criteria.

### [Evaluation Criteria for Film Heat Resistance]

A: No defect in the film was visually observed.

C: Defects in the film were visually observed (including disappearance of the film).

TABLE 5

| | | Solu- | Refractive | Absorption | Film heat |
|---|---|---|---|---|---|
| | Compound | bility | index | constant | resistance |
| Example 1 | BiP-1 | A | A | A | A |
| Example 2 | BiP-2 | A | A | A | A |
| Example 3 | XiN-1 | A | A | A | A |
| Example 4 | XiN-3 | A | A | A | A |
| Example 5 | XBiN 3 | A | A | A | A |
| Example 6 | XBisN 2 | A | A | A | A |
| Example 7 | XBisN-3 | A | A | A | A |
| Example 8 | XBisN-4 | A | A | A | A |
| Example 9 | XBisN-5 | A | A | A | A |
| Example 10 | XBisN-6 | A | A | A | A |
| Example 11 | XBisN-7 | A | A | A | A |
| Example 12 | XBisN-8 | A | A | A | A |
| Example 13 | XBisN-9 | A | A | A | A |
| Example 14 | P-5 | A | A | A | A |
| Example 15 | P-6 | A | A | A | A |
| Example 16 | P-7 | A | A | A | A |
| Example 17 | R1-XiN-1 | A | A | A | A |
| Example 18 | R2-XiN-1 | A | A | A | A |

As is evident from Table 5, the optical member forming composition containing the compound according to the present embodiment has high refractive index. Also, the optical member forming composition has high heat resistance attributed to its rigidity of the structure despite a relatively low molecular weight and can therefore be used even under high temperature baking conditions. Furthermore, the optical member forming composition has high solubility in a safe solvent, exhibits suppressed crystallinity, has good heat resistance. The optical member forming composition of the present embodiment imparts a good shape to an optical member.

### INDUSTRIAL APPLICABILITY

The present invention is used in, for example, electrical insulating materials, resins for resists, encapsulation resins for semiconductors, adhesives for printed circuit boards, electrical laminates mounted in electric equipment, electronic equipment, industrial equipment, and the like, matrix resins of prepregs mounted in electric equipment, electronic equipment, industrial equipment, and the like, buildup laminate materials, resins for fiber-reinforced plastics, resins for encapsulation of liquid crystal display panels, coating materials, various coating agents, adhesives, coating agents for semiconductors, resins for resists for semiconductors, and resins for underlayer film formation required to have optical properties such as high refractive index or high transparency, or in a film form or a sheet form, and furthermore, is applicable widely and effectively to optical components such as plastic lenses (prism lens, lenticular lens, microlens, Fresnel lens, viewing angle control lens, contrast improving lens, etc.), phase difference films, films for electromagnetic wave shielding, prisms, optical fibers, solder resists for flexible printed wiring, plating resists, interlayer insulating films for multilayer printed circuit boards, and photosensitive optical waveguides.

1. An optical member forming composition comprising a compound represented by the following formula (0): 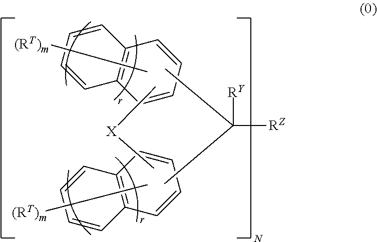
wherein
R^{Y} is a hydrogen atom;
R^{Z} is an N-valent group of 1 to 60 carbon atoms or a single bond;
each R^{T} is independently an alkyl group of 1 to 30 carbon atoms optionally having a substituent, an aryl group of 6 to 40 carbon atoms optionally having a substituent, an alkenyl group of 2 to 30 carbon atoms optionally having a substituent, an alkoxy group of 1 to 30 carbon atoms optionally having a substituent, a halogen atom, a nitro group, an amino group, a thiol group, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond;
X is an oxygen atom, a sulfur atom, a single bond or non-crosslinked state;
each m is independently an integer of 0 to 9, wherein at least one of m is an integer of 1 to 9;
N is an integer of 1 to 4, wherein when N is an integer of 2 or larger, structural formulae indicated within N parentheses are the same or different; and
each r is independently an integer of 0 to 2.

2. The optical member forming composition according to claim 1, wherein the compound represented by the above formula (0) is a compound represented by the following formula (0-1): 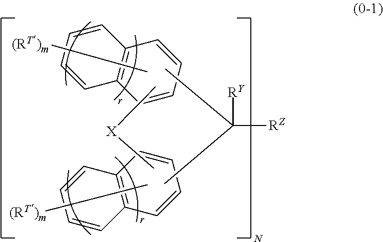
wherein
R^{Y} is a hydrogen atom;
R^{Z} is an N-valent group of 1 to 60 carbon atoms or a single bond;
each R^{T′} is independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a thiol group, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond, and at least one of R^{T′} is a hydroxy group or a group in which a hydrogen atom of a hydroxy group is substituted with an acid crosslinking group or an acid dissociation group;
X is an oxygen atom, a sulfur atom, a single bond or non-crosslinked state;
each m is independently an integer of 0 to 9, wherein at least one of m is an integer of 1 to 9;
N is an integer of 1 to 4, wherein when N is an integer of 2 or larger, structural formulae indicated within N parentheses are the same or different; and
each r is independently an integer of 0 to 2.

3. The optical member forming composition according to claim 2, wherein the compound represented by the above formula (0-1) is a compound represented by the following formula (1): 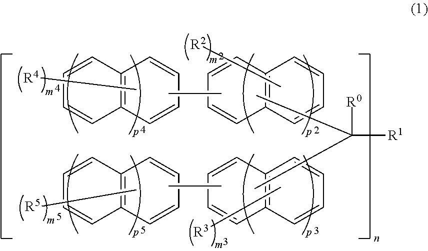
wherein
R⁰ is as defined in the R^{Y};
R¹ is an n-valent group of 1 to 60 carbon atoms or a single bond;
R² to R⁵ are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a thiol group, a hydroxy group or a group in which a hydrogen atom of a hydroxy group is substituted with an acid dissociation group, wherein the alkyl group, the alkenyl group and the aryl group each optionally have an ether bond, a ketone bond or an ester bond, and at least one of R² to R⁵ is a hydroxy group or a group in which a hydrogen atom of a hydroxy group is substituted with an acid dissociation group;
m² and m³ are each independently an integer of 0 to 8;
m⁴ and m⁵ are each independently an integer of 0 to 9,
provided that m², m³, m⁴ and m⁵ are not 0 at the same time;
n is as defined in the N, wherein when n is an integer of 2 or larger, structural formulae indicated within n parentheses are the same or different; and
p² to p⁵ are each as defined in the r.

4. The optical member forming composition according to claim 2, wherein the compound represented by the above formula (0-1) is a compound represented by the following formula (2): 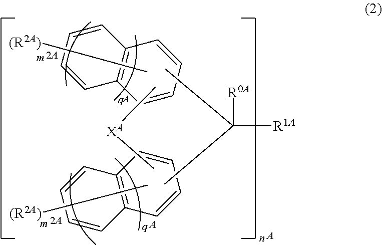
wherein
R^{0A} is as defined in the R^{Y};
R^{1A} is an n^{A}-valent group of 1 to 60 carbon atoms or a single bond;
each R^{2A} is independently an alkyl group of 1 to 30 carbon atoms optionally having a substituent, an aryl group of 6 to 30 carbon atoms optionally having a substituent, an alkenyl group of 2 to 10 carbon atoms optionally having a substituent, a halogen atom, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond, and at least one of R^{2A} is a hydroxy group or a group in which a hydrogen atom of a hydroxy group is substituted with an acid crosslinking group or an acid dissociation group;
n^{A} is as defined in the N, wherein when n^{A} is an integer of 2 or larger, structural formulae indicated within n^{A} parentheses are the same or different;
X^{A} is an oxygen atom, a sulfur atom, a single bond or non-crosslinked state;
each m^{2A} is independently an integer of 0 to 7, provided that at least one of m^{2A} is an integer of 1 to 7; and
each q^{A} is independently 0 or 1.

5. The optical member forming composition according to claim 3, wherein the compound represented by the above formula (1) is a compound represented by the following formula (1-1): 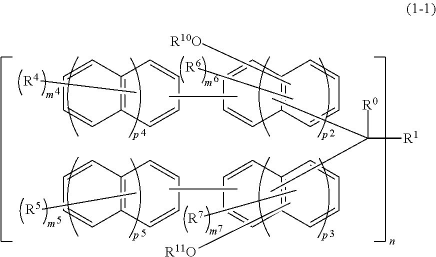
wherein
R⁰, R¹, R⁴, R⁵, n, p² to p⁵, m⁴ and m⁵ are as defined above;
R⁶ and R⁷ are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, or a thiol group, wherein the alkyl group, the alkenyl group and the aryl group each optionally have an ether bond, a ketone bond or an ester bond;
R¹⁰ and R¹¹ are each independently a hydrogen atom, an acid crosslinking group or an acid dissociation group; and
m⁶ and m⁷ are each independently an integer of 0 to 7,
provided that m⁴, m⁵, m⁶ and m⁷ are not 0 at the same time.

6. The optical member forming composition according to claim 5, wherein the compound represented by the above formula (1-1) is a compound represented by the following formula (1-2): 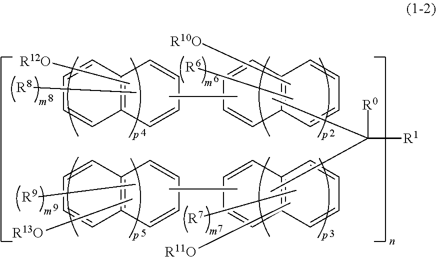
wherein
R⁰, R¹, R⁶, R⁷, R¹⁰, R¹¹, n, p² to p⁵, m⁶ and m⁷ are as defined above;
R⁸ and R⁹ are as defined in the R⁶ and the R⁷;
R¹² and R¹³ are as defined in the R¹⁰ and the R¹¹; and
m⁸ and m⁹ are each independently an integer of 0 to 8,
provided that m⁶, m⁷, m⁸ and m⁹ are not 0 at the same time.

7. The optical member forming composition according to claim 4, wherein the compound represented by the above formula (2) is a compound represented by the following formula (2-1): 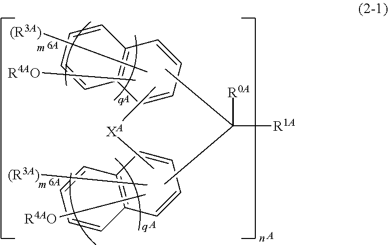
wherein
R^{0A}, R^{1A}, n^{A}, q^{A} and X^{A} are as defined above;
each R^{3A} is independently a halogen atom, an alkyl group of 1 to 30 carbon atoms optionally having a substituent, an aryl group of 6 to 30 carbon atoms optionally having a substituent, or an alkenyl group of 2 to 30 carbon atoms optionally having a substituent, wherein the alkyl group, the alkenyl group and the aryl group each optionally have an ether bond, a ketone bond or an ester bond;
each R^{4A} is independently a hydrogen atom, an acid crosslinking group or an acid dissociation group; and
each m^{6A} is independently an integer of 0 to 5.

8. An optical member forming composition comprising a resin obtained with a compound represented by the following formula (0) as a monomer: 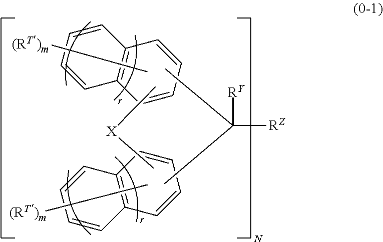
wherein
R^{Y} is a hydrogen atom;
R^{Z} is an N-valent group of 1 to 60 carbon atoms or a single bond;
each R^{T} is independently an alkyl group of 1 to 30 carbon atoms optionally having a substituent, an aryl group of 6 to 40 carbon atoms optionally having a substituent, an alkenyl group of 2 to 30 carbon atoms optionally having a substituent, an alkoxy group of 1 to 30 carbon atoms optionally having a substituent, a halogen atom, a nitro group, an amino group, a thiol group, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond;
X is an oxygen atom, a sulfur atom, a single bond or non-crosslinked state;
each m is independently an integer of 0 to 9, wherein at least one of m is an integer of 1 to 9;
N is an integer of 1 to 4, wherein when N is an integer of 2 or larger, structural formulae indicated within N parentheses are the same or different; and
each r is independently an integer of 0 to 2.

9. An optical member forming composition comprising a resin obtained with a compound represented by the following formula (1) as a monomer: 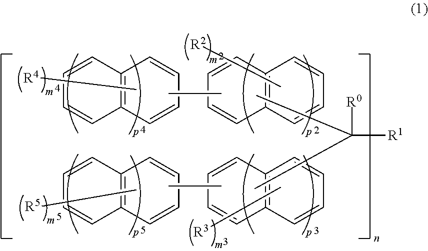
wherein
R⁰ is a hydrogen atom;
R¹ is an n-valent group of 1 to 60 carbon atoms or a single bond;
R² to R⁵ are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a thiol group, a hydroxy group or a group in which a hydrogen atom of a hydroxy group is substituted with an acid dissociation group, wherein the alkyl group, the alkenyl group and the aryl group each optionally have an ether bond, a ketone bond or an ester bond, and at least one of R² to R⁵ is a hydroxy group or a group in which a hydrogen atom of a hydroxy group is substituted with an acid dissociation group;
m² and m³ are each independently an integer of 0 to 8;
m⁴ and m⁵ are each independently an integer of 0 to 9,
provided that m², m³, m⁴ and m⁵ are not 0 at the same time;
n is an integer of 1 to 4, wherein when n is an integer of 2 or larger, structural formulae indicated within n parentheses are the same or different; and
p² to p⁵ are each independently an integer of 0 to 2.

10. The optical member forming composition according to claim 9, wherein the resin obtained with the compound represented by the above formula (1) as a monomer is a resin having a structure represented by the following formula (3): 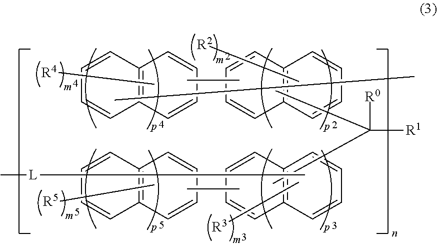
wherein
L is a linear or branched alkylene group of 1 to 30 carbon atoms optionally having a substituent or a single bond;
R⁰ is a hydrogen atom;
R¹ is an n-valent group of 1 to 60 carbon atoms or a single bond;
R² to R⁵ are each independently an alkyl group of 1 to 30 carbon atoms optionally having a substituent, an aryl group of 6 to 30 carbon atoms optionally having a substituent, an alkenyl group of 2 to 30 carbon atoms optionally having a substituent, an alkoxy group of 1 to 30 carbon atoms optionally having a substituent, a halogen atom, a thiol group, a hydroxy group or a group in which a hydrogen atom of a hydroxy group is substituted with an acid dissociation group, wherein the alkyl group, the alkenyl group and the aryl group each optionally have an ether bond, a ketone bond or an ester bond, and at least one of R² to R⁵ is a hydroxy group or a group in which a hydrogen atom of a hydroxy group is substituted with an acid dissociation group;
m² and m³ are each independently an integer of 0 to 8;
m⁴ and m^{s5} are each independently an integer of 0 to 9,
provided that m², m³, m⁴ and m⁵ are not 0 at the same time;
n is an integer of 1 to 4, wherein when n is an integer of 2 or larger, structural formulae indicated within n parentheses are the same or different; and
p² to p⁵ are each independently an integer of 0 to 2.

11. An optical member forming composition comprising a resin obtained with a compound represented by the following formula (2) as a monomer: 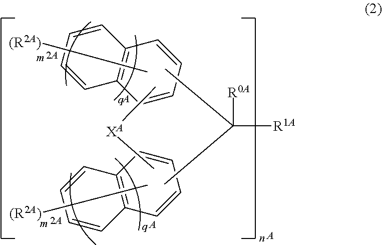
wherein
R^{0A} is a hydrogen atom;
R^{1A} is an n^{A}-valent group of 1 to 60 carbon atoms or a single bond;
each R^{2A} is independently an alkyl group of 1 to 30 carbon atoms optionally having a substituent, an aryl group of 6 to 30 carbon atoms optionally having a substituent, an alkenyl group of 2 to 10 carbon atoms optionally having a substituent, a halogen atom, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond, and at least one of R^{2A} is a hydroxy group or a group in which a hydrogen atom of a hydroxy group is substituted with an acid crosslinking group or an acid dissociation group;
n^{A} is an integer of 1 to 4, wherein when n^{A} is an integer of 2 or larger, structural formulae indicated within n^{A} parentheses are the same or different;
X^{A} is an oxygen atom, a sulfur atom, a single bond or non-crosslinked state;
each m^{2A} is independently an integer of 0 to 7, provided that at least one of m^{2A} is an integer of 1 to 7; and
each q^{A} is independently 0 or 1.

12. The optical member forming composition according to claim 11, wherein the resin obtained with the compound represented by the above formula (2) as a monomer is a resin having a structure represented by the following formula (4): 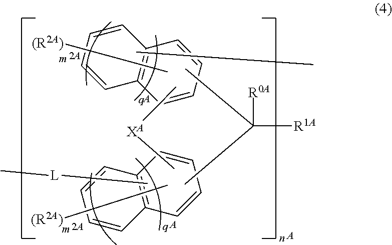
wherein
L is a linear or branched alkylene group of 1 to 30 carbon atoms optionally having a substituent or a single bond;
R^{0A} is a hydrogen atom;
R^{1A} is an n^{A}-valent group of 1 to 30 carbon atoms or a single bond;
each R^{2A} is independently an alkyl group of 1 to 30 carbon atoms optionally having a substituent, an aryl group of 6 to 30 carbon atoms optionally having a substituent, an alkenyl group of 2 to 10 carbon atoms optionally having a substituent, a halogen atom, a hydroxy group or a group in which a hydrogen atom of a hydroxy group is substituted with an acid crosslinking group or an acid dissociation group, wherein the alkyl group, the alkenyl group and the aryl group each optionally have an ether bond, a ketone bond or an ester bond, and at least one of R^{2A} is a hydroxy group or a group in which a hydrogen atom of a hydroxy group is substituted with an acid crosslinking group or an acid dissociation group;
n^{A} is an integer of 1 to 4, wherein when n^{A} is an integer of 2 or larger, structural formulae indicated within n^{A} parentheses are the same or different;
X^{A} is an oxygen atom, a sulfur atom, a single bond or non-crosslinked state;
each m^{2A} is independently an integer of 0 to 6, provided that at least one of m^{2A} is an integer of 1 to 6; and
each q^{A} is independently 0 or 1.

13. The optical member forming composition according to claim 1, further comprising a solvent.

14. The optical member forming composition according to claim 1, further comprising an acid generating agent.

15. The optical member forming composition according to claim 13, further comprising a crosslinking agent.

16. The optical member forming composition according to claim 15, wherein the crosslinking agent is at least one selected from the group consisting of a phenol compound, an epoxy compound, a cyanate compound, an amino compound, a benzoxazine compound, a melamine compound, a guanamine compound, a glycoluril compound, a urea compound, an isocyanate compound and an azide compound.

17. The optical member forming composition according to claim 15, wherein the crosslinking agent has at least one allyl group.

18. The optical member forming composition according to claim 15, wherein a content of the crosslinking agent is 0.1 to 50% by mass of the total mass of the solid components.

19. The optical member forming composition according to claim 15, further comprising a crosslinking promoting agent.

20. The optical member forming composition according to claim 19, wherein the crosslinking promoting agent is at least one selected from the group consisting of an amine, an imidazole, an organic phosphine, and a Lewis acid.

21. The optical member forming composition according to claim 19, wherein a content of the crosslinking promoting agent is 0.1 to 10% by mass of the total mass of the solid components.

22. The optical member forming composition according to claim 13, further comprising a radical polymerization initiator.

23. The optical member forming composition according to claim 22, wherein the radical polymerization initiator is at least one selected from the group consisting of a ketone based photopolymerization initiator, an organic peroxide based polymerization initiator and an azo based polymerization initiator.

24. The optical member forming composition according to claim 22, wherein a content of the radical polymerization initiator is 0.1 to 10% by mass of the total mass of the solid components.
